(19) United States
(12) Patent Application Publication
(10) Pub. No.: US 2011/0250663 A1
(43) Pub. Date: Oct. 13, 2011

(54) METHODS AND COMPOSITIONS RELATED TO FATTY ALCOHOL BIOSYNTHETIC ENZYMES

(75) Inventors: Andreas W. Schirmer, South San Francisco, CA (US); Na M. Trinh, South San Francisco, CA (US)

(73) Assignee: LS9, INC., South San Francisco, CA (US)

(21) Appl. No.: 13/083,066

(22) Filed: Apr. 8, 2011

Related U.S. Application Data
(60) Provisional application No. 61/321,877, filed on Apr. 8, 2010.
(60) Provisional application No. 61/321,878, filed on Apr. 8, 2010.

(51) Int. Cl. C12P 7/04; C07C 11/00; C12N 1/00; C12P 5/00; C12P 5/02

(52) U.S. Cl. 435/157; 435/166; 435/167; 435/243; 585/16

(57) ABSTRACT

Compositions and methods for producing fatty acid derivatives using recombinant microorganisms are described herein.

### CROSS-REFERENCE TO RELATED APPLICATIONS

This application claims priority benefit to U.S. Provisional Application Ser. Nos. 61/321,877, and 61/321,878, filed on Apr. 8, 2010, which are expressly incorporated by reference herein in their entirety.

### FIELD OF THE INVENTION

Compositions, methods and systems effective to produce fatty acid derivatives.

### BACKGROUND OF THE TECHNOLOGY

Petroleum is a limited, natural resource found in the Earth in liquid, gaseous, or solid forms. Petroleum is a valuable resource for producing various industrial materials. But petroleum products are developed at considerable costs, both financial and environmental. In addition to the economic cost, petroleum exploration carries a high environmental cost. In its natural form, crude petroleum extracted from the Earth has few commercial uses. It is a mixture of hydrocarbons (e.g., paraffins (or alkanes), olefins (or alkenes), alkynes, napthenes (or cycloalkanes), aliphatic compounds, aromatic compounds, etc.) of varying length and complexity. Hence, crude petroleum must be refined and purified before it can be used commercially. Crude petroleum is also a primary source of raw materials for producing petrochemicals. The two main classes of raw materials derived from petroleum are short chain olefins (e.g., ethylene and propylene) and aromatics (e.g., benzene and xylene isomers). These raw materials are derived from longer chain hydrocarbons in crude petroleum by cracking it at considerable expense using a variety of methods, such as catalytic cracking, steam cracking, or catalytic reforming. These raw materials are used to make petrochemicals, which cannot be directly refined from crude petroleum, such as monomers, solvents, detergents, or adhesives.

These petrochemicals can then be used to make specialty chemicals, such as plastics, resins, fibers, elastomers, pharmaceuticals, lubricants, or gels. Particular specialty chemicals that can be produced from petrochemical raw materials are fatty acids, hydrocarbons (e.g., long chain, branched chain, saturated, unsaturated, etc.), fatty alcohols, esters, fatty aldehydes, ketones, lubricants, etc.

Fatty alcohols have many commercial uses. The shorter chain fatty alcohols are used in the cosmetic and food industries as emulsifiers, emollients, and thickeners. Due to their amphiphilic nature, fatty alcohols behave as nonionic surfactants, which are useful in personal care and household products, for example, detergents. In addition, fatty alcohols are used in waxes, gums, resins, pharmaceutical salves and lotions, lubricating oil additives, textile antistatic and finishing agents, plasticizers, cosmetics, industrial solvents, and solvents for fats.

Hydrocarbons have many commercial uses. For example, shorter chain alkanes are used as fuels. Longer chain alkanes (e.g., from five to sixteen carbons) are used as transportation fuels (e.g., gasoline, diesel, or aviation fuel). Alkanes having more than sixteen carbon atoms are important components of fuel oils and lubricating oils. Even longer alkanes, which are solid at room temperature, can be used, for example, as a paraffin wax. In addition, longer chain alkanes can be cracked to produce commercially valuable shorter chain hydrocarbons.

Like short chain alkanes, short chain alkenes are used in transportation fuels. Longer chain alkenes are used in plastics, lubricants, and synthetic lubricants. In addition, alkenes are used as a feedstock to produce alcohols, esters, plasticizers, surfactants, tertiary amines, enhanced oil recovery agents, fatty acids, thiols, alkenylsuccinic anhydrides, epoxides, chlorinated alkanes, chlorinated alkenes, waxes, fuel additives, and drag flow reducers.

Esters have many commercial uses. For example, biodiesel, an alternative fuel, is comprised of esters (e.g., fatty acid methyl esters, fatty acid ethyl esters, etc). Some low molecular weight esters are volatile with a pleasant odor, which makes them useful as fragrances or flavoring agents. In addition, esters are used as solvents for lacquers, paints, and varnishes. Furthermore, some naturally occurring substances, such as waxes, fats, and oils are comprised of esters. Esters are also used as softening agents in resins and plasticizers, flame retardants, and additives in gasoline and oil. In addition, esters can be used in the manufacture of polymers, films, textiles, dyes, and pharmaceuticals.

Aldehydes are used to produce many specialty chemicals. For example, aldehydes are used to produce polymers, resins (e.g., Bakelite), dyes, flavorings, plasticizers, perfumes, pharmaceuticals, and other chemicals. Some are used as solvents, preservatives, or disinfectants. Some natural and synthetic compounds, such as vitamins and hormones, are aldehydes.

Obtaining specialty chemicals from crude petroleum requires a significant financial investment as well as a great deal of energy. It is also an inefficient process because frequently the long chain hydrocarbons in crude petroleum are cracked to produce smaller monomers. These monomers are then used as the raw material to manufacture the more complex specialty chemicals.

Finally, the burning of petroleum based fuels releases greenhouse gases (e.g., carbon dioxide) and other forms of air pollution (e.g., carbon monoxide, sulfur dioxide, etc.). As the world's demand for fuel increases, the emission of greenhouse gases and other forms of air pollution also increases. The accumulation of greenhouse gases in the atmosphere can lead to an increase global warming. Hence, in addition to damaging the environment locally (e.g., oil spills, dredging of marine environments, etc.), burning petroleum also damages the environment globally.

Due to the inherent challenges posed by petroleum, there is a need for a renewable petroleum source. For similar reasons, there is also a need for a renewable source of chemicals which are typically derived from petroleum. The current invention addresses these needs.

### BRIEF SUMMARY OF THE INVENTION

The invention provides recombinant microorganisms engineered to produce fatty acid derivatives and methods of use wherein the recombinant microorganisms comprise polynucleotide sequences encoding: (a) a fatty aldehyde biosynthetic polypeptide and (b) a fatty alcohol biosynthetic polypeptide, wherein the expression of the polypeptides is modified relative to the corresponding wild type polypeptides and the microorganism produces an increased titer of the fatty acid derivative relative to a wild type microorganism. The recombinant microorganisms may further comprise a thioesterase (EC 3.1.1.5).

Exemplary fatty aldehyde biosynthetic polypeptides: (a) have at least 90% sequence identity to the amino acid sequence presented as SEQ ID NO: 41, 43, 45, 47, 49, 51, 53, 55, 57, 59, 61, 63, 65, 69, 71, 73, 75, 77, 79, 81, 83, 85, 87, 89, 91, 93, 97, 99, 101, 103, 105, 107, 109, 111, 113, 115, 117, 119, 121, 123, 125, or 127; (b) comprise an amino acid sequence motif with a sequence presented as (1) SEQ ID NO:129, SEQ ID NO:130, SEQ ID NO:131, and SEQ ID NO:132; (2) SEQ ID NO:133; SEQ ID NO:134; SEQ ID NO:135; SEQ ID NO: 136; or (3) SEQ ID NO:129, SEQ ID NO:131, SEQ ID NO:132 or SEQ ID NO:133; or (c) are encoded by a polynucleotide having at least 90% sequence identity to the nucleotide sequence presented as SEQ ID NO: 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, or 128.

Methods for producing a fatty alcohol, comprising culturing such an engineered microorganism in the presence of a carbon source, under conditions wherein the fatty alcohol is produced at a titer of at least 300 mg/L, are further provided.

In practicing the claimed methods, the engineered microorganism may be modified: (a) to express an attenuated level of an acyl-CoA synthase (EC 2.3.1.86) or (b) to further comprise an acyl-ACP reductase polypeptide, wherein (i) the acyl-ACP reductase polypeptide has amino acid sequence with at least 90% sequence identity to SEQ ID NO: 137, 139, 141, 143, 145, 147, 149, 151, or 153, (ii) the acyl-ACP reductase polypeptide has an amino acid motif presented as SEQ ID NO:155, 156, 157, 158, 159, 160, 161, 162, 163, 164, or 165, or (iii) the acyl-ACP reductase polypeptide is encoded by a polynucleotide having at least 90% sequence identity to SEQ ID NO: 138, 140, 142, 144, 146, 148, 150, 152, or 154.

In practicing the claimed methods, expression of a fatty alcohol biosynthetic polypeptide (e.g., fatty aldehyde reductase or alcohol dehydrogenase (EC 1.1.1.1)) in the engineered microorganism may be increased or attenuated relative to the corresponding wild type polypeptide, or the gene encoding the fatty alcohol biosynthetic polypeptide may be knocked-out.

The fatty alcohol biosynthetic polypeptide may (a) have at least 90% sequence identity to a polypeptide sequence selected from the group consisting of SEQ ID NO:1, 3, 5, 7, 9, 11, 13, 15, 17, 19, 21, 23, 25, 27, 29, 31, 33, 35, 37, and 39, or (b) be encoded by a polynucleotide having at least 90% sequence identity to the nucleotide sequence of SEQ ID NO: 2, 4, 6, 8, 10, 12, 14, 16, 18, 20, 22, 24, 26, 28, 30, 32, 34, 36, 38, or 40.

The fatty alcohol produced by the claimed method may (a) comprise a C₆-C₁₈ fatty alcohol (e.g., a C₆, C₈, C₁₀, C₁₂, C₁₃, C₁₄, C₁₅, C₁₆, C₁₇, or C₁₈ fatty alcohol); (b) have the hydroxyl group is in the primary (C₁) position; (c) be a saturated or unsaturated fatty alcohol; (d) be unsaturated at the omega-7 position; or (e) comprise a cis double bond.

The invention further provides recombinant microorganisms engineered to produce hydrocarbons and methods of use wherein the recombinant microorganism further comprises (a) a hydrocarbon biosynthetic polypeptide having the amino acid sequence of SEQ ID NO:166, 168, 170, 172, 174, 176, 178, 180, 182, 184, 186, 188, 190, 192, 194, 196, 198, or 200 with one or more amino acid substitutions, additions, deletions, or insertions; (b) a polynucleotide sequence encoding a hydrocarbon biosynthetic polypeptide, having at least 90% sequence identity to the amino acid sequence of SEQ ID NO:166, 168, 170, 172, 174, 176, 178, 180, 182, 184, 186, 188, 190, 192, 194, 196, 198, or 200, or (c) a hydrocarbon biosynthetic polypeptide having the amino acid motif sequences presented as (1) SEQ ID NO:202; (2) SEQ ID NO:203 or SEQ ID NO:204, or SEQ ID NO:205; (3) SEQ ID NO:206, and any one of SEQ ID NO:203, SEQ ID NO:204, SEQ ID NO:205; or (4) SEQ ID NO:207 or SEQ ID NO:208, or SEQ ID NO:209, or SEQ ID NO:210, wherein the hydrocarbon biosynthetic polypeptide has decarbonylase activity.

Methods for producing a hydrocarbon, comprising culturing such engineered microorganisms in the presence of a carbon source, under conditions wherein the hydrocarbon is produced, are further provided.

The hydrocarbon produced by the claimed methods may (a) be an alkane or an alkene, e.g., a C₁₃-C₂₁ alkane or alkene, (b) have a δ¹³C of −15.4 or greater, or (c) have a f_{M}¹⁴C of at least 1.003.

The hydrocarbon produced by the claimed methods may be used in a biofuel, for example, a diesel, gasoline, or jet fuel.

The invention further provides the use of microorganisms such as a yeast cell, a fungus cell, a filamentous fungi cell, or a bacterial cell in practicing the claimed methods.

### BRIEF DESCRIPTION OF THE FIGURES

FIG. 1A is a graphic representation of pathways for fatty alcohol production. FIG. 1B is a graphic representation of pathways for hydrocarbon production.

FIG. 2 includes a table listing exemplary homologs of E. coli K-12 MG 1655 ethanol-active dehydrogenase/acetaldehyde-active reductase AdhP [GenBank Accession No. NP_{—}415995.4].

FIG. 3 includes a table listing exemplary homologs of E. coli K-12 MG 1655 2,5-diketo-D-gluconate reductase A, DkgA [GenBank Accession No. NP_{—}417485.4].

FIG. 4 includes a table listing exemplary homologs of E. coli K-12 MG 1655 2,5-diketo-D-gluconate reductase B, DkgB [GenBank Accession No. NP_{—}414743.1].

FIG. 5 includes a table listing exemplary homologs of E. coli K-12 MG 1655 E. coli K-12 MG 1655 aldo-keto reductase Tas [GenBank Accession No. NP_{—}417311.1].

FIG. 6 includes a table listing exemplary homologs of E. coli K-12 MG 1655 Zn-dependent and NAD(P)-binding oxidoreductase RspB [GenBank Accession No. NP_{—}416097.1].

FIG. 7 includes a table listing exemplary homologs of E. coli K-12 MG 1655 Zn-dependent and NAD(P)-binding oxidoreductase YahK [GenBank Accession No. NP_{—}414859.1].

FIG. 8 includes a table listing exemplary homologs of E. coli K-12 MG 1655 NAD(P)-binding oxidoreductase YbbO [GenBank Accession No. NP_{—}415026.1].

FIG. 9 includes a table listing exemplary homologs of E. coli K-12 MG 1655 oxidoreductase YbdH [GenBank Accession No. NP_{—}415132.1].

FIG. 10 includes a table listing exemplary homologs of E. coli K-12 MG 1655 Zn-dependent and NAD(P)-binding oxidoreductase YbdR [GenBank Accession No. NP_{—}4155141.1].

FIG. 11 includes a table listing exemplary homologs of E. coli K-12 MG 1655 NAD(P)-binding oxidoreductase YgfF [GenBank Accession No. NP_{—}417378.1].

FIG. 12 includes a table listing exemplary homologs of E. coli K-12 MG 1655 Zn-dependent and NAD(P)-binding oxidoreductase YhdH [Genbank Accession No. NP_{—}417719.1].

FIG. 13 includes a table listing exemplary homologs of E. coli K-12 MG 1655 Zn-dependent and NAD(P)-binding alcohol dehydrogenase YjgB [GenBank Accession No. NP_{—}418690.4].

FIG. 14 includes a table listing exemplary homologs of E. coli K-12 MG 1655 3-dehroquinate synthase AroB [GenBank Accession No. NP_{—}417848.1].

FIG. 15 includes a table listing exemplary homologs of E. coli K-12 MG 1655 Zn-dependent and NAD(P)-binding oxidoreductase YcjQ [GenBank Accession No. NP_{—}415829.1].

FIG. 16 includes a table listing exemplary homologs of E. coli K-12 MG 1655 NAD(P)-binding oxidoreductase YdbC [GenBank Accession No. NP_{—}415924.1].

FIG. 17 includes a table listing exemplary homologs of E. coli K-12 MG 1655 NADH-dependent alpha-keto reductase YdjG [GenBank Accession No. NP_{—}416285.1].

FIG. 18 includes a table listing exemplary homologs of E. coli K-12 MG 1655 NADPH-dependent aldo-keto reductase YeaE [GenBank Accession No. NP_{—}416295.1].

FIG. 19 includes a table listing exemplary homologs of E. coli K-12 MG 1655 NADP-dependent, Zn-dependent oxidoreductase YncB [GenBank Accession No. NP_{—}415966.6].

FIG. 20 includes a table listing exemplary homologs of E. coli K-12 MG 1655 NAD(P)-dependent alcohol dehydrogenase YqhD [GenBank Accession No. NP_{—}417484.1].

FIG. 21 includes a table listing exemplary homologs of E. coli K-12 MG 1655 Zn-dependent and NAD(P)-binding oxidoreductase YdjL [GenBank Accession No. NP_{—}416290.1].

FIG. 22 includes tables listing E. coli dehydratase/isomerase enzymes and dehydratase/isomerase enzymes from other organisms.

FIG. 23 includes table listing E. coli keto-ACP synthase enzymes and keto-ACP synthase enzymes from other organisms.

FIG. 24A is a graphic representation of fatty alcohols produced by a recombinant E. coli strain transformed with pETDuet-1-'tesA-alrAadp1 and pACYCDuet-1-CarB. FIG. 24B is a GC/MS trace of fatty alcohol produced by a recombinant E. coli strain transformed with pETDuet-1-'tesA-alrAadp1 and pACYCDuet-1-CarB as compared to the control strain, which did not express an alrAadp1.

FIG. 25 is a graphic representation of fatty alcohols produced by a recombinant E. coli strain transformed with pETDuet-1-'tesA-yjgB and pACYCDuet-1-CarB.

FIG. 26A is a GC/MS trace of fatty alcohol production in MG1655 (DE3, ΔfadD)/pETDuet-1-tesA and pACYCDuet-1-CarB cells. FIG. 26B is a GC/MS trace of fatty alcohol production in MG1655 (DE3, ΔfadD, yjgB::kan)/pETDuet-1-tesA and pACYCDuet-1-CarB cells. FIG. 26C is a GC/MS trace of fatty alcohol production in MG1655 (DE3, ΔfadD, yjgB::kan)/pETDuet-1-'tesA-yjgB and pACYCDuet-1-CarB cells. The arrows in FIGS. 26A, 26B, and 26C indicate the absence of C12:0 fatty aldehydes.

FIG. 27 is a graphic representation of fatty alcohol production in various deletion mutants of E. coli.

FIG. 28 is a graphic representation of fatty alcohol production in various deletion mutants of E. coli.

FIGS. 29A-29X are graphs depicting of the amount of fatty aldehydes converted to fatty alcohol using the enzymatic assays as described in Example 5. The title of each graph indicates the co-factor and substrate that were used in the assay. “C12” indicates a dodecanal substrate. “C16:1” indicates a 11-cis-hexadecenal substrate. The tables accompanying the graphs indicate the percentages of fatty aldehydes that were converted into fatty alcohols at the marked concentrations, as measured by GC-FID. The tables also indicate the p-values for the samples' capacity to catalyze the conversion of fatty aldehydes to fatty alcohols.

### DETAILED DESCRIPTION OF THE INVENTION

Unless otherwise defined, all technical and scientific terms used herein have the same meaning as commonly understood by one of the ordinary skill in the art to which this invention belongs. Although methods and materials similar or equivalent to those described herein can be used in the practice or testing of the present invention, suitable methods and materials are described below. All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

The invention is based, at least in part, on the discovery that altering the level of expression of one or more of a fatty alcohol biosynthetic polypeptide, a fatty aldehyde biosynthetic polypeptide, an acyl-ACP reductase polypeptide (EC 6.4.1.2) and a hydrocarbon biosynthetic polypeptide, e.g., a decarbonylase, in the microorganism host cell facilitates enhanced production of fatty acids and fatty acid derivatives by the microorganism.

### DEFINITIONS

Throughout the specification, a reference may be made using an abbreviated gene name or polypeptide name, but it is understood that such an abbreviated gene or polypeptide name represents the genus of genes or polypeptides. Such gene names include all genes encoding the same polypeptide and homologous polypeptides having the same physiological function. Polypeptide names include all polypeptides that have the same activity (e.g., that catalyze the same fundamental chemical reaction).

Unless otherwise indicated, the accession numbers referenced herein are derived from the NCBI database (National Center for Biotechnology Information) maintained by the National Institute of Health, U.S.A. Unless otherwise indicated, the accession numbers are as provided in the database as of October 2008.

EC numbers are established by the Nomenclature Committee of the International Union of Biochemistry and Molecular Biology (NC-IUBMB) (available at http://www.chem.qmul.ac.uldiubmb/enzyme/). The EC numbers referenced herein are derived from the KEGG Ligand database, maintained by the Kyoto Encyclopedia of Genes and Genomics, sponsored in part by the University of Tokyo. Unless otherwise indicated, the EC numbers are as provided in the database as of October 2008.

The articles “a” and “an” are used herein to refer to one or to more than one (i.e., to at least one) of the grammatical object of the article. By way of example, “an element” means one element or more than one element.

As used herein “acyl CoA” refers to an acyl thioester formed between the carbonyl carbon of alkyl chain and the sulfydryl group of the 4′-phosphopantethionyl moiety of coenzyme A (CoA), which has the formula R—C(O)S—CoA, where R is any alkyl group having at least 4 carbon atoms. In some instances an acyl CoA will be an intermediate in the synthesis of fully saturated acyl CoAs, including, but not limited to 3-keto-acyl CoA, a 3-hydroxy acyl CoA, a delta-2-trans-enoyl-CoA, or an alkyl acyl CoA. In some embodiments, the carbon chain will have about 5, 6, 7, 8, 9, 10, 11, 12, 13, 14, 15, 16, 17, 18, 19, 20, 21, 22, 23, 24, 25, or 26 carbons. In other embodiments the acyl CoA will be branched. In one embodiment the branched acyl CoA is an isoacyl CoA, in another it is an anti-isoacyl CoA. Each of these “acyl CoAs” are substrates for enzymes that convert them to fatty acid derivatives such as those described herein.

As used herein, the term “alcohol dehydrogenase” (EC1.1.1.*) is a peptide capable of catalyzing the conversion of a fatty aldehyde to an alcohol (e.g., fatty alcohol). Additionally, one of ordinary skill in the art will appreciate that some alcohol dehydrogenases will catalyze other reactions as well. For example, some alcohol dehydrogenases will accept other substrates in addition to fatty aldehydes. Such non-specific alcohol dehydrogenases are, therefore, also included in this definition. Nucleic acid sequences encoding alcohol dehydrogenases are known in the art, and such alcohol dehydrogenases are publicly available. Exemplary GenBank Accession Numbers include those provided in the figures.

As used herein, the term “aldehyde” means a hydrocarbon having the formula RCHO characterized by an unsaturated carbonyl group (C═O). In a preferred embodiment, the aldehyde is any aldehyde made from a fatty acid or fatty acid derivative. In one embodiment, the R group is at least about 1, 2, 3, 4, 5, 6, 7, 8, 9, 10, 11, 12, 13, 14, 15, 16, 17, 18, 19, or 20 carbons in length.

As used herein, an “aldehyde biosynthetic gene” or an “aldehyde biosynthetic polynucleotide” is a nucleic acid that encodes an aldehyde biosynthetic polypeptide.

As used herein, an “aldehyde biosynthetic polypeptide is a polypeptide that is a part of the biosynthetic pathway of an aldehyde. Such polypeptide can act on a biological substrate to yield an aldehyde. In some instances, the aldehyde biosynthetic polypeptide has reductase activity.

As used herein, the term “alkane” means saturated hydrocarbons or compounds that consist only of carbon (C) and hydrogen (H), wherein these atoms are linked together by single bonds (i.e., they are saturated compounds).

The terms “altered level of expression” and “modified level of expression” are used interchangeably and mean that a polynucleotide, polypeptide, or hydrocarbon is present in a different concentration in an engineered microorganism as compared to its concentration in a corresponding wild-type cell under the same conditions.

As used herein, the term “attenuate” means to weaken, reduce or diminish. For example, a polypeptide can be attenuated by modifying the polypeptide to reduce its activity (e.g., by modifying a nucleotide sequence that encodes the polypeptide).

In other embodiments, the polypeptide, polynucleotide, or hydrocarbon having an altered level of expression is “attenuated” or has a “decreased level of expression.” As used herein, “attenuate” and “decreasing the level of expression” mean to express or cause to be expressed a polynucleotide, polypeptide, or hydrocarbon in a cell at a lesser concentration than is normally expressed in a corresponding wild-type cell under the same conditions. The degree of overexpression or attenuation can be 1.5-fold or more, e.g., 2-fold or more, 3-fold or more, 5-fold or more, 10-fold or more, or 15-fold or more. Alternatively, or in addition, the degree of overexpression or attenuation can be 500-fold or less, e.g., 100-fold or less, 50-fold or less, 25-fold or less, or 20-fold or less. Thus, the degree of overexpression or attenuation can be bounded by any two of the above endpoints. For example, the degree of overexpression or attenuation can be 1.5-500-fold, 2-50-fold, 10-25-fold, or 15-20-fold.

As used herein, the term “biodiesel” means a biofuel that can be a substitute of diesel, which is derived from petroleum. Biodiesel can be used in internal combustion diesel engines in either a pure form, which is referred to as “neat” biodiesel, or as a mixture in any concentration with petroleum-based diesel. Biodiesel can include esters or hydrocarbons, such as alcohols.

As used therein, the term “biofuel” refers to any fuel derived from biomass. Biofuels can be substituted for petroleum based fuels. For example, biofuels are inclusive of transportation fuels (e.g., gasoline, diesel, jet fuel, etc.), heating fuels, and electricity-generating fuels. Biofuels are a renewable energy source.

As used herein, the term “biomass” refers to any biological material from which a carbon source is derived. In some embodiments, a biomass is processed into a carbon source, which is suitable for bioconversion. In other embodiments, the biomass does not require further processing into a carbon source. The carbon source can be converted into a biofuel. An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, fermentation waste, ensilage, straw, lumber, sewage, garbage, cellulosic urban waste, and food leftovers. The term “biomass” also can refer to sources of carbon, such as carbohydrates (e.g., monosaccharides, disaccharides, or polysaccharides).

“Branched chains” may have more than one point of branching and may include cyclic branches. In some embodiments, the branched fatty acid, branched fatty aldehyde, or branched fatty alcohol comprises a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₋₆ branched fatty acid, branched fatty aldehyde, or branched fatty alcohol. In particular embodiments, the branched fatty acid, branched fatty aldehyde, or branched fatty alcohol is a C₆, C₈, C₁₀, C₁₂, C₁₃, C₁₄, C₁₅, C₁₆, C₁₇, or C₁₋₈ branched fatty acid, branched fatty aldehyde, or branched fatty alcohol. In certain embodiments, the hydroxyl group of the branched fatty acid, branched fatty aldehyde, or branched fatty alcohol is in the primary (C₁) position. In certain embodiments, the branched fatty acid, branched fatty aldehyde, or branched fatty alcohol is an iso-fatty acid, iso-fatty aldehyde, or iso-fatty alcohol, or an antesio-fatty acid, an anteiso-fatty aldehyde, or anteiso-fatty alcohol. In exemplary embodiments, the branched fatty acid, branched fatty aldehyde, or branched fatty alcohol is selected from iso-C_{7:0}, iso-C_{8:0}, iso-C_{9:0}, iso-C_{10:0}, iso-C_{12:0}, iso-C_{13:0}, iso-C_{14:0}, iso-C_{15:0}, iso-C_{16:0}, iso-C_{17:0}, iso-C_{18:0}, iso-C_{19:0}, anteiso-C_{7:0}, anteiso-C_{8:0}, anteiso-C_{9:0}, anteiso-C_{10:0}, anteiso-C_{11:0}, anteiso-C_{12:0}, anteiso-C_{13:0}, anteiso-C_{14:0}, anteiso-C_{15:0}, anteiso-C_{16:0}, anteiso-C_{17:0}, anteiso-C_{18:0}, and anteiso-C_{19:0} branched fatty acid, branched fatty aldehyde or branched fatty alcohol.

As used herein, the phrase “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, and turanose; cellulosic material and variants such as methyl cellulose and sodium carboxymethyl cellulose; saturated or unsaturated fatty acid esters, succinate, lactate, and acetate; alcohols, such as ethanol, methanol, and glycerol, or mixtures thereof. The carbon source can also be a product of photosynthesis, such as glucose. In certain preferred embodiments, the carbon source is biomass. In other preferred embodiments, the carbon source is glucose.

As used herein, a “cloud point lowering additive” is an additive added to a composition to decrease or lower the cloud point of a solution.

As used herein, the phrase “cloud point of a fluid” means the temperature at which dissolved solids are no longer completely soluble. Below this temperature, solids begin precipitating as a second phase giving the fluid a cloudy appearance. In the petroleum industry, cloud point refers to the temperature below which a solidified material or other heavy hydrocarbon crystallizes in a crude oil, refined oil, or fuel to form a cloudy appearance. The presence of solidified materials influences the flowing behavior of the fluid, the tendency of the fluid to clog fuel filters, injectors, etc., the accumulation of solidified materials on cold surfaces (e.g., a pipeline or heat exchanger fouling), and the emulsion characteristics of the fluid with water.

A nucleotide sequence is “complementary” to another nucleotide sequence if each of the bases of the two sequences matches (i.e., is capable of forming Watson Crick base pairs). The term “complementary strand” is used herein interchangeably with the term “complement”. The complement of a nucleic acid strand can be the complement of a coding strand or the complement of a non-coding strand.

As used herein, the term “conditions sufficient to allow expression” means any conditions that allow a microorganism host cell to produce a desired product, such as a polypeptide or fatty aldehyde described herein. Suitable conditions include, for example, fermentation conditions. Fermentation conditions can comprise many parameters, such as temperature ranges, levels of aeration, and media composition. Each of these conditions, individually and in combination, allows the host cell to grow. Exemplary culture media include broths or gels. Generally, the medium includes a carbon source, such as glucose, fructose, cellulose, or the like, that can be metabolized by a host cell directly. In addition, enzymes can be used in the medium to facilitate the mobilization (e.g., the depolymerization of starch or cellulose to fermentable sugars) and subsequent metabolism of the carbon source. To determine if conditions are sufficient to allow expression, a host cell can be cultured, for example, for about 4, 8, 12, 24, 36, or 48 hours. During and/or after culturing, samples can be obtained and analyzed to determine if the conditions allow expression. For example, the host cells in the sample or the medium in which the host cells were grown can be tested for the presence of a desired product. When testing for the presence of a product, assays, such as, but not limited to, TLC, HPLC, GC/FID, GC/MS, LC/MS, MS, can be used.

As used herein, “control element” means a transcriptional control element. Control elements include promoters and enhancers. The term “promoter element,” “promoter,” or “promoter sequence” refers to a DNA sequence that functions as a switch that activates the expression of a gene. If the gene is activated, it is said to be transcribed or participating in transcription. Transcription involves the synthesis of mRNA from the gene. A promoter, therefore, serves as a transcriptional regulatory element and also provides a site for initiation of transcription of the gene into mRNA. Control elements interact specifically with cellular proteins involved in transcription (Maniatis et al., Science 236:1237, 1987).

As used herein, the term “fatty acid” means a carboxylic acid having the formula RCOOH. R represents an aliphatic group, preferably an alkyl group. R can comprise between about 4 and about 22 carbon atoms. Fatty acids can be saturated, monounsaturated, or polyunsaturated. In a preferred embodiment, the fatty acid is made from a fatty acid biosynthetic pathway.

As used herein, the term “fatty acid biosynthetic pathway” means a biosynthetic pathway that produces fatty acids. The fatty acid biosynthetic pathway includes fatty acid synthases that can be engineered, as described herein, to produce fatty acids, and in some embodiments can be expressed with additional enzymes to produce fatty acids having desired carbon chain characteristics.

As used herein, the term “fatty acid degradation enzyme” means an enzyme involved in the breakdown or conversion of a fatty acid or fatty acid derivative into another product. A nonlimiting example of a fatty acid degradation enzyme is an acyl-CoA synthase (EC 2.3.1.86). Additional examples of fatty acid degradation enzymes are described herein.

As used herein, the term “fatty acid derivative” means products made in part from the fatty acid biosynthetic pathway of the production host organism. “Fatty acid derivative” also includes products made in part from acyl-ACP or acyl-ACP derivatives. The fatty acid biosynthetic pathway includes fatty acid synthase enzymes which can be engineered as described herein to produce fatty acid derivatives, and in some examples can be expressed with additional enzymes to produce fatty acid derivatives having desired carbon chain characteristics. Exemplary fatty acid derivatives include for example, fatty acids, acyl-CoA, fatty aldehyde, short and long chain alcohols, hydrocarbons, fatty alcohols, and esters (e.g., waxes, fatty acid esters, or fatty esters).

As used herein, the term “fatty acid derivative enzyme” means any enzyme that may be expressed or overexpressed in the production of fatty acid derivatives. These enzymes may be part of the fatty acid biosynthetic pathway. Non-limiting examples of fatty acid derivative enzymes include fatty acid synthases, thioesterases (EC 3.1. 2.14 or EC 3.1.1.5), acyl-CoA synthases (EC 2.3.1.86), acyl-CoA reductases, alcohol dehydrogenases, alcohol acyltransferases, fatty alcohol-forming acyl-CoA reductases, fatty acid (carboxylic acid) reductases, acyl-ACP reductases (EC 6.4.1.2), fatty acid hydroxylases, acyl-CoA desaturases, acyl-ACP desaturases, acyl-CoA oxidases, acyl-CoA dehydrogenases, ester synthases, and alkane biosynthetic polypeptides, etc. Fatty acid derivative enzymes can convert a substrate into a fatty acid derivative. In some examples, the substrate may be a fatty acid derivative that the fatty acid derivative enzyme converts into a different fatty acid derivative. Exemplary suitable substrates include, C₆-C₂₆ fatty aldehydes.

As used herein, “fatty acid enzyme” means any enzyme involved in fatty acid biosynthesis. Fatty acid enzymes can be modified in host cells to produce fatty acids. Non-limiting examples of fatty acid enzymes include fatty acid synthases and thioesterases (EC 3.1. 2.14 or EC 3.1.1.5). Additional examples of fatty acid enzymes are described herein.

As used herein, the term “fatty acid or derivative thereof” means a “fatty acid” or a “fatty acid derivative.” The term “fatty acid” means a carboxylic acid having the formula RCOOH. R represents an aliphatic group, preferably an alkyl group. R can comprise between about 4 and about 22 carbon atoms. Fatty acids can be saturated, monounsaturated, or polyunsaturated. In a preferred embodiment, the fatty acid is made from a fatty acid biosynthetic pathway.

As used herein, “fatty alcohol” means an alcohol having the formula ROH. In some embodiments, the fatty alcohol is any alcohol made from a fatty acid or fatty acid derivative. In certain embodiments, the R group of a fatty acid, fatty aldehyde, or fatty alcohol is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty acid, fatty aldehyde, or fatty alcohol is a C6, C7, C8, C9, C10, C11, C12, C13, C14, C15, C16, C17, C18, C19, C20, C21, C22, C23, C24, C25, or a C26 fatty acid, fatty aldehyde, or fatty alcohol. In certain embodiments, the fatty acid, fatty aldehyde, or fatty alcohol is a C6, C8, C10, C12, C13, C14, C15, C16, C17, or C18 fatty acid, fatty aldehyde, or fatty alcohol. The R group of a fatty acid, fatty aldehyde, or fatty alcohol can be a straight chain or a branched chain.

As used herein, “fatty aldehyde” means an aldehyde having the formula RCHO characterized by an unsaturated carbonyl group (C═O). In a preferred embodiment, the fatty aldehyde is any aldehyde made from a fatty acid or fatty acid derivative. In one embodiment, the R group is at least about 1, 2, 3, 4, 5, 6, 7, 8, 9, 10, 11, 12, 13, 14, 15, 16, 17, 18, 19, or 20 carbons in length. R can be straight or branched chain. The branched chains may have one or more points of branching. In addition, the branched chains may include cyclic branches. Furthermore, R can be saturated or unsaturated. If unsaturated, the R can have one or more points of unsaturation. In one embodiment, the fatty aldehyde is produced biosynthetically. Fatty aldehydes have many uses. For example, fatty aldehydes can be used to produce many specialty chemicals. For example, fatty aldehydes are used to produce polymers, resins, dyes, flavorings, plasticizers, perfumes, pharmaceuticals, and other chemicals. Some are used as solvents, preservatives, or disinfectants. Some natural and synthetic compounds, such as vitamins and hormones, are aldehydes.

As used herein, the term “fatty ester” may be used in reference to an ester. In a preferred embodiment, a fatty ester is any ester made from a fatty acid, for example a fatty acid ester. In some embodiments, a fatty ester contains an A side and a B side. As used herein, an “A side” of an ester refers to the carbon chain attached to the carboxylate oxygen of the ester. As used herein, a “B side” of an ester refers to the carbon chain comprising the parent carboxylate of the ester. In embodiments where the fatty ester is derived from the fatty acid biosynthetic pathway, the A side is contributed by an alcohol, and the B side is contributed by a fatty acid. Any alcohol can be used to form the A side of the fatty esters. For example, the alcohol can be derived from the fatty acid biosynthetic pathway. Alternatively, the alcohol can be produced through non-fatty acid biosynthetic pathways. Moreover, the alcohol can be provided exogenously. For example, the alcohol can be supplied in the fermentation broth in instances where the fatty ester is produced by an organism. Alternatively, a carboxylic acid, such as a fatty acid or acetic acid, can be supplied exogenously in instances where the fatty ester is produced by an organism that can also produce alcohol. The carbon chains comprising the A side or B side can be of any length. In one embodiment, the A side of the ester is at least about 1, 2, 3, 4, 5, 6, 7, 8, 10, 12, 14, 16, or 18 carbons in length. When the fatty ester is a fatty acid methyl ester, the A side of the ester is 1 carbon in length. When the fatty ester is a fatty acid ethyl ester, the A side of the ester is 2 carbons in length. The B side of the ester can be at least about 4, 6, 8, 10, 12, 14, 16, 18, 20, 22, 24, or 26 carbons in length. The A side and/or the B side can be straight or branched chain. The branched chains can have one or more points of branching. In addition, the branched chains can include cyclic branches. Furthermore, the A side and/or B side can be saturated or unsaturated. If unsaturated, the A side and/or B side can have one or more points of unsaturation. In some embodiments, the fatty acid ester is a fatty acid methyl ester (FAME) or a fatty acid ethyl ester (FAEE). In certain embodiments, the FAME is a beta-hydroxy (B—OH) FAME. In one embodiment, the fatty ester is a wax. The wax can be derived from a long chain alcohol and a long chain fatty acid. In another embodiment, the fatty ester is a fatty acid thioester, for example fatty acyl Coenzyme A (CoA). In other embodiments, the fatty ester is a fatty acyl pantothenate, an acyl carrier protein (ACP), or a fatty phosphate ester.

As used herein, “fraction of modern carbon” or “f_{M}” has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

“Gene knockout”, as used herein, refers to a procedure by which a gene encoding a target protein is modified or inactivated so to reduce or eliminate the function of the intact protein. Inactivation of the gene may be performed by general methods such as mutagenesis by UV irradiation or treatment with N-methyl-N′-nitro-N-nitrosoguanidine, site-directed mutagenesis, homologous recombination, insertion-deletion mutagenesis, or “Red-driven integration” (Datsenko et al., Proc. Natl. Acad. Sci. USA, 97:6640-45, 2000). For example, in one embodiment, a construct is introduced into a host cell, such that it is possible to select for homologous recombination events in the host cell. One of skill in the art can readily design a knock-out construct including both positive and negative selection genes for efficiently selecting transfected cells that undergo a homologous recombination event with the construct. The alteration in the host cell may be obtained, for example, by replacing through a single or double crossover recombination a wild type DNA sequence by a DNA sequence containing the alteration. For convenient selection of transformants, the alteration may, for example, be a DNA sequence encoding an antibiotic resistance marker or a gene complementing a possible auxotrophy of the host cell. Mutations include, but are not limited to, deletion-insertion mutations. An example of such an alteration includes a gene disruption, i.e., a perturbation of a gene such that the product that is normally produced from this gene is not produced in a functional form. This could be due to a complete deletion, a deletion and insertion of a selective marker, an insertion of a selective marker, a frameshift mutation, an in-frame deletion, or a point mutation that leads to premature termination. In some instances, the entire mRNA for the gene is absent. In other situations, the amount of mRNA produced varies.

As used herein, a “host cell” is a cell used to produce a product described herein (e.g., a fatty alcohol described herein). A host cell can be modified to express or overexpress selected genes or to have attenuated expression of selected genes. Non-limiting examples of host cells include plant, animal, human, bacteria, yeast, or filamentous fungi cells.

In some embodiments, a polypeptide described herein has “increased level of activity.” By “increased level of activity” is meant that a polypeptide has a higher level of biochemical or biological function (e.g., DNA binding or enzymatic activity) in an engineered host cell as compared to its level of biochemical and/or biological function in a corresponding wild-type host cell under the same conditions. The degree of enhanced activity can be about 10% or more, about 20% or more, about 50% or more, about 75% or more, about 100% or more, about 200% or more, about 500% or more, about 1000% or more, or any range therein.

The term “isolated” as used herein with respect to nucleic acids, such as DNA or RNA, refers to molecules separated from other DNAs or RNAs, respectively that are present in the natural source of the nucleic acid. Moreover, by an “isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides. The term “isolated” as used herein also refers to a nucleic acid or peptide that is substantially free of cellular material, viral material, or culture medium when produced by recombinant DNA techniques. The term “isolated” as used herein also refers to a nucleic acid or peptide that is substantially free of chemical precursors or other chemicals when chemically synthesized. The teen “isolated”, as used herein with respect to products, such as fatty alcohols, refers to products that are isolated from cellular components, cell culture media, or chemical or synthetic precursors.

As used herein, the “level of expression of a gene” refers to the level of mRNA, pre-mRNA nascent transcript(s), transcript processing intermediates, mature mRNA(s), and degradation products encoded by the gene.

As used herein, the term “microorganism” means prokaryotic and eukaryotic microbial species from the domains Archaea, Bacteria and Eucarya, the latter including yeast and filamentous fungi, protozoa, algae, or higher Protista. The terms “microbial cells” (i.e., cells from microbes) and “microbes” are used interchangeably and refer to cells or small organisms that can only be seen with the aid of a microscope.

As used herein, the term “nucleic acid” refers to polynucleotides, such as deoxyribonucleic acid (DNA), and, where appropriate, ribonucleic acid (RNA). The term should also be understood to include, as equivalents, analogs of RNAs or DNAs made from nucleotide analogs, and, as applicable to the embodiment being described, single (sense or antisense) and double-stranded polynucleotides, ESTs, chromosomes, cDNAs, mRNAs, and rRNAs.

The term “nucleotide” as used herein refers to a monomeric unit of a polynucleotide that consists of a heterocyclic base, a sugar, and one or more phosphate groups. The naturally occurring bases (guanine, (G), adenine, (A), cytosine, (C), thymine, (T), and uracil (U)) are typically derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleic acids are typically linked via phosphate bonds to form nucleic acids or polynucleotides, though many other linkages are known in the art (e.g., phosphorothioates, boranophosphates, and the like). Polynucleotides described herein may comprise degenerate nucleotides which are defined according to the IUPAC code for nucleotide degeneracy wherein B is C, G, or T; D is A, G, or T; H is A, C, or T; K is G or T; M is A or C; N is A, C, G, or T; R is A or G; S is C or G; V is A, C, or G; W is A or T; and Y is C or T.

The terms “olefin” and “alkene” are used interchangeably herein, and refer to hydrocarbons containing at least one carbon-to-carbon double bond (i.e., they are unsaturated compounds).

As used herein, the teim “operably linked” means that selected nucleotide sequence (e.g., encoding a polypeptide described herein) is in proximity with a promoter to allow the promoter to regulate expression of the selected DNA. In addition, the promoter is located upstream of the selected nucleotide sequence in terms of the direction of transcription and translation. By “operably linked” is meant that a nucleotide sequence and a regulatory sequence(s) are connected in such a way as to permit gene expression when the appropriate molecules (e.g., transcriptional activator proteins) are bound to the regulatory sequence(s).

The term “or” is used herein to mean, and is used interchangeably with, the term “and/or,” unless context clearly indicates otherwise.

In some embodiments, the polypeptide, polynucleotide, or hydrocarbon having an altered or modified level of expression is “overexpressed” or has an “increased level of expression.” As used herein, “overexpress” and “increasing the level of expression” mean to express or cause to be expressed a polynucleotide, polypeptide, or hydrocarbon in a cell at a greater concentration than is noiinally expressed in a corresponding wild-type cell under the same conditions. For example, a polypeptide can be “overexpressed” in an engineered host cell when the polypeptide is present in a greater concentration in the engineered host cell as compared to its concentration in a non-engineered host cell of the same species under the same conditions.

As used herein, “partition coefficient” or “P,” is defined as the equilibrium concentration of a compound in an organic phase divided by the concentration at equilibrium in an aqueous phase (e.g., fermentation broth). In one embodiment of a bi-phasic system described herein, the organic phase is formed by the fatty aldehyde during the production process. However, in some examples, an organic phase can be provided, such as by providing a layer of octane, to facilitate product separation. When describing a two phase system, the partition characteristics of a compound can be described as logP. For example, a compound with a logP of 1 would partition 10:1 to the organic phase. A compound with a logP of −1 would partition 1:10 to the organic phase. By choosing an appropriate fermentation broth and organic phase, a fatty aldehyde with a high logP value can separate into the organic phase even at very low concentrations in the fermentation vessel.

“Polynucleotide” refers to a polymer of DNA or RNA, which can be single-stranded or double-stranded and which can contain non-natural or altered nucleotides. The terms “polynucleotide,” “nucleic acid,” and “nucleic acid molecule” are used herein interchangeably to refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides. The polynucleotide can be in any form, including but not limited to plasmid, viral, chromosomal, EST, cDNA, mRNA, and rRNA.

The terms “polypeptide” and “protein” refer to a polymer of amino acid residues. The term “recombinant polypeptide” refers to a polypeptide that is produced by recombinant DNA techniques, wherein generally DNA encoding the expressed protein or RNA is inserted into a suitable expression vector that is in turn used to transform a host cell to produce the polypeptide or RNA.

As used herein, the term “purify,” “purified,” or “purification” means the removal or isolation of a molecule from its environment by, for example, isolation or separation. “Substantially purified” molecules are at least about 60% free, preferably at least about 75% free, and more preferably at least about 90% free from other components with which they are associated. As used herein, these terms also refer to the removal of contaminants from a sample. For example, the removal of contaminants can result in an increase in the percentage of fatty alcohol in a sample. For example, when fatty alcohols are produced in a host cell, the fatty alcohols can be purified by the removal of host cell proteins. After purification, the percentage of fatty alcohols in the sample is increased. The terms “purify,” “purified,” and “purification” do not require absolute purity. They are relative terms. Thus, for example, when fatty alcohols are produced in host cells, a purified fatty alcohol is one that is substantially separated from other cellular components (e.g., nucleic acids, polypeptides, lipids, carbohydrates, or other hydrocarbons). In another example, a purified fatty alcohol preparation is one in which the fatty alcohol is substantially free from contaminants, such as those that might be present following fermentation. In some embodiments, a fatty alcohol is purified when at least about 50% by weight of a sample is composed of the fatty alcohol. In other embodiments, a fatty alcohol is purified when at least about 60%, 70%, 80%, 85%, 90%, 92%, 95%, 98%, or 99% or more by weight of a sample is composed of the fatty alcohol.

As used herein, the term “recombinant polypeptide” refers to a polypeptide that is produced by recombinant DNA techniques, wherein generally DNA encoding the expressed protein or RNA is inserted into a suitable expression vector and that is in turn used to transform a host cell to produce the polypeptide or RNA.

The R group of a branched or unbranched fatty acid, branched or unbranched fatty aldehyde, or branched or unbranched fatty alcohol can be “saturated” or “unsaturated”. If unsaturated, the R group can have one or more than one point of unsaturation. In some embodiments, the unsaturated fatty acid, unsaturated fatty aldehyde, or unsaturated fatty alcohol is a monounsaturated fatty acid, monounsaturated fatty aldehyde, or monounsaturated fatty alcohol. In certain embodiments, the unsaturated fatty acid, unsaturated fatty aldehyde, or unsaturated fatty alcohol is a C6:1, C7:1, C8:1, C9:1, C10:1, C11:1, C12:1, C13:1, C14:1, C15:1, C16:1, C17:1, C18:1, C19:1, C20:1, C21:1, C22:1, C23:1, C24:1, C25:1, or a C26:1 unsaturated fatty acid, unsaturated fatty aldehyde, or unsaturated fatty alcohol. In certain preferred embodiments, the unsaturated fatty acid, unsaturated fatty aldehyde, or unsaturated fatty alcohol is C10:1, C12:1, C14:1, C16:1, or C18:1. In yet other embodiments, the unsaturated fatty acid, unsaturated fatty aldehyde, or unsaturated fatty alcohol is unsaturated at the omega-7 position. In certain embodiments, the unsaturated fatty acid, unsaturated fatty aldehyde, or unsaturated fatty alcohol comprises a cis double bond.

As used herein, the term “substantially identical” (or “substantially homologous”) is used to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities.

As used herein, the term “synthase” means an enzyme which catalyzes a synthesis process. As used herein, the term synthase includes synthases, synthetases, and ligases.

The terms “terminal olefin,” “α-olefin”, “terminal alkene” and “1-alkene” are used interchangeably herein with reference to α-olefins or alkenes with a chemical formula CₓH2ₓ, distinguished from other olefins with a similar molecular formula by linearity of the hydrocarbon chain and the position of the double bond at the primary or alpha position.

As used herein, the term “transfection” means the introduction of a nucleic acid (e.g., via an expression vector) into a recipient cell by nucleic acid-mediated gene transfer.

As used herein, “transformation” refers to a process in which a cell's genotype is changed as a result of the cellular uptake of exogenous DNA or RNA. This may result in the transformed cell expressing a recombinant form of an RNA or polypeptide. In the case of antisense expression from the transferred gene, the expression of a naturally-occurring form of the polypeptide is disrupted.

As used herein, a “transport protein” is a polypeptide that facilitates the movement of one or more compounds in and/or out of a cellular organelle and/or a cell.

As used herein, the term “vector” refers to a nucleic acid molecule capable of transporting another nucleic acid to which it has been linked. One type of useful vector is an episome (i.e., a nucleic acid capable of extra-chromosomal replication). Useful vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids,” which refer generally to circular double stranded DNA loops that, in their vector form, are not bound to the chromosome. In the present specification, “plasmid” and “vector” are used interchangeably, as the plasmid is the most commonly used form of vector. However, also included are such other forms of expression vectors that serve equivalent functions and that become known in the art subsequently hereto.

### DESCRIPTION OF EXEMPLARY EMBODIMENTS

### Production of Fatty Alcohols

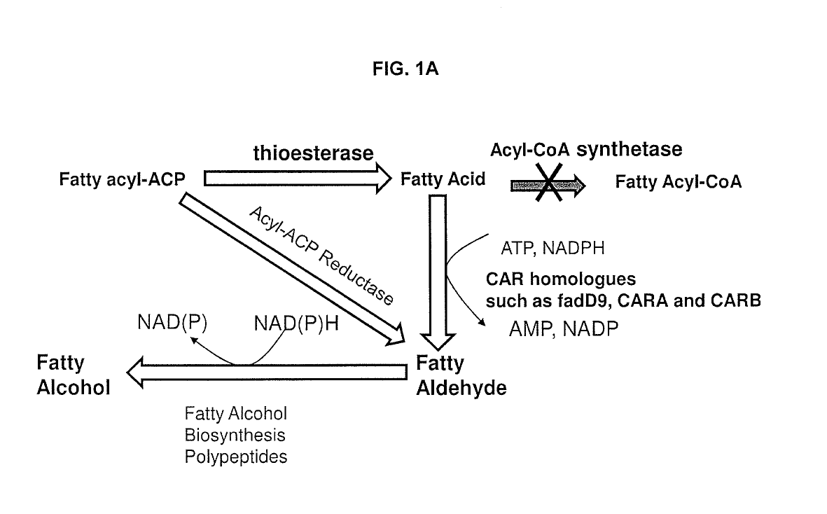

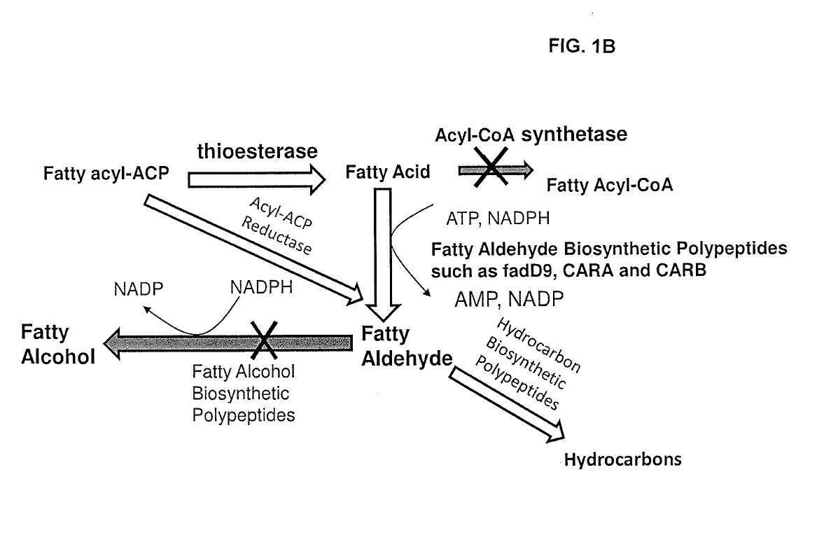

The invention is based, at least in part, on the identification of a number of fatty alcohol biosynthetic enzymes or polypeptides that are capable of catalyzing the conversion of a fatty aldehyde to a fatty alcohol under suitable conditions, for example, in the presence of suitable substrates and/or co-factors. The fatty alcohols can be produced by one or more or all of the fatty alcohol biosynthesis pathways in E. coli that utilize, in part, genes that encode fatty aldehyde biosynthetic polypeptides, acyl-ACP reductases (EC 6.4.1.2), or the fatty alcohol biosynthetic enzymes of the present invention. In certain embodiments, the fatty alcohols are produced by a biosynthetic pathway depicted in FIG. 1A In this pathway, a fatty acid is first activated by ATP and then reduced to generate a fatty aldehyde. The fatty aldehyde can then be further reduced into a fatty alcohol by a fatty alcohol biosynthetic polypeptide of the present invention, such as, for example, a fatty aldehyde reductase, an alcohol dehydrogenase, an oxidoreductase, an aldo-keto reductase, or a short-chain dehydrogenase. In certain other embodiments, the fatty alcohols are produced by an alternative biosynthesis pathway depicted in FIG. 1A. In this pathway, an acyl-ACP is converted into a fatty aldehyde catalyzed by an acyl-ACP reductase (EC 6.4.1.2). The fatty aldehyde is further reduced into a fatty alcohol by a fatty alcohol biosynthetic polypeptide of the present invention, for example, by a fatty aldehyde reductase, an alcohol dehydrogenase, an oxidoreductase, an aldo-keto reductase, or a short-chain dehydrogenase. Exemplary embodiments of fatty alcohol biosynthetic enzymes of the present invention includes, without limitation, adhP, dkgA, dkgB, rspB, yahK, ybbO, ybdH, ybdR, ygfF, yhdH, yjgB, aroB, ycjQ, ydbC, ydjG, yeaE, yncB, yghD, ydjL, Tas, among others. Suitable substrates of these enzymes include fatty aldehydes, for example fatty aldehydes with carbon chain lengths from C₁₀ to C₁₈. Suitable co-factors include, without limitation, NAD, NAD(P), NADH, or NADPH.

The methods described herein can be used to produce fatty alcohols in an engineered microorganism by conversion of fatty aldehydes into fatty alcohols. In some instances, the fatty alcohol is produced by a fatty alcohol biosynthetic polypeptide having an amino acid sequence listed provided herein, as well as polypeptide variant thereof.

In other instances, the methods described herein can be used to produce fatty alcohols in an engineered microorganism using an acyl-ACP reductase polypeptide having an amino acid sequence provided herein, as well as a polypeptide variant thereof. In some instances, an acyl-ACP reductase polypeptide is one that includes one or more of the amino acid motifs disclosed herein. For example, the polypeptide can comprise one or more of SEQ ID NO:155, 156, 157, 158, 159, 160, 161, 162, 163, 164, or 165.

### Fatty Alcohol Biosynthetic Genes and Polypeptides.

In some instances, a fatty alcohol is produced by expressing a gene encoding a fatty alcohol biosynthetic polypeptide that is capable of catalyzing the enzymatic conversion of a fatty aldehyde to a fatty alcohol.

In some embodiments, the method further includes isolating the fatty alcohol from the host cell. In some embodiments, the fatty alcohol is present in the extracellular environment. In certain embodiments, the fatty alcohol is isolated from the extracellular environment. In certain embodiments, the fatty alcohol is spontaneously secreted, partially or completely, from the host cell. In alternative embodiments, the fatty alcohol is transported into the extracellular environment. In other embodiments, the fatty alcohol is passively transported into the extracellular environment. In some embodiments, the method further includes purifying the fatty alcohol.

In some embodiments, the fatty alcohol biosynthetic polypeptide is about 200 amino acids to about 800 amino acids in length. In certain embodiments, the polypeptide is about 250 amino acids to about 700 amino acids in length, for example, is about 300 to about 600 amino acids in length, about 350 to about 500 amino acids in length, or about 350 to about 450 amino acids in length. In other embodiments, the fatty alcohol biosynthetic polypeptide is up to about 800 amino acids in length, for example, up to about 700 amino acids in length, about 600 amino acids in length, about 500 amino acids in length, about 450 amino acids in length, about 400 amino acids in length, about 350 amino acids in length, about 300 amino acids in length, about 250 amino acids in length, or about 200 amino acids in length. In other embodiments, the fatty alcohol biosynthetic polypeptide is more than about 200 amino acids in length, for example, more than about 250 amino acids in length, about 300 amino acids in length, about 350 amino acids in length, about 400 amino acids in length, about 450 amino acids in length, about 500 amino acids in length, about 600 amino acids in length, about 700 amino acids in length, or about 800 amino acids in length.

In some embodiments, the fatty alcohol biosynthetic polypeptide comprises the amino acid sequence of SEQ ID NO: 1, 3, 5, 7, 9, 11, 13, 15, 17, 19, 21, 23, 25, 27, 29, 31, 33, 35, 37, or 39, with one or more amino acid substitutions, additions, insertions, or deletions, wherein the polypeptide is capable of catalyzing the enzymatic conversion of a fatty aldehyde to a fatty alcohol under suitable conditions, for example, in the presence of suitable substrates and/or co-factors. In certain embodiments, the polypeptide is capable of catalyzing the enzymatic conversion of a fatty aldehyde into a fatty alcohol under suitable conditions, for example, in the presence of suitable substrates and/or co-factors. In certain embodiments, the polypeptide is a fatty aldehyde reductase and/or has fatty aldehyde reductase activity (EC1.1.1.1). In some embodiments, the polypeptide is an alcohol dehydrogenase and/or has alcohol dehydrogenase activity. In certain embodiments, the polypeptide is an aldo-keto reductase and/or has aldo-keto reductase activity. In certain other embodiments, the polypeptide is a short-chain dehydrogenase and/or has short-chain dehydrogenase activity. In yet other embodiments, the polypeptide is an oxidoreductase and/or has oxidoreductase activity. In certain further embodiments, the polypeptide comprises one or more NAD(P)- or NAD(P)H-binding domains and/or is associated with an NAD(P) or NAD(P)H co-factor. In yet further embodiments, the three-dimensional or the predicted three-dimensional structure of the polypeptide comprises a Rossman fold.

In some embodiments, the fatty alcohol biosynthetic is a mutant or variant.

Various known activity assays can be used to determine the enzymatic activity of a putative fatty alcohol biosynthetic polypeptide. These assays can be suitable or useful for determining, for example, the expression or level of various fatty alcohol biosynthetic polypeptides in an engineered host cell or microorganism. For example, the capacity of a polypeptide to convert a fatty aldehyde into a fatty alcohol can be determined by measuring the rate of increase or decrease of NAD(P)H at 340 nm (ε=6.22 nM⁻¹ cm⁻¹) using aldehydes as substrates at 25° C. See, e.g., Schweiger et al., Appl. Microbiol. Biotechnol. (published online 31 Jul. 2009). Specifically, a 1.0 mL reaction mixture consisting of 5 mM aldehyde substrate, 40 mM potassium phosphate buffer, pH7.0, 125 μM NADPH and enzyme can be prepared. One unit can be defined as the amount of enzyme activity catalyzing the conversion of 1.0 μmol of pyridine nucleotide per minute. Alternatively, a similar assay with somewhat different conditions can be carried out to determine the fatty alcohol biosynthetic enzymatic activity. See, e.g., Wahlen et al., App. Environ. Microbiol. 75(9):2758-2764 (2009). Specifically, about 50 μg of purified enzyme can be added to a reaction mixture containing 100 mM Tris buffer at pH 7.9, 100 mM NaCl, 2.4 mM of either NADPH or NADH as a reactant, and decanal, oleic acid, and hexadecanol as possible substrates. Optionally the assay can be run under an argon atmosphere in septum-sealed vials overnight at room temperature with constant and gentle mixing. The products of the reaction can then be extracted from the buffer by adding an equal volume of hexane, and organic layer components can be analyzed by gas chromatography equipped with a flame ionization detector (30 m by 0.32 mm inner diameter with 0.5 μm film thickness, with argon as a carrier and a temperature ramp of for example, from 60° C. to 360° C., increasing at 10° C. per minute). A continuous spectrophotometric assay can also be developed to determine a given polypeptide's capacity to convert a fatty aldehyde into a fatty alcohol. The activity assays and conditions described in the examples herein are also suitable for this determination.

In some embodiments, the fatty alcohol biosynthetic polypeptide has an amino acid sequence that is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1, 3, 5, 7, 9, 11, 13, 15, 17, 19, 21, 23, 25, 27, 29, 31, 33, 35, 37, or 39. In some embodiments, the polypeptide has the amino acid sequence of SEQ ID NO: 1, 3, 5, 7, 9, 11, 13, 15, 17, 19, 21, 23, 25, 27, 29, 31, 33, 35, 37, or 39.

In some embodiments, the nucleotide sequence has at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO:2, 4, 6, 8, 10, 12, 14, 16, 18, 20, 22, 24, 26, 28, 30, 32, 34, 36, 38, or 40. In some embodiments, the nucleotide sequence is SEQ ID NO:2, 4, 6, 8, 10, 12, 14, 16, 18, 20, 22, 24, 26, 28, 30, 32, 34, 36, 38, or 40.

In other embodiments, the nucleotide sequence hybridizes to a complement of SEQ ID NO: 2, 4, 6, 8, 10, 12, 14, 16, 18, 20, 22, 24, 26, 28, 30, 32, 34, 36, 38, or 40, or to a fragment thereof, for example, under low stringency, medium stringency, high stringency, or very high stringency conditions, wherein the polynucleotide encodes a polypeptide that is capable of catalyzing the enzymatic conversion of a fatty aldehyde to a fatty alcohol under suitable conditions, for example, in the presence of suitable substrates and/or co-factors. In some embodiments, the polynucleotide encodes a fatty alcohol biosynthetic enzyme. In certain embodiments, the polynucleotide encodes a fatty aldehyde reductase and/or encodes a polypeptide having fatty aldehyde reductase activity. In some embodiments, the polynucleotides encodes an alcohol dehydrogenase and/or encodes a polypeptide having alcohol dehydrogenase activity. In other embodiments, the polynucleotide encodes an oxidoreductase and/or a polypeptide having oxidoreductase activity. In certain embodiments, the polynucleotide encodes an aldo-keto reductase and/or a polypeptide having aldo-keto reductase activity. In certain other embodiments, the polynucleotide encodes a short-chain dehydrogenase and/or a polypeptide having short-chain dehydrogenase activity. In yet further embodiments, the polypeptide comprises one or more NAD(P)- or NAD(P)H-binding domains or is associated with an NAD(P) or NAD(P)H co-factors. In other embodiments, the three-dimensional structure or the predicted three-dimensional structure of the polypeptide comprises a Rossman fold.

In any of the aspects of the invention described herein, the method can produce fatty alcohols comprising a C₆-C₂₆ fatty alcohol. In some embodiments, the fatty alcohol comprises a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty alcohol. In particular embodiments, the fatty alcohol is a C₆, C₈, C₁₀, C₁₂, C₁₃, C₁₄, C₁₅, C₁₆, C₁₇, or C₁₈ fatty alcohol. In certain embodiments, the hydroxyl group of the fatty alcohol is in the primary (C₁) position. In other embodiments, the fatty alcohol comprises a straight chain fatty alcohol. In other embodiments, the fatty alcohol comprises a branched chain fatty alcohol. In yet other embodiments, the fatty alcohol comprises a cyclic moiety.

In some embodiments, the fatty alcohol is an unsaturated fatty alcohol. In other embodiments, the fatty alcohol is a monounsaturated fatty alcohol. In certain embodiments, the unsaturated fatty alcohol is a C6:1, C7:1, C8:1, C9:1, C10:1, C11:1, C12:1, C13:1, C14:1, C15:1, C16:1, C17:1, C18:1, C19:1, C20:1, C21:1, C22:1, C23:1, C24:1, C25:1, or a C26:1 unsaturated fatty alcohol. In yet other embodiments, the fatty alcohol is unsaturated at the omega-7 position. In certain embodiments, the unsaturated fatty alcohol comprises a cis double bond.

In yet other embodiments, the fatty alcohol is a saturated fatty alcohol. In any of the aspects of the invention described herein, a suitable substrate for the polypeptide can be a fatty aldehyde. In some embodiments, the fatty aldehyde comprises a C₆-C₂₆ fatty aldehyde. In some embodiments, the fatty aldehyde comprises a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty aldehyde. In particular embodiments, the fatty aldehyde is a C₆, C₈, C₁₀, C₁₂, C₁₃, C₁₄, C₁₅, C₁₆, C₁₇, or C₁₈ fatty aldehyde.

In other embodiments, the fatty aldehyde comprises a straight chain fatty aldehyde. In other embodiments, the fatty aldehyde comprises a branched chain fatty aldehyde. In yet other embodiments, the fatty aldehyde comprise one or more cyclic moieties.

In some embodiments, the fatty aldehyde is an unsaturated fatty aldehyde. In other embodiments, the fatty aldehyde substrate is a monounsaturated fatty aldehyde. In yet other embodiments, the fatty aldehyde is a saturated fatty aldehyde.

In any of the aspects of the invention described herein, a suitable co-factor for the fatty alcohol biosynthetic polypeptide can be, for example, NAD, NADP, NADH, and/or NADPH. In some embodiments, the polypeptide comprises a co-factor binding domain or is associated with one of more of the co-factors. In particular embodiments, the three-dimensional structure or the predicted three-dimensional structure of the polypeptide comprises a Rossman fold.

In another aspect, the invention features an engineered microorganism comprising an exogenous control sequence stably incorporated into the genomic DNA of the microorganism upstream of a fatty alcohol biosynthetic polynucleotide comprising a nucleotide sequence having at least about 50% sequence identity to the nucleotide sequence of SEQ ID NO: 2, 4, 6, 8, 10, 12, 14, 16, 18, 20, 22, 24, 26, 28, 30, 32, 34, 36, 38, or 40, wherein the microorganism produces an increased level of a fatty alcohol relative to a wild-type microorganism.

In some embodiments, the nucleotide sequence has at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO:2, 4, 6, 8, 10, 12, 14, 16, 18, 20, 22, 24, 26, 28, 30, 32, 34, 36, 38, or 40. In some embodiments, the nucleotide sequence is SEQ ID NO: 2, 4, 6, 8, 10, 12, 14, 16, 18, 20, 22, 24, 26, 28, 30, 32, 34, 36, 38, or 40.

In some embodiments, the fatty alcohol biosynthetic polynucleotide is endogenous to the microorganism.

In other embodiments, the microorganism is engineered to express a modified level of a gene encoding a fatty acid derivative enzyme. In certain embodiments, modifying the expression of a gene encoding a fatty acid derivative enzyme includes expressing a gene encoding a fatty acid derivative enzyme and/or increasing the expression or activity of an endogenous fatty acid derivative enzyme. In alternative embodiments, modifying the expression of a gene encoding a fatty acid derivative enzyme includes attenuating a gene encoding a fatty acid derivative enzyme and/or decreasing the expression or activity of an endogenous fatty acid derivative enzyme. In some embodiments, the fatty acid derivative enzyme is a fatty acid synthase. In other embodiments, the fatty acid derivative enzyme is a thioesterase (EC 3.1. 2.14 or EC 3.1.1.5). In particular embodiments, the thioesterase is encoded by tesA, tesA without leader sequence, tesB, fatB, fatB2, fatB3, fatA, or fatA1.

In certain embodiments, one or more of the fatty alcohol biosynthetic polypeptides are overexpressed relative to expression in a wild type host cell.

While not wishing to be bound by theory, it is believed that the fatty alcohol biosynthetic polypeptide described herein produce fatty alcohols from substrate via a reduction mechanism. In some instances, the substrate is a fatty aldehyde or a derivative thereof, a fatty alcohol having particular branching patterns and carbon chain lengths can be produced from a fatty aldehyde having those characteristics that would result in a particular fatty alcohol. The fatty aldehyde substrates can, in turn, be obtained from another reaction mechanism, including, for example, via a reaction converting a fatty acid catalyzed by a fatty aldehyde biosynthetic enzyme or via a reaction converting an acyl-ACP substrate catalyzed by an acyl-ACP reductase.

In addition, each step within a biosynthetic pathway that leads to the production of a fatty aldehyde derivative substrate can be modified to produce or overproduce the substrate of interest. For example, known genes involved in the fatty acid biosynthetic pathway or the fatty aldehyde pathway can be expressed, overexpressed, or attenuated in host cells to produce a desired substrate (see, e.g., various enzymes described in PCT/US08/058,788, incorporated by reference herein).

A suitable fatty acid substrate can be converted into a fatty aldehyde substrate by, for example, a fatty aldehyde biosynthetic polypeptide such as a carboxylic acid reductase, or an acyl-ACP reductase. For example, the fatty aldehyde biosynthetic polypeptide can be selected from those described herein, or variants thereof. Alternatively, the acyl-ACP reductase can be one selected from those described herein, or a variant thereof. Then, the fatty aldehyde substrate can be converted into a fatty alcohol by, for example, a gene encoding a fatty alcohol biosynthetic polypeptide of the present invention. In some example, a gene encoding a fatty alcohol biosynthetic polypeptide described herein can be expressed in a host cell that expresses an endogenous fatty alcohol biosynthetic polypeptide capable of converting a fatty aldehyde produced by the fatty aldehyde biosynthetic polypeptide into a corresponding fatty alcohol. In other instances, a gene encoding a fatty alcohol biosynthetic polypeptide described herein, such as an amino acid sequence selected from SEQ ID NO:1, 3, 5, 7, 9, 11, 13, 15, 17, 19, 21, 23, 25, 27, 29, 31, 33, 35, 37, or 39, or a variant thereof. In certain embodiments, the fatty alcohol biosynthetic polypeptide described herein can be encoded by a polynucleotide comprising a sequence of SEQ ID NO: 2, 4, 6, 8, 10, 12, 14, 16, 18, 20, 22, 24, 26, 28, 30, 32, 34, 36, 38, or 40, or a variant thereof.

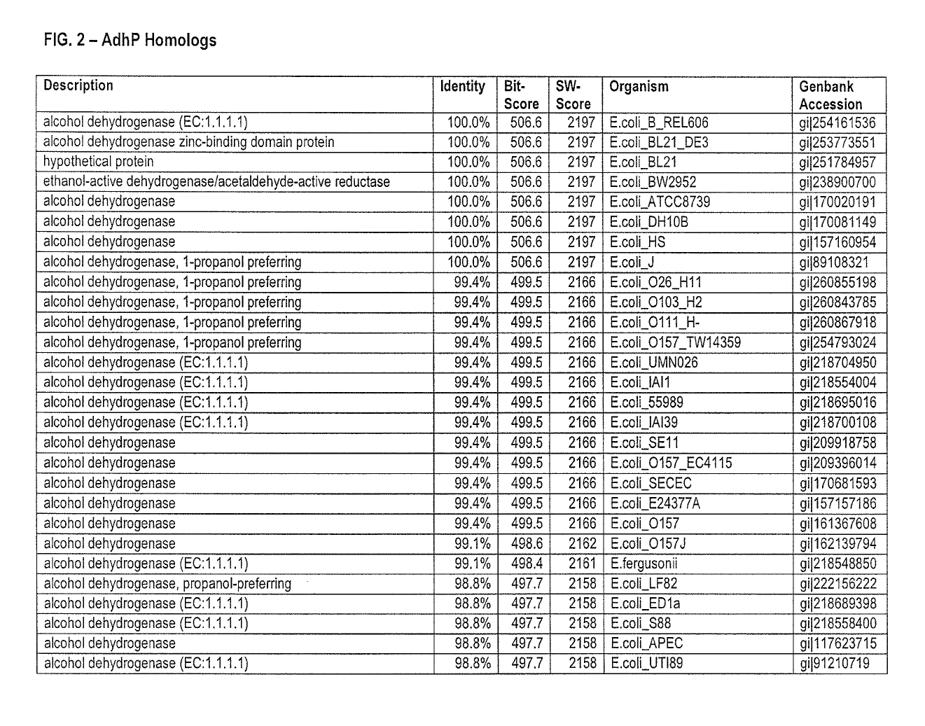

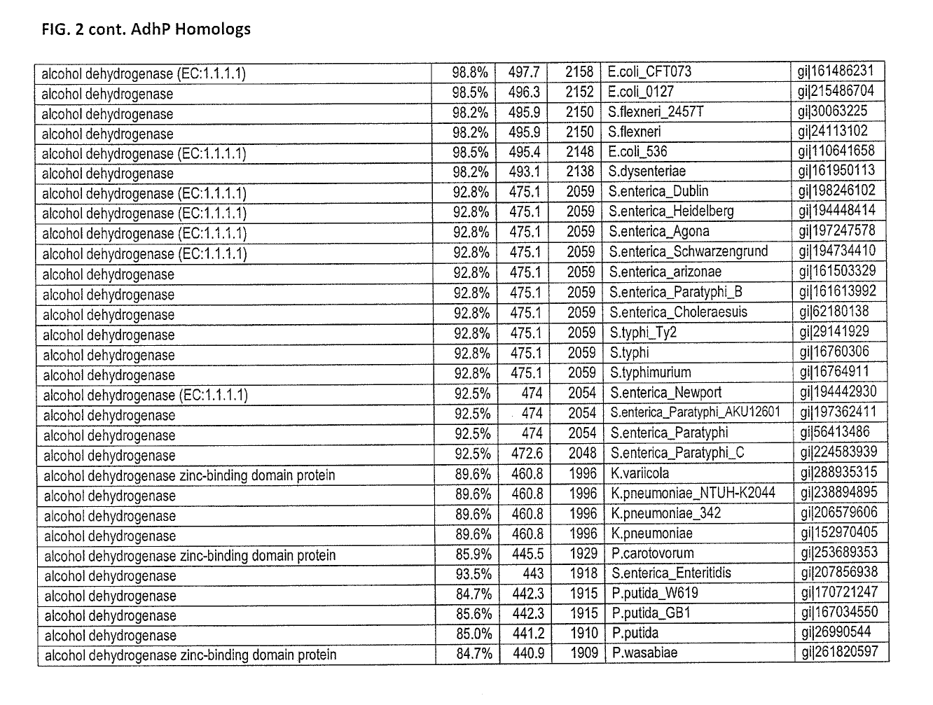

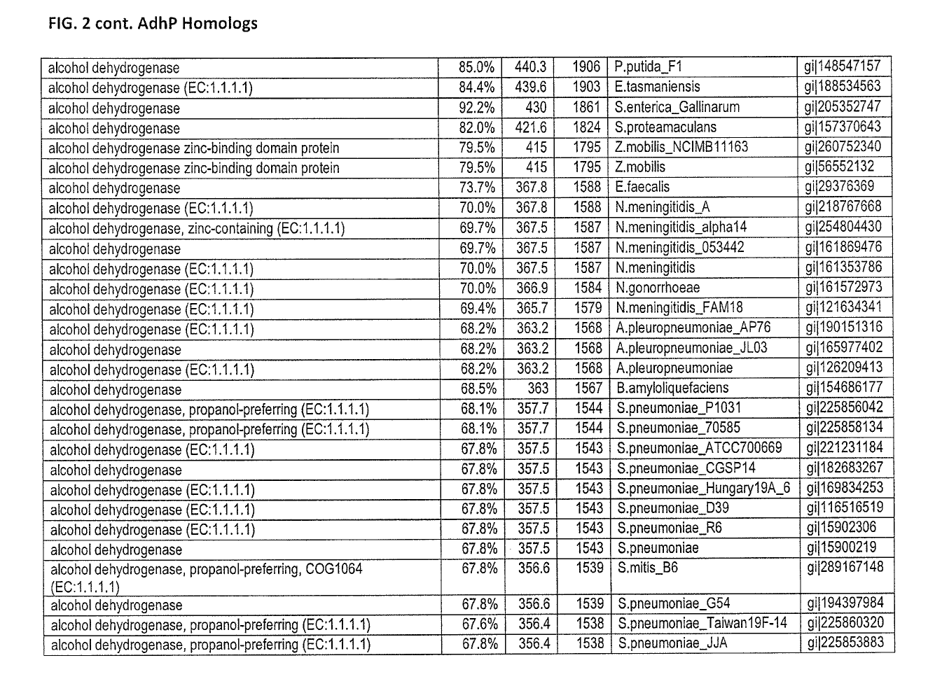

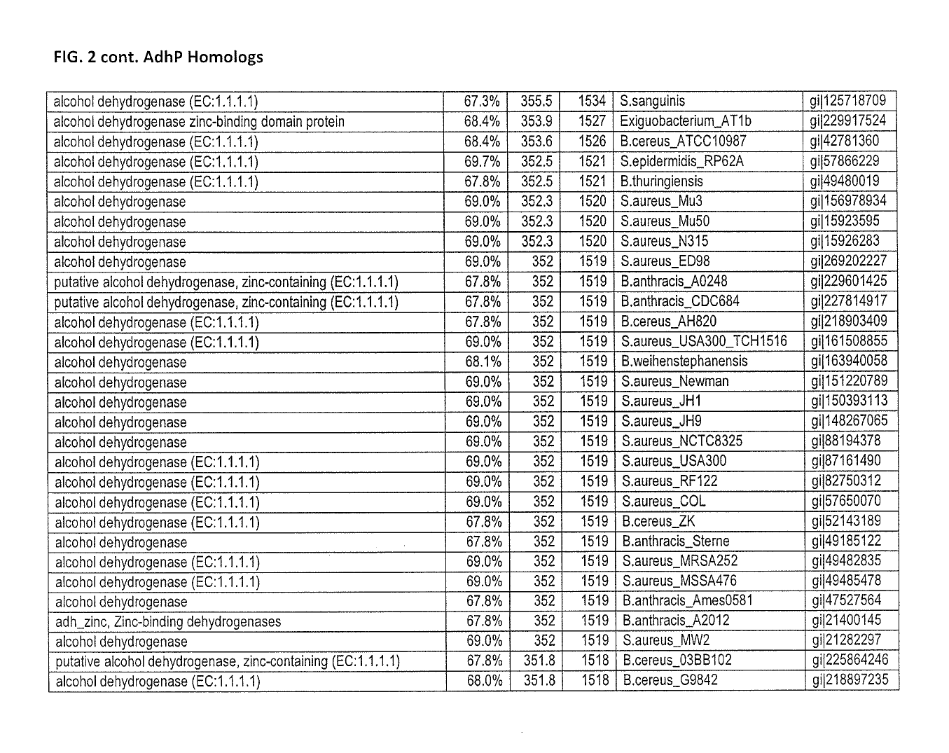

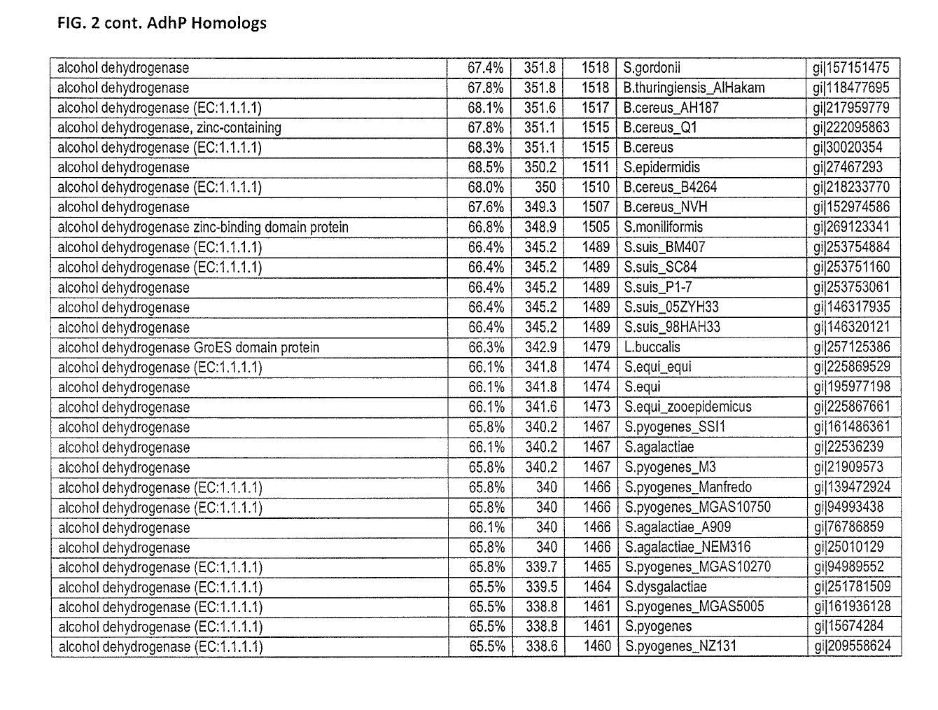

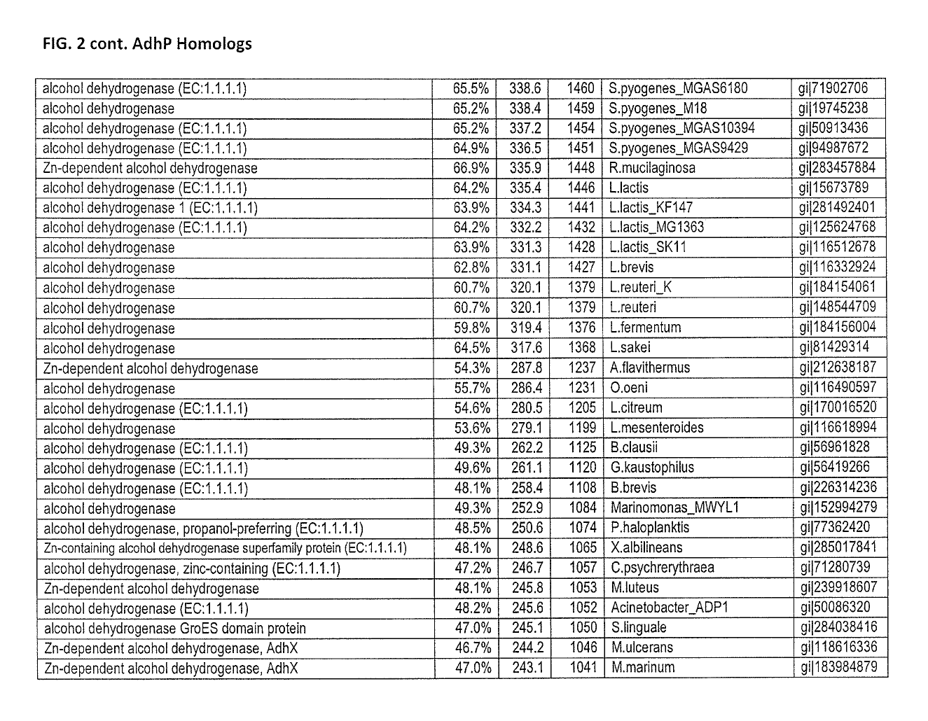

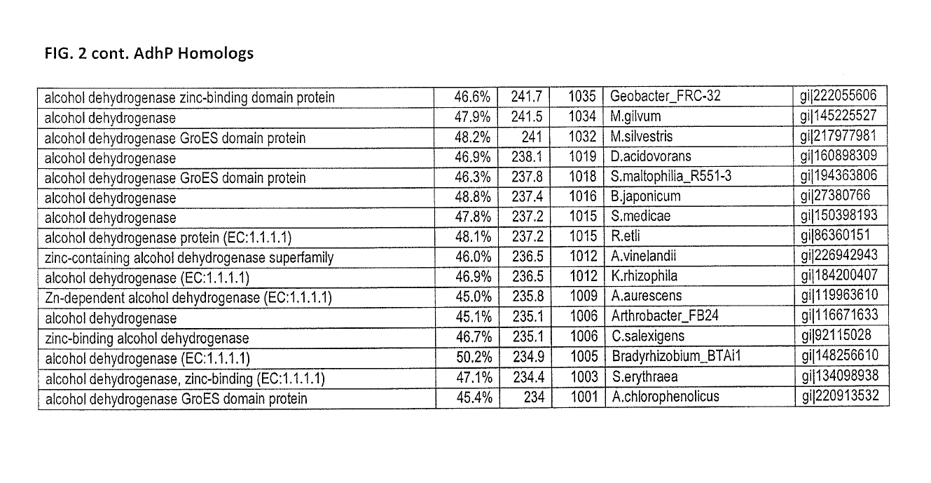

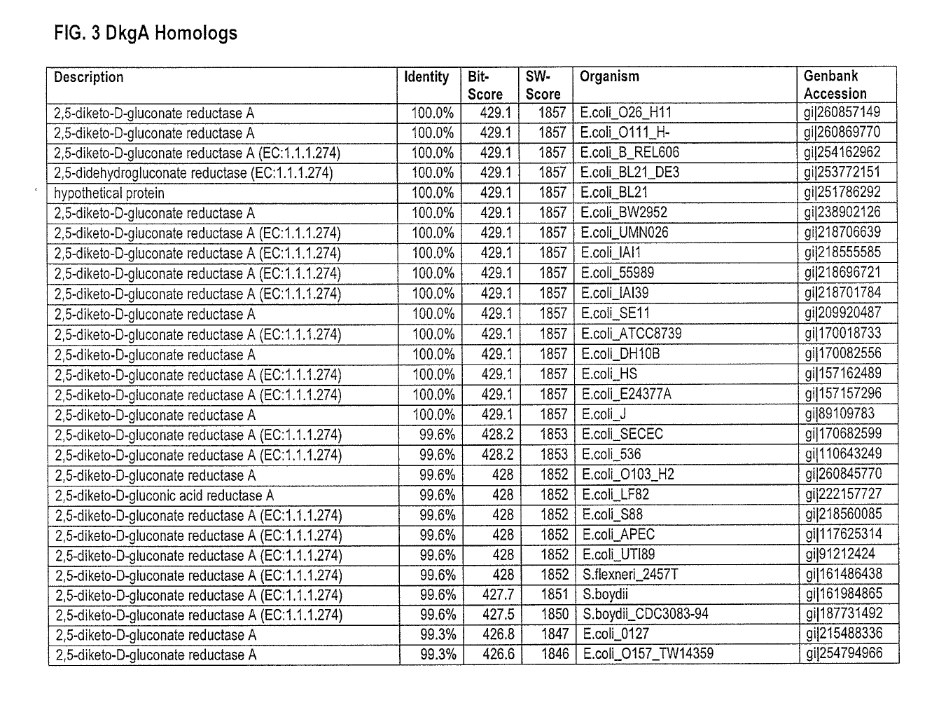

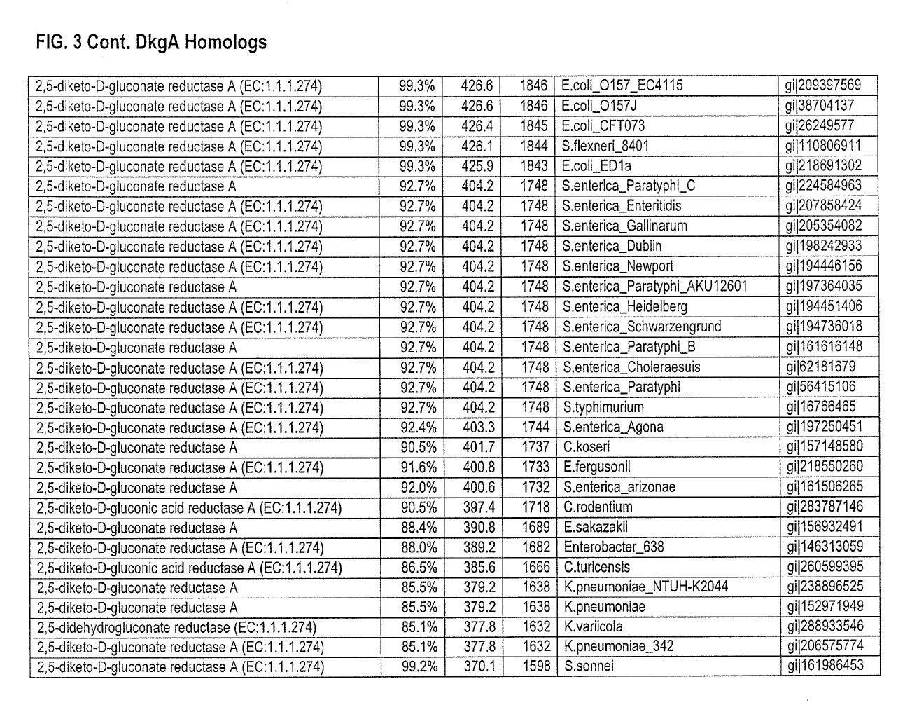

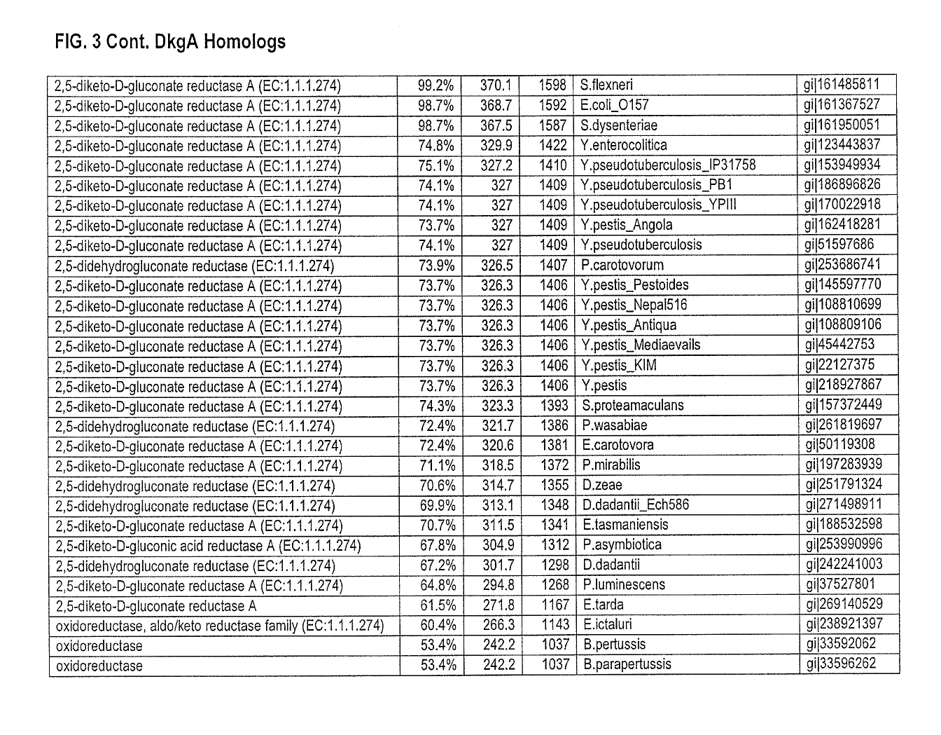

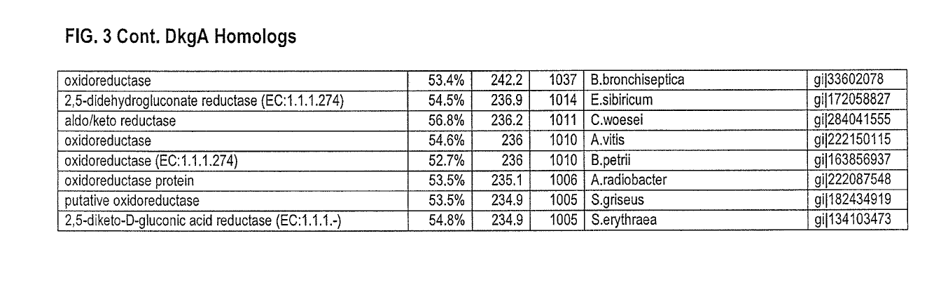

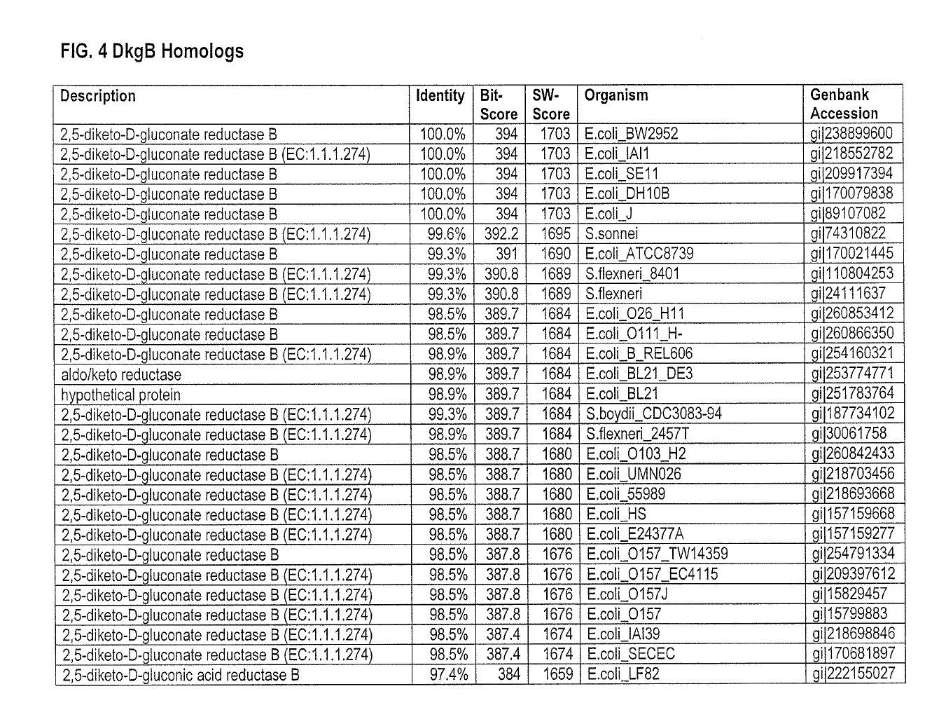

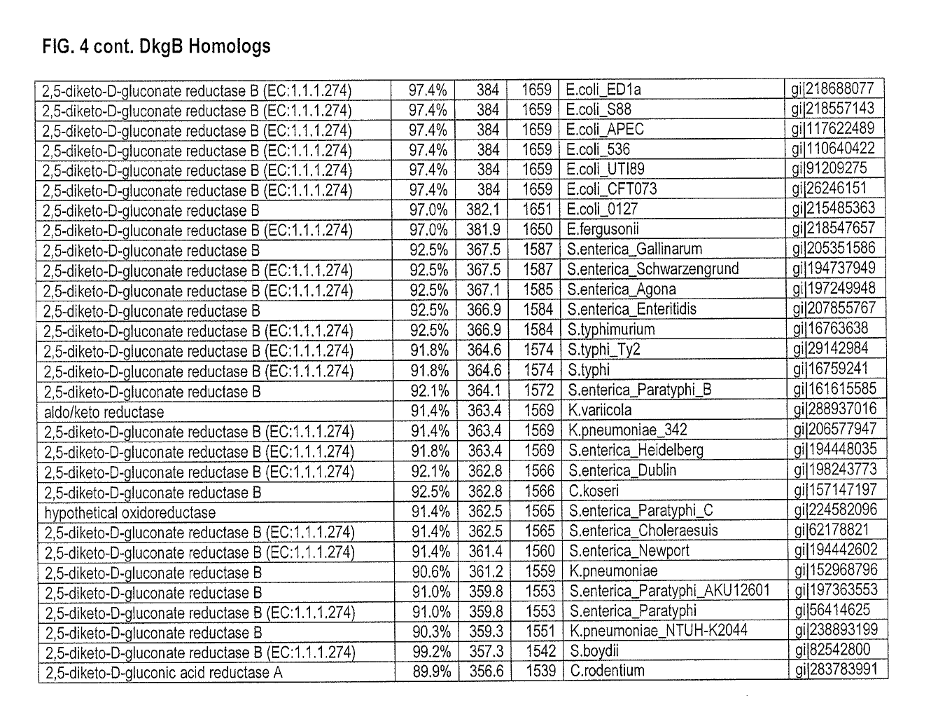

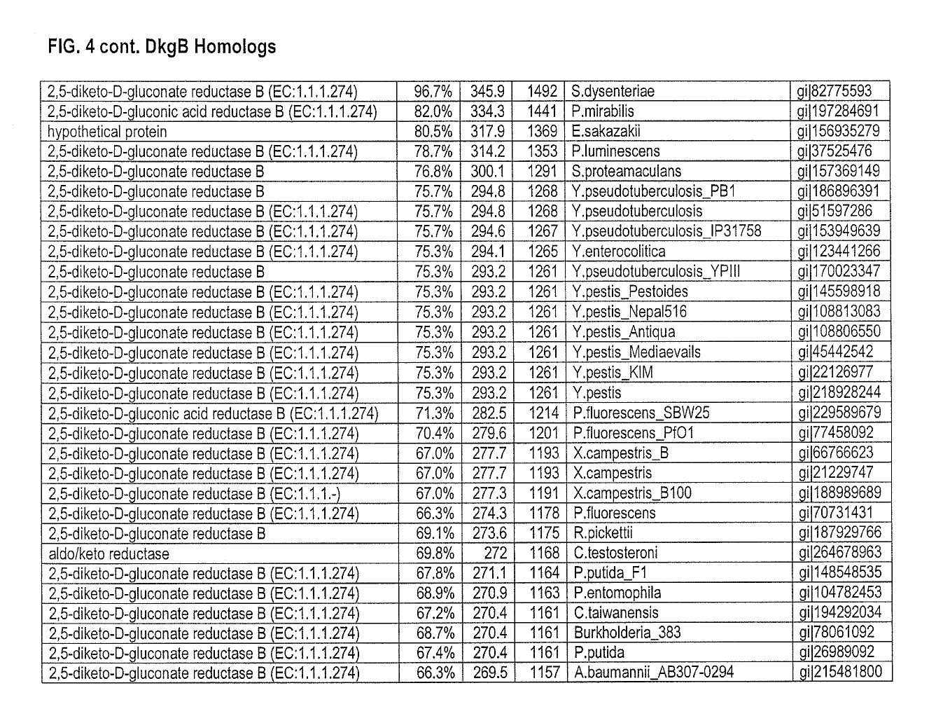

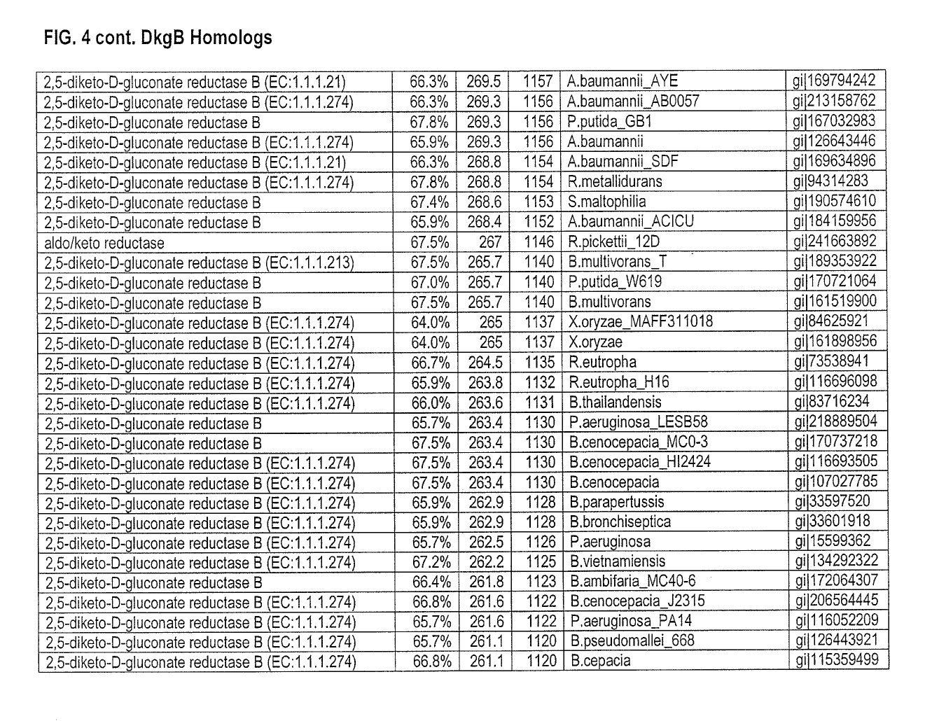

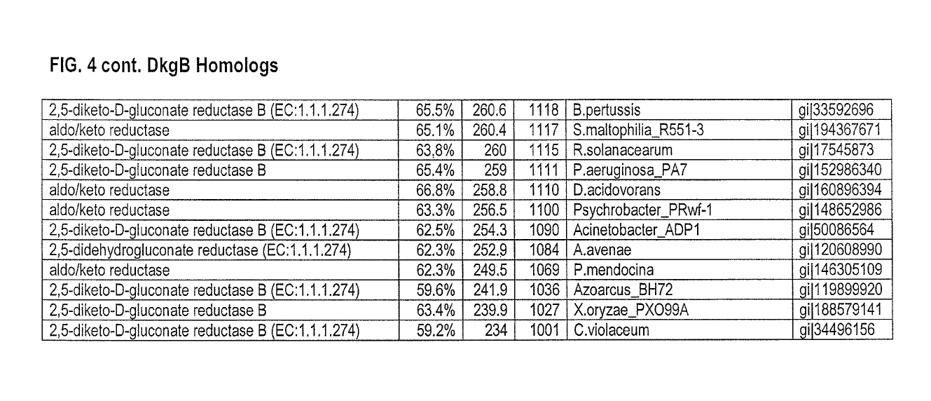

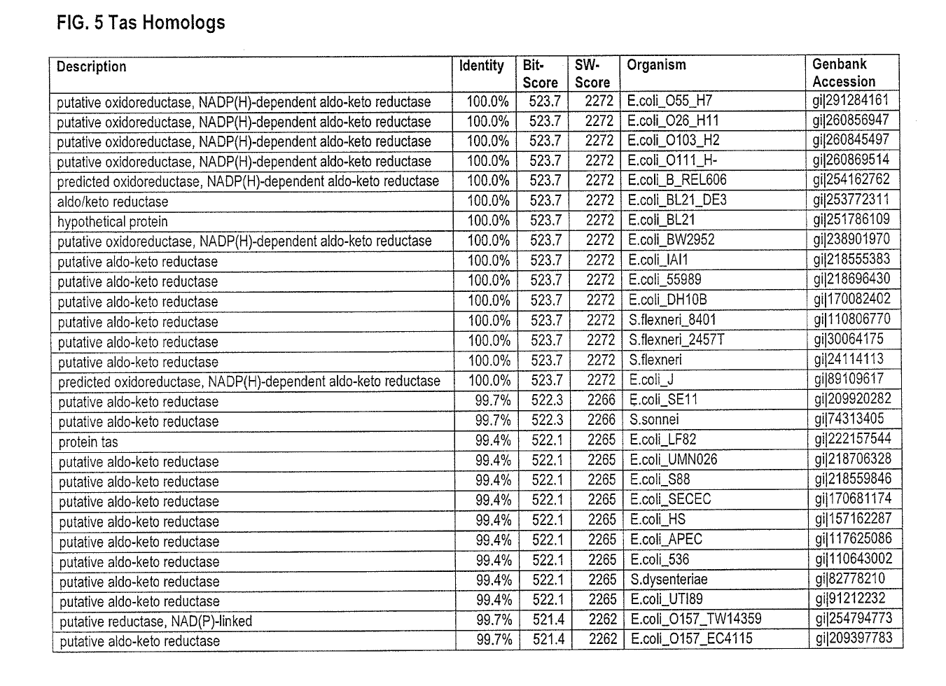

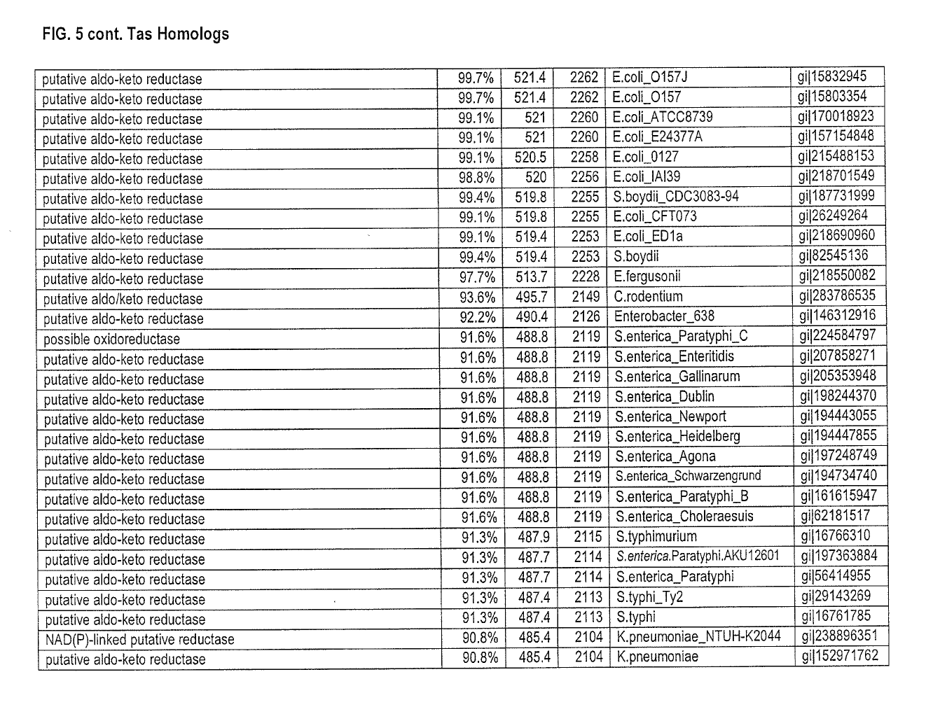

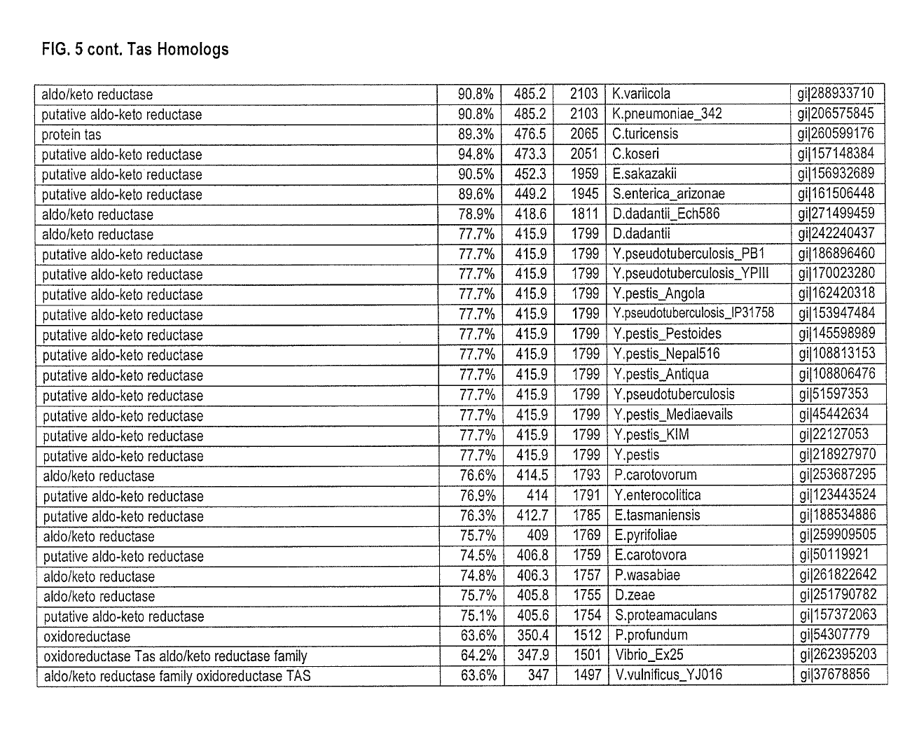

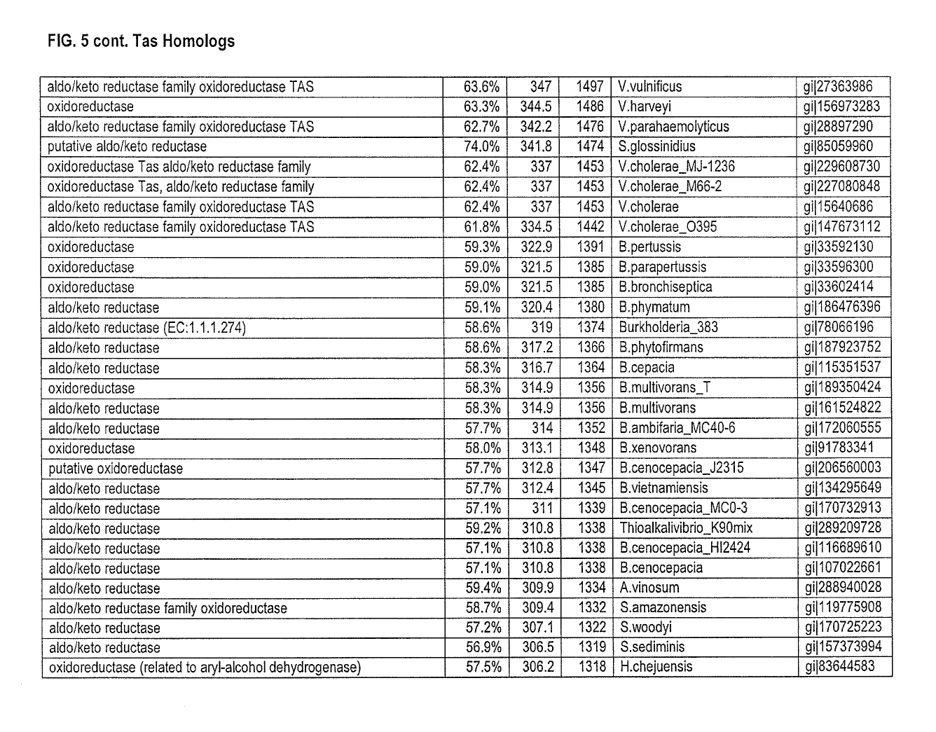

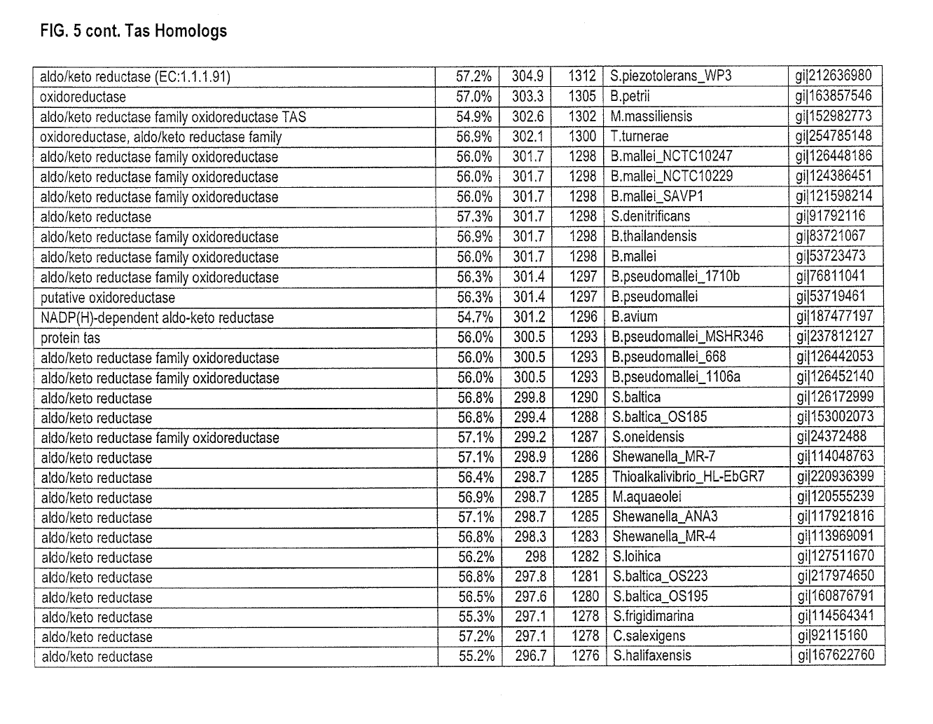

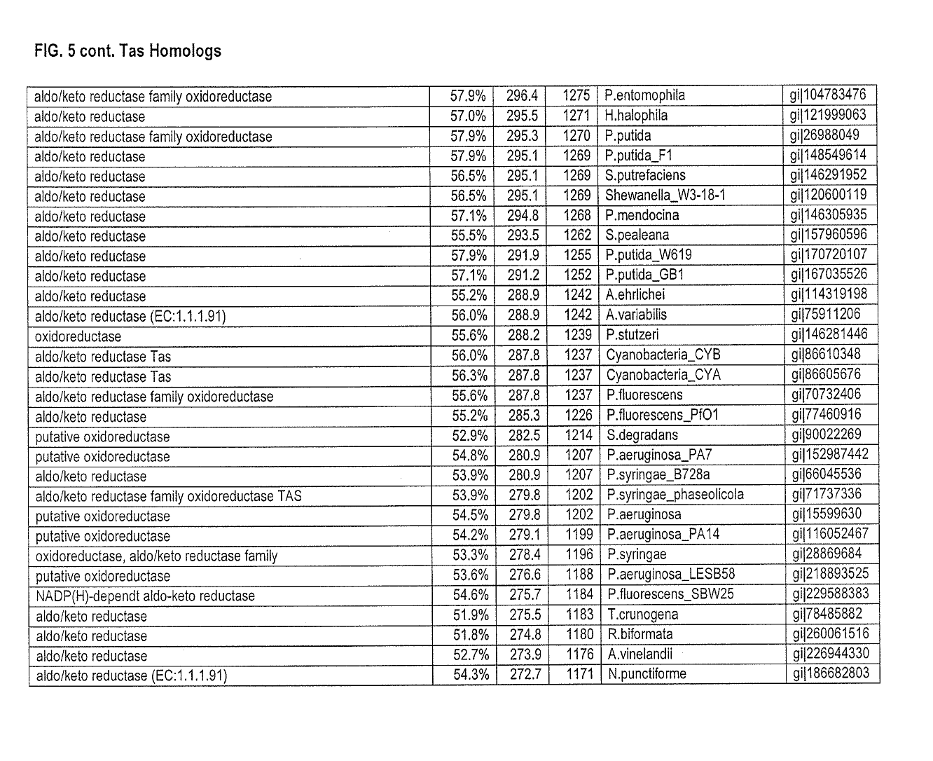

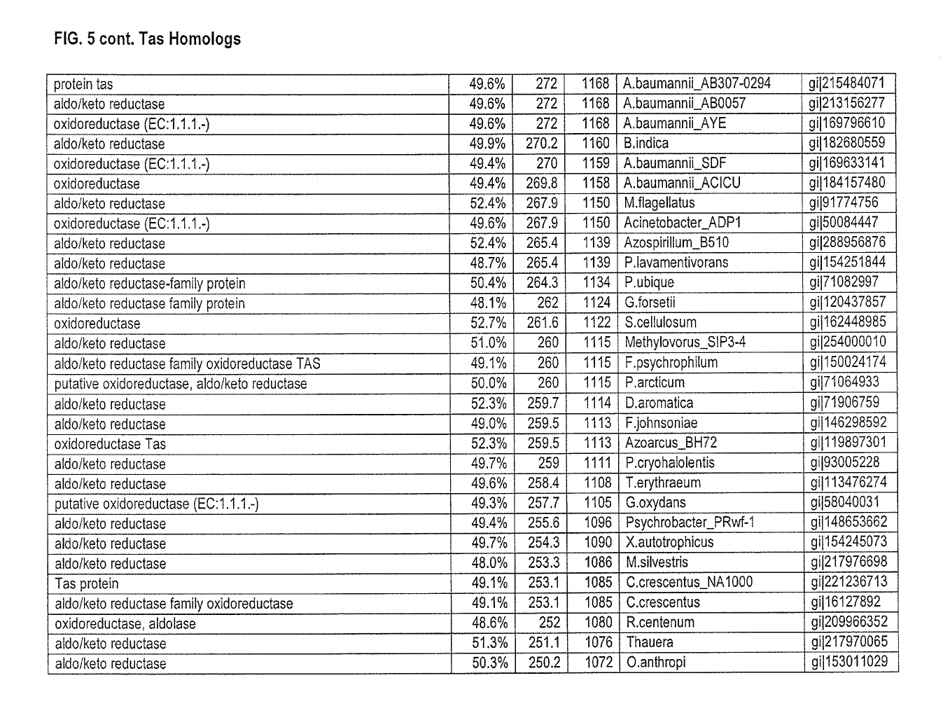

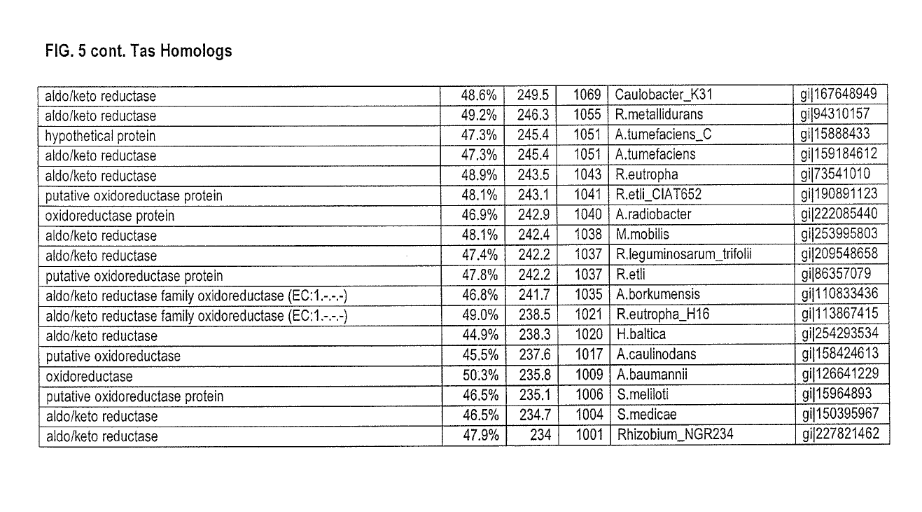

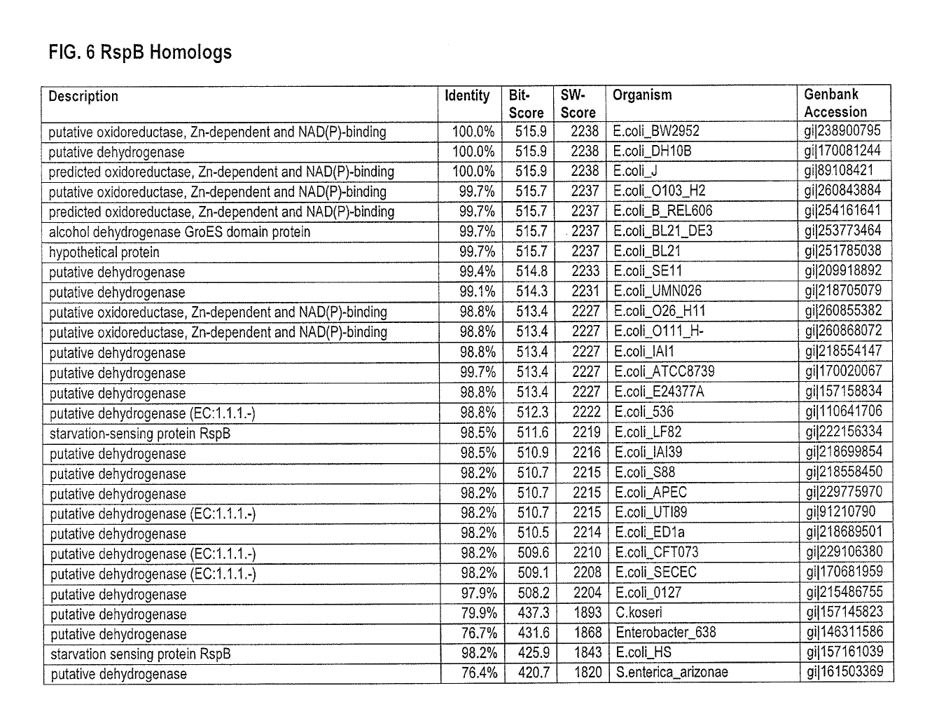

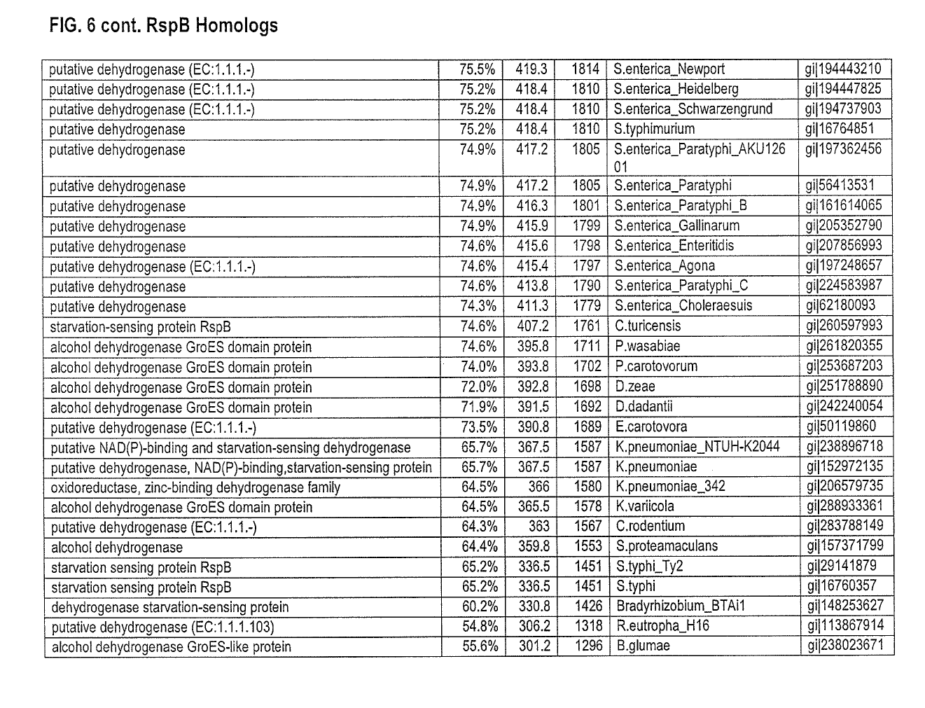

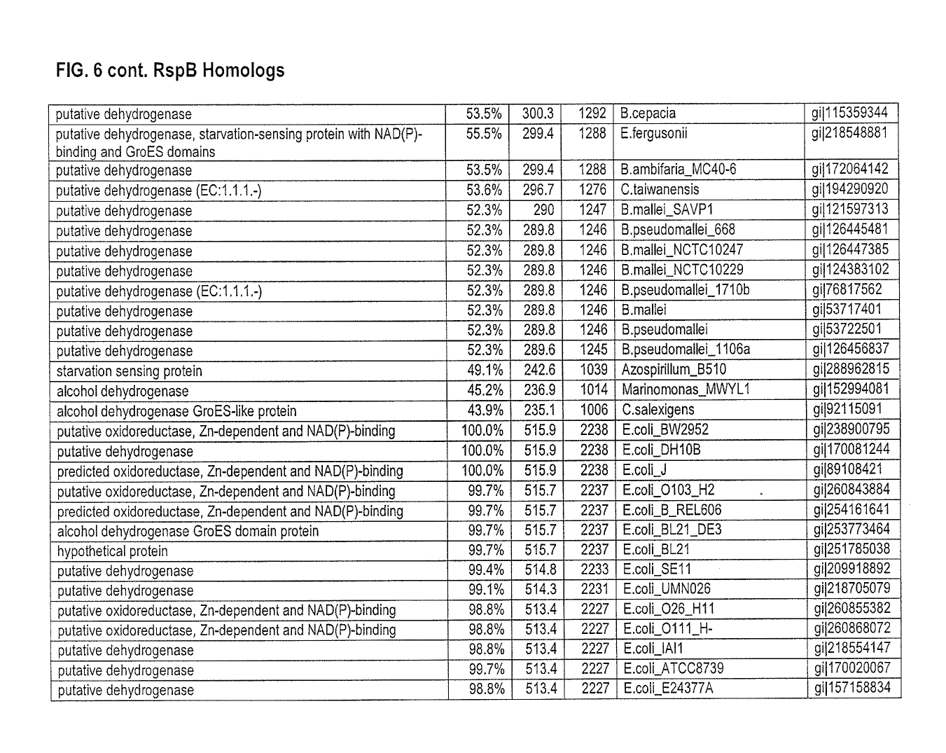

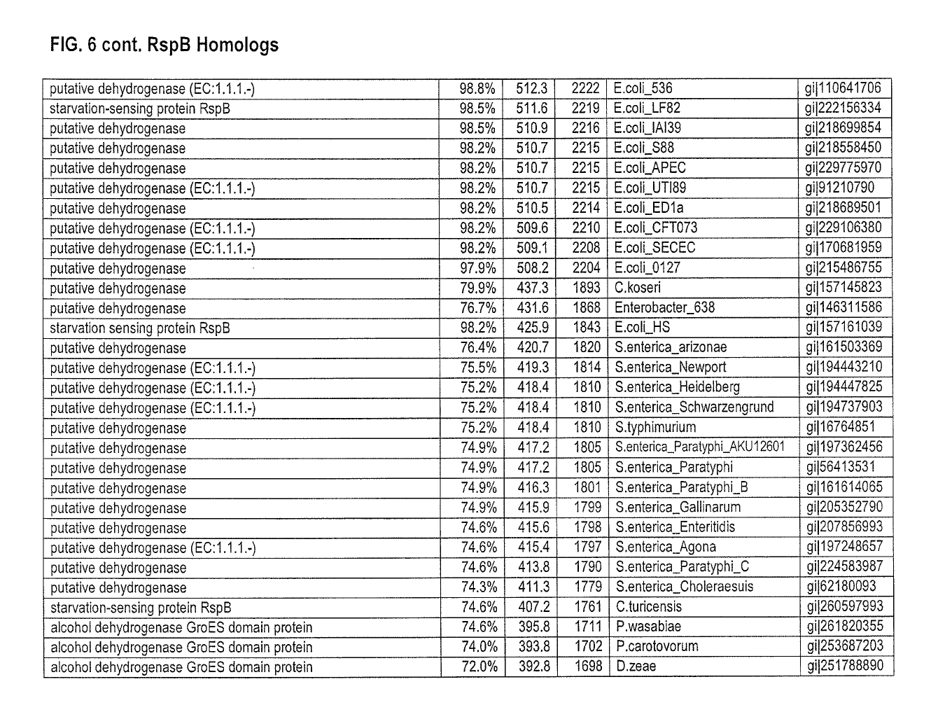

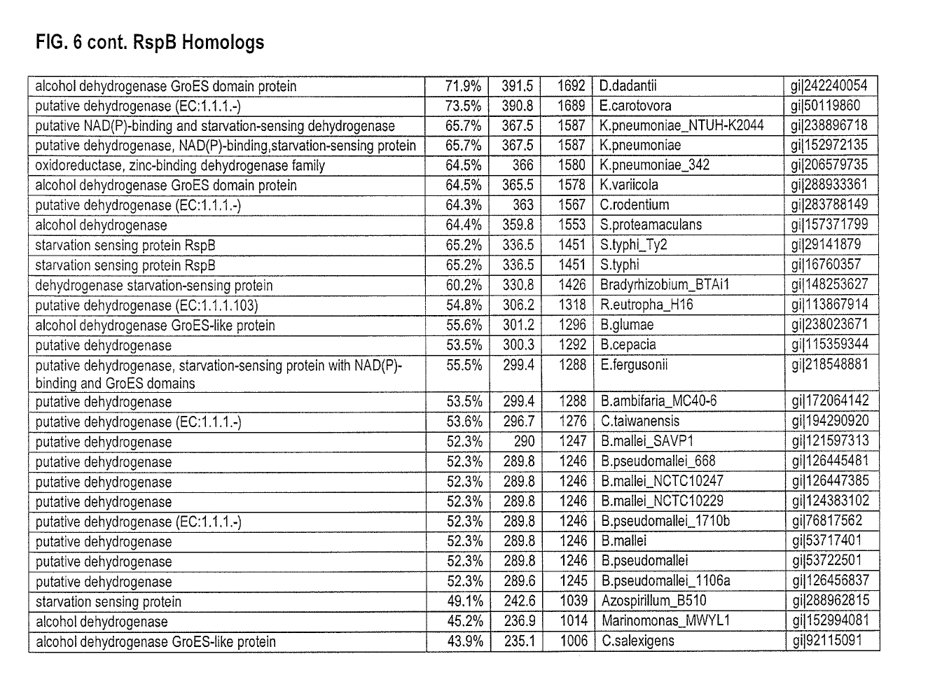

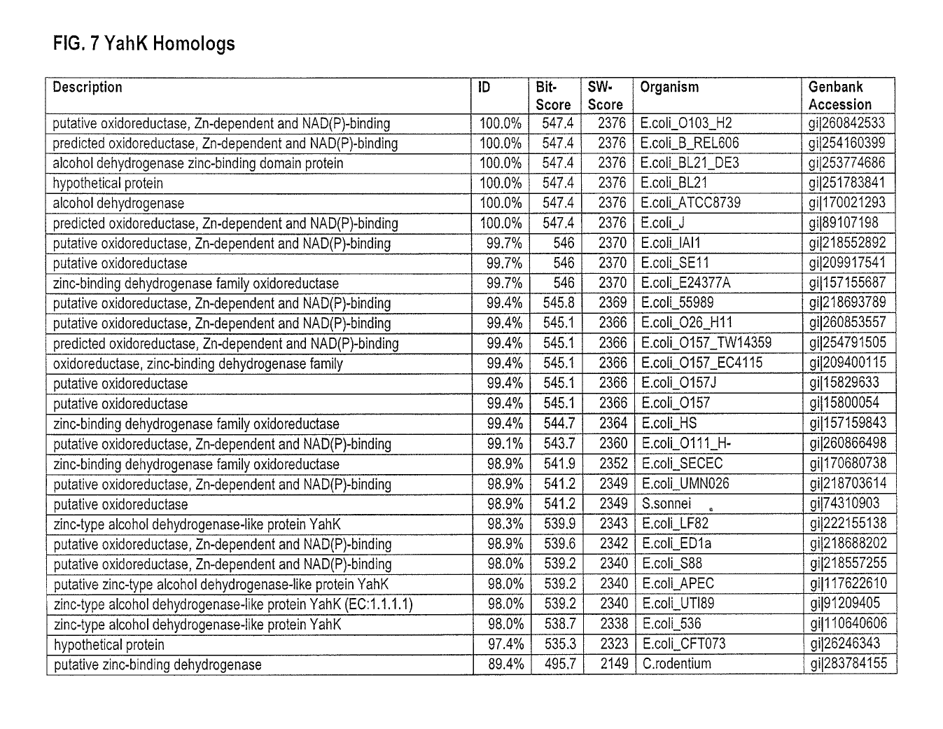

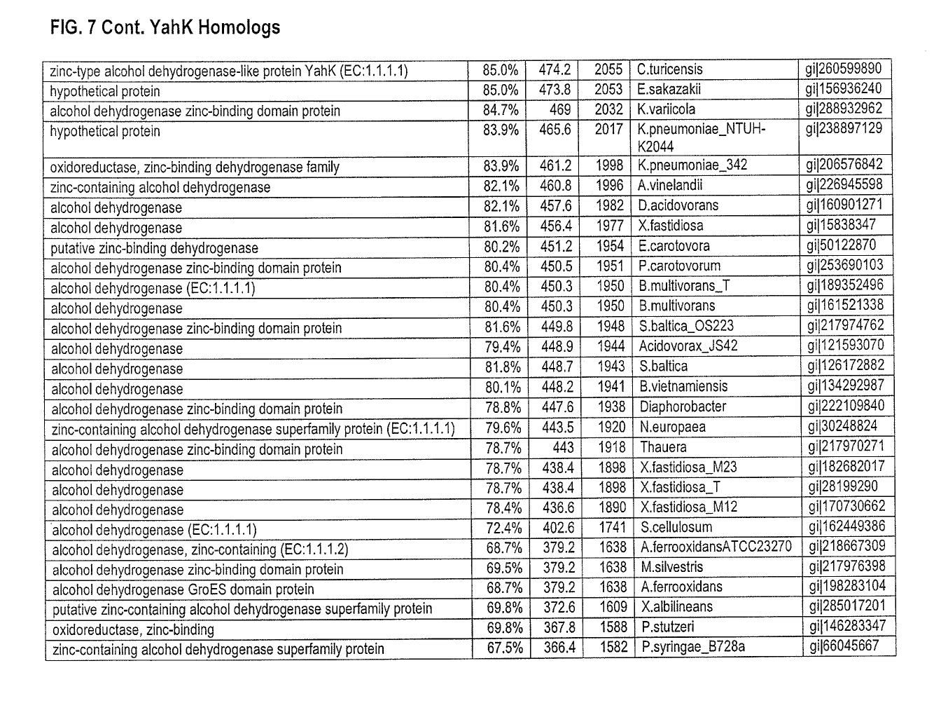

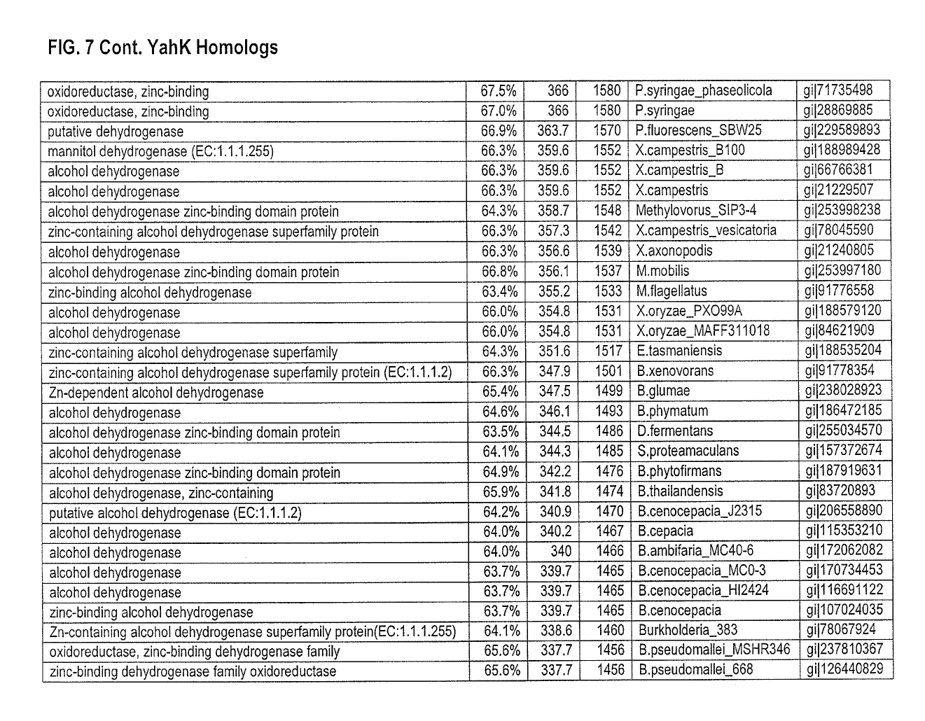

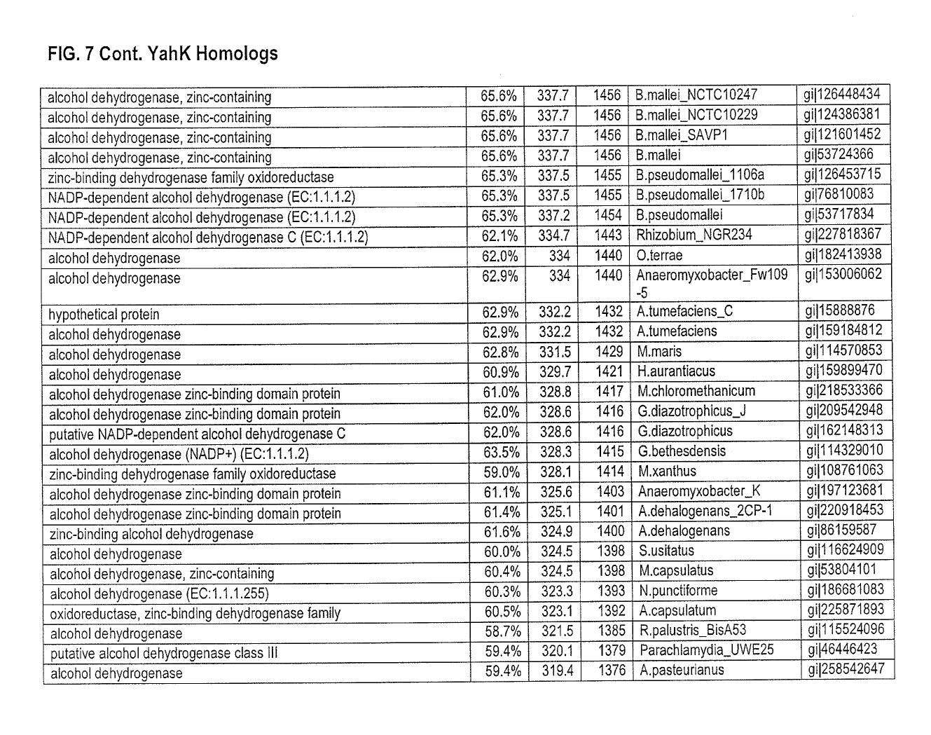

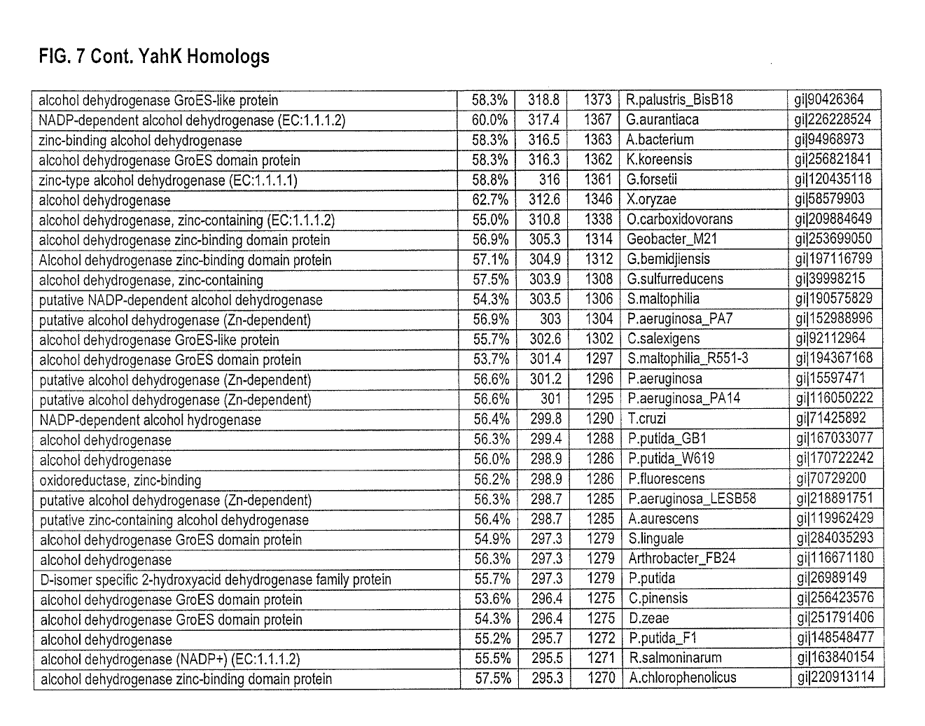

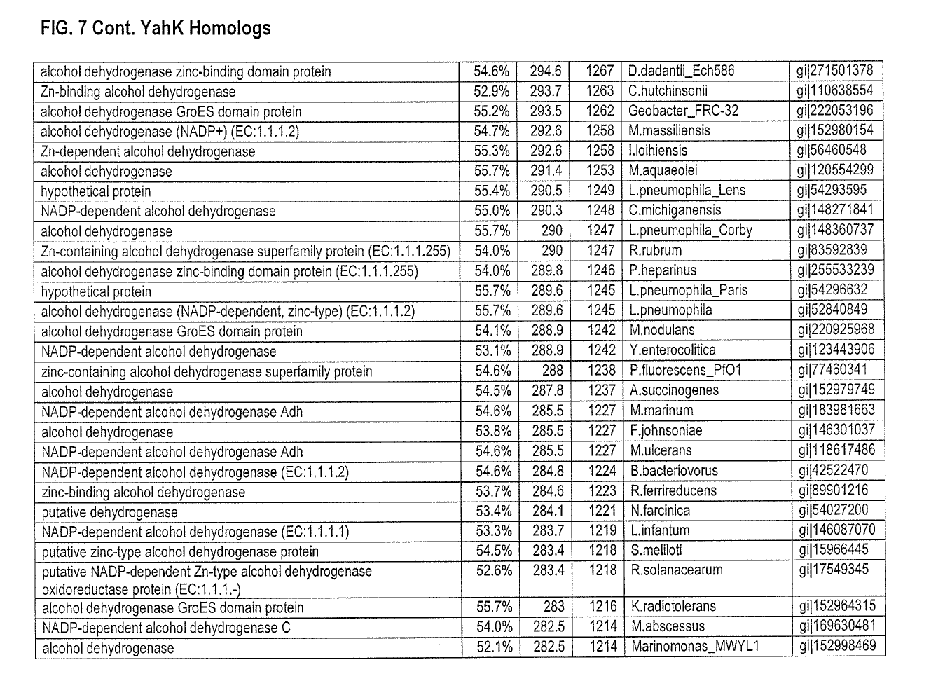

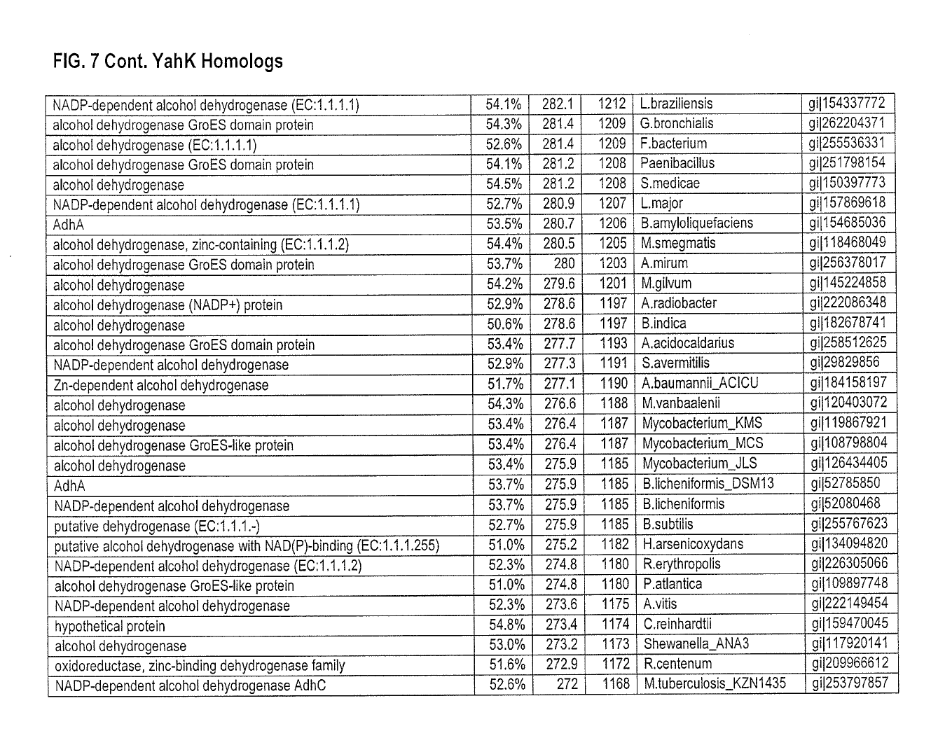

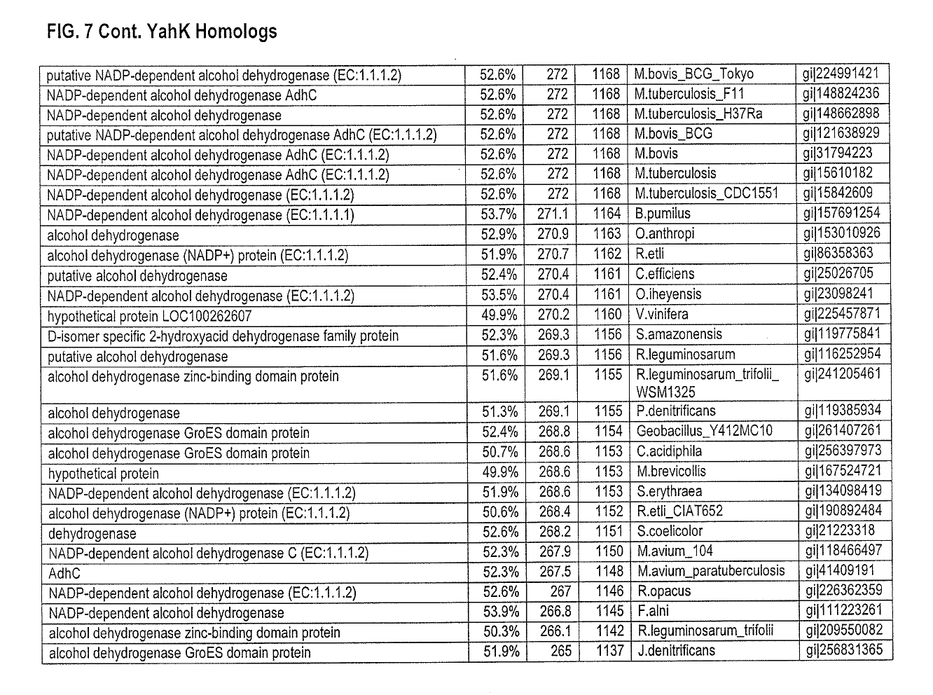

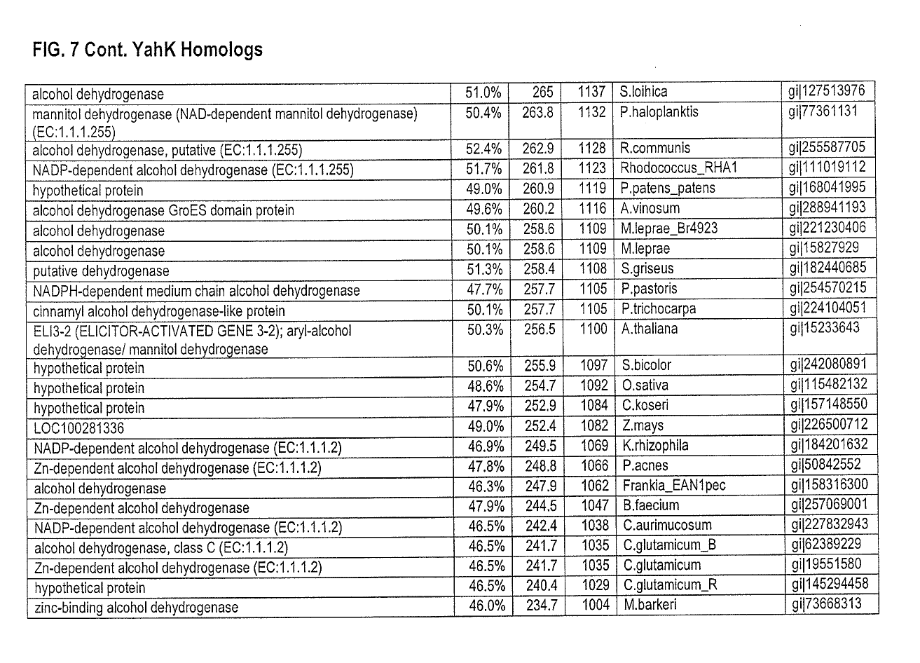

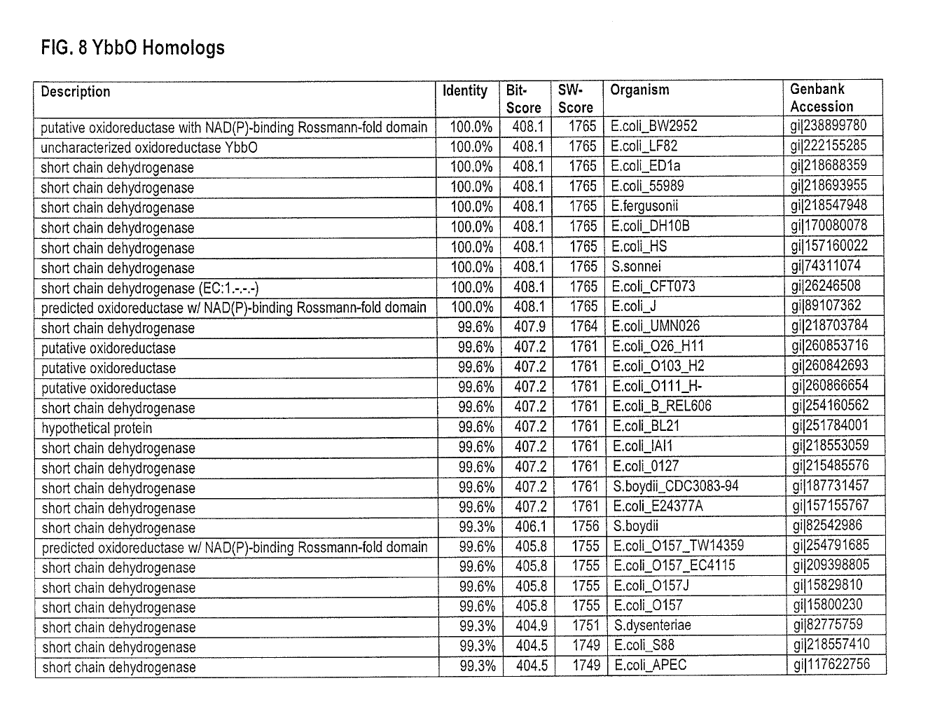

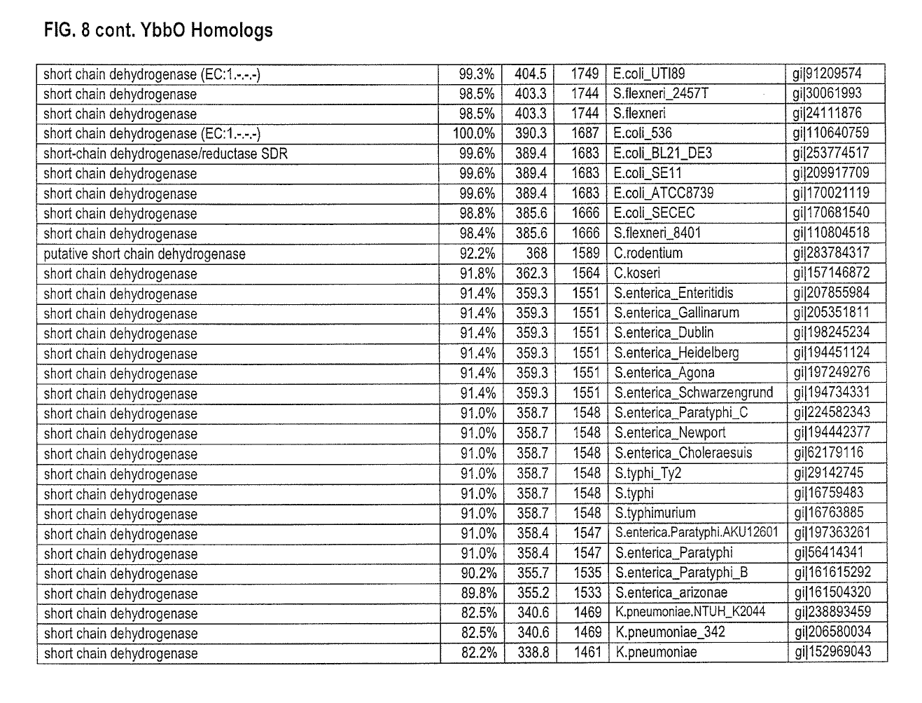

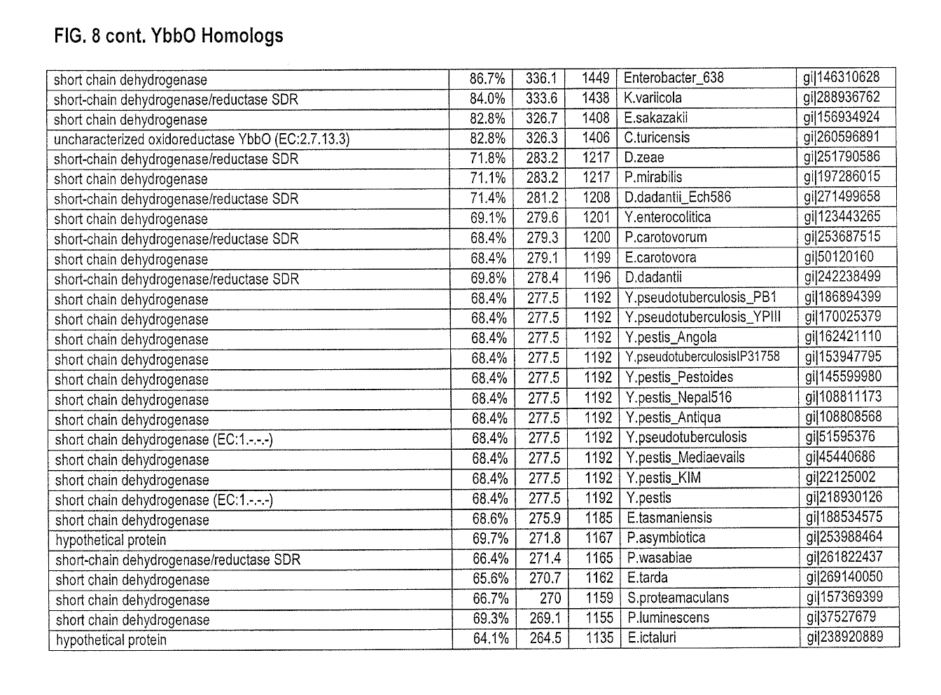

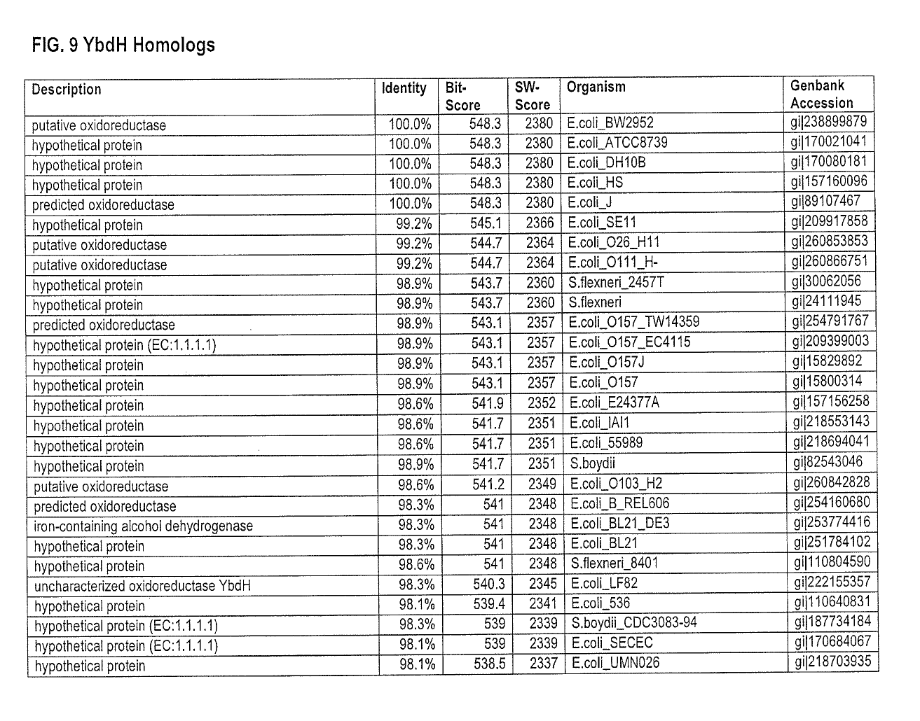

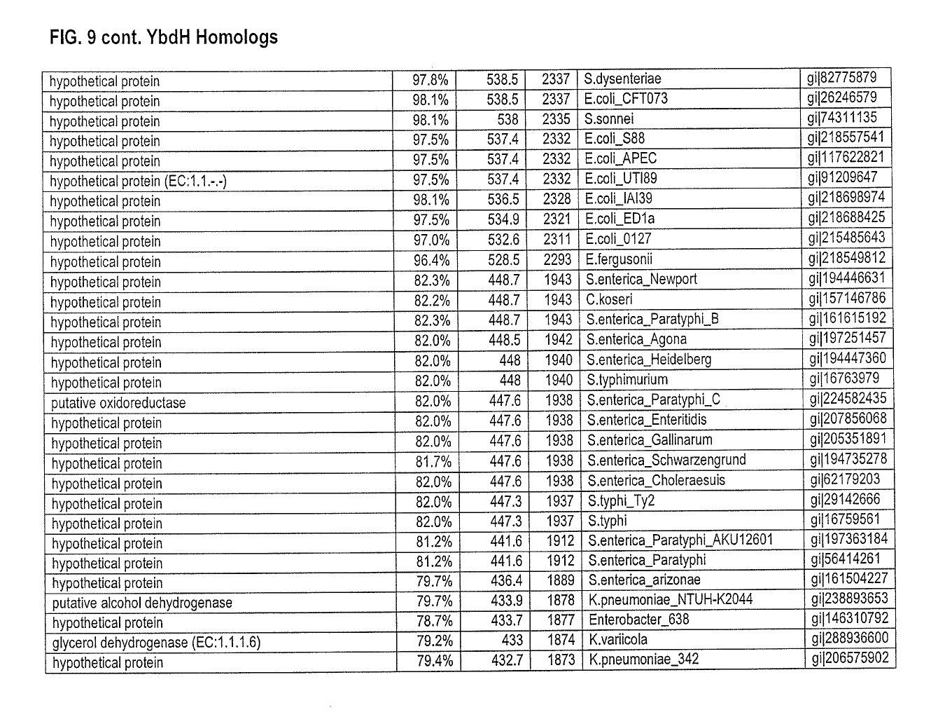

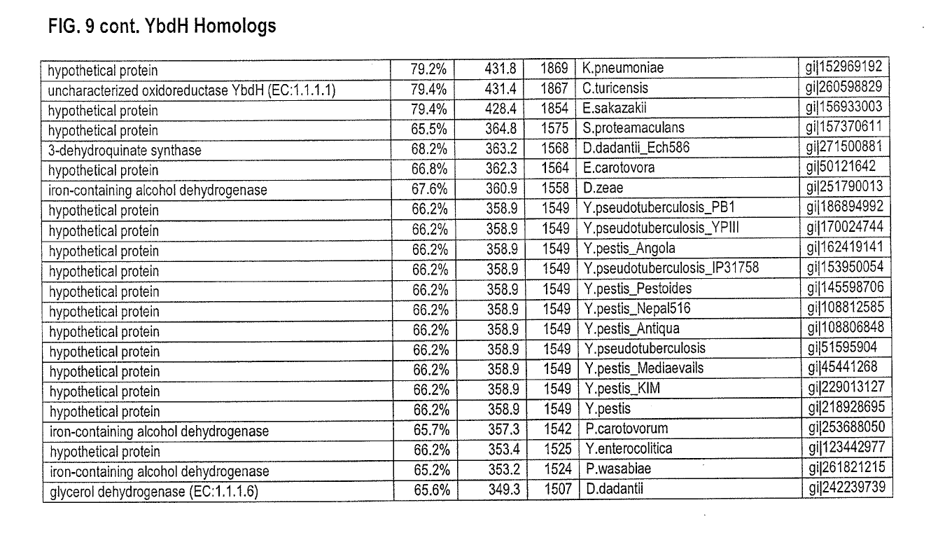

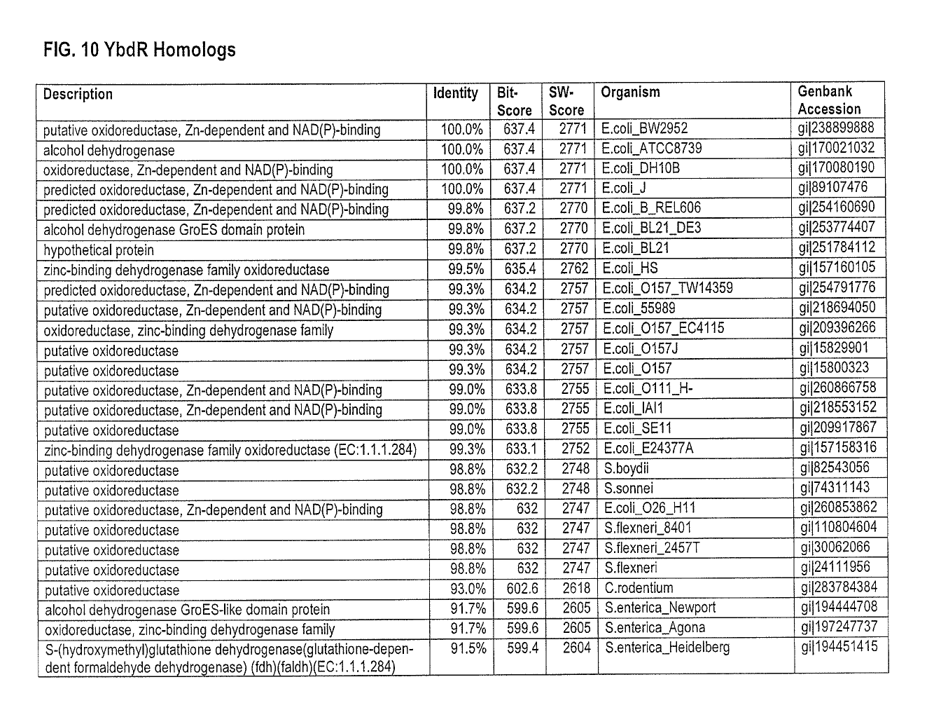

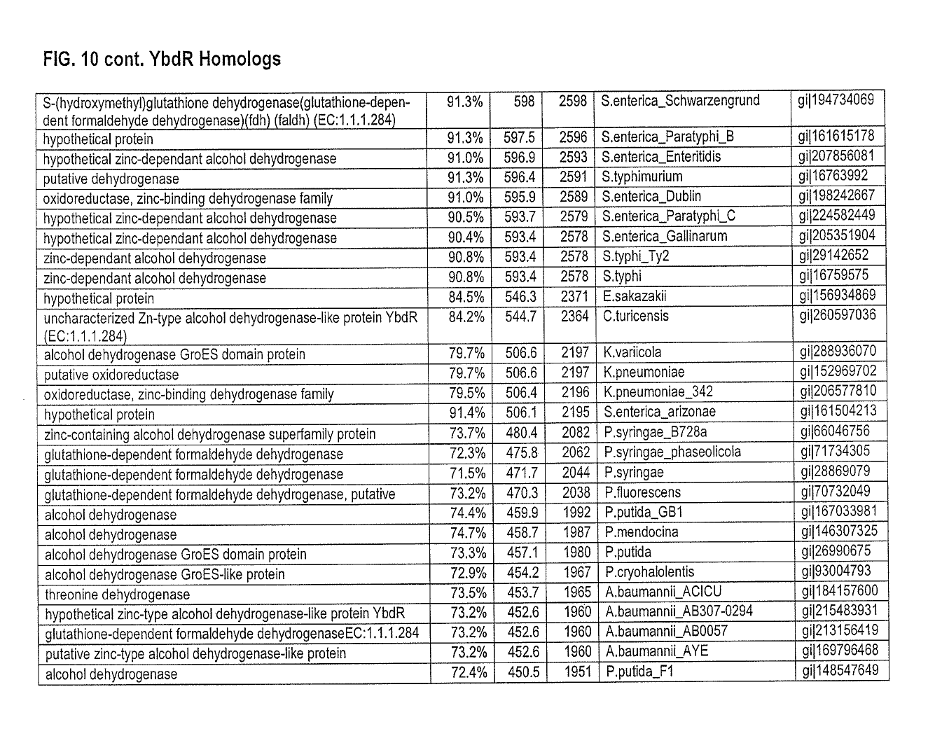

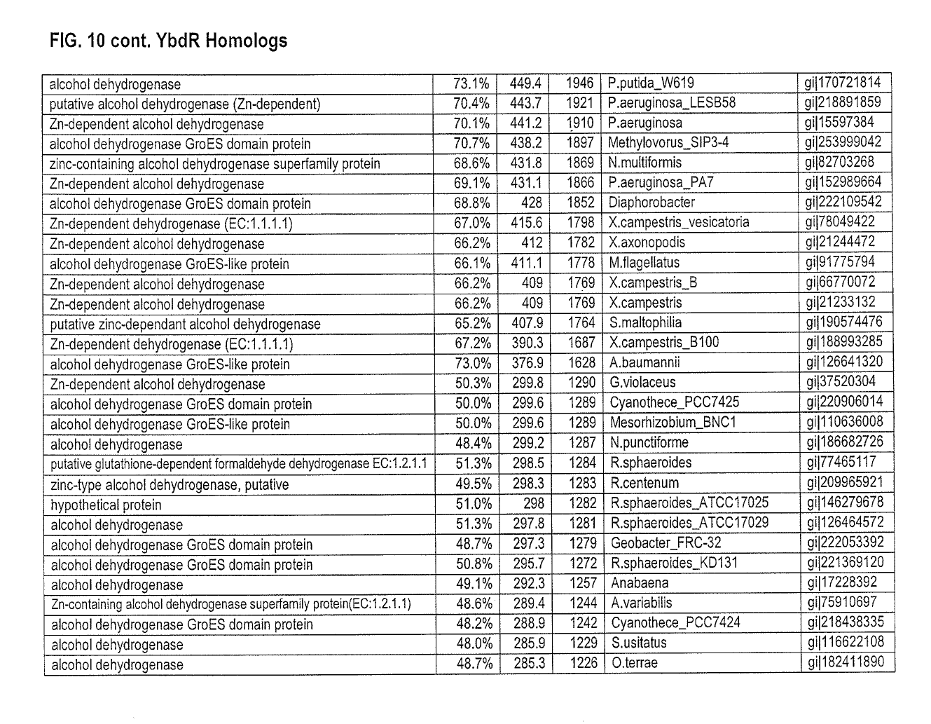

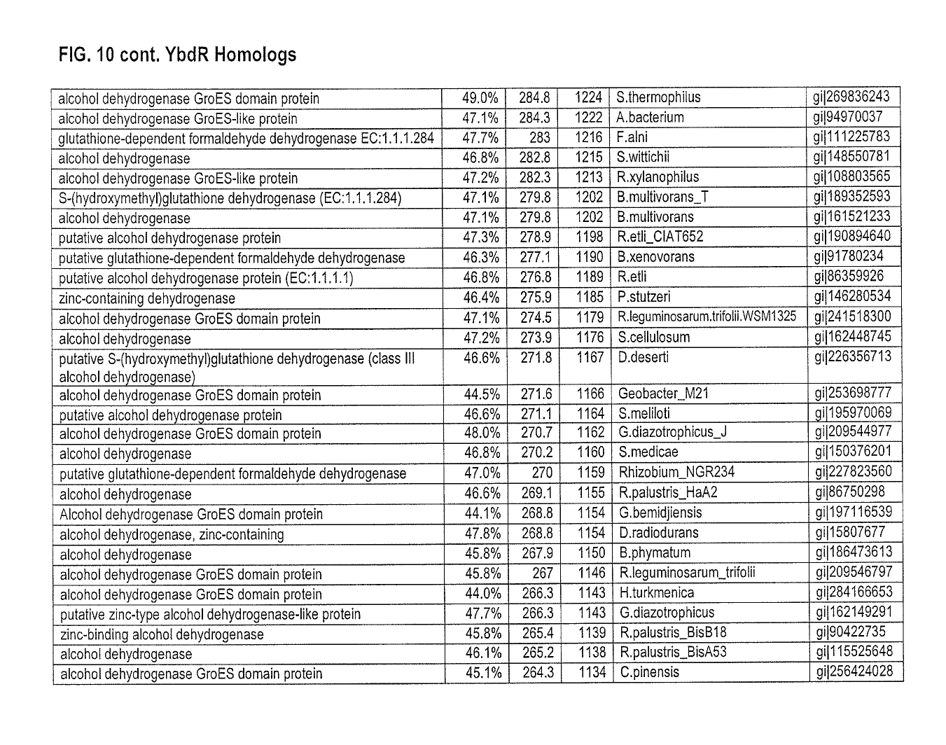

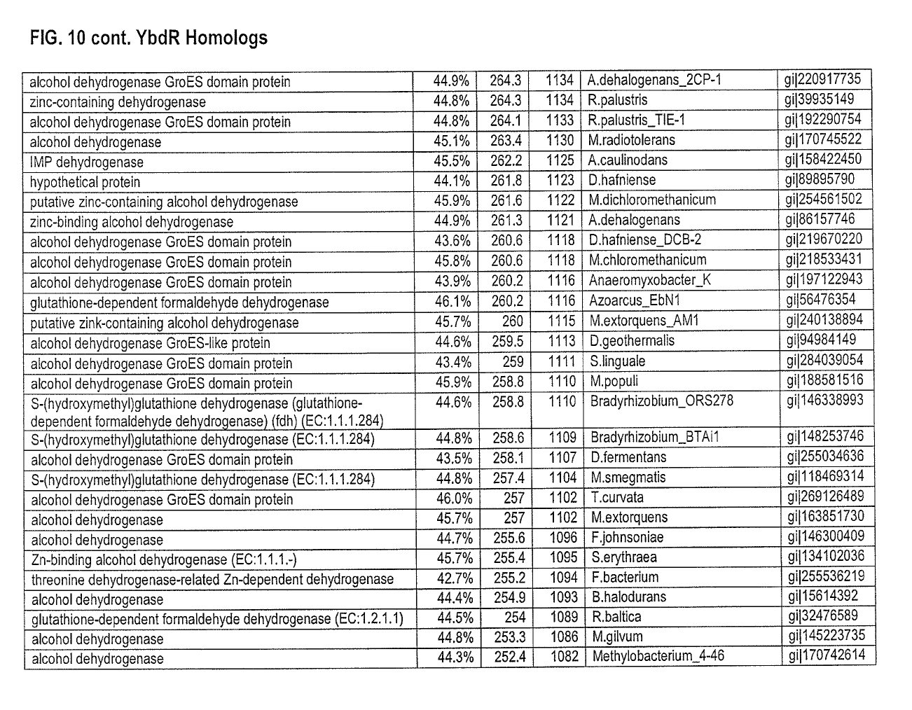

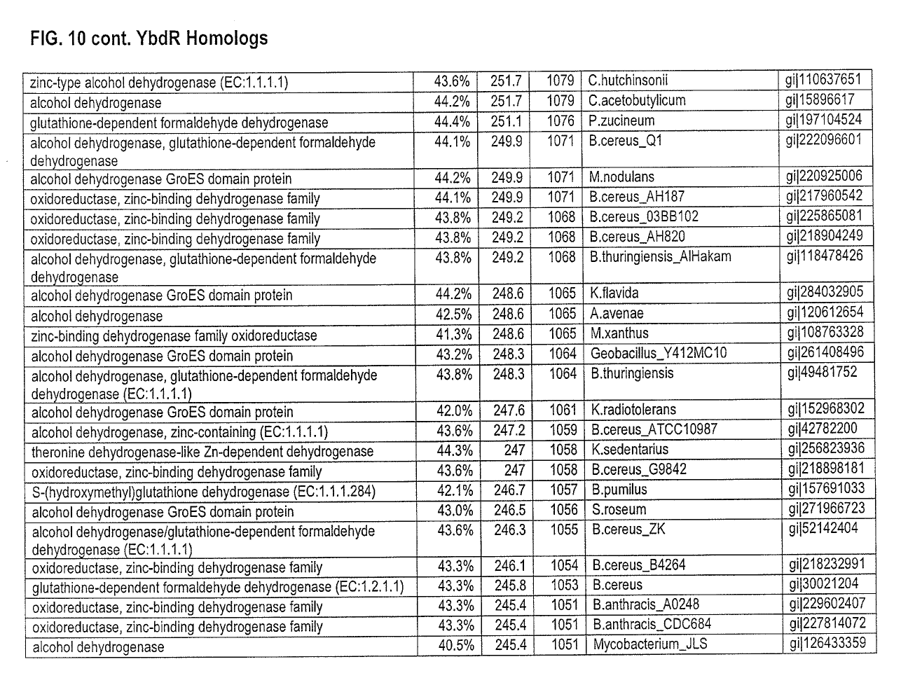

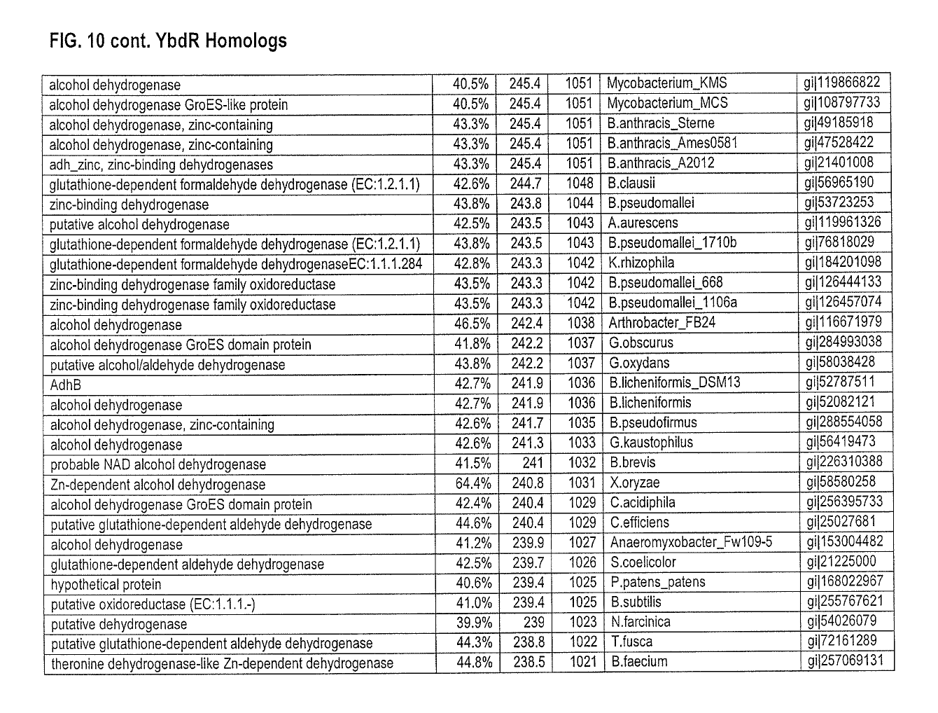

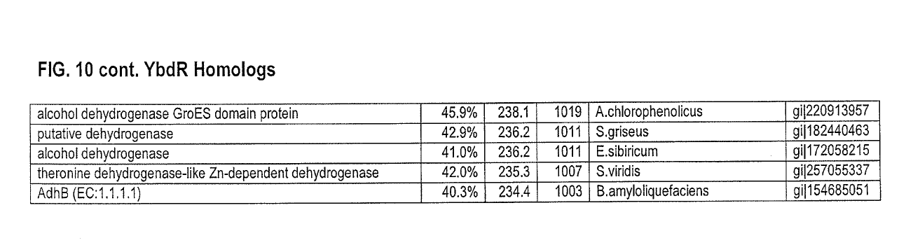

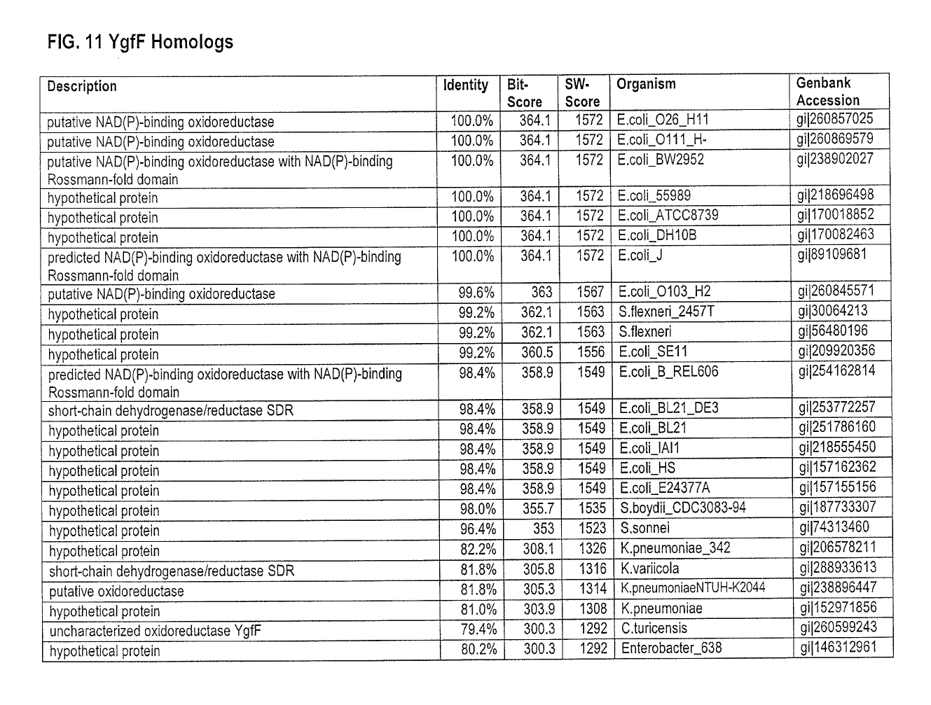

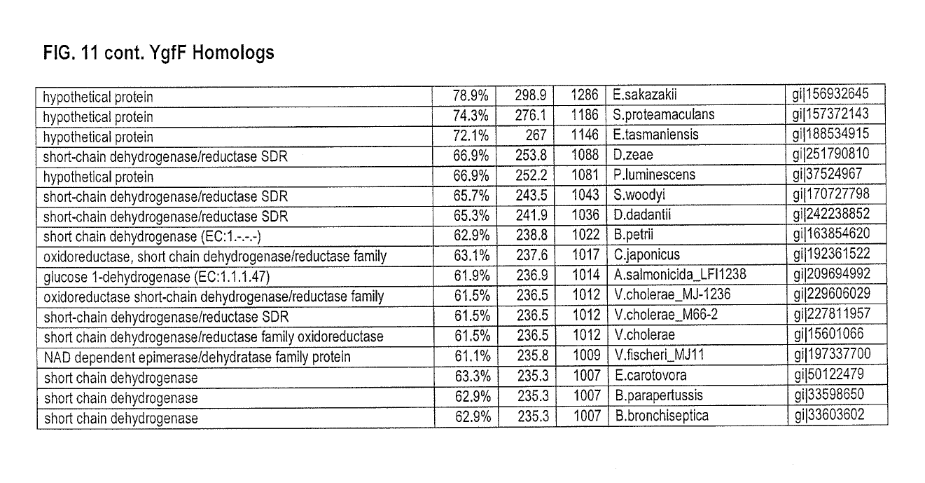

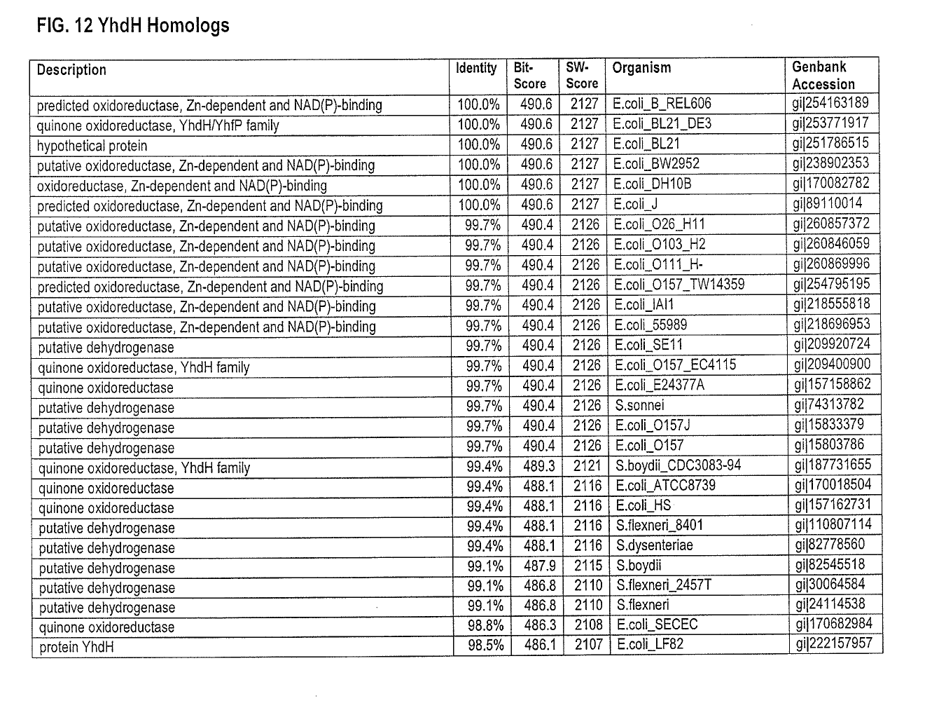

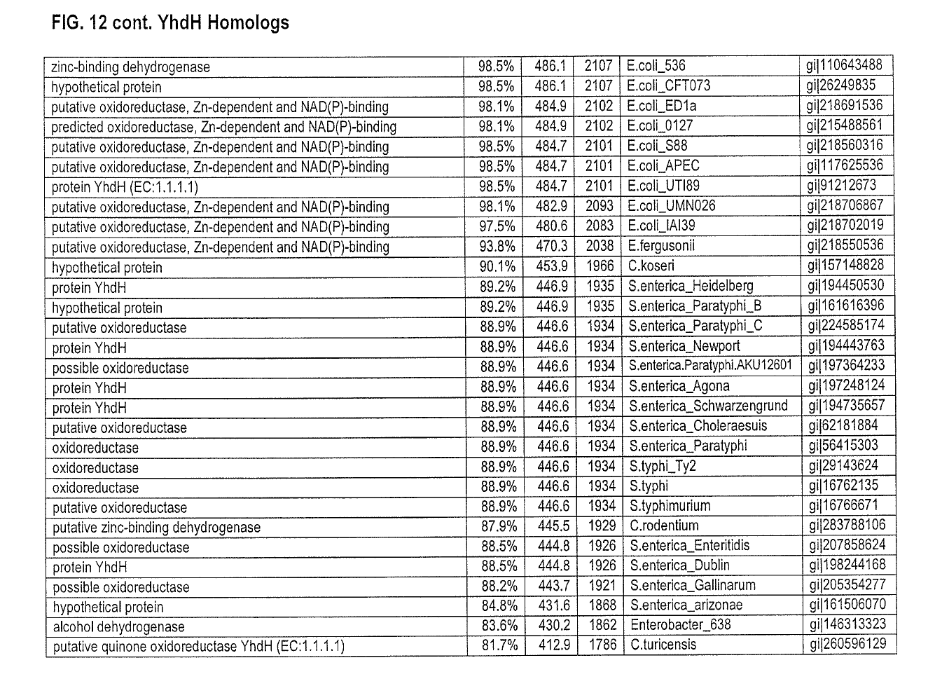

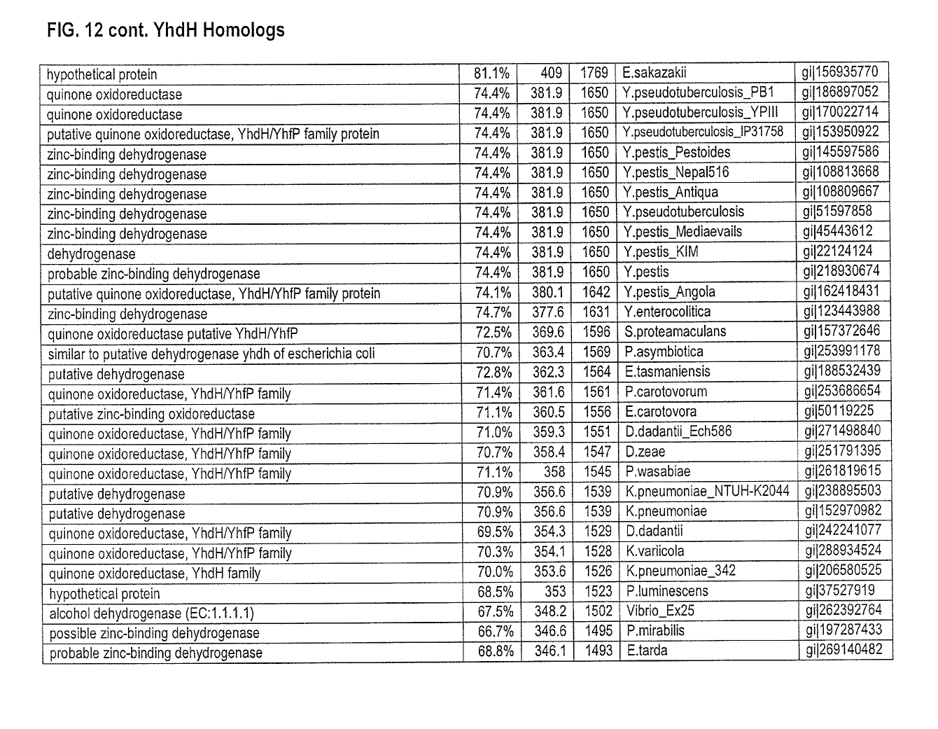

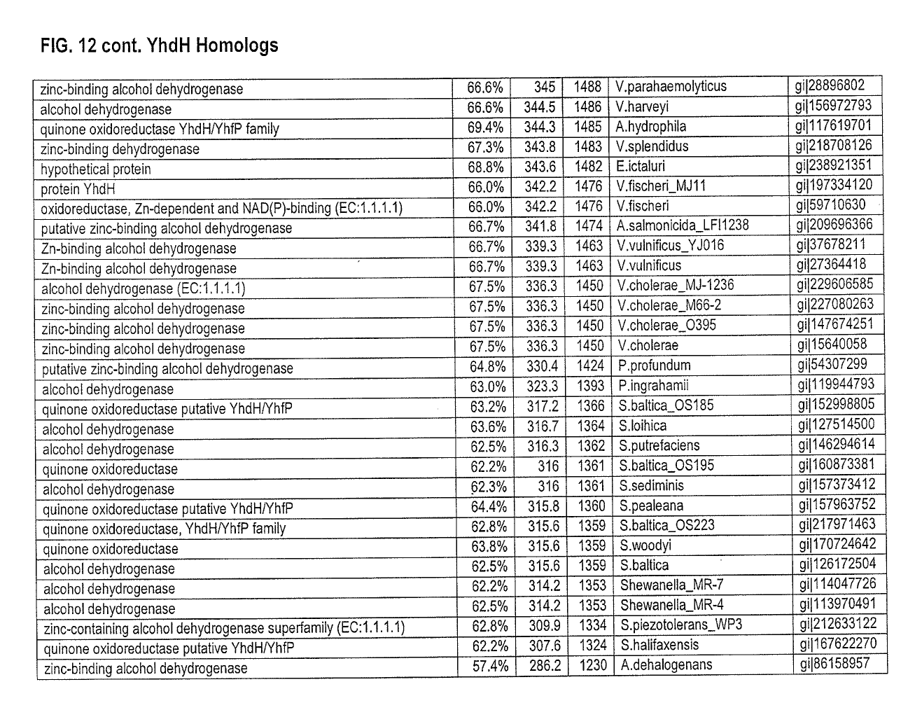

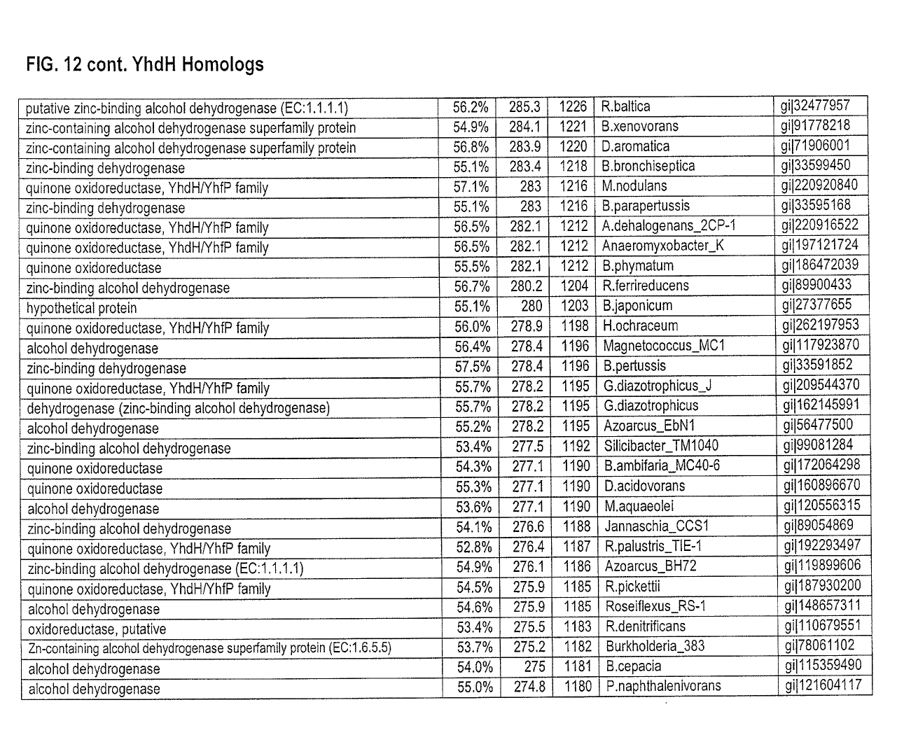

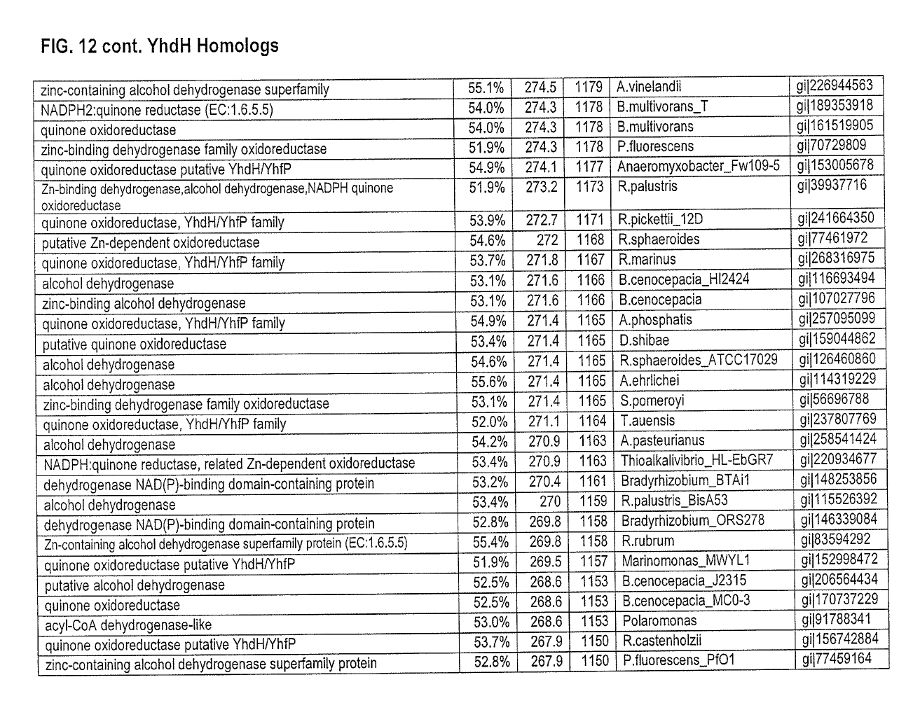

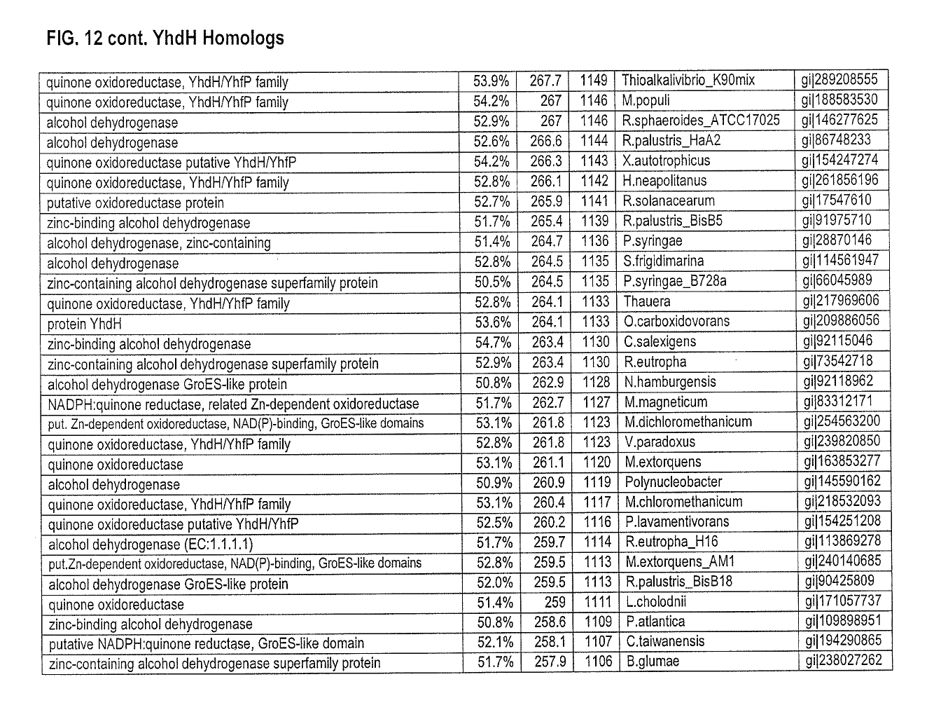

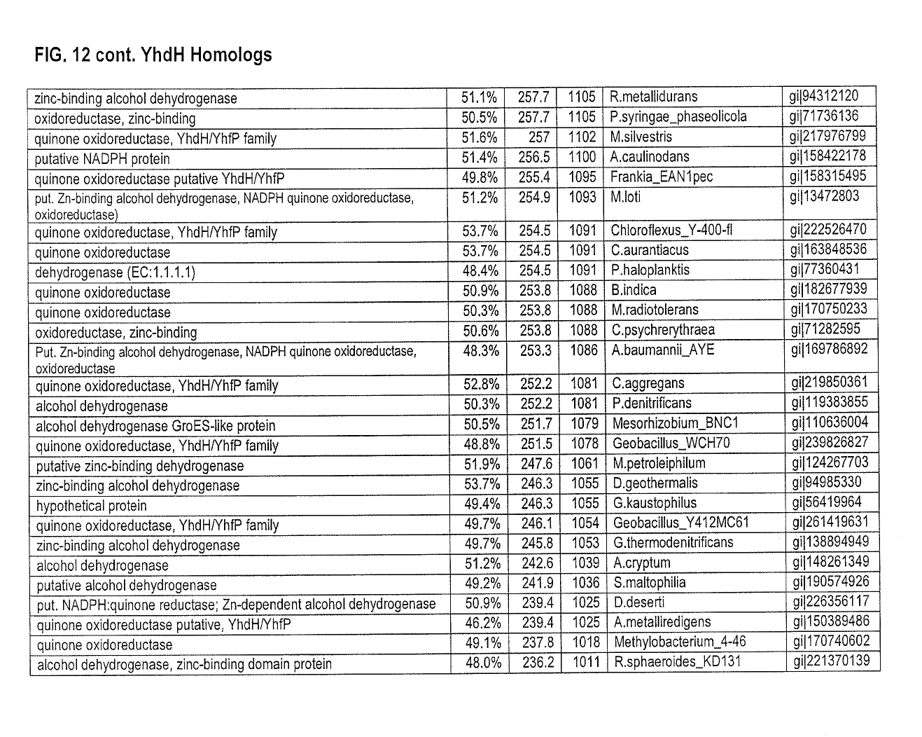

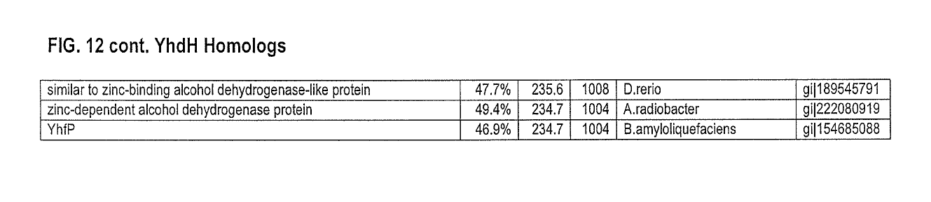

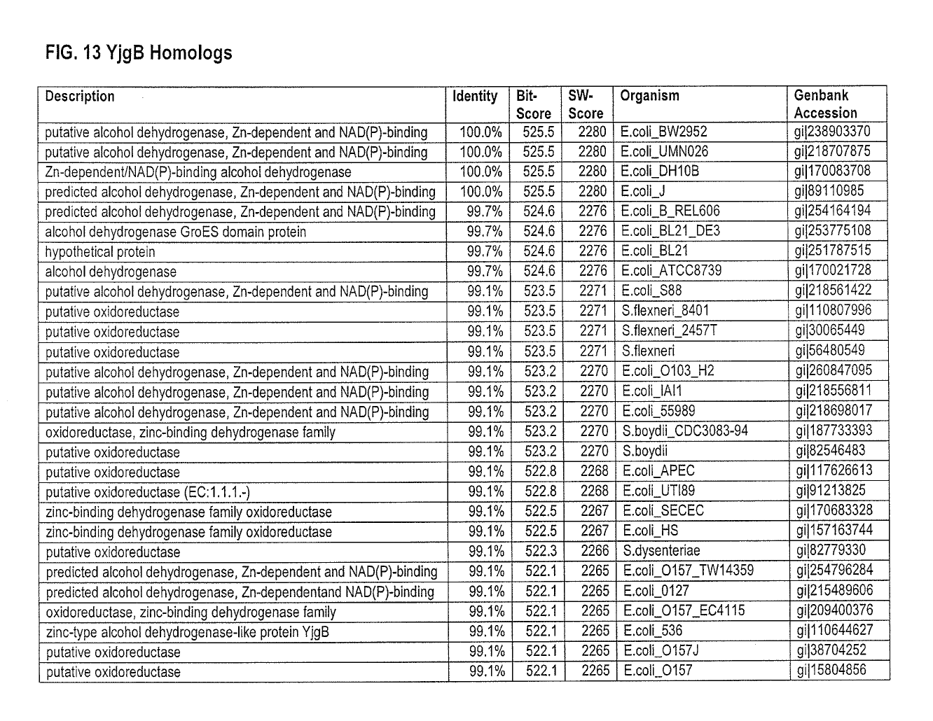

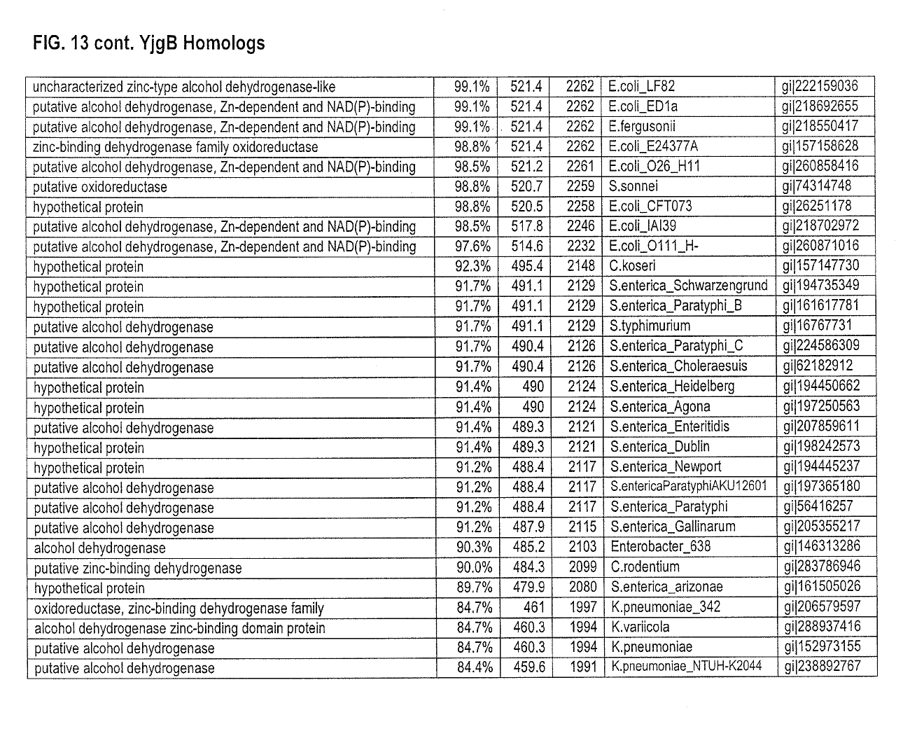

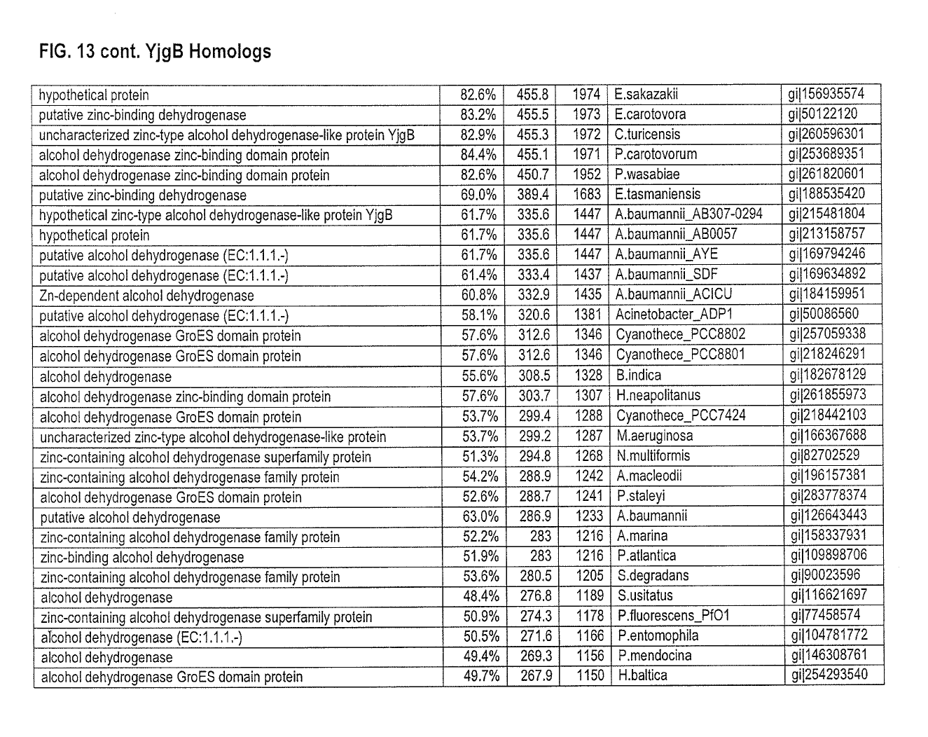

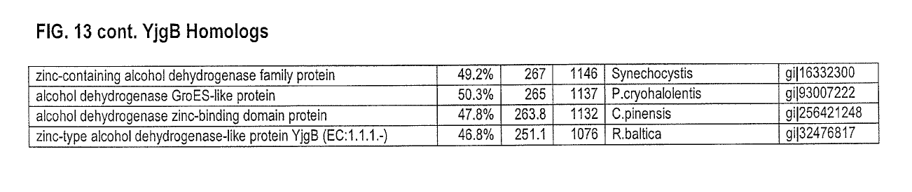

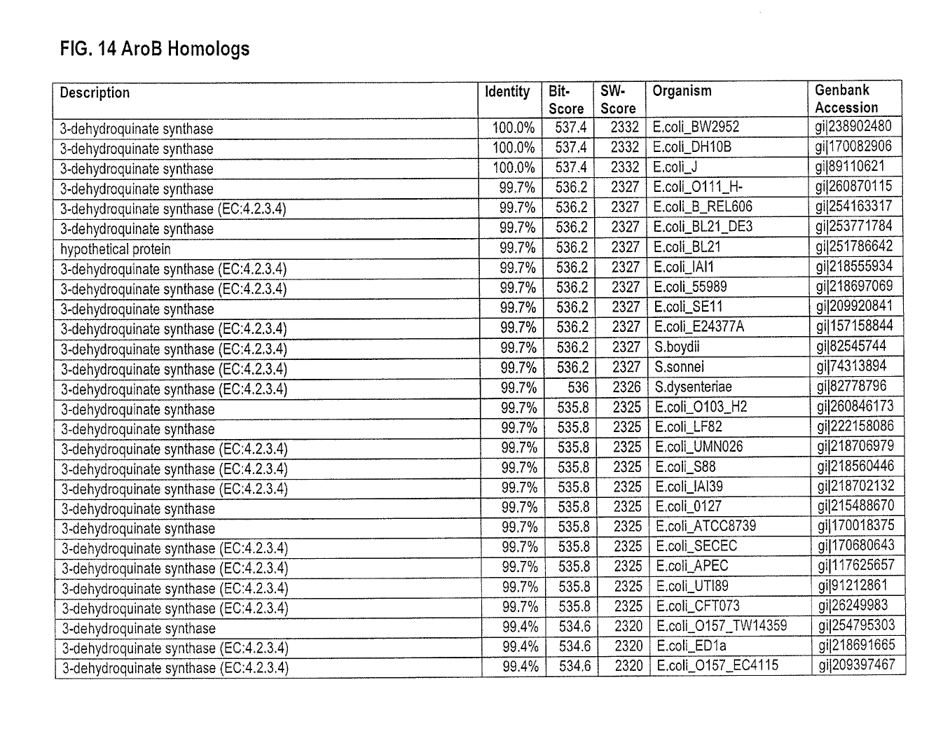

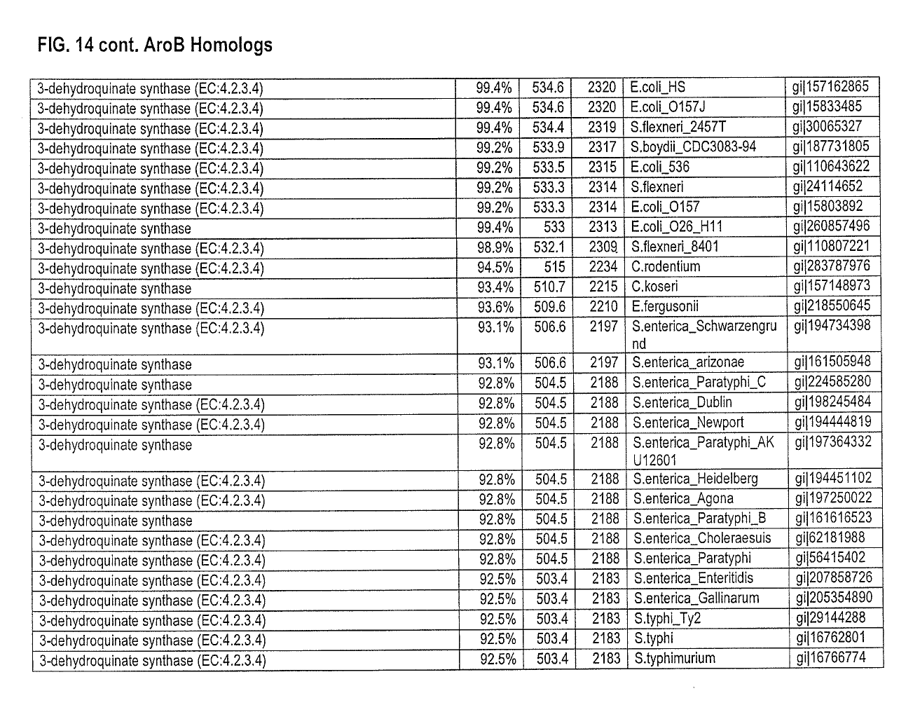

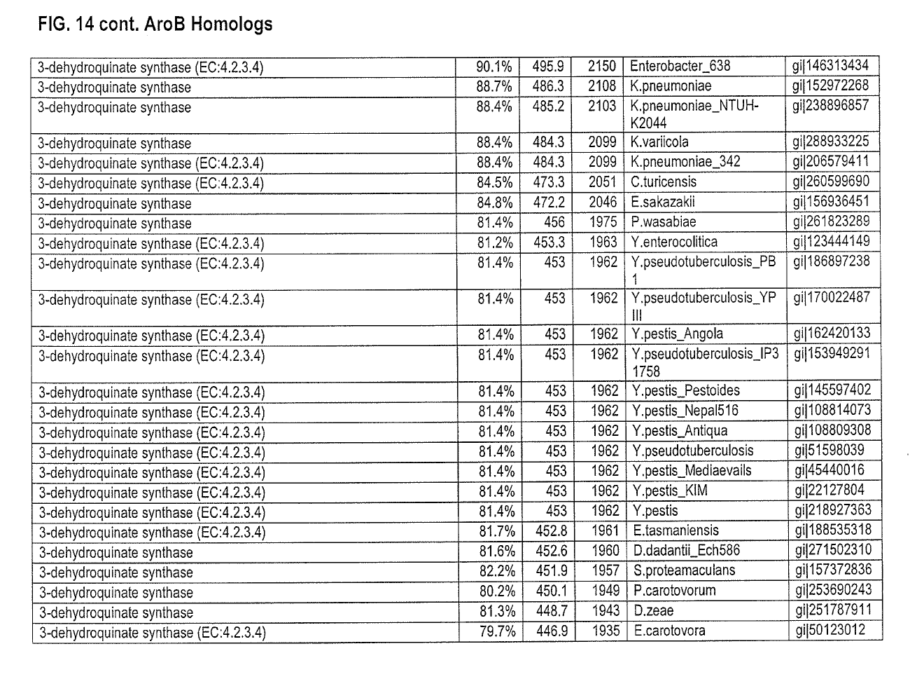

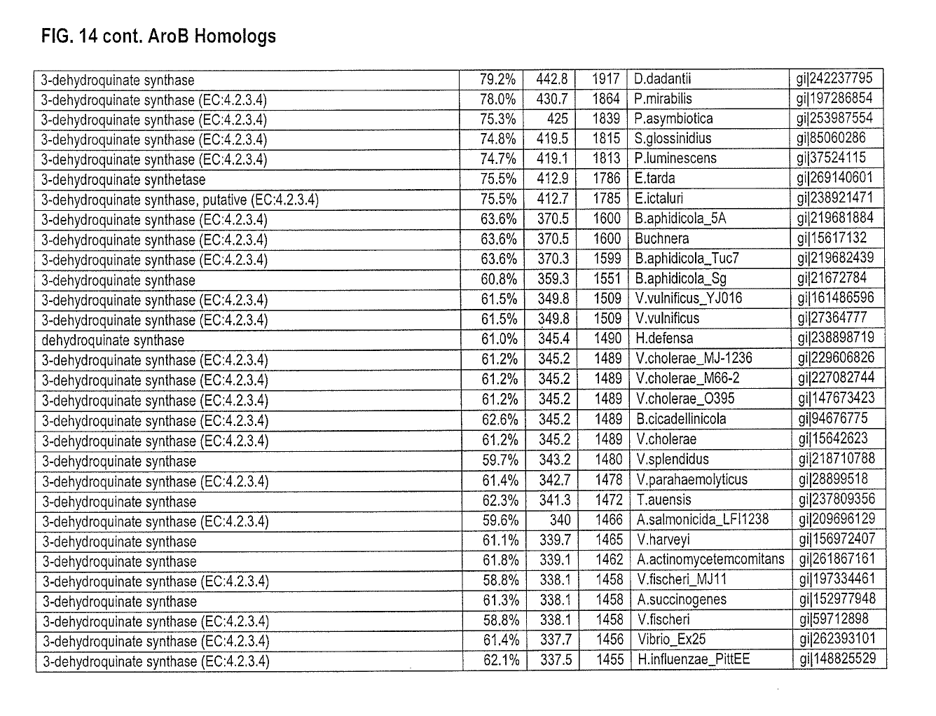

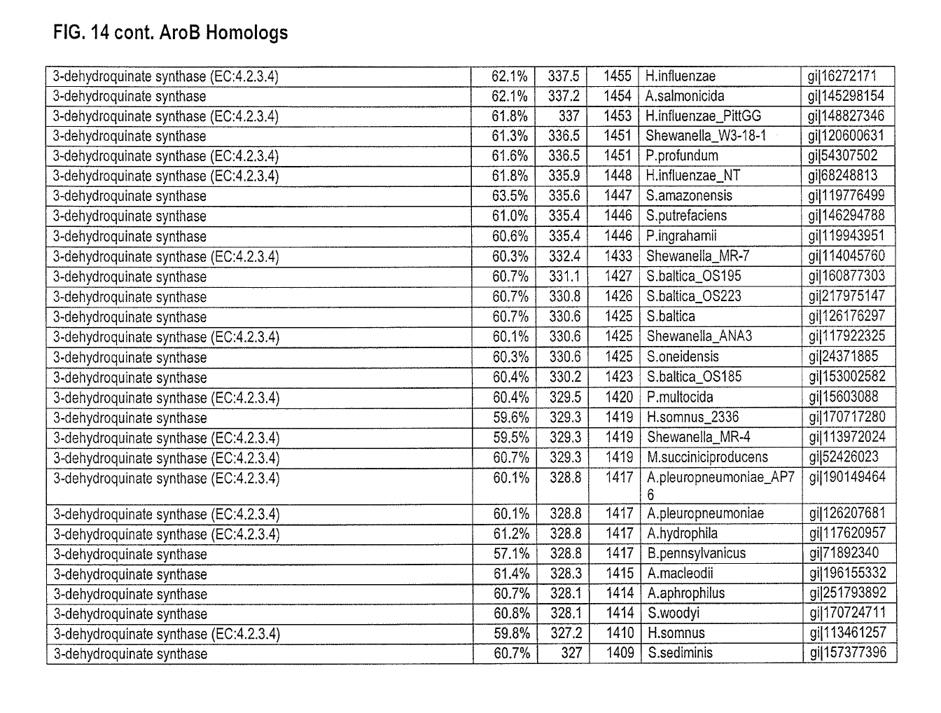

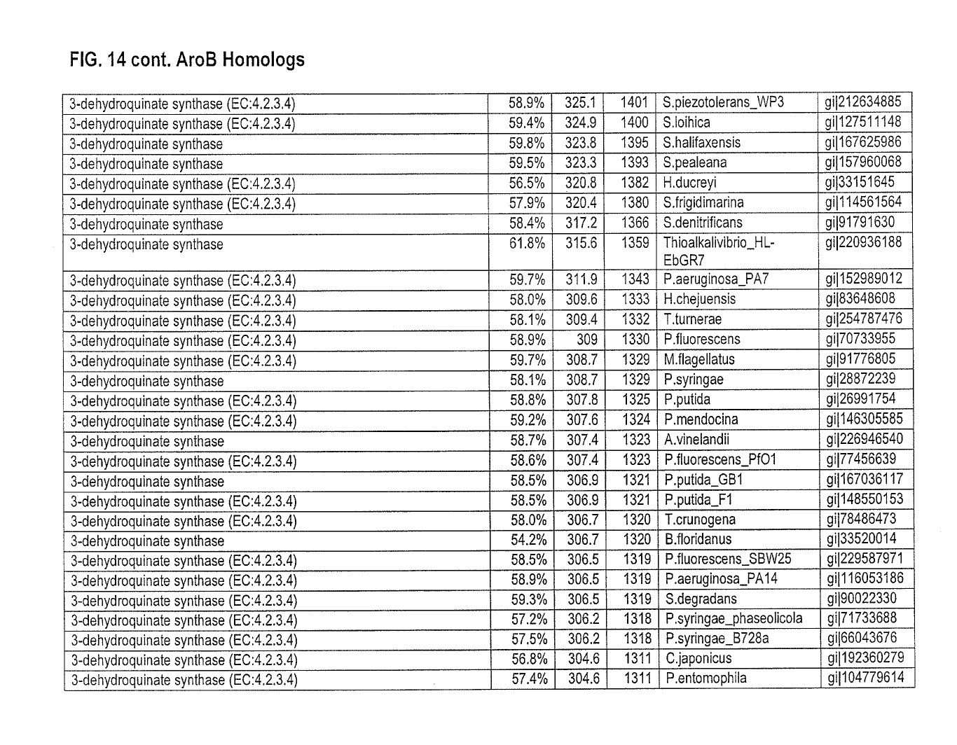

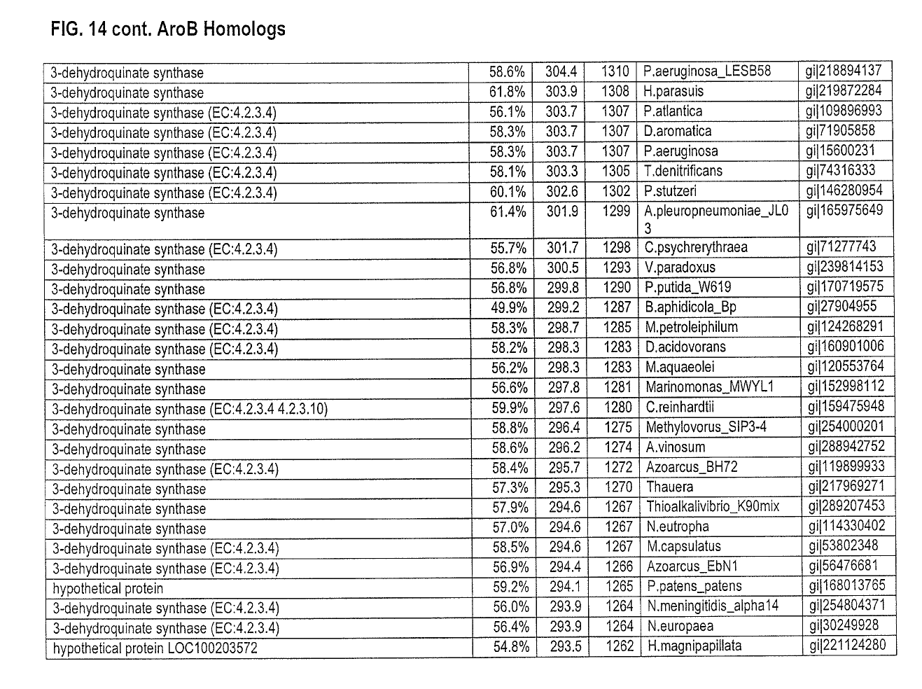

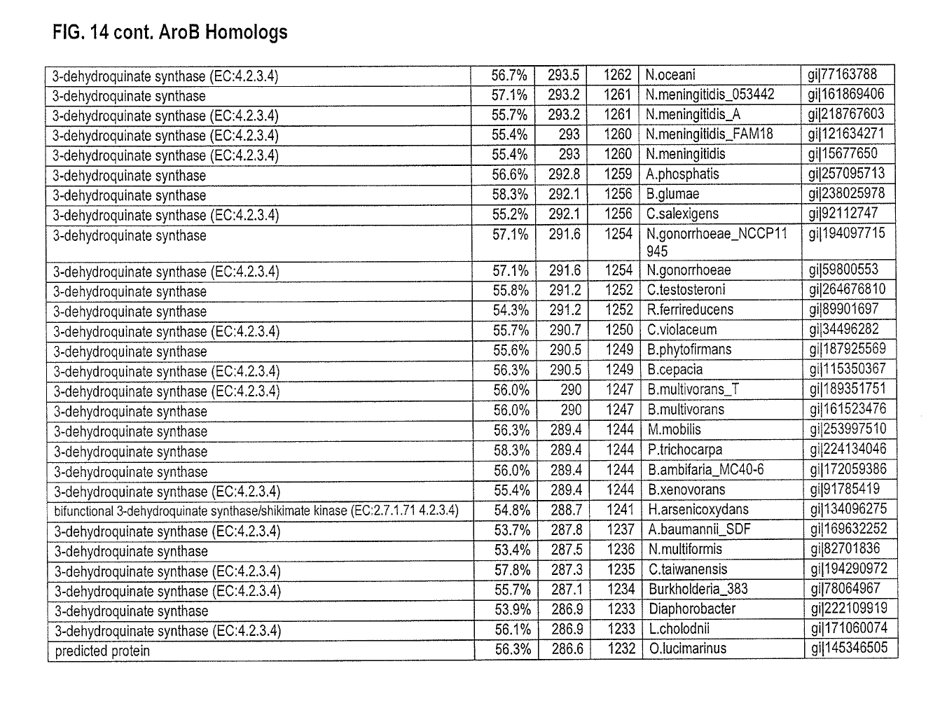

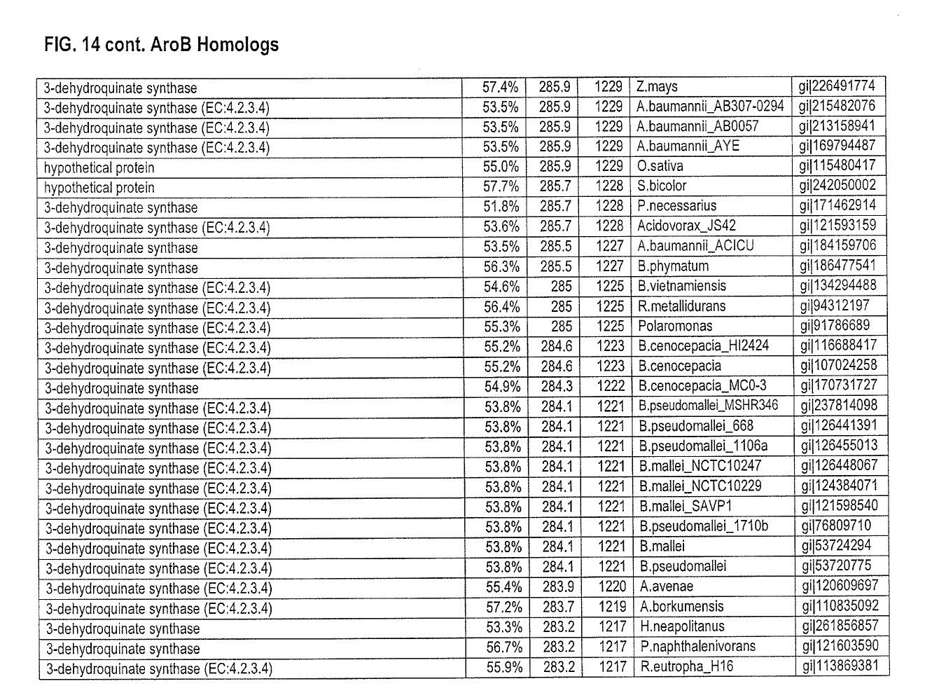

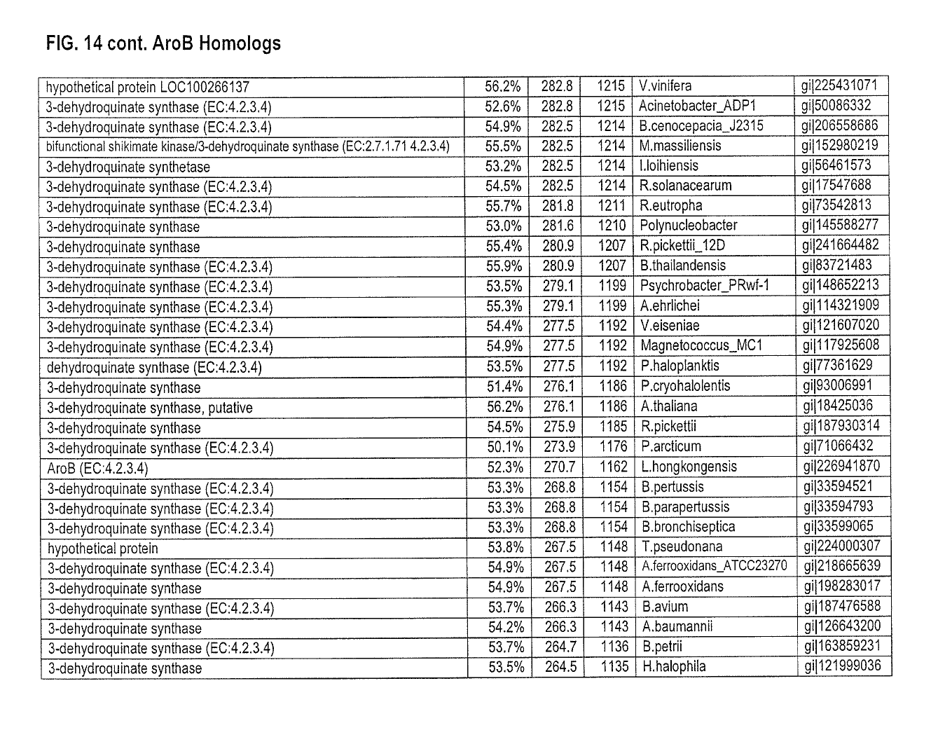

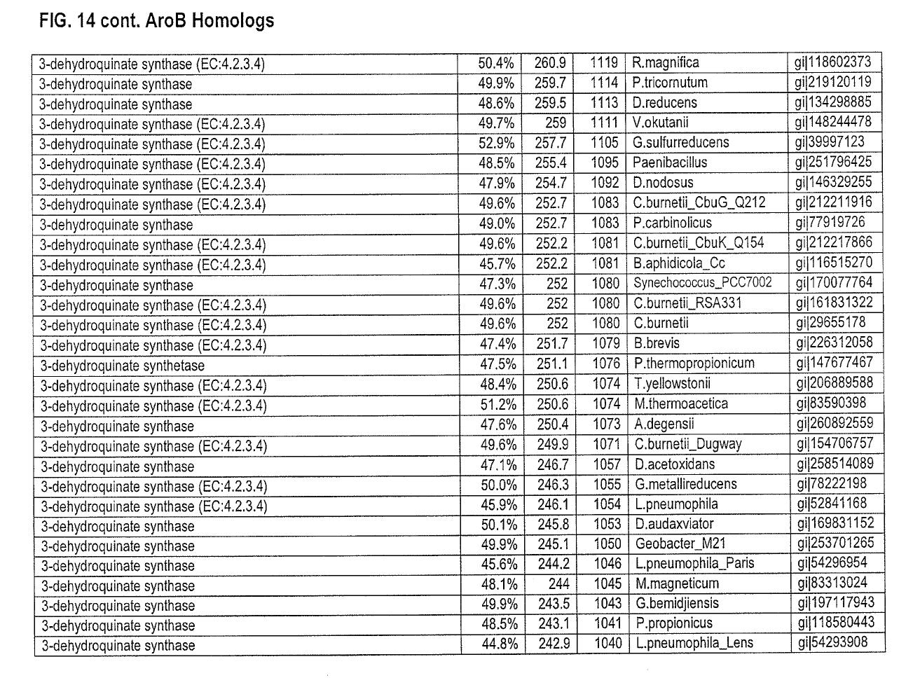

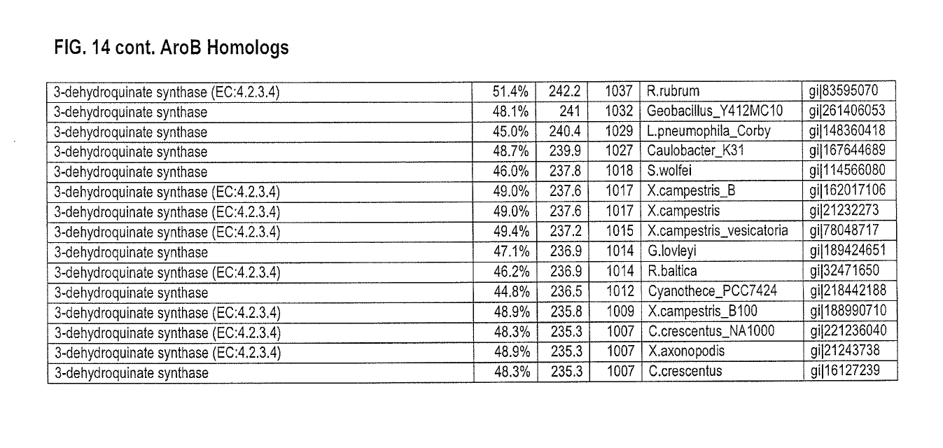

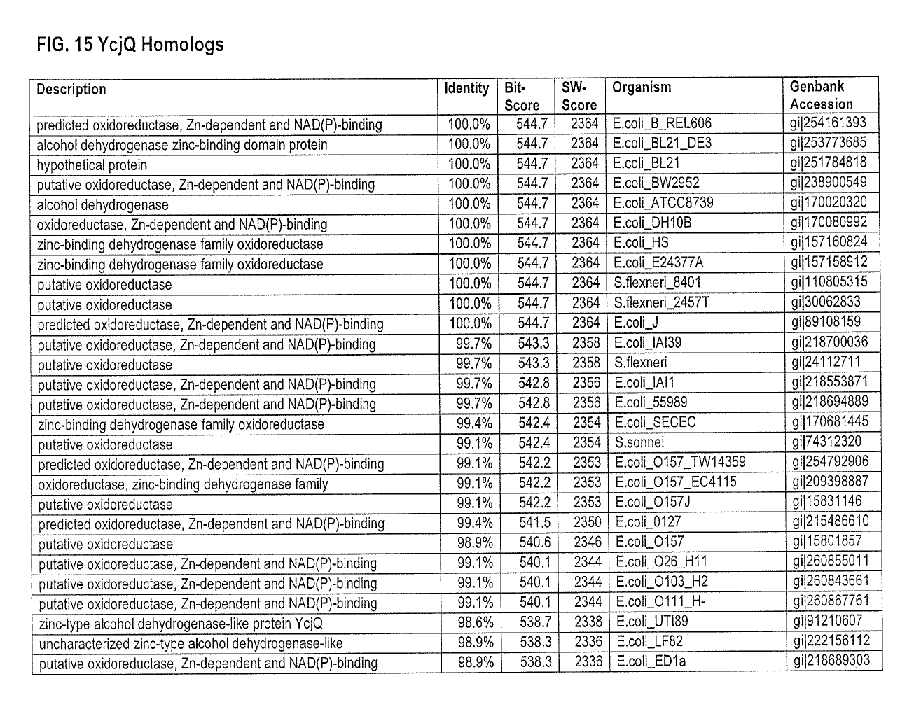

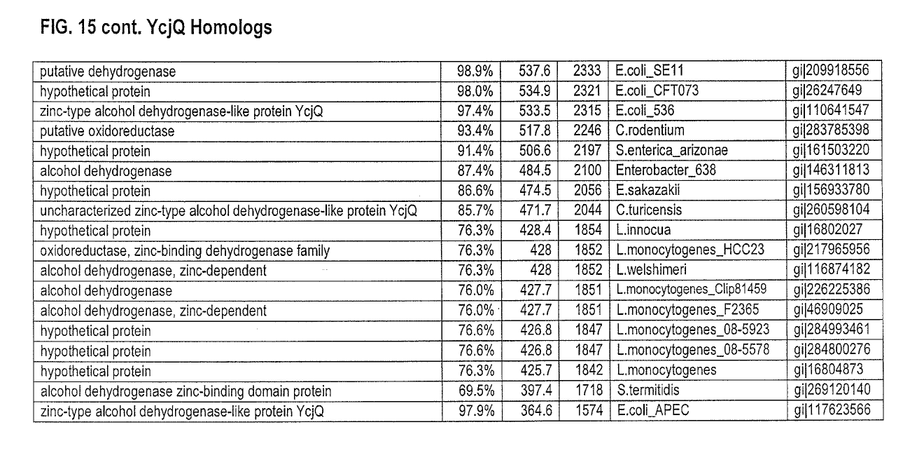

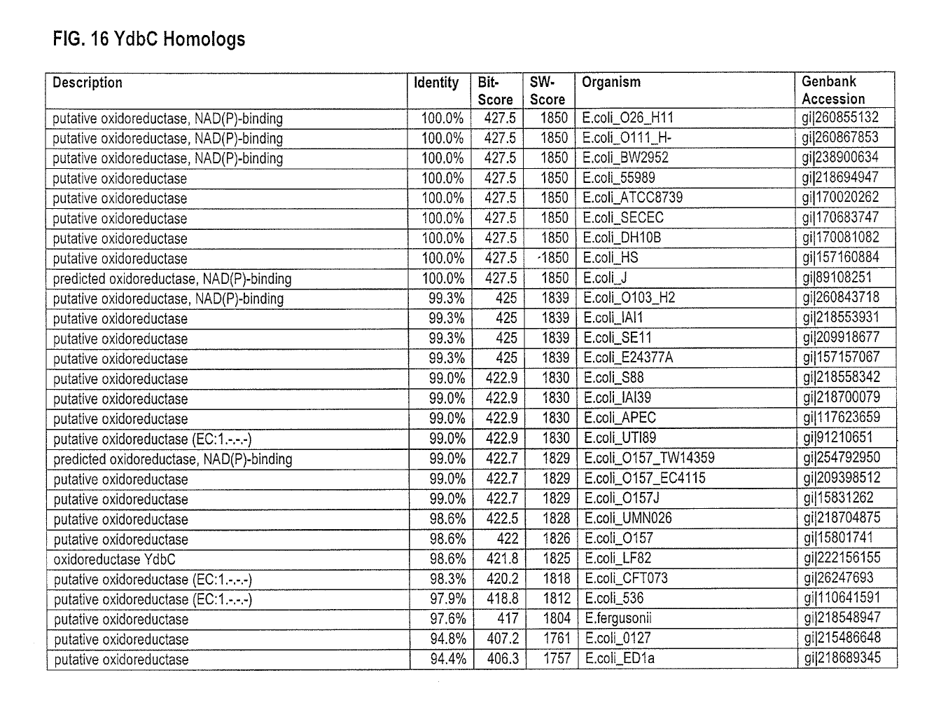

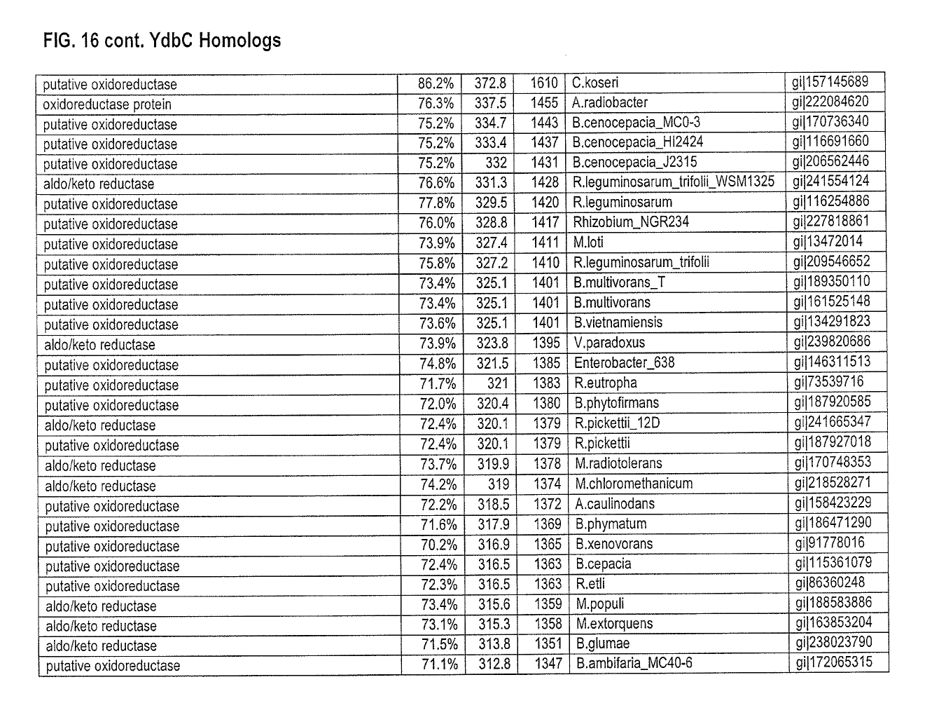

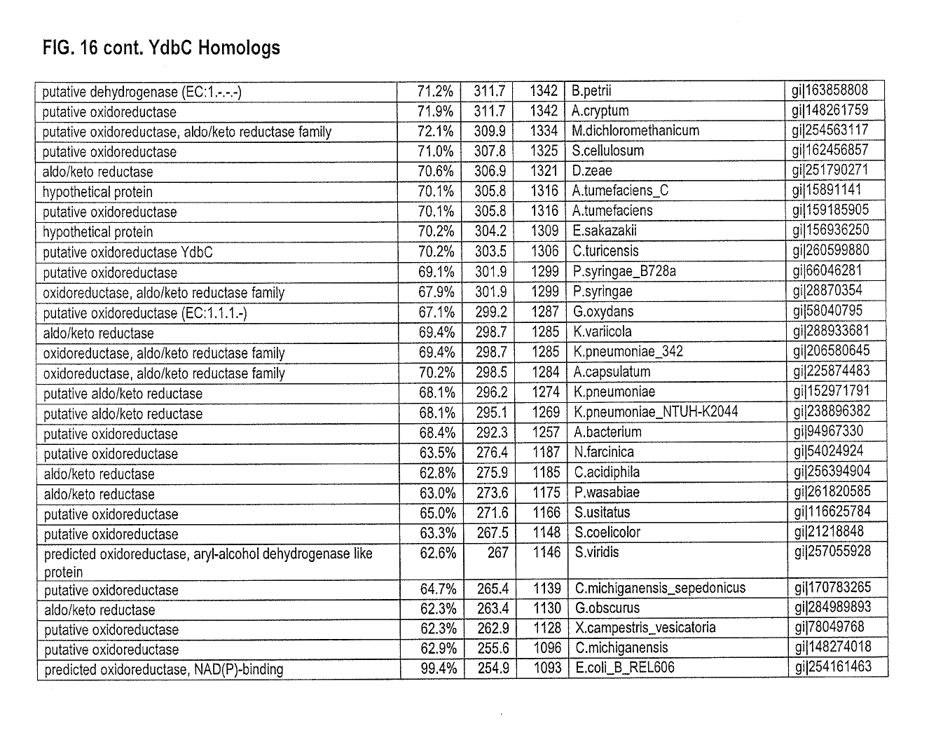

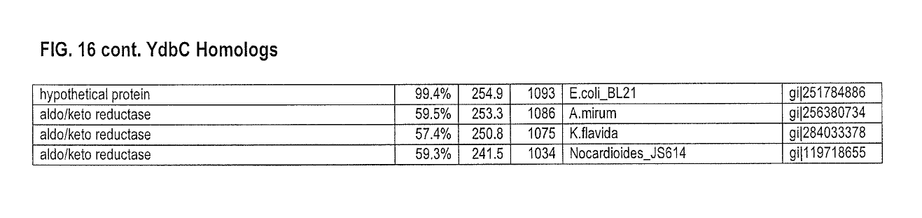

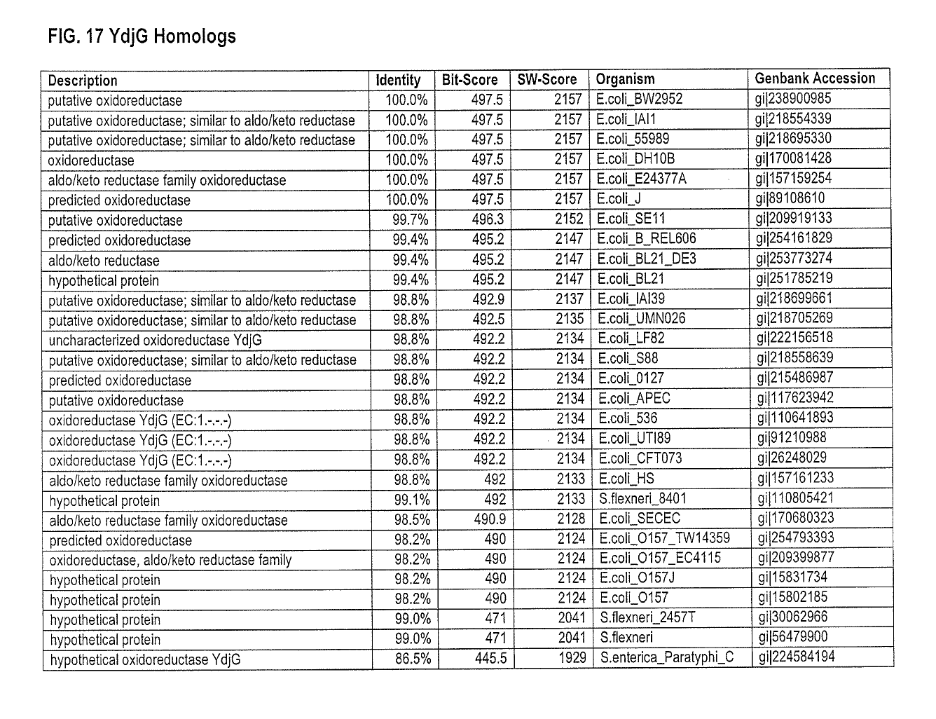

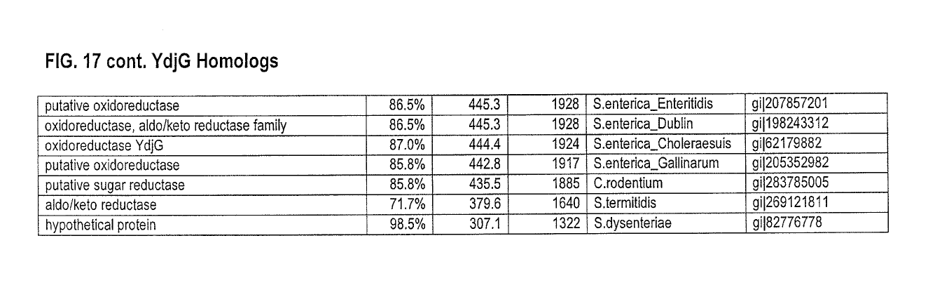

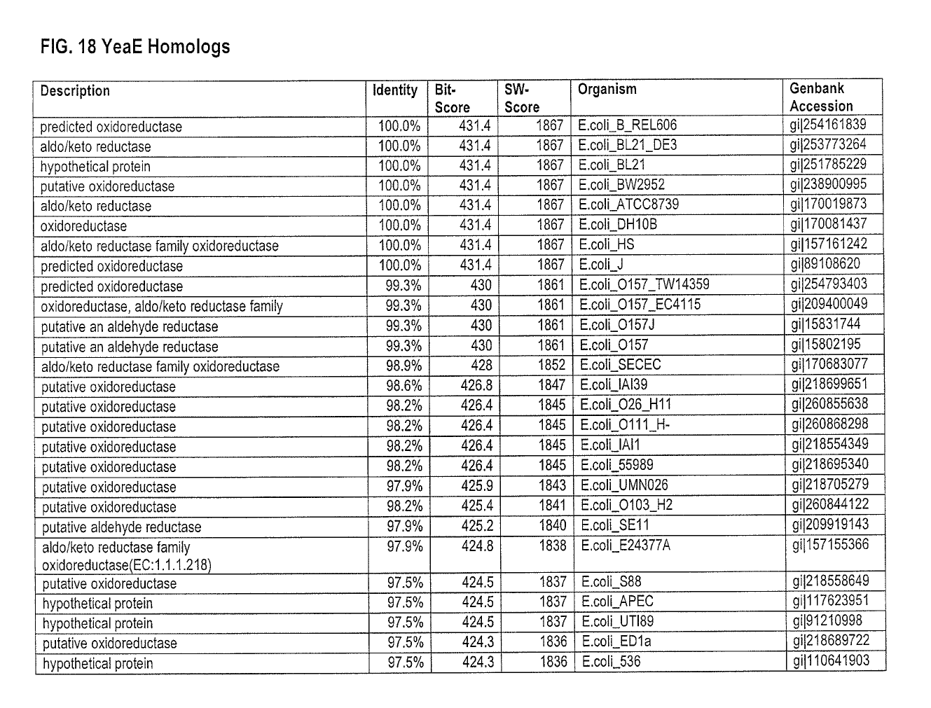

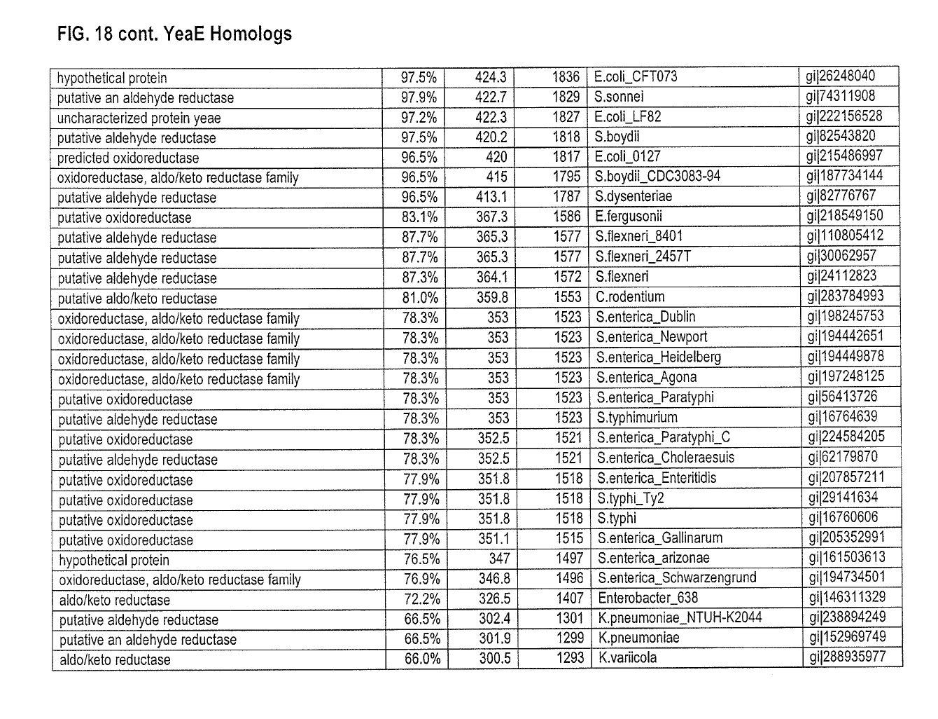

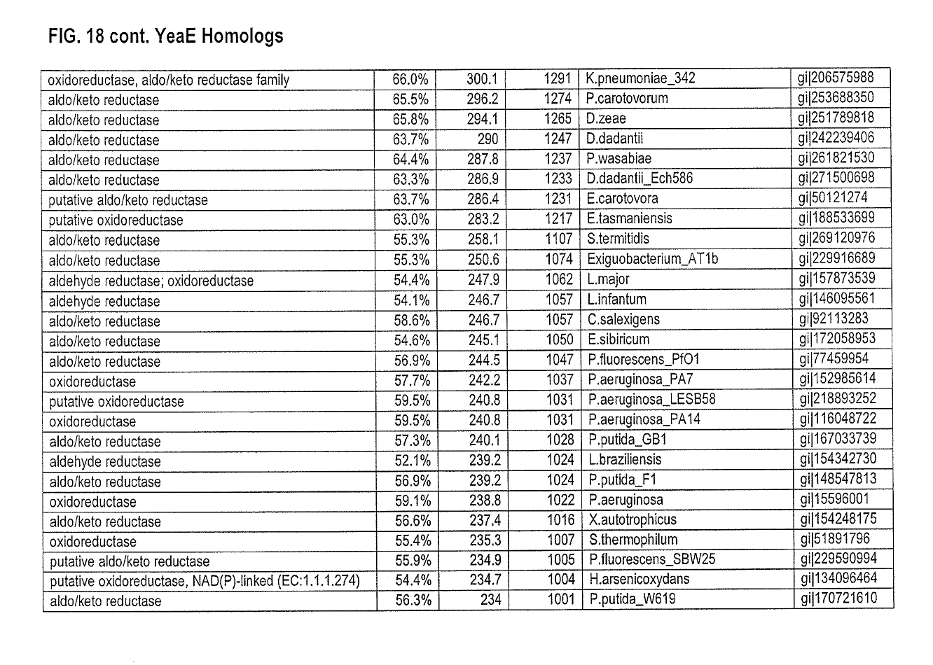

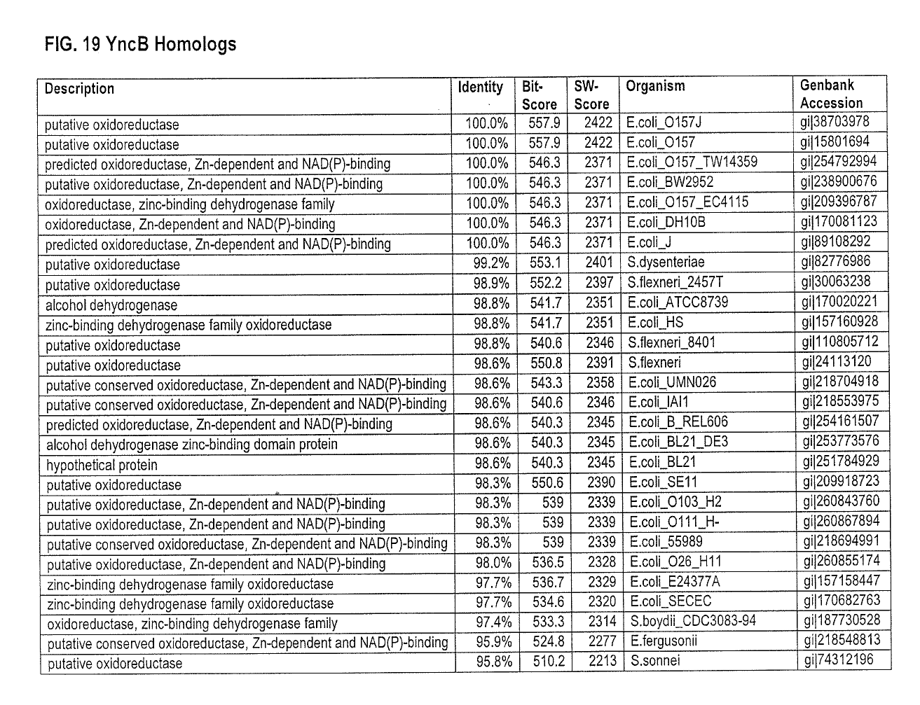

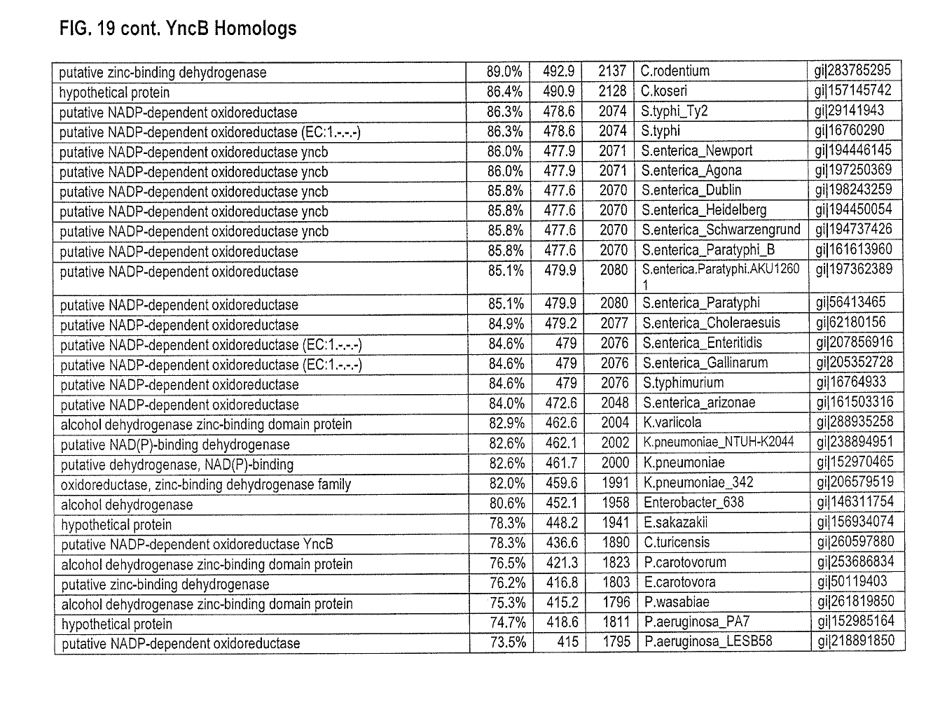

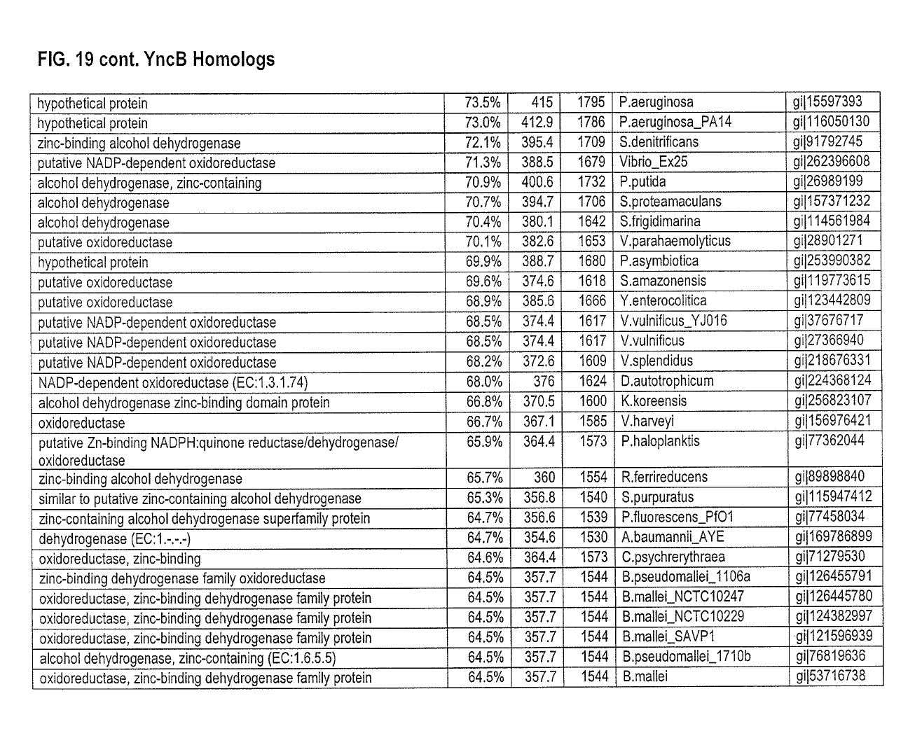

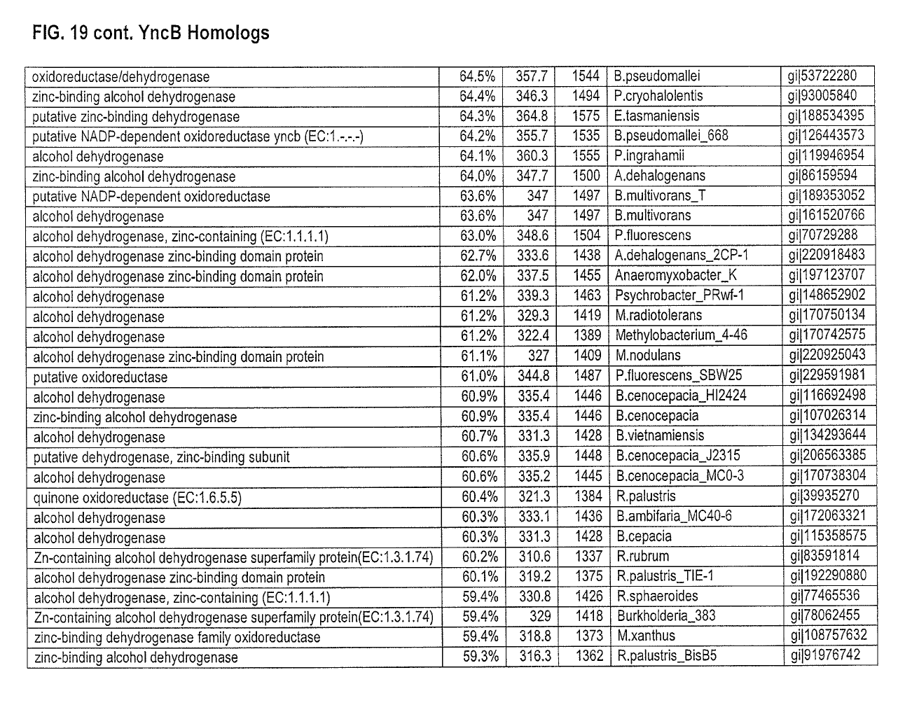

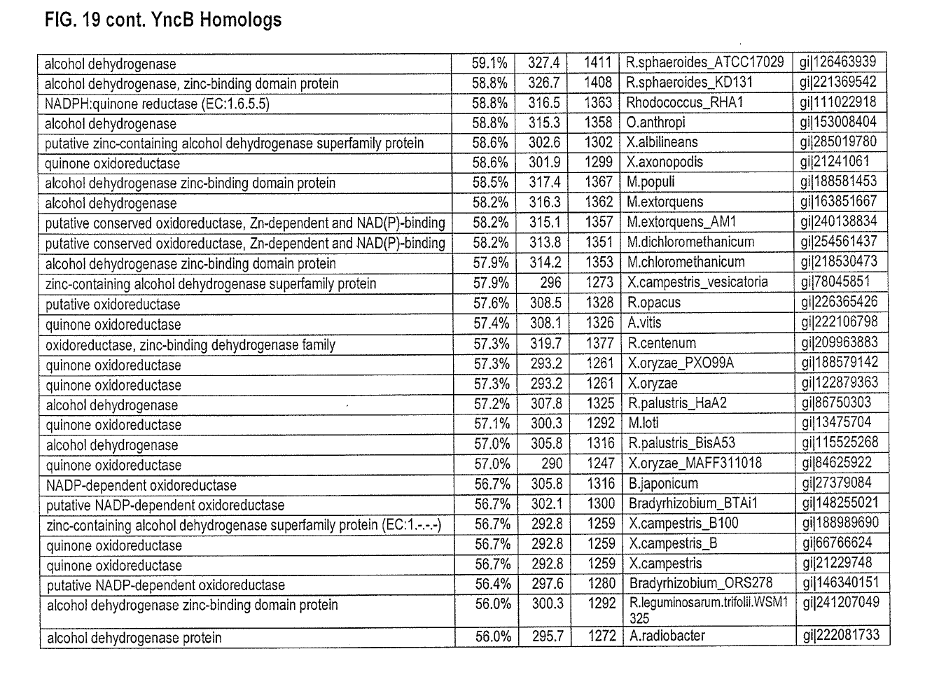

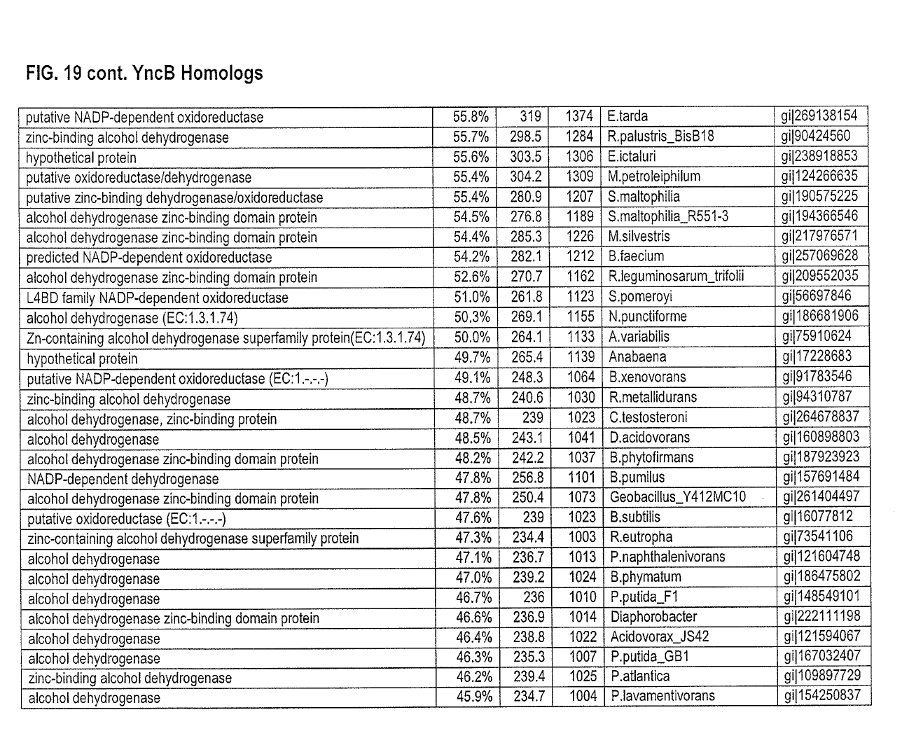

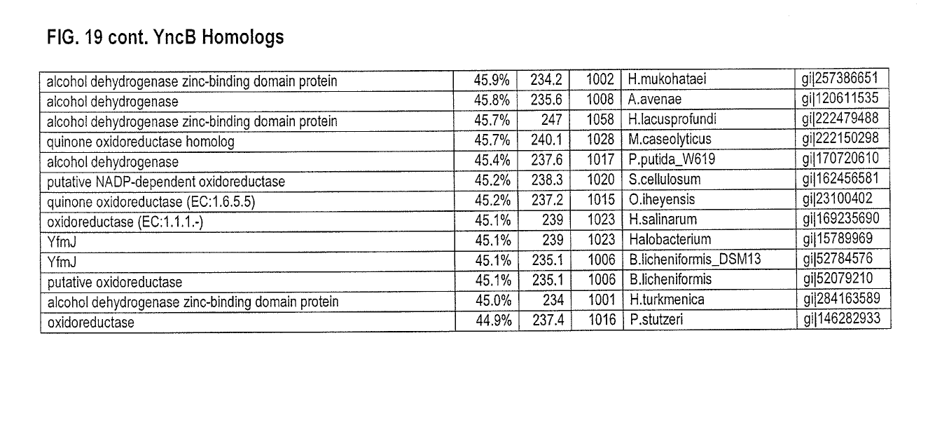

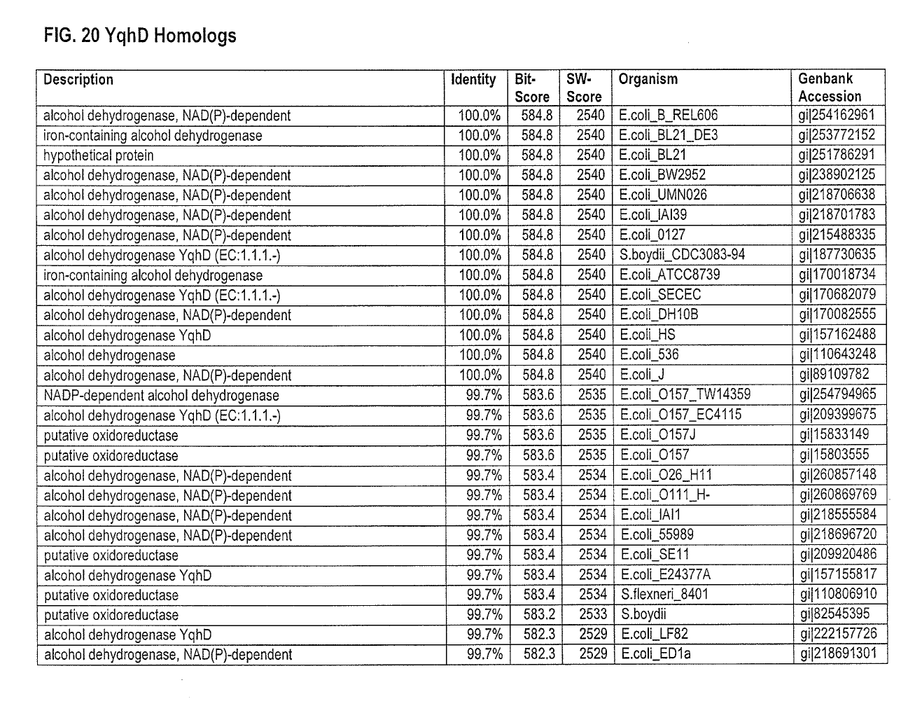

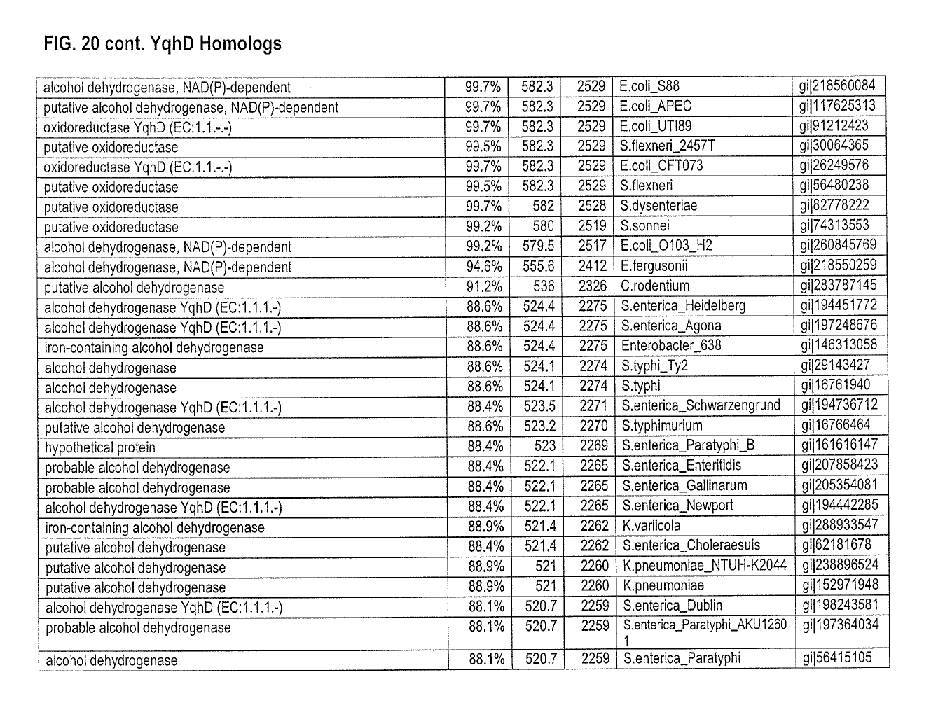

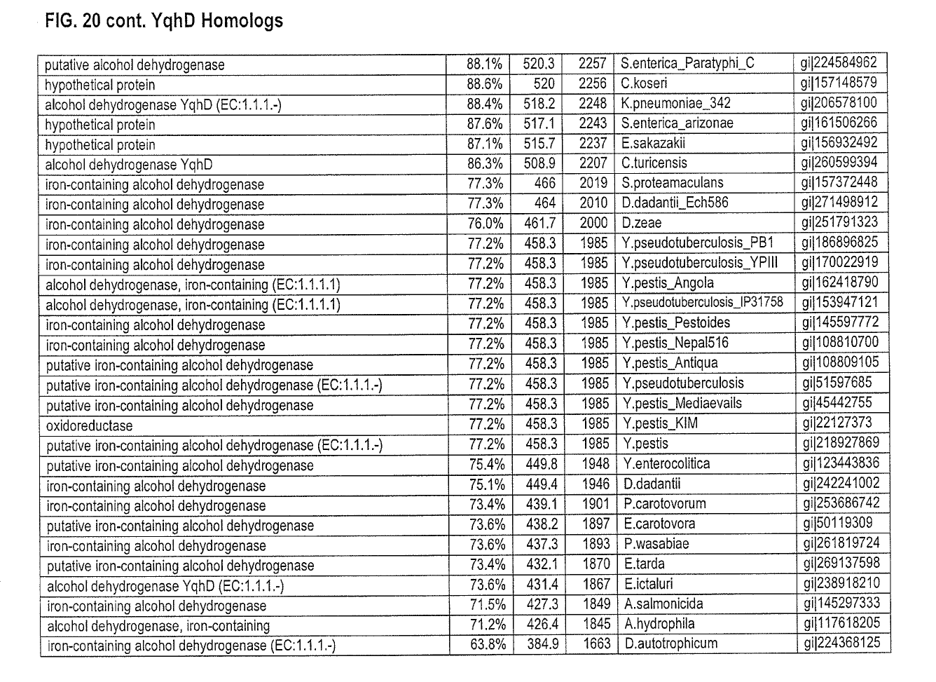

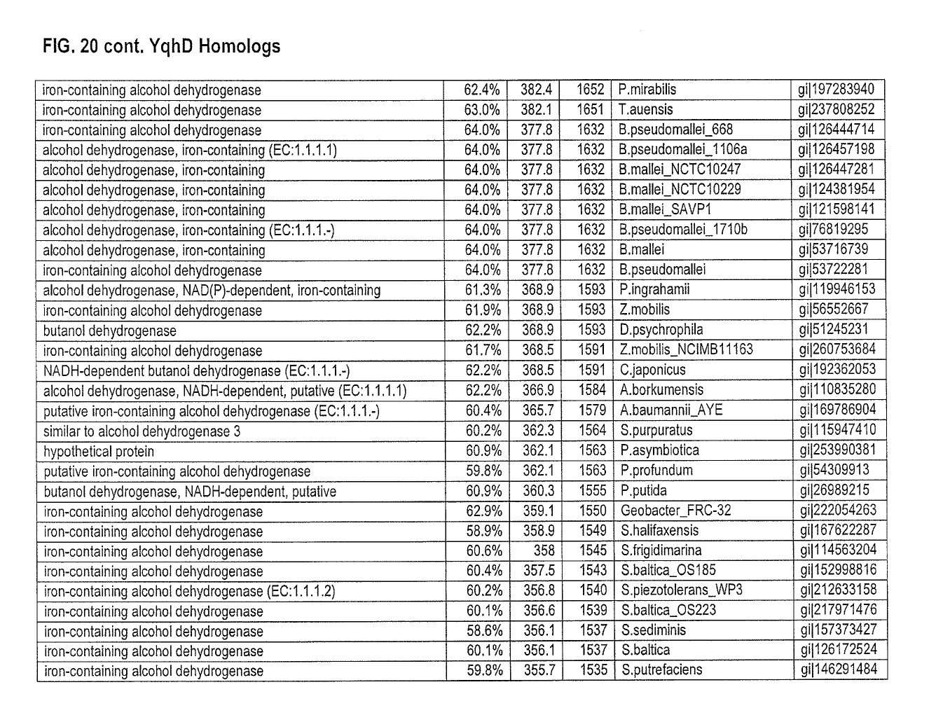

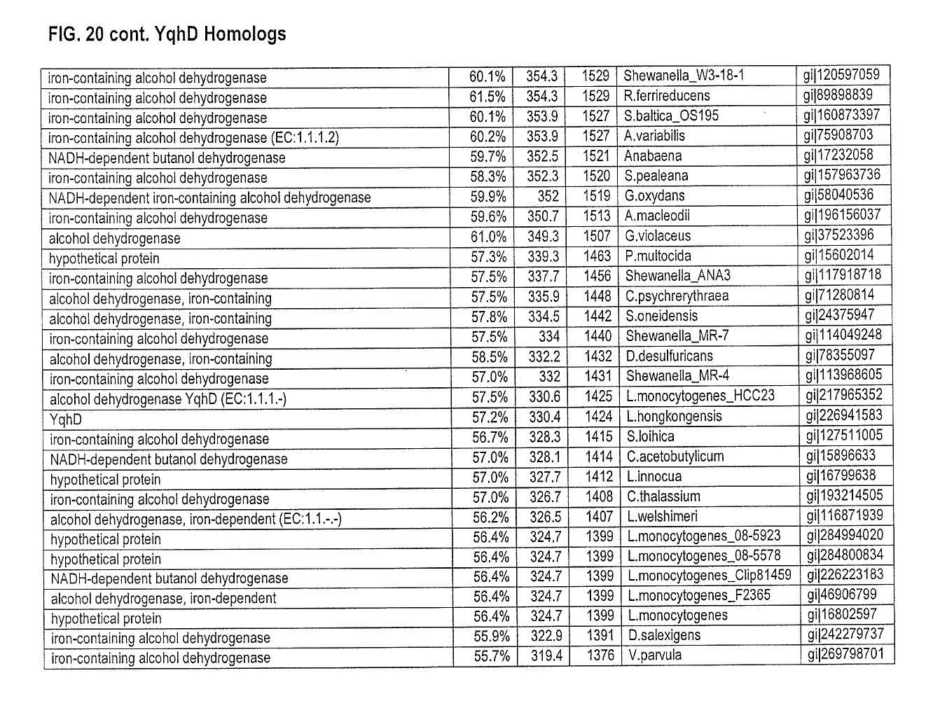

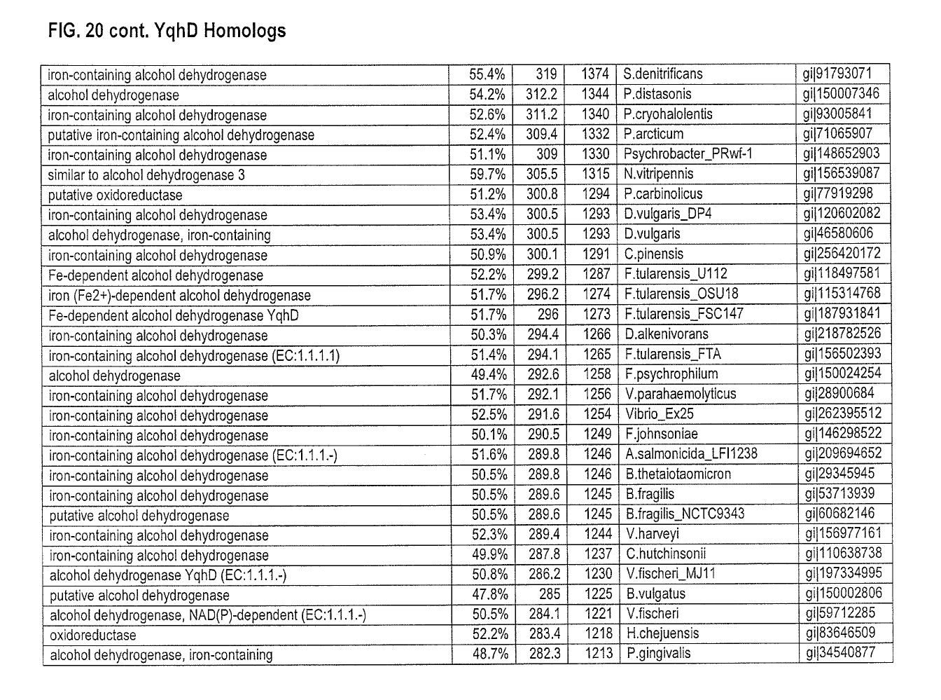

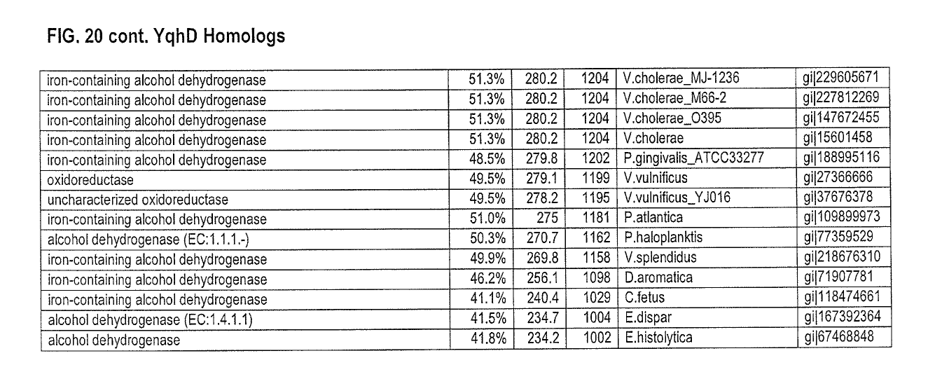

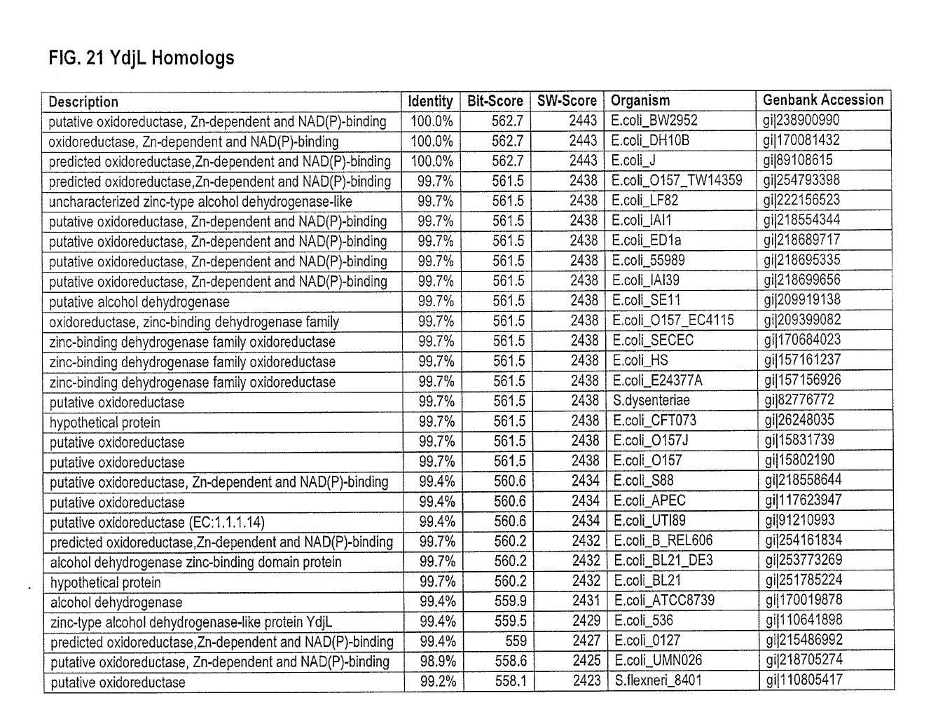

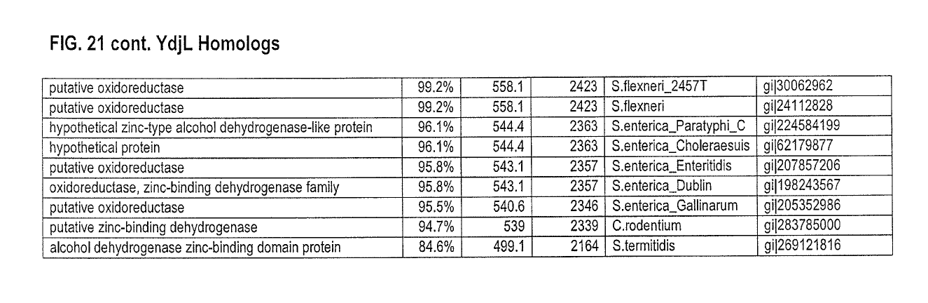

In yet a further embodiment, the fatty alcohol biosynthetic polypeptide can be one selected from an AdhP homolog of FIG. 2, a DkgA homolog of FIG. 3, a DkgB homolog of FIG. 4, a Tas homolog of FIG. 5, an RspB homolog of FIG. 6, a YahK homolog of FIG. 7, a YbbO homolog of FIG. 8, a YbdH homolog of FIG. 9, a YbdR homolog of FIG. 10, a YgfF homolog of FIG. 11, a YhdH homomolg of FIG. 12, a YjgB homolog of FIG. 13, an AroB homolog of FIG. 14, a YcjQ homolog of FIG. 15, a YdbC Homolog of FIG. 16, a YdjG homolog of FIG. 17, a YeaE homolog of FIG. 18, aYncB homolog of FIG. 19, a YqhD homolog of FIG. 20, a YdjL homolog of FIG. 21, or a variant thereof. In certain embodiments, the gene encoding a fatty alcohol biosynthetic polypeptide can be co-expressed in a host cell with a gene encoding a fatty aldehyde biosynthetic polypeptide or with a gene encoding an acyl-ACP reductase polypeptide described herein.

In certain embodiment, the gene has a nucleotide sequence selected from those described herein, as well as polynucleotide variants thereof. In exemplary embodiments, the fatty alcohol biosynthetic gene is one encoding an AdhP homolog of FIG. 2, as well as polynucleotide variants thereof. In other exemplary embodiments, the fatty alcohol biosynthetic gene is one encoding a DkgA homolog of FIG. 3, or one encoding a DkgB homolog of FIG. 4, or one encoding a Tas homolog of FIG. 5, or one encoding a RspB homolog of FIG. 6, or one encoding a YahK homolog of FIG. 7, or one encoding a YbbO homolog of FIG. 8, or one encoding a YbdH homolog of FIG. 9, or one encoding a YbdR homolog of FIG. 10, or one encoding a YgfF homolog of FIG. 11, or one encoding a YhdH homolog of FIG. 12, or one encoding a YjgB homolog of FIG. 13, or one encoding an AroB homolog of FIG. 14, or one encoding the YcjQ homolog of FIG. 15, or one encoding a YdbC homolog of FIG. 16, or one encoding a YdjG homolog of FIG. 17, or one encoding a YeaE homolog of FIG. 18, or one encoding a YncB homolog of FIG. 19, or one encoding a YqhD homolog of FIG. 20, or one encoding a YdjL homolog of FIG. 21, or a variant thereof, can be used as a fatty alcohol biosynthetic polynucleotide in the methods described herein.

Suitable variants, such as those listed in, for example, FIGS. 2-21, can be identified using bioinformatic tools such as described hereinbelow.

### Synthesis of Substrates

Fatty acid synthase (FAS) is a group of polypeptides that catalyze the initiation and elongation of acyl chains (Marrakchi et al., Biochemical Society, 30:1050-1055, 2002). The acyl carrier protein (ACP) along with the enzymes in the FAS pathway control the length, degree of saturation, and branching of the fatty acid derivatives produced. The fatty acid biosynthetic pathway involves the precursors acetyl-CoA and malonyl-CoA. The steps in this pathway are catalyzed by enzymes of the fatty acid biosynthesis (fab) and acetyl-CoA carboxylase (acc) gene families (see, e.g., Heath et al., Prog. Lipid Res. 40(6):467-97 (2001)).

Host cells can be engineered to express fatty acid derivative substrates by recombinantly expressing or overexpressing one or more fatty acid synthase genes, such as acetyl-CoA and/or malonyl-CoA synthase genes. For example, to increase acetyl-CoA production, one or more of the following genes can be expressed in a host cell: pdh (a multienzyme complex comprising aceEF (which encodes the E1 p dehydrogenase component, the E2p dihydrolipoamide acyltransferase component of the pyruvate and 2-oxoglutarate dehydrogenase complexes, and lpd), panK, fabH, fabB, fabD, fabG, acpP, and fabF. Exemplary GenBank accession numbers for these genes are: pdh (BAB34380, AAC73227, AAC73226), panK (also known as CoA, AAC76952), aceEF (AAC73227, AAC73226), fabH (AAC74175), fabB (P0A953), fabD (AAC74176), fabG (AAC74177), acpP (AAC74178), fabF (AAC74179). Additionally, the expression levels of fadE, gpsA, ldhA, pflb, adhE, pta, poxB, ackA, and/or ackB can be attenuated or knocked-out in an engineered host cell by transformation with conditionally replicative or non-replicative plasmids containing null or deletion mutations of the corresponding genes or by substituting promoter or enhancer sequences. Exemplary GenBank accession numbers for these genes are: fadE (AAC73325), gspA (AAC76632), ldhA (AAC74462), pflb (AAC73989), adhE (AAC74323), pta (AAC75357), poxB (AAC73958), ackA (AAC75356), and ackB (BAB81430). The resulting host cells will have increased acetyl-CoA production levels when grown in an appropriate environment.

Malonyl-CoA overexpression can be affected by introducing accABCD (e.g., accession number AAC73296, EC 6.4.1.2) into a host cell. Fatty acids can be further overexpressed in host cells by introducing into the host cell a DNA sequence encoding a lipase (e.g., accession numbers CAA89087, CAA98876).

In addition, inhibiting P1sB can lead to an increase in the levels of long chain acyl-ACP, which will inhibit early steps in the pathway (e.g., accABCD, fabH, and fabl). The plsB (e.g., accession number AAC77011) D311E mutation can be used to increase the amount of available fatty acids.

In addition, a host cell can be engineered to overexpress a sfa gene (suppressor of fabA, e.g., accession number AAN79592) to increase production of monounsaturated fatty acids (Rock et al., J. Bacteriology 178:5382-5387, 1996).

The chain length of a fatty acid derivative substrate can be selected for by modifying the expression of selected thioesterases (EC 3.1. 2.14 or EC 3.1.1.5). The thioesterase influences the chain length of fatty acids produced. Hence, host cells can be engineered to express, overexpress, have attenuated expression, or not to express one or more selected thioesterases to increase the production of a preferred fatty acid derivative substrate. For example, C₁₀ fatty acids can be produced by expressing a thioesterase that has a preference for producing C₁₀ fatty acids and attenuating thioesterases that have a preference for producing fatty acids other than C₁₀ fatty acids (e.g., a thioesterase which prefers to produce C₁₄ fatty acids). This would result in a relatively homogeneous population of fatty acids that have a carbon chain length of 10. In other instances, C₁₄ fatty acids can be produced by attenuating endogenous thioesterases that produce non-C₁₄ fatty acids and expressing the thioesterases that use C₁₄-ACP. In some situations, C₁₂ fatty acids can be produced by expressing thioesterases that use C₁₂-ACP and attenuating thioesterases that produce non-C₁₂ fatty acids. Acetyl-CoA, malonyl-CoA, and fatty acid overproduction can be verified using methods known in the art, for example, by using radioactive precursors, HPLC, or GC-MS subsequent to cell lysis. Non-limiting examples of thioesterases that can be used in the methods described herein are listed in Table 1.

Table 1: Thioesterases

Mayer et al., BMC Plant Biology 7:1-11, 2007

In other instances, a fatty alcohol biosynthetic polypeptide, variant, or a fragment thereof, is expressed in a host cell that contains a naturally occurring mutation that results in an increased level of fatty acids in the host cell. In some instances, the host cell is genetically engineered to increase the level of fatty acids in the host cell relative to a corresponding wild-type host cell. For example, the host cell can be genetically engineered to express a reduced level of an acyl-CoA synthase (EC 2.3.1.86) relative to a corresponding wild-type host cell. For example, the host cell can be genetically engineered to express a reduced level of an acyl-CoA synthase relative to a corresponding wild-type host cell. In one embodiment, the level of expression of one or more genes (e.g., an acyl-CoA synthase gene) is reduced by genetically engineering a “knock out” host cell.

Any known acyl-CoA synthase gene can be reduced or knocked out in a host cell. Non-limiting examples of acyl-CoA synthase genes include fadD, fadK, BH3103, yhfL, Pfl-4354, EAV15023, fadD1, fadD2, RPC_{—}4074, fadDD35, fadDD22, faa3p or the gene encoding the protein ZP_{—}01644857. Specific examples of acyl-CoA synthase genes include fadDD35 from M. tuberculosis H37Rv [NP_{—}217021], AdDD22 from M. tuberculosis H37Rv [NP_{—}217464], fadD from E. coli [NP_{—}416319], fadK from E. coli [YP_{—}416216], fadD from Acinetobacter sp. ADP1 [YP_{—}045024], fadD from Haemophilus influenza RdkW20 [NP_{—}438551], fadD from Rhodopseudomonas palustris Bis B18 [YP_{—}533919], BH3101 from Bacillus halodurans C-125 [NP_{—}243969], Pfl-4354 from Pseudomonas fluorescens Pfo-1 [YP_{—}350082], EAV15023 from Comamonas testosterone KF-1 [ZP_{—}01520072], yhfL from B. subtilis [NP_{—}388908], fadD1 from P. aeruginosa PAO1 [NP_{—}251989], fadD1 from Ralstonia solanacearum GM1 1000 [NP_{—}520978], fadD2 from P. aeruginosa PAO1 [NP_{—}251990], the gene encoding the protein ZP_{—}01644857 from Stenotrophomonas maltophilia R551-3, faa3p from Saccharomyces cerevisiae [NP_{—}012257], faalp from Saccharomyces cerevisiae [NP_{—}014962], lcfA from Bacillus subtilis [CAA99571], or those described in Shockey et al., Plant. Physiol. 129:1710-1722, 2002; Caviglia et al., J. Biol. Chem. 279:1163-1169, 2004; Knoll et al., J. Biol. Chem. 269(23):16348-56, 1994; Johnson et al., J. Biol. Chem. 269: 18037-18046, 1994; and Black et al., J. Biol. Chem. 267: 25513-25520, 1992.

### Fatty Aldehyde Substrates

Fatty aldehyde biosynthetic polypeptides refer to a group of polypeptides that can catalyze the enzymatic conversion of suitable fatty acid substrates into fatty aldehydes. Host cells can be engineered to express fatty aldehyde substrates by recombinantly expressing or overexpressing one or more fatty aldehyde biosynthetic genes, such as carboxylic acid reductases or fatty acid reductases.

In this pathway, a fatty acid is first activated by ATP and then reduced by a carboxylic acid reductase (CAR)-like enzyme to generate a fatty aldehyde. In some embodiments, a fatty aldehyde is produced by expressing a fatty aldehyde biosynthetic gene, for example, a carboxylic acid reductase gene (car gene), having a nucleotide sequence provided herein, as well as nucleotide variants thereof. Examplary genes encode a polypeptide comprising SEQ ID NO: 41, 43, 45, 47, 49, 51, 53, 55, 57, 59, 61, 63, 65, 69, 71, 73, 75, 77, 79, 81, 83, 85, 87, 89, 91, 93, 97, 99, 101, 103, 105, 107, 109, 111, 113, 115, 117, 119, 121, 123, 125, 127, or a variant thereof. In another example, the gene can comprise a polynucleotide sequence of SEQ ID NO: 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, or 128, or a variant thereof. In further embodiments, the fatty aldehyde biosynthetic polypeptide can comprise one or more of the amino acid motifs depicted herein in SEQ ID NO: 129-135. For example, the fatty aldehyde biosynthetic gene can encode a polypeptide comprising SEQ ID NO:129, SEQ ID NO:130, SEQ ID NO:131, and SEQ ID NO:132; SEQ ID NO:133; SEQ ID NO:134; SEQ ID NO:135; SEQ ID NO: 136; and/or SEQ ID NO:129, SEQ ID NO:131, SEQ ID NO:132, and SEQ ID NO:133.

Alternatively, fatty aldehyde substrates can be produced using an enzymatic pathway involving an acyl-ACP reductase. In some embodiments, a fatty aldehyde can be produced from a suitable substrate, including, for example, an acyl-ACP, an acyl-CoA, or others, by expressing an acyl-ACP reductase gene (aar gene), having a nucleotide sequence provided herein, as well as nucleotide variants thereof. For example, the acyl-ACP reductase gene can encode a polypeptide comprising SEQ ID NO: 155, 156, 157, 158, 159, 160, 161, 162, 163, 164, or 165.

Other substrates that can be used to produce fatty aldehydes and fatty alcohols in the methods described herein are acyl-ACP, acyl-CoA, fatty aldehydes, or fatty alcohols, which are described in, for example, PCT/US08/058,788, the disclosure of which is incorporated herein by reference.

### Fatty Acid Degradation Enzymes

In some embodiments, the host cell is genetically engineered to express an attenuated level of a fatty acid degradation enzyme relative to a wild type host cell. In some embodiments, the host cell is genetically engineered to express an attenuated level of an acyl-CoA synthase (EC 2.3.1.86) relative to a wild type host cell. In particular embodiments, the host cell expresses an attenuated level of an acyl-CoA synthase encoded by fadD, fadK, BH3103, yhfL, Pfl-4354, EAV15023, fadD1, fadD2, RPC 4074, fadDD35, fadDD22, faa3p or the gene encoding the protein ZP_{—}01644857. In certain embodiments, the genetically engineered host cell comprises a knockout of one or more genes encoding a fatty acid degradation enzyme, such as the aforementioned acyl-CoA synthase genes.

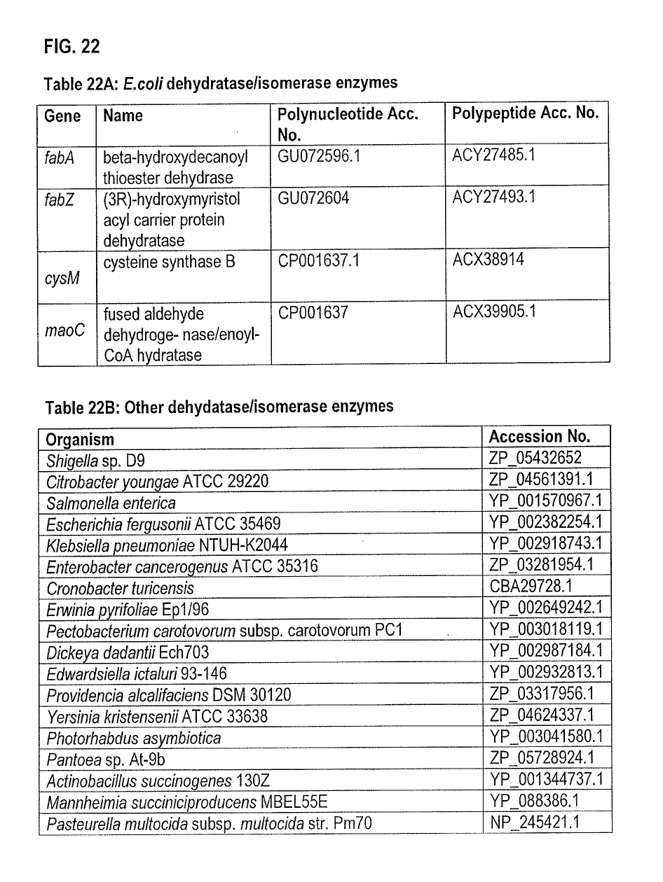

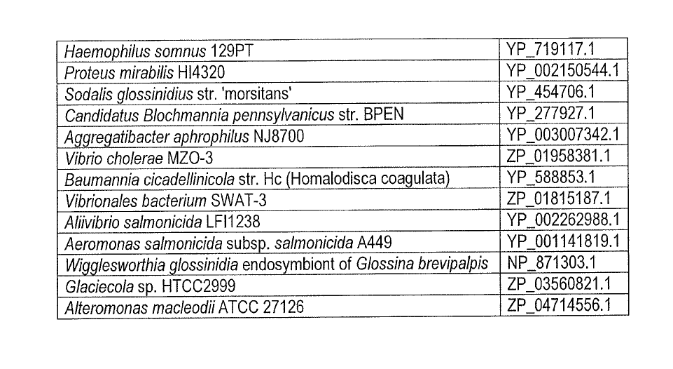

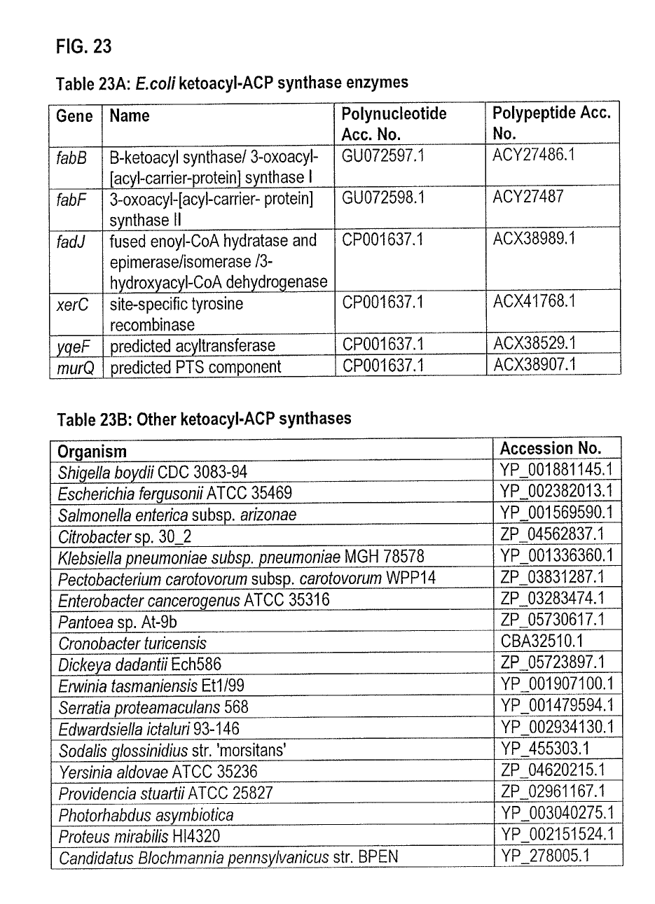

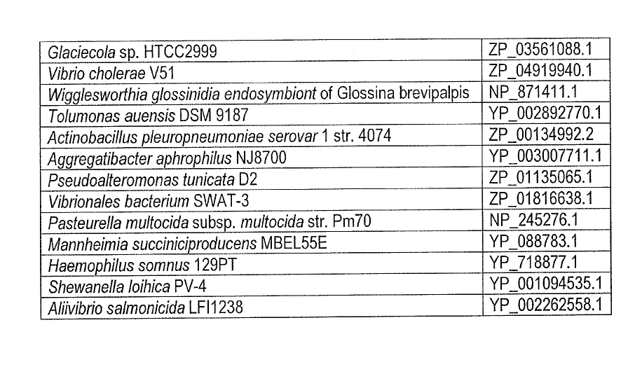

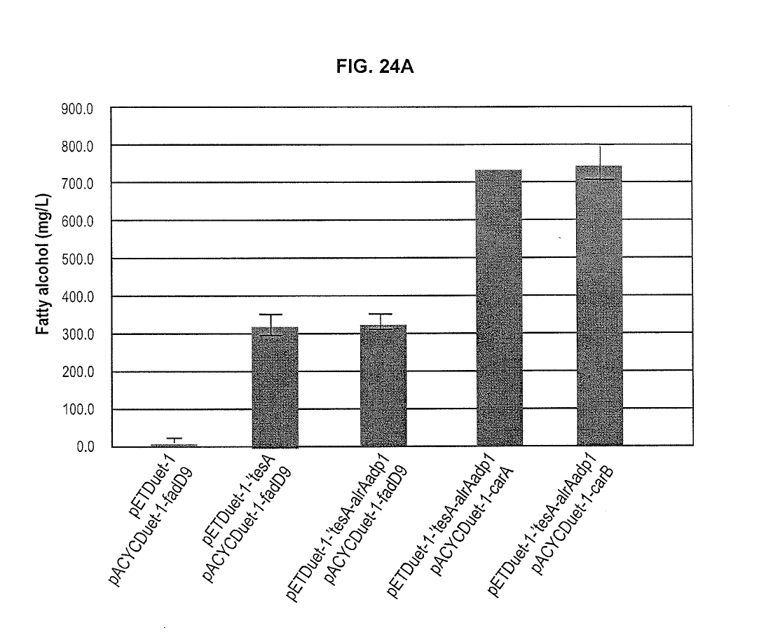

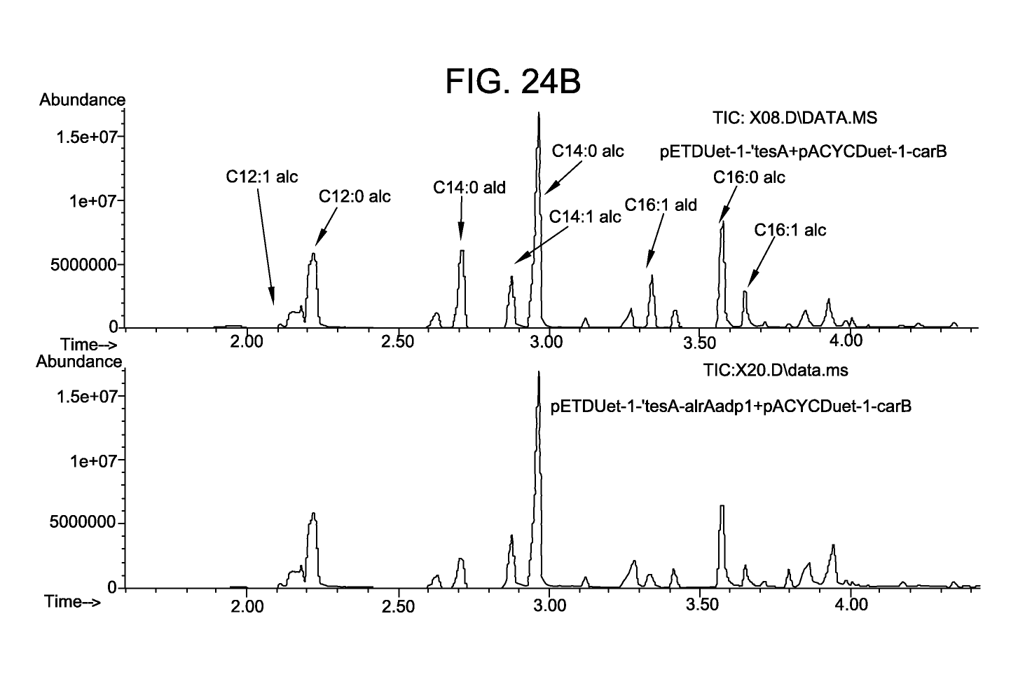

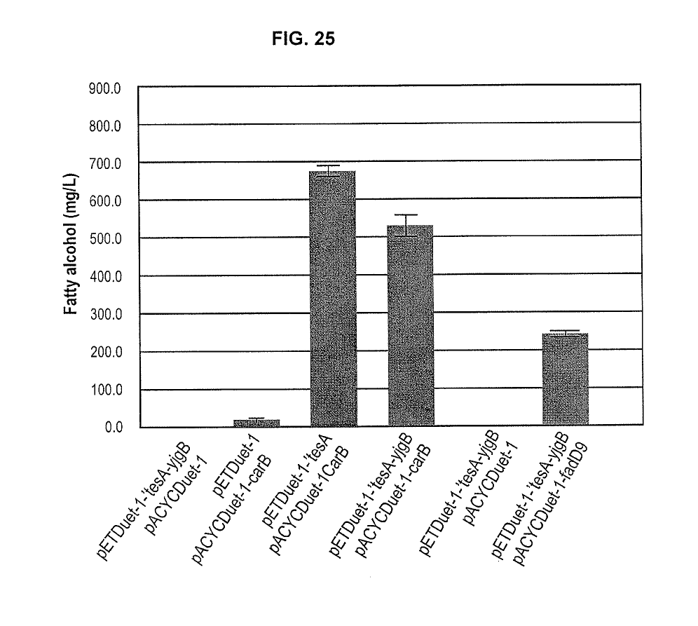

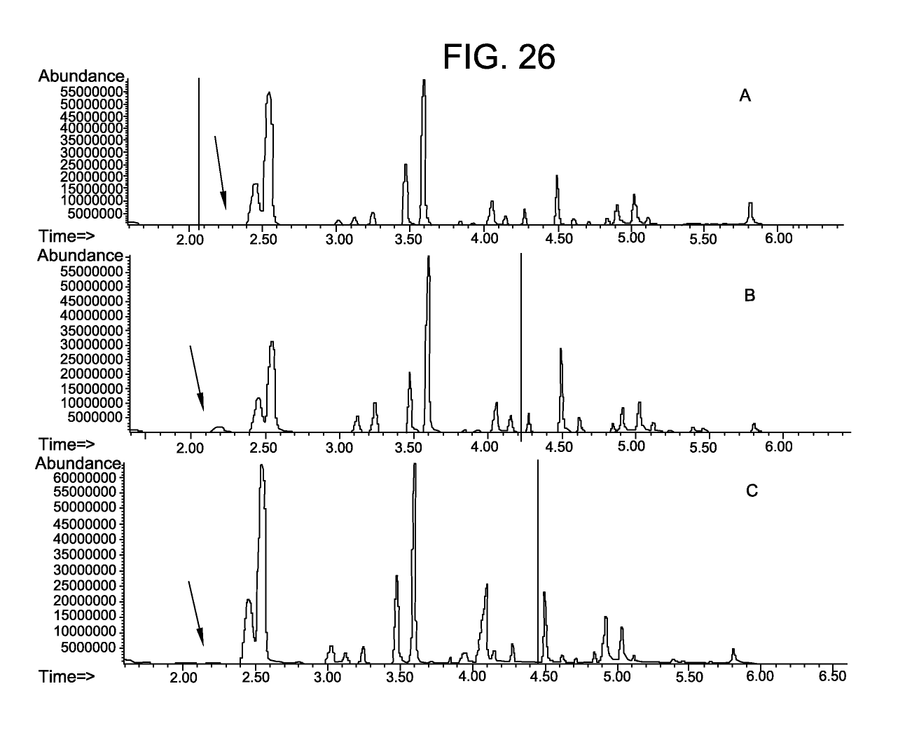

In yet other embodiments, the host cell is genetically engineered to express an attenuated level of a dehydratase/isomerase enzyme, such as an enzyme encoded by fabA or by a gene listed in the table of FIG. 22. In some embodiments, the host cell comprises a knockout of fabA or a gene listed in the table of FIG. 22. In other embodiments, the host cell is genetically engineered to express an attenuated level of a ketoacyl-ACP synthase, such as an enzyme encoded by fabB or by a gene listed in the table of FIG. 23. In certain embodiments, the host cell comprises a knockout of fabB or a gene listed in the table of FIG. 23. In yet other embodiments, the host cell is genetically engineered to express a modified level of a gene encoding a desaturase enzyme, such as desA.

### Formation of Branched Fatty Alcohols

Fatty alcohols can be produced from fatty aldehydes substrates that contain branched points by using a fatty alcohol biosynthetic polypeptide as described herein. In turn, the branched fatty aldehydes can be made from branched fatty acid derivatives as substrates for a fatty aldehyde biosynthetic polypeptide as described herein. For example, although E. coli naturally produces straight chain fatty acids (sFAs), E. coli can be engineered to produce branched chain fatty acids (brFAs) by introducing and expressing or overexpressing genes that provide branched precursors in the E. coli (e.g., bkd, ilv, icm, and fab gene families). Additionally, a host cell can be engineered to express or overexpress genes encoding proteins for the elongation of brFAs (e.g., ACP, FabF, etc.) and/or to delete or attenuate the corresponding host cell genes that normally lead to sFAs.

### Fatty Alcohol Saturation Levels

The degree of saturation in fatty acids (which can then be converted into fatty aldehydes and then fatty alcohols as described herein) can be controlled by regulating the degree of saturation of fatty acid intermediates. For example, the sfa, gns, and fab families of genes can be expressed, overexpressed, or expressed at reduced levels, to control the saturation of fatty acids. Non-limiting examples of these genes include sfa [GenBank Accession No. AAN 79592, AAC 44390], gnsA [GenBank Accession No. ABD 18647.1], gnsB [GenBank Accession No. AAC 74076.1], fabB [GenBank Accession No. BAA 16180, EC 2.3.1.41], fabK [GenBank Accession No. AAF 98273, EC1.3.1.9], fabL [GenBank Accession No. AAG 39821, EC 1.3.1.9], orfabM [GenBank Accession No. DAA 05501, EC 4.2.1.17].

For example, host cells can be engineered to produce unsaturated fatty acids by engineering the production host to overexpress fabB or by growing the production host at low temperatures (e.g., less than 37° C.). FabB has preference to cis-δ3decenoyl-ACP and results in unsaturated fatty acid production in E. coli. Overexpression of fabB results in the production of a significant percentage of unsaturated fatty acids (de Mendoza et al., J. Biol. Chem. 258:2098-2101, 1983). The gene fabB may be inserted into and expressed in host cells not naturally having the gene. These unsaturated fatty acids can then be used as intermediates in host cells that are engineered to produce fatty acid derivatives, such as fatty aldehydes.

In other instances, a repressor of fatty acid biosynthesis, for example, fabR (GenBank accession NP_{—}418398), can be deleted, which will also result in increased unsaturated fatty acid production in E. coli (Zhang et al., J. Biol. Chem. 277:15558, 2002). Similar deletions may be made in other host cells. A further increase in unsaturated fatty acids may be achieved, for example, by overexpressing fabM (trans-2, cis-3-decenoyl-ACP isomerase, GenBank accession DAA05501) and controlled expression of fabK (trans-2-enoyl-ACP reductase II, GenBank accession NP_{—} 357969) from Streptococcus pneumoniae (Marrakchi et al., J. Biol. Chem. 277: 44809, 2002), while deleting E. coli fabl (trans-2-enoyl-ACP reductase, GenBank accession NP_{—}415804). In some examples, the endogenous fabF gene can be attenuated, thus increasing the percentage of palmitoleate (C16:1) produced.

In yet other examples, host cells can be engineered to produce saturated fatty acids by reducing the expression of an sfa, gns, and/or fab gene.

### Formation of Cyclic Fatty Alcohols

Cyclic fatty alcohols can be produced from cyclic fatty aldehydes using cyclic fatty acid derivatives as substrates for a fatty aldehyde biosynthetic polypeptide described herein. To produce cyclic fatty acid derivative substrates, genes that provide cyclic precursors (e.g., the ans, chc, and plm gene families) can be introduced into the host cell and expressed to allow initiation of fatty acid biosynthesis from cyclic precursors.

### Fatty Aldehyde Biosynthetic Genes and Polypeptides.

In some embodiments, the microorganism is further engineered to express a modified level of a gene encoding a fatty aldehyde biosynthesis polypeptide. In certain embodiments, modifying the expression of a gene encoding a fatty aldehyde biosynthesis polypeptide includes expressing a gene encoding a fatty aldehyde biosynthetic enzyme and/or increasing the expression or activity of an endogenous fatty aldehyde biosynthetic enzyme. In some embodiments, the fatty aldehyde biosynthesis gene encodes a carboxylic acid reductase. In further embodiments, the fatty aldehyde biosynthetic gene encodes a fatty acid reductase.

In particular embodiments, the fatty aldehyde biosynthetic polypeptide comprises the amino acid sequence of SEQ ID NO: 41, 43, 45, 47, 49, 51, 53, 55, 57, 59, 61, 63, 65, 69, 71, 73, 75, 77, 79, 81, 83, 85, 87, 89, 91, 93, 97, 99, 101, 103, 105, 107, 109, 111, 113, 115, 117, 119, 121, 123, 125, 127, or a variant thereof. In some embodiments, the fatty aldehyde biosynthetic polypeptide comprises an amino acid sequence having at least about 80% (e.g., at least about 80%, at least about 85%, at least about 90%, at least about 91%, at least about 92%, at least about 93%, at least about 94%, at least about 95%, at least about 96%, at least about 97%, at least about 98%, at least about 99%) sequence identity to the amino acid sequence of SEQ ID NO: 41, 43, 45, 47, 49, 51, 53, 55, 57, 59, 61, 63, 65, 69, 71, 73, 75, 77, 79, 81, 83, 85, 87, 89, 91, 93, 97, 99, 101, 103, 105, 107, 109, 111, 113, 115, 117, 119, 121, 123, 125, or 127.

In another embodiment, the fatty aldehyde biosynthetic polypeptide is encoded by a polynucleotide having the sequence of SEQ ID NO:42, 44, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, or 128, or by a variant thereof. In some embodiments, the fatty aldehyde biosynthetic polypeptide is encoded by a polynucleotide having at least 80% sequence identity to the sequence of SEQ ID NO:42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, or 128. In some embodiments, the method further comprises expressing a gene encoding a fatty aldehyde biosynthesis polypeptide in the host cell. In particular embodiments, the fatty aldehyde biosynthetic polypeptide comprises the amino acid sequence of SEQ ID NO: 41, 43, 45, 47, 49, 51, 53, 55, 57, 59, 61, 63, 65, 69, 71, 73, 75, 77, 79, 81, 83, 85, 87, 89, 91, 93, 97, 99, 101, 103, 105, 107, 109, 111, 113, 115, 117, 119, 121, 123, 125, 127, or a variant thereof. In some embodiment, the fatty aldehyde biosynthetic polypeptide comprises an amino acid sequence having at least about 80% sequence identity to SEQ ID NO: 41, 43, 45, 47, 49, 51, 53, 55, 57, 59, 61, 63, 65, 69, 71, 73, 75, 77, 79, 81, 83, 85, 87, 89, 91, 93, 97, 99, 101, 103, 105, 107, 109, 111, 113, 115, 117, 119, 121, 123, 125, or 127. In another embodiment, the fatty aldehyde biosynthetic polypeptide is encoded by a polynucleotide having the sequence of SEQ ID NO: 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, or by a variant thereof. In some embodiments, the fatty aldehyde biosynthetic polypeptide is encoded by a polynucleotide having at least about 80% sequence identity to SEQ ID NO: 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, or 128. In further embodiments, the method comprises expressing a gene encoding a fatty aldehyde biosynthetic polypeptide comprising one or more of the amino acid motifs provided herein. For example, the fatty aldehyde biosynthetic gene can encode a polypeptide comprising SEQ ID NO:129, SEQ ID NO:130, SEQ ID NO:131, and SEQ ID NO:132; SEQ ID NO:133; SEQ ID NO:134; SEQ ID NO:135; SEQ ID NO: 136; and/or SEQ ID NO:129, SEQ ID NO:131, SEQ ID NO:132 and SEQ ID NO:133. SEQ ID NO:131 includes a reductase domain; SEQ ID NO:132 includes an NADP-binding domain; SEQ ID NO:133 includes a phosphopantetheine attachment site; and SEQ ID NO:134 includes an AMP-binding domain.

### Acyl-ACP Reductase Genes and Polypeptides.

In certain other embodiments, the invention further includes expressing in a host cell a gene encoding an acyl-ACP reductase polypeptide in the host cell. In some embodiments, the acyl-ACP reductase polypeptide comprises the amino acid sequence of SEQ ID NO: 137, 139, 141, 143, 145, 147, 149, 151, 153, or a variant thereof. In some embodiments, the acyl-ACP reductase polypeptide comprises an amino acid sequence that has at least about 70% (e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99%) sequence identity to SEQ ID NO: 137, 139, 141, 143, 145, 147, 149, 151, or 153. In another embodiment, the acyl-ACP reductase polypeptide is encoded by a polynucleotide having the sequence of SEQ ID NO: 138, 140, 142, 144, 146, 148, 150, 152, or 154, or by a variant thereof. In some embodiments, the acyl-ACP reductase polypeptide is encoded by a polynucleotide having at least about 70% sequence identity to the sequence of SEQ ID NO: 138, 140, 142, 144, 146, 148, 150, 152, or 154.

In yet further embodiments, the method includes expressing in a host cell an acyl-ACP reductase gene encoding a polypeptide comprising one or more of the amino acid motifs disclosed herein. For example, the polypeptide can comprise one or more of SEQ ID NO:155, 156, 157, 158, 159, 160, 161, 162, 163, 164, or 165.

### Hydrocarbon Biosynthetic Genes and Polypeptides.

The compositions and methods described herein can be used to produce hydrocarbons, including, for example, alkanes and alkenes, from an appropriate substrate.

The invention is based, at least in part, on the identification of a number of fatty alcohol biosynthetic enzymes or polypeptides that are capable of catalyzing the conversion of a fatty aldehyde to a fatty alcohol under suitable conditions, for example, in the presence of suitable substrates and/or co-factors. One or more of these fatty alcohol biosynthetic polypeptides can be attenuated or deleted from the host cell, which expresses or overexpresses one or more hydrocarbon biosynthetic polypeptides, optionally also expresses or overexpresses one or more fatty aldehyde biosynthetic polypeptides or one or more acyl-ACP reductases. The resulting host cell can be used to produce hydrocarbons such as, for example, alkanes or alkenes. In certain embodiments, the hydrocarbons are produced by a biosynthetic pathway depicted in FIG. 1B. In this pathway, a fatty acid is first activated by ATP and then reduced by a fatty aldehyde biosynthetic polypeptide such as a carboxylic acid reductase (CAR)-like enzyme to generate a fatty aldehyde. The fatty aldehyde can then be subject to a hydrocarbon biosynthetic polypeptide such as a decarbonylase and be reduced into a hydrocarbon. In certain other embodiments, hydrocarbons are produced by an alternative biosynthesis pathway depicted in FIG. 1B. In this pathway, an acyl-ACP is converted into a fatty aldehyde catalyzed by an acyl-ACP reductase. The fatty aldehyde is further subject to a hydrocarbon biosynthetic polypeptide and converts to a hydrocarbon such as an alkane or an alkene. In both of these pathways, the fatty aldehydes can, in the presence of endogenous fatty alcohol biosynthetic enzyme activity, be converted into fatty alcohols. Therefore, attenuating one or more fatty alcohol biosynthetic polypeptides, or in particular embodiments, deleting one or more fatty alcohol biosynthetic polypeptides from the host cell can improve the production of hydrocarbons. In some embodiments, the method further includes culturing the host cell in the presence of at least one biological substrate of the hydrocarbon biosynthetic polypeptide, the fatty aldehyde biosynthetic polypeptide, and/or the acyl-ACP reductase polypeptide. Exemplary suitable substrates include, without limitation, a fatty acid derivative, an acyl-ACP, a fatty acid, an acyl-CoA, a fatty aldehyde, a fatty alcohol, or a fatty ester.

In another aspect, the invention features a method of producing a hydrocarbon, the method comprising expressing an attenuated level of one or more fatty alcohol biosynthetic genes or a mutant and variant thereof in a host cell. In certain embodiments, the method further comprises deleting one or more fatty alcohol biosynthetic genes or a mutant and variant thereof from the host cell. Fatty alcohol biosynthetic genes, polypeptides, sequence motifs, mutants and variants thereof, are described hereinabove.

In certain other embodiments, the host cell is engineered such that it comprises no detectable level of fatty alcohol biosynthetic enzyme activity, for example, a fatty aldehyde reductase activity, an alcohol dehydrogenase activity, an aldo-keto reductase activity, an oxidoreductase activity, or a short-chain dehydrogenase activity.

In some embodiments, the method further comprises expressing a gene encoding a hydrocarbon biosynthetic polypeptide in the host cell. In particular embodiments, the hydrocarbon biosynthetic polypeptide comprises the amino acid sequence of SEQ ID NO: 166, 168, 170, 172, 174, 176, 178, 180, 182, 184, 186, 188, 190, 192, 194, 196, 198, 200, or a variant thereof. In some embodiments, the hydrocarbon biosynthetic polypeptide comprises at least about 70% sequence identity to SEQ ID NO: 166, 168, 170, 172, 174, 176, 178, 180, 182, 184, 186, 188, 190, 192, 194, 196, 198, or 200. In another embodiment, the hydrocarbon biosynthetic polypeptide is encoded by a polynucleotide having the sequence of SEQ ID NO: 167, 169, 171, 173, 175, 177, 179, 181, 183, 185, 187, 189, 191, 193, 195, 197, 199, or 201, or by a variant thereof. In some embodiments, the hydrocarbon biosynthetic polypeptide is encoded by a polynucleotide having at least about 70% sequence identity to SEQ ID NO: 167, 169, 171, 173, 175, 177, 179, 181, 183, 185, 187, 189, 191, 193, 195, 197, 199, or 201. In further embodiments, the method comprises expressing a gene encoding a hydrocarbon biosynthetic polypeptide comprising one or more amino acid motifs disclosed herein. For example, the hydrocarbon biosynthetic polypeptide can comprise the amino acid sequence motifs of: (1) SEQ ID NO: 202; or (2) SEQ ID NO: 203 or SEQ ID NO:204, or SEQ ID NO:205; or (3) SEQ ID NO:206, and any one of SEQ ID NO:203, SEQ ID NO:204, SEQ ID NO:205; or (4) SEQ ID NO:207 or SEQ ID NO:208, or SEQ ID NO:209, or SEQ ID NO:210. In certain embodiments, the hydrocarbon biosynthetic polypeptide has decarbonylase activity. In some embodiments, the method further comprises isolating the hydrocarbon from the host cell.

In some embodiments, the method further comprises expressing a gene encoding a fatty aldehyde biosynthesis polypeptide in the host cell. Fatty aldehyde biosynthetic genes, polypeptides, sequence motifs, mutants, and variants thereof are described hereinabove.

In any of the aspects of the invention described herein, the method can produce hydrocarbons. In some embodiments, the hydrocarbon produced is an alkane. In some embodiments, the alkane is a C₃-C₂₅ alkane. For example, the alkane is a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₋₅ alkane. In some embodiments, the alkane is tridecane, methyltridecane, nonadecane, methylnonadecane, heptadecane, methylheptadecane, pentadecane, or methylpentadecane.

In certain embodiments, the method further comprising culturing the host cell in the presence of a saturated fatty acid derivative, and the hydrocarbon produced is an alkane. In certain embodiments, the saturated fatty acid derivative is a C₆-C₂₆ fatty acid derivative substrate. For example, the fatty acid derivative substrate is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty acid derivative substrate. In particular embodiments, the fatty acid derivative substrate is 2-methylicosanal, icosanal, octadecanal, tetradecanal, 2-methyloctadecanal, stearaldehyde, or palmitaldehyde.

In some embodiments, the method further includes isolating the alkane from the host cell or from the culture medium. In certain embodiments, the method further includes cracking or refining the alkane.

In any of the aspects of the invention herein, the hydrocarbon carbon produced can be an alkene. In some embodiments, the alkene is a C₃-C₂₅ alkene. For example, the alkene is a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₋₅ alkene. In some embodiments, the alkene is pentadecene, heptadecene, methylpentadecene, or methylheptadecene.

In some embodiments, the alkene is a straight chain alkene, a branched chain alkene, or a cyclic alkene.

In certain embodiments, the method further comprises culturing the host cell in the presence of an unsaturated fatty acid derivative, and the hydrocarbon produced is an alkene. In certain embodiments, the unsaturated fatty acid derivative is a C₆-C₂₆ fatty acid derivative substrate. For example, the fatty acid derivative substrate is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ unsaturated fatty acid derivative substrate. In particular embodiments, the fatty acid derivative substrate is octadecenal, hexadecenal, methylhexadecenal, or methyloctadecenal.

In another aspect, the invention features a genetically engineered microorganism wherein the microorganism produces an increased level of a hydrocarbon relative to a wild-type microorganism.

In another aspect, the invention features a method of making a hydrocarbon, the method comprising culturing a genetically engineered microorganism described herein under conditions suitable for gene expression, and isolating the hydrocarbon. In certain embodiments, the method comprising culturing the genetically engineered microorganism in the presence of a suitable biological substrate for the hydrocarbon biosynthetic polypeptide, the fatty aldehyde biosynthetic polypeptide, and/or the acyl-ACP reductase.

In some embodiments, the biological substrate is a fatty acid derivative, an acyl-ACP, a fatty acid, an acyl-CoA, a fatty aldehyde, a fatty alcohol, or a fatty ester.

In some embodiments, the substrate is a saturated fatty acid derivative, and the hydrocarbon produced is an alkane, for example, a C₃-C₂₅ alkane. For example, the alkane is a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₋₅ alkane. In some embodiments, the alkane is tridecane, methyltridecane, nonadecane, methylnonadecane, heptadecane, methylheptadecane, pentadecane, or methylpentadecane.

In some embodiments, the alkane is a straight chain alkane, a branched chain alkane, or a cyclic alkane.

In some embodiments, the saturated fatty acid derivative is 2-methylicosanal, icosanal, octadecanal, tetradecanal, 2-methyloctadecanal, stearaldehyde, or palmitaldehyde.

In other embodiments, the biological substrate is an unsaturated fatty acid derivative and the hydrocarbon produced by the microorganism is an alkene, for example, a C₃-C₂₅ alkene. For example, the alkene is a C₃, C₄, C₅, C₆, C₇, C₈, C₉, C₁₀, C₁₁, C₁₂, C₁₃, C₁₄, C₁₅, C₁₄, C₁₅, C16, C17, C18, C₁₉, C₂₀, C₂₁, C₂₂, C₂₃, C₂₄, or C₂₋₅ alkene. In some embodiments, the alkene is pentadecene, heptadecene, methylpentadecene, or methylheptadecene.

In some embodiments, the alkene is a straight chain alkene, a branched chain alkene, or a cyclic alkene. In some embodiments, the unsaturated fatty acid derivative is octadecenal, hexadecenal, methylhexadecenal, or methyloctadecenal.

In another aspect, the invention features a hydrocarbon produced by any of the methods or microorganisms described herein. In particular embodiments, the hydrocarbon is an alkane or an alkene having a δ¹³C of about −15.4 or greater. In certain embodiments, the alkane or alkene has a δ¹³C of about −15.4 to about −10.9, or of about −13.92 to about −13.84.

In other embodiments, the alkane or alkene has an f_{M}¹⁴C of at least about 1.003. In certain embodiments, the alkene or alkene has an f_{M}¹⁴C of at least about 1.01 or at least about 1.5. In some embodiments, the alkane or alkene has an f_{M}¹⁴C of about 1.111 to about 1.124.

In another aspect, the invention features a biofuel comprising a hydrocarbon produced by any of the methods or microorganisms described herein. In particular embodiments, the hydrocarbon is an alkane or an alkene having a δ¹³C of about −15.4 or greater. In exemplary embodiments, the alkane or alkene has a δ¹³C of about −15.4 to about −10.9, or of about −13.92 to about −13.84. In other embodiments, the alkane or alkene has an f_{M}¹⁴C of at least about 1.003. For example, the alkane or alkene has an f_{M}¹⁴C of at least about 1.003. For example, the alkane or alkene has an f_{M}¹⁴C of at least about 1.01 or at least about 1.5. In some embodiments, the alkane or alkene has an f_{M}¹⁴C of about 1.111 to about 1.124.

In any of the aspects described herein, a hydrocarbon is produced in a host cell or a microorganism described herein from a carbon source.

### Variants

As used herein, a “variant” of polypeptide X refers to a polypeptide having the amino acid sequence of peptide X in which one or more amino acid residues is altered. The variant may have conservative changes or nonconservative changes. Guidance in determining which amino acid residues may be substituted, inserted, or deleted without affecting biological activity may be found using computer programs well known in the art, for example, LASERGENE software (DNASTAR). The term “variant,” when used in the context of a polynucleotide sequence, may encompass a polynucleotide sequence related to that of a gene or the coding sequence thereof. This definition may also include, for example, “allelic,” “splice,” “species,” or “polymorphic” variants. A splice variant may have significant identity to a reference polynucleotide, but will generally have a greater or fewer number of polynucleotides due to alternative splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species.

Suitable variants, such as those described herein, for example in FIGS. 2-21, can be identified using bioinformatic tools such as searching for the “bidirectional best hits” against the public databases, such as for example, the Kyoto Encyclopedia of Gene & Genomes (KEGG) database, and selecting bidirectional best hits having a Smith-Waterman score of for example, above 1000. Other bioinformatics tools known to those skilled in the art, including for example, a bi-directional blast against known genome databases and the E. coli genome, can also be used for this purpose to identify homologs.

Variants can be naturally occurring or created in vitro. In particular, such variants can be created using genetic engineering techniques, such as site directed mutagenesis, random chemical mutagenesis, Exonuclease III deletion procedures, or standard cloning techniques. Alternatively, such variants, fragments, analogs, or derivatives can be created using chemical synthesis or modification procedures.

Methods of making variants are well known in the art. These include procedures in which nucleic acid sequences obtained from natural isolates are modified to generate nucleic acids that encode polypeptides having characteristics that enhance their value in industrial or laboratory applications. In such procedures, a large number of variant sequences having one or more nucleotide differences with respect to the sequence obtained from the natural isolate are generated and characterized. Typically, these nucleotide differences result in amino acid changes with respect to the polypeptides encoded by the nucleic acids from the natural isolates.

For example, variants can be created using error prone PCR (see, e.g., Leung et al., Technique 1:11-15, 1989; and Caldwell et al., PCR Methods Applic. 2:28-33, 1992). In error prone PCR, PCR is performed under conditions where the copying fidelity of the DNA polymerase is low, such that a high rate of point mutations is obtained along the entire length of the PCR product. Briefly, in such procedures, nucleic acids to be mutagenized (e.g., a fatty aldehyde biosynthetic polynucleotide sequence), are mixed with PCR primers, reaction buffer, MgCl₂, MnCl₂, Taq polymerase, and an appropriate concentration of dNTPs for achieving a high rate of point mutation along the entire length of the PCR product. For example, the reaction can be performed using 20 fmoles of nucleic acid to be mutagenized (e.g., a fatty aldehyde biosynthetic polynucleotide sequence), 30 pmole of each PCR primer, a reaction buffer comprising 50 mM KCl, 10 mM Tris HCl (pH 8.3), and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR can be performed for 30 cycles of 94° C. for 1 min, 45° C. for 1 min, and 72° C. for 1 min. However, it will be appreciated that these parameters can be varied as appropriate. The mutagenized nucleic acids are then cloned into an appropriate vector and the activities of the polypeptides encoded by the mutagenized nucleic acids are evaluated.

Variants can also be created using oligonucleotide directed mutagenesis to generate site-specific mutations in any cloned DNA of interest. Oligonucleotide mutagenesis is described in, for example, Reidhaar-Olson et al., Science 241:53-57, 1988. Briefly, in such procedures a plurality of double stranded oligonucleotides bearing one or more mutations to be introduced into the cloned DNA are synthesized and inserted into the cloned DNA to be mutagenized (e.g., a fatty aldehyde biosynthetic polynucleotide sequence). Clones containing the mutagenized DNA are recovered, and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, for example, U.S. Pat. No. 5,965,408.

Still another method of generating variants is sexual PCR mutagenesis. In sexual PCR mutagenesis, forced homologous recombination occurs between DNA molecules of different, but highly related, DNA sequence in vitro as a result of random fragmentation of the DNA molecule based on sequence homology. This is followed by fixation of the crossover by primer extension in a PCR reaction. Sexual PCR mutagenesis is described in, for example, Stemmer, PNAS, USA 91:10747-10751, 1994.

Recursive ensemble mutagenesis can also be used to generate variants. Recursive ensemble mutagenesis is an algorithm for protein engineering (i.e., protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described in, for example, Arkin et al., PNAS, USA 89:7811-7815, 1992.

In some embodiments, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described in, for example, Delegrave et al., Biotech. Res. 11:1548-1552, 1993. Random and site-directed mutageneses are described in, for example, Arnold, Curr. Opin. Biotech. 4:450-455, 1993.

In some embodiments, variants are created using shuffling procedures wherein portions of a plurality of nucleic acids that encode distinct polypeptides are fused together to create chimeric nucleic acid sequences that encode chimeric polypeptides as described in, for example, U.S. Pat. Nos. 5,965,408 and 5,939,250.

Polynucleotide variants also include nucleic acid analogs. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine and 5-methyl-2′-deoxycytidine or 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone and the four bases are retained. (See, e.g., Summerton et al., Antisense Nucleic Acid Drug Dev. (1997) 7:187-195; and Hyrup et al., Bioorgan. Med. Chem. (1996) 4:5-23.) In addition, the deoxyphosphate backbone can be replaced with, for example, a phosphorothioate or phosphorodithioate backbone, a phosphoroamidite, or an alkyl phosphotriester backbone.

Biosynthetic polypeptide variants can be variants in which one or more amino acid residues are substituted with a conserved or non-conserved amino acid residues. In preferred embodiments, biosynthetic polypeptide variants are variants in which one or more amino acid residues are substituted with a conserved amino acid residue. Such substituted amino acid residue may or may not be one encoded by a genetic code.

Conservative substitutions are those that substitute a given amino acid in a polypeptide by another amino acid of similar characteristics. Typical conservative substitutions are the following replacements: replacement of an aliphatic amino acid, such as alanine, valine, leucine, and isoleucine, with another aliphatic amino acid; replacement of a serine with a threonine or vice versa; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

Other polypeptide variants are those in which one or more amino acid residues include a substituent group. Still other polypeptide variants are those in which the polypeptide is associated with another compound, such as a compound to increase the half-life of the polypeptide (e.g., polyethylene glycol).

Additional polypeptide variants are those in which additional amino acids are fused to the polypeptide, such as a leader sequence, a secretory sequence, a proprotein sequence, or a sequence which facilitates purification, enrichment, or stabilization of the polypeptide.

In some instances, the polypeptide variants retain the same biological function as a the native polypeptide, for example, retain fatty alcohol biosynthetic activity, such as fatty aldehyde reductase, alcohol dehydrogenase, aldo-keto reductase, short-chain alcohol dehydrogenases, or oxidoreductase activity or retain fatty aldehyde biosynthetic activity, such as carboxylic acid or fatty acid reductase activity, and have amino acid sequences substantially identical thereto.

In other instances, the polypeptide variants have at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, or more than about 95% homology to the native or wild-type sequence. In another embodiment, the polypeptide variants include a fragment comprising at least about 5, 10, 15, 20, 25, 30, 35, 40, 50, 75, 100, or 150 consecutive amino acids thereof.

The polypeptide variants or fragments thereof can be obtained by isolating nucleic acids encoding them using techniques described herein or by expressing synthetic nucleic acids encoding them. Alternatively, polypeptide variants or fragments thereof can be obtained through biochemical enrichment or purification procedures. The sequence of polypeptide variants or fragments can be determined by proteolytic digestion, gel electrophoresis, and/or microsequencing. The sequence of the polypeptide variants or fragments can then be compared to the native or wild-type sequence using any of the programs described herein.

The polypeptide variants and fragments thereof can be assayed for fatty aldehydes producing activity, fatty alcohol producing activity or hydrocarbon producing activity using routine methods. For example, the polypeptide variants or fragment can be contacted with a substrate (e.g., a fatty acid or fatty aldehyde substrate) under conditions that allow the polypeptide variant to function. A decreased in the level of the substrate or an increase in the level of a fatty aldehydes, fatty alcohol or hydrocarbon, respectively, can be measured to determine the biological activity of the variant or fragment.

The terms “homolog,” “homologue,” and “homologous” as used herein refer to a polynucleotide or a polypeptide comprising a sequence that is at least about 80% homologous to the corresponding polynucleotide or polypeptide sequence. One of ordinary skill in the art is well aware of methods to determine homology between two or more sequences. Briefly, calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a first sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of a second sequence. The amino acid residues or nucleotides at corresponding amino acid positions or nucleotide positions of the first and second sequences are then compared. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps and the length of each gap, which need to be introduced for optimal alignment of the two sequences.

The comparison of sequences and determination of percent homology between two sequences can be accomplished using a mathematical algorithm, such as BLAST (Altschul et al., J. Mol. Biol., 215(3): 403-410 (1990)). The percent homology between two amino acid sequences also can be determined using the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 (Needleman and Wunsch, J. Mol. Biol., 48: 444-453 (1970)). The percent homology between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial homology calculations and adjust the algorithm parameters accordingly. A preferred set of parameters (and the one that should be used if a practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5. Additional methods of sequence alignment are known in the biotechnology arts (see, e.g., Rosenberg, BMC Bioinformatics, 6: 278 (2005); Altschul et al., FEBS J., 272(20): 5101-5109 (2005)).

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either method can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2.×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions unless otherwise specified.

In some embodiments, the polypeptide is a fragment of any of the polypeptides described herein. The term “fragment” refers to a shorter portion of a full-length polypeptide or protein ranging in size from four amino acid residues to the entire amino acid sequence minus one amino acid residue. In certain embodiments of the invention, a fragment refers to the entire amino acid sequence of a domain of a polypeptide or protein (e.g., a substrate binding domain or a catalytic domain).

In some embodiments, the polypeptide is a mutant or a variant of any of the polypeptides described herein. The terms “mutant” and “variant” as used herein refer to a polypeptide having an amino acid sequence that differs from a wild-type polypeptide by at least one amino acid. For example, the mutant or variant can comprise one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue. In some embodiments, the mutant polypeptide has about 1, 2, 3, 4, 5, 6, 7, 8, 9, 10, 15, 20, 30, 40, 50, 60, 70, 80, 90, 100, or more amino acid substitutions, additions, insertions, or deletions.

Preferred fragments or mutants of a polypeptide retain some or all of the biological function (e.g., enzymatic activity) of the corresponding wild-type polypeptide. In some embodiments, the fragment or mutant retains at least 75%, at least 80%, at least 90%, at least 95%, or at least 98% or more of the biological function of the corresponding wild-type polypeptide. In other embodiments, the fragment or mutant retains about 100% of the biological function of the corresponding wild-type polypeptide. Guidance in determining which amino acid residues may be substituted, inserted, or deleted without affecting biological activity may be found using computer programs well known in the art, for example, LASERGENE™ software (DNASTAR, Inc., Madison, Wis.).

In yet other embodiments, a fragment or mutant exhibits increased biological function as compared to a corresponding wild-type polypeptide. For example, a fragment or mutant may display at least a 10%, at least a 25%, at least a 50%, at least a 75%, or at least a 90% improvement in enzymatic activity as compared to the corresponding wild-type polypeptide. In other embodiments, the fragment or mutant displays at least 100% (e.g., at least 200%, or at least 500%) improvement in enzymatic activity as compared to the corresponding wild-type polypeptide.

It is understood that the polypeptides described herein may have additional conservative or non-essential amino acid substitutions, which do not have a substantial effect on the polypeptide function. Whether or not a particular substitution will be tolerated (i.e., will not adversely affect desired biological function, such as DNA binding or enzyme activity) can be determined as described in Bowie et al. (Science, 247: 1306-1310 (1990)).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some embodiments, the fatty acid or fatty acid derivative biosynthetic polypeptide or polynucleotide is from a bacterium, a cyanobacterium, an algae, a plant, an insect, a yeast, a fungus, or a mammal. In certain embodiments, the polypeptide is from a mammalian cell, plant cell, insect cell, fungus cell, cyanobacterial cell, algal cell, bacterial cell, or any other organisms described herein.

### Vectors and Expression

In some embodiments, a polynucleotide (or gene) sequence is provided to the host cell by way of a recombinant vector, which comprises a promoter operably linked to the polynucleotide sequence. In certain embodiments, the promoter is a developmentally-regulated, an organelle-specific, a tissue-specific, an inducible, a constitutive, or a cell-specific promoter.

In some embodiments, the recombinant vector comprises at least one sequence selected from the group consisting of (a) an expression control sequence operatively coupled to the polynucleotide sequence; (b) a selection marker operatively coupled to the polynucleotide sequence; (c) a marker sequence operatively coupled to the polynucleotide sequence; (d) a purification moiety operatively coupled to the polynucleotide sequence; (e) a secretion sequence operatively coupled to the polynucleotide sequence; and (f) a targeting sequence operatively coupled to the polynucleotide sequence.

The expression vectors described herein include a polynucleotide sequence described herein in a form suitable for expression of the polynucleotide sequence in a host cell. It will be appreciated by those skilled in the art that the design of the expression vector can depend on such factors as the choice of the host cell to be transformed, the level of expression of polypeptide desired, etc. The expression vectors described herein can be introduced into host cells to produce polypeptides, including fusion polypeptides, encoded by the polynucleotide sequences as described herein.

Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino- or carboxy-terminus of the recombinant polypeptide. Such fusion vectors typically serve one or more of the following three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech, Inc., Piscataway, N.J.; Smith et al., Gene, 67: 31-40 (1988)), pMAL (New England Biolabs, Beverly, Mass.), and pRITS (Pharmacia Biotech, Inc., Piscataway, N.J.), which fuse glutathione S-transferase (GST), maltose E binding protein, or protein A, respectively, to the target recombinant polypeptide.

Examples of inducible, non-fusion E. coli expression vectors include pTrc (Amann et al., Gene (1988) 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

Suitable expression systems for both prokaryotic and eukaryotic cells are well known in the art; see, e.g., Sambrook et al., “Molecular Cloning: A Laboratory Manual,” second edition, Cold Spring Harbor Laboratory, (1989). Examples of inducible, non-fusion E. coli expression vectors include pTrc (Amann et al., Gene, 69: 301-315 (1988)) and PET 11d (Studier et al., Gene Expression Technology Methods in Enzymology 185, Academic Press, San Diego, Calif., pp. 60-89 (1990)). In certain embodiments, a polynucleotide sequence of the invention is operably linked to a promoter derived from bacteriophage T5.

In another embodiment, the host cell is a yeast cell. In this embodiment, the expression vector is a yeast expression vector. Examples of vectors for expression in yeast include pYepSec1 (Baldari et al., EMBO J., 6: 229-234 (1987)), pMFa (Kurjan et al., Cell, 30: 933-943 (1982)), pJRY88 (Schultz et al., Gene, 54: 113-123 (1987)), pYES2 (Invitrogen Corp., San Diego, Calif.), and picZ (Invitrogen Corp., San Diego, Calif.).

Alternatively, a polypeptide described herein can be expressed in insect cells using baculovirus expression vectors. Baculovirus vectors available for expression of proteins in cultured insect cells (e.g., Sf9 cells) include, for example, the pAc series (Smith et al., Mol. Cell. Biol. (1983) 3:2156-2165) and the pVL series (Lucklow et al., Virology (1989) 170:31-39).

In yet another embodiment, the nucleic acids described herein can be expressed in mammalian cells using a mammalian expression vector. Examples of mammalian expression vectors include pCDM8 (Seed, Nature (1987) 329:840) and pMT2PC (Kaufman et al., EMBO J. (1987) 6:187-195). When used in mammalian cells, the expression vector's control functions can be provided by viral regulatory elements. For example, commonly used promoters are derived from polyoma, Adenovirus 2, cytomegalovirus, and Simian Virus 40. Other suitable expression systems for both prokaryotic and eukaryotic cells are described in chapters 16 and 17 of Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989.

Vectors can be introduced into prokaryotic or eukaryotic cells via a variety of art-recognized techniques for introducing foreign nucleic acid (e.g., DNA) into a host cell. Suitable methods for transforming or transfecting host cells can be found in, for example, Sambrook et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to an antibiotic) can be introduced into the host cells along with the gene of interest. Selectable markers include those that confer resistance to drugs such as, but not limited to, ampicillin, kanamycin, chloramphenicol, or tetracycline. Nucleic acids encoding a selectable marker can be introduced into a host cell on the same vector as that encoding a polypeptide described herein or can be introduced on a separate vector. Cells stably transformed with the introduced nucleic acid can be identified by growth in the presence of an appropriate selection drug.

Similarly, for stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to an antibiotic) can be introduced into the host cells along with the gene of interest. Preferred selectable markers include those which confer resistance to drugs, such as G418, hygromycin, and methotrexate. Nucleic acids encoding a selectable marker can be introduced into a host cell on the same vector as that encoding a polypeptide described herein or can be introduced on a separate vector. Cells stably transfected with the introduced nucleic acid can be identified by growth in the presence of an appropriate selection drug.

### Host Cells

As used herein, a “host cell” is a cell used to produce a product described herein (e.g., a fatty aldehydes, a fatty alcohol or a hydrocarbon).

A host cell is referred to as an “engineered host cell” or a “recombinant host cell” if the expression of one or more polynucleotides or polypeptides in the host cell are altered or modified as compared to their expression in a corresponding wild-type host cell under the same conditions.

In any of the aspects of the invention described herein, the host cell can be selected from the group consisting of a eukaryotic plant, algae, cyanobacterium, green-sulfur bacterium, green non-sulfur bacterium, purple sulfur bacterium, purple non-sulfur bacterium, extremophile, yeast, fungus, engineered organisms thereof, or a synthetic organism. In some embodiments, the host cell is light dependent or fixes carbon. In some embodiments, the host cell is light dependent or fixes carbon. In some embodiments, the host cell has autotrophic activity.

Various host cells can be used to produce fatty aldehydes, fatty alcohols and hydrocarbons, as described herein. A host cell can be any prokaryotic or eukaryotic cell. For example, a gene encoding a polypeptide described herein (e.g., a fatty aldehyde biosynthetic polypeptide, or an acyl-ACP reductase polypeptide, and/or a fatty alcohol biosynthetic polypeptide) can be expressed in bacterial cells (such as E. coli), insect cells, yeast, or mammalian cells (such as Chinese hamster ovary cells (CHO) cells, COS cells, VERO cells, BHK cells, HeLa cells, Cv1 cells, MDCK cells, 293 cells, 3T3 cells, or PC12 cells).

Exemplary host cells can be from the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Schizosaccharomyces, Yarrowia, or Streptomyces.

In some embodiments, the host cell is a Gram-positive bacterial cell. In other embodiments, the host cell is a Gram-negative bacterial cell.

In some embodiments, the host cell is selected from the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

In certain embodiments, the host cell is a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell.

In yet other embodiments, the host cell is a Streptomyces lividans cell or a Streptomyces murinus cell.

In yet other embodiments, the host cell is an Actinomycetes cell.

In some embodiments, the host cell is a Saccharomyces cerevisiae cell.

Additional host cells that can be used in the methods described herein are described in WO2009/111513 and WO2009/111672.

### Transport Proteins

Transport proteins can export polypeptides and organic compounds (e.g., fatty alcohols) out of a host cell. Many transport and efflux proteins serve to excrete a wide variety of compounds and can be naturally modified to be selective for particular types of hydrocarbons.

Non-limiting examples of suitable transport proteins are ATP-Binding Cassette (ABC) transport proteins, efflux proteins, and fatty acid transporter proteins (FATP). Additional non-limiting examples of suitable transport proteins include the ABC transport proteins from organisms such as Caenorhabditis elegans, Arabidopsis thalania, Alkaligenes eutrophus, and Rhodococcus erythropolis. Exemplary ABC transport proteins include, without limitation, CER5 [Accession No: At1g 51510, AY734542, At3g 2190, or At1g51460], AtMRP5 [Accession No. NP_{—}171908], AmiS2 [Accession No: JC5491], and AtPGP1 [Accession No: NP_{—}181228]. Host cells can also be chosen for their endogenous ability to secrete organic compounds. The efficiency of organic compound production and secretion into the host cell environment (e.g., culture medium, fermentation broth) can be expressed as a ratio of intracellular product to extracellular product. In some examples, the ratio can be about 5:1, 4:1, 3:1, 2:1, 1:1, 1:2, 1:3, 1:4, or 1:5.

### Fermentation

The production and isolation of fatty alcohols can be enhanced by employing beneficial fermentation techniques. One method for maximizing production while reducing costs is increasing the percentage of the carbon source that is converted to hydrocarbon products.

During normal cellular lifecycles, carbon is used in cellular functions, such as producing lipids, saccharides, proteins, organic acids, and nucleic acids. Reducing the amount of carbon necessary for growth-related activities can increase the efficiency of carbon source conversion to product. This can be achieved by, for example, first growing host cells to a desired density (for example, a density achieved at the peak of the log phase of growth). At such a point, replication checkpoint genes can be harnessed to stop the growth of cells. Specifically, quorum sensing mechanisms (reviewed in Camilli et al., Science 311:1113, 2006; Venturi FEMS Microbio. Rev. 30:274-291, 2006; and Reading et al., FEMS Microbiol. Lett. 254:1-11, 2006) can be used to activate checkpoint genes, such as p. 5.3, p21, or other checkpoint genes.

Genes that can be activated to stop cell replication and growth in E. coli include umuDC genes. The overexpression of umuDC genes stops the progression from stationary phase to exponential growth (Murli et al., J. of Bact. 182:1127, 2000). UmuC is a DNA polymerase that can carry out translesion synthesis over non-coding lesions—the mechanistic basis of most UV and chemical mutagenesis. The umuDC gene products are involved in the process of translesion synthesis and also serve as a DNA sequence damage checkpoint. The umuDC gene products include UmuC, UmuD, umuD′, UmuD′₂C, UmuD′₂, and UmuD₂. Simultaneously, product-producing genes can be activated, thus minimizing the need for replication and maintenance pathways to be used while a fatty aldehyde is being made. Host cells can also be engineered to express umuC and umuD from E. coli in pBAD24 under the prpBCDE promoter system through de novo synthesis of this gene with the appropriate end-product production genes.

The percentage of input carbons converted to fatty alcohols can be a cost driver. The more efficient the process is (i.e., the higher the percentage of input carbons converted to fatty alcohols), the less expensive the process will be. For oxygen-containing carbon sources (e.g., glucose and other carbohydrate based sources), the oxygen must be released in the form of carbon dioxide. For every 2 oxygen atoms released, a carbon atom is also released leading to a maximal theoretical metabolic efficiency of approximately 34% (w/w) (for fatty acid derived products). This figure, however, changes for other organic compounds and carbon sources. Typical efficiencies in the literature are approximately less than 5%. Host cells engineered to produce fatty alcohols can have greater than about 1, 3, 5, 10, 15, 20, 25, and 30% efficiency. In one example, host cells can exhibit an efficiency of about 10% to about 25%. In other examples, such host cells can exhibit an efficiency of about 25% to about 30%. In other examples, host cells can exhibit greater than 30% efficiency.

The host cell can be additionally engineered to express recombinant cellulosomes, such as those described in PCT application number PCT/US2007/003736. These cellulosomes can allow the host cell to use cellulosic material as a carbon source. For example, the host cell can be additionally engineered to express invertases (EC 3.2.1.26) so that sucrose can be used as a carbon source. Similarly, the host cell can be engineered using the teachings described in U.S. Pat. Nos. 5,000,000; 5,028,539; 5,424,202; 5,482,846; and 5,602,030; so that the host cell can assimilate carbon efficiently and use cellulosic materials as carbon sources.

In one example, the fermentation chamber can enclose a fermentation that is undergoing a continuous reduction. In this instance, a stable reductive environment can be created. The electron balance can be maintained by the release of carbon dioxide (in gaseous form). Efforts to augment the NAD/H and NADP/H balance can also facilitate in stabilizing the electron balance. The availability of intracellular NADPH can also be enhanced by engineering the host cell to express an NADH:NADPH transhydrogenase. The expression of one or more NADH:NADPH transhydrogenases converts the NADH produced in glycolysis to NADPH, which can enhance the production of fatty alcohols.

For small scale production, the engineered host cells can be grown in batches of, for example, about 100 mL, 500 mL, 1 L, 2 L, 5 L, or 10 L; fermented; and induced to express desired fatty aldehyde biosynthetic genes and/or an alcohol dehydrogenase genes based on the specific genes encoded in the appropriate plasmids. For large scale production, the engineered host cells can be grown in batches of about 10 L, 100 L, 1000 L, 10,000 L, 100,000 L, 1,000,000 L or larger; fermented; and induced to express desired fatty aldehyde biosynthetic genes and/or alcohol dehydrogenase genes based on the specific genes encoded in the appropriate plasmids or incorporated into the host cell's genome.

For example, a suitable production host, such as E. coli cells, harboring plasmids containing the desired genes or having the genes integrated in its chromosome can be incubated in a suitable reactor, for example a 1 L reactor, for 20 hours at 37° C. in M9 medium supplemented with 2% glucose, carbenicillin, and chloramphenicol. When the OD₆₀₀ of the culture reaches 0.9, the production host can be induced with IPTG alcohol. After incubation, the spent media can be extracted and the organic phase can be examined for the presence of fatty alcohols using GC-MS.

In some instances, after the first hour of induction, aliquots of no more than about 10% of the total cell volume can be removed each hour and allowed to sit without agitation to allow the fatty alcohols to rise to the surface and undergo a spontaneous phase separation or precipitation. The fatty alcohol component can then be collected, and the aqueous phase returned to the reaction chamber. The reaction chamber can be operated continuously. When the OD₆₀₀ drops below 0.6, the cells can be replaced with a new batch grown from a seed culture.

### Producing Fatty Alcohols Using Cell-Free Methods

In some methods described herein, a fatty alcohol can be produced using a purified polypeptide (e.g., a fatty alcohol biosynthetic polypeptide) described herein and a substrate (e.g., fatty aldehyde), produced, for example, by a method described herein. For example, a host cell can be engineered to express a fatty alcohol biosynthetic polypeptide or variant as described herein. The host cell can be cultured under conditions suitable to allow expression of the polypeptide. Cell free extracts can then be generated using known methods. For example, the host cells can be lysed using detergents or by sonication. The expressed polypeptides can be purified using known methods. After obtaining the cell free extracts, substrates described herein can be added to the cell free extracts and maintained under conditions to allow conversion of the substrates (e.g., fatty aldehydes) to fatty alcohols. The fatty alcohols can then be separated and purified using known techniques.

In some instances, a fatty aldehyde can be converted into a fatty alcohol by contacting the fatty aldehyde with a fatty alcohol biosynthetic polypeptide provided herein, or a variant thereof. In other instances, a fatty aldehyde can be converted into a fatty alcohol by contacting the fatty aldehyde with a fatty alcohol biosynthetic polypeptide that is an AdhP homolog of FIG. 2, a DkgA homolog of FIG. 3, a DkgB homolog of FIG. 4, a Tas homolog of FIG. 5, an RspB homolog of FIG. 6, a YahK homolog of FIG. 7, a YbbO homolog of FIG. 8, a YbdH homolog of FIG. 9, a YbdR homolog of FIG. 10, a YgfF homolog of FIG. 11, a YhdH homolog of FIG. 12, a YjgB homolog of FIG. 13, an AroB homolog of FIG. 14, a YcjQ homolog of FIG. 15, a YdbC homolog of FIG. 16, a YdjG homolog of FIG. 17, a YeaE homolog of FIG. 18, a YncB homolog of FIG. 19, a YqhD homolog of FIG. 20, a YdjL homolog of FIG. 21, or a variant thereof.

### Post-Production Processing

The fatty alcohols produced during fermentation can be separated from the fermentation media. Any known technique for separating fatty alcohols from aqueous media can be used. One exemplary separation process is a two phase (bi-phasic) separation process. This process involves fermenting the genetically engineered host cells under conditions sufficient to produce fatty alcohols, allowing the fatty alcohol to collect in an organic phase, and separating the organic phase from the aqueous fermentation broth. This method can be practiced in both a batch and continuous fermentation processes.

Bi-phasic separation uses the relative immiscibility of fatty alcohols to facilitate separation. Immiscible refers to the relative inability of a compound to dissolve in water and is defined by the compound's partition coefficient. One of ordinary skill in the art will appreciate that by choosing a fermentation broth and organic phase, such that the fatty alcohol being produced has a high logP value, the fatty alcohol can separate into the organic phase, even at very low concentrations, in the fermentation vessel.

The fatty alcohols produced by the methods described herein can be relatively immiscible in the fermentation broth, as well as in the cytoplasm. Therefore, the fatty alcohol can collect in an organic phase either intracellularly or extracellularly. The collection of the products in the organic phase can lessen the impact of the fatty alcohol on cellular function and can allow the host cell to produce more product.

The methods described herein can result in the production of homogeneous compounds wherein at least about 60%, 70%, 80%, 90%, or 95% of the fatty alcohols produced will have carbon chain lengths that vary by less than about 6 carbons, less than about 4 carbons, or less than about 2 carbons. These compounds can also be produced with a relatively uniform degree of saturation. These compounds can be used directly as fuels, fuel additives, starting materials for production of other chemical compounds (e.g., polymers, surfactants, plastics, textiles, solvents, adhesives, etc.), or personal care additives. These compounds can also be used as feedstock for subsequent reactions, for example, hydrogenation, catalytic cracking (e.g., via hydrogenation, pyrolisis, or both), to make other products.

In some embodiments, the fatty alcohols produced using methods described herein can contain between about 50% and about 90% carbon; or between about 5% and about 25% hydrogen. In other embodiments, the fatty alcohols produced using methods described herein can contain between about 65% and about 85% carbon; or between about 10% and about 15% hydrogen.

In some embodiments, the host cell is a Gram-positive bacterial cell. In other embodiments, the host cell is a Gram-negative bacterial cell.

In some embodiments, the host cell is selected from the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

In other embodiments, the host cell is a Bacillus lentus cell, a Bacillus brevis cell, a Bacillus stearothermophilus cell, a Bacillus lichen formis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell.

In yet other embodiments, the host cell is a Streptomyces lividans cell or a Streptomyces murinus cell.

In yet other embodiments, the host cell is an Actinomycetes cell.

In some embodiments, the host cell is a Saccharomyces cerevisiae cell. In some embodiments, the host cell is a Saccharomyces cerevisiae cell.

In still other embodiments, the host cell is a CHO cell, a COS cell, a VERO cell, a BHK cell, a HeLa cell, a Cvl cell, an MDCK cell, a 293 cell, a 3T3 cell, or a PC12 cell.

In other embodiments, the host cell is a cell from an eukaryotic plant, algae, cyanolacterium, green-sulfur bacterium, green non-sulfur bacterium, purple sulfur bacterium, purple non-sulfur bacterium, extremophile, yeast, fungus, an engineered organism thereof, or a synthetic organism. In some embodiments, the host cell is light-dependent or fixes carbon. In some embodiments, the host cell is light-dependent or fixes carbon. In some embodiments, the host cell has autotrophic activity. In some embodiments, the host cell has photoautotrophic activity, such as in the presence of light. In some embodiments, the host cell is heterotrophic or mixotrophic in the absence of light. In certain embodiments, the host cell is a cell from Avabidopsis thaliana, Panicum virgatum, Miscanthus giganteus, Zea mays, Botryococcuse braunii, Chlamydomonas reinhardtii, Dunaliela salina, Synechococcus Sp. PCC 7002, Synechococcus Sp. PCC 7942, Synechocystis Sp. FCC 6803, Thermosynechococcus elongates BP-1, Chlorobium tepidum, Chlorojlexus auranticus, Chromatiumm vinosum, Rhodospirillum rubrum, Rhodobacter capsulatus, Rhodopseudomonas palusris, Clostridium ljungdahlii, Clostridiuthermocellum, Penicillium chrysogenum, Pichia pastoris, Saccharomyces cerevisiae, Schizosaccharomyces pombe, Pseudomonas fluorescens, or Zymomonas mobilis.

In certain preferred embodiments, the host cell is an E. coli cell. In some embodiments, the E. coli cell is a strain B, a strain C, a strain K, or a strain W E. coli cell.

In other embodiments, the host cell is a Pantoea citrea cell.

### Production of Fatty Acids Deriviatives in Host Cells.

As used herein, the term “conditions permissive for the production” means any conditions that allow a host cell to produce a desired product, such as a fatty acid or a fatty acid derivative. Similarly, the term “conditions in which the polynucleotide sequence of a vector is expressed” means any conditions that allow a host cell to synthesize a polypeptide. Suitable conditions include, for example, fermentation conditions. Fermentation conditions can comprise many parameters, such as temperature ranges, levels of aeration, and media composition. Each of these conditions, individually and in combination, allows the host cell to grow. Exemplary culture media include broths or gels. Generally, the medium includes a carbon source that can be metabolized by a host cell directly

To determine if conditions are sufficient to allow production of a product or expression of a polypeptide, a host cell can be cultured, for example, for about 4, 8, 12, 24, 36, 48, 72, or more hours. During and/or after culturing, samples can be obtained and analyzed to determine if the conditions allow production or expression. For example, the host cells in the sample or the medium in which the host cells were grown can be tested for the presence of a desired product. When testing for the presence of a fatty acid or fatty acid derivative, assays, such as, but not limited to, MS, thin layer chromatography (TLC), high-performance liquid chromatography (HPLC), liquid chromatography (LC), GC coupled with a flame ionization detector (FID), GC-MS, and LC-MS can be used. When testing for the expression of a polypeptide, techniques such as, but not limited to, Western blotting and dot blotting may be used.

In the compositions and methods of the invention, the production and isolation of fatty acids and fatty acid derivatives can be enhanced by optimizing fermentation conditions. In some embodiments, fermentation conditions are optimized to increase the percentage of the carbon source that is converted to hydrocarbon products. During normal cellular lifecycles, carbon is used in cellular functions, such as producing lipids, saccharides, proteins, organic acids, and nucleic acids. Reducing the amount of carbon necessary for growth-related activities can increase the efficiency of carbon source conversion to product. This can be achieved by, for example, first growing host cells to a desired density (for example, a density achieved at the peak of the log phase of growth). At such a point, replication checkpoint genes can be harnessed to stop the growth of cells. Specifically, quorum sensing mechanisms (reviewed in Camilli et al., Science 311: 1113 (2006); Venturi, FEMS Microbiol. Rev., 30: 274-291 (2006); and Reading et al., FEMS Microbiol. Lett., 254: 1-11 (2006)) can be used to activate checkpoint genes, such as p53, p21, or other checkpoint genes.

The host cell can be additionally engineered to express a recombinant cellulosome, which can allow the host cell to use cellulosic material as a carbon source. Exemplary cellulosomes suitable for use in the methods of the invention include, e.g., the cellulosomes described in International Patent Application Publication WO 2008/100251. The host cell also can be engineered to assimilate carbon efficiently and use cellulosic materials as carbon sources according to methods described in U.S. Pat. Nos. 5,000,000; 5,028,539; 5,424,202; 5,482,846; and 5,602,030. In addition, the host cell can be engineered to express an invertase so that sucrose can be used as a carbon source.

In some embodiments of the fermentation methods of the invention, the fermentation chamber encloses a fermentation that is undergoing a continuous reduction, thereby creating a stable reductive environment. The electron balance can be maintained by the release of carbon dioxide (in gaseous foam). Efforts to augment the NAD/H and NADP/H balance can also facilitate in stabilizing the electron balance. The availability of intracellular NADPH can also be enhanced by engineering the host cell to express an NADH:NADPH transhydrogenase. The expression of one or more NADH:NADPH transhydrogenases converts the NADH produced in glycolysis to NADPH, which can enhance the production of fatty aldehydes and fatty alcohols.

For small scale production, the engineered host cells can be grown in batches of, for example, about 100 mL, 500 mL, 1 L, 2 L, 5 L, or 10 L; fermented; and induced to express a desired polynucleotide sequence, such as a polynucleotide sequence encoding a PPTase. For large scale production, the engineered host cells can be grown in batches of about 10 L, 100 L, 1000 L, 10,000 L, 100,000 L, 1,000,000 L or larger; fennented; and induced to express a desired polynucleotide sequence.

The fatty acids and derivatives thereof produced by the methods of invention generally are isolated from the host cell. The term “isolated” as used herein with respect to products, such as fatty acids and derivatives thereof, refers to products that are separated from cellular components, cell culture media, or chemical or synthetic precursors. The fatty acids and derivatives thereof produced by the methods described herein can be relatively immiscible in the fermentation broth, as well as in the cytoplasm. Therefore, the fatty acids and derivatives thereof can collect in an organic phase either intracellularly or extracellularly. The collection of the products in the organic phase can lessen the impact of the fatty acid or fatty acid derivative on cellular function and can allow the host cell to produce more product.

In some embodiments, the fatty acids and fatty acid derivatives produced by the methods of invention are purified. As used herein, the term “purify,” “purified,” or “purification” means the removal or isolation of a molecule from its environment by, for example, isolation or separation. “Substantially purified” molecules are at least about 60% free (e.g., at least about 70% free, at least about 75% free, at least about 85% free, at least about 90% free, at least about 95% free, at least about 97% free, at least about 99% free) from other components with which they are associated. As used herein, these terms also refer to the removal of contaminants from a sample. For example, the removal of contaminants can result in an increase in the percentage of a fatty aldehyde or a fatty alcohol in a sample. For example, when a fatty aldehyde or a fatty alcohol is produced in a host cell, the fatty aldehyde or fatty alcohol can be purified by the removal of host cell proteins. After purification, the percentage of a fatty acid or derivative thereof in the sample is increased.

As used herein, the terms “purify,” “purified,” and “purification” are relative terms which do not require absolute purity. Thus, for example, when a fatty acid or derivative thereof is produced in host cells, a purified fatty acid or derivative thereof is a fatty acid or derivative thereof that is substantially separated from other cellular components (e.g., nucleic acids, polypeptides, lipids, carbohydrates, or other hydrocarbons).

Additionally, a purified fatty acid preparation or a purified fatty acid derivative preparation is a fatty acid preparation or a fatty acid derivative preparation in which the fatty acid or derivative thereof is substantially free from contaminants, such as those that might be present following fermentation. In some embodiments, a fatty acid or derivative thereof is purified when at least about 50% by weight of a sample is composed of the fatty acid or fatty acid derivative. In other embodiments, a fatty acid or derivative thereof is purified when at least about 60%, e.g., at least about 70%, at least about 80%, at least about 85%, at least about 90%, at least about 92% or more by weight of a sample is composed of the fatty acid or derivative thereof. Alternatively, or in addition, a fatty acid or derivative thereof is purified when less than about 100%, e.g., less than about 99%, less than about 98%, less than about 95%, less than about 90%, or less than about 80% by weight of a sample is composed of the fatty acid or derivative thereof. Thus, a purified fatty acid or derivative thereof can have a purity level bounded by any two of the above endpoints. For example, a fatty acid or derivative thereof can be purified when at least about 80%-95%, at least about 85%-99%, or at least about 90%-98% of a sample is composed of the fatty acid or fatty acid derivative.

The fatty acid or derivative thereof may be present in the extracellular environment, or it may be isolated from the extracellular environment of the host cell. In certain embodiments, a fatty acid or derivative thereof is secreted from the host cell. In other embodiments, a fatty acid or derivative thereof is transported into the extracellular environment. In yet other embodiments, the fatty acid or derivative thereof is passively transported into the extracellular environment.

A fatty acid or derivative thereof can be isolated from a host cell using methods known in the art, such as those disclosed in International Patent Application Publications WO 2010/042664 and WO 2010/062480.

The methods described herein can result in the production of homogeneous compounds wherein at least about 60%, at least about 70%, at least about 80%, at least about 90%, or at least about 95%, of the fatty acids or fatty acid derivatives produced will have carbon chain lengths that vary by less than 6 carbons, less than 5 carbons, less than 4 carbons, less than 3 carbons, or less than about 2 carbons. Alternatively, or in addition, the methods described herein can result in the production of homogeneous compounds wherein less than about 98%, less than about 95%, less than about 90%, less than about 80%, or less than about 70% of the fatty acids or fatty acid derivatives produced will have carbon chain lengths that vary by less than 6 carbons, less than 5 carbons, less than 4 carbons, less than 3 carbons, or less than about 2 carbons. Thus, the fatty acids or fatty acid derivatives can have a degree of homogeneity bounded by any two of the above endpoints. For example, the fatty acid or fatty acid derivative can have a degree of homogeneity wherein about 70%-95%, about 80%-98%, or about 90%-95% of the fatty acids or fatty acid derivatives produced will have carbon chain lengths that vary by less than 6 carbons, less than 5 carbons, less than 4 carbons, less than 3 carbons, or less than about 2 carbons. These compounds can also be produced with a relatively uniform degree of saturation.

As a result of the methods of the present invention, one or more of the titer, yield, or productivity of the fatty acid or derivative thereof produced by the engineered host cell having an altered level of expression of a FadR polypeptide is increased relative to that of the corresponding wild-type host cell.

The term “titer” refers to the quantity of fatty acid or fatty acid derivative produced per unit volume of host cell culture. In any aspect of the compositions and methods described herein, a fatty acid or a fatty acid derivative such as a terminal olefin, a fatty aldehyde, a fatty alcohol, an alkane, a fatty ester, a ketone or an internal olefins is produced at a titer of about 25 mg/L, about 50 mg/L, about 75 mg/L, about 100 mg/L, about 125 mg/L, about 150 mg/L, about 175 mg/L, about 200 mg/L, about 225 mg/L, about 250 mg/L, about 275 mg/L, about 300 mg/L, about 325 mg/L, about 350 mg/L, about 375 mg/L, about 400 mg/L, about 425 mg/L, about 450 mg/L, about 475 mg/L, about 500 mg/L, about 525 mg/L, about 550 mg/L, about 575 mg/L, about 600 mg/L, about 625 mg/L, about 650 mg/L, about 675 mg/L, about 700 mg/L, about 725 mg/L, about 750 mg/L, about 775 mg/L, about 800 mg/L, about 825 mg/L, about 850 mg/L, about 875 mg/L, about 900 mg/L, about 925 mg/L, about 950 mg/L, about 975 mg/L, about 1000 g/L, about 1050 mg/L, about 1075 mg/L, about 1100 mg/L, about 1125 mg/L, about 1150 mg/L, about 1175 mg/L, about 1200 mg/L, about 1225 mg/L, about 1250 mg/L, about 1275 mg/L, about 1300 mg/L, about 1325 mg/L, about 1350 mg/L, about 1375 mg/L, about 1400 mg/L, about 1425 mg/L, about 1450 mg/L, about 1475 mg/L, about 1500 mg/L, about 1525 mg/L, about 1550 mg/L, about 1575 mg/L, about 1600 mg/L, about 1625 mg/L, about 1650 mg/L, about 1675 mg/L, about 1700 mg/L, about 1725 mg/L, about 1750 mg/L, about 1775 mg/L, about 1800 mg/L, about 1825 mg/L, about 1850 mg/L, about 1875 mg/L, about 1900 mg/L, about 1925 mg/L, about 1950 mg/L, about 1975 mg/L, about 2000 mg/L, or a range bounded by any two of the foregoing values. In other embodiments, a fatty acid or fatty acid derivative is produced at a titer of more than 2000 mg/L, more than 5000 mg/L, more than 10,000 mg/L, or higher, such as 50 g/L, 70 g/L, 100 g/L, 120 g/L, 150 g/L, or 200 g/L.

The term “yield of the fatty acid or derivative thereof produced by a host cell” refers to the efficiency by which an input carbon source is converted to product (i.e., fatty acid or fatty acid derivative such as fatty alcohol or fatty ester) in a host cell. For oxygen-containing carbon sources (e.g., glucose and other carbohydrate based sources), the oxygen must be released in the form of carbon dioxide. For every 2 oxygen atoms released, a carbon atom is also released leading to a maximal theoretical metabolic efficiency of approximately 34% (w/w) (for fatty acid derived products). This figure, however, changes for other organic compounds and carbon sources. Typical yield reported in the literature are approximately less than 5%. Host cells engineered to produce fatty acids and fatty acid derivatives according to the methods of the invention can have a yield of at least about 3%, at least about 5%, at least about 10%, at least about 15%, at least about 18%, or at least about 20%. Alternatively, or in addition, the yield is about 30% or less, about 27% or less, about 25% or less, or about 22% or less. Thus, the yield can be bounded by any two of the above endpoints. For example, the yield of the fatty acid or derivative thereof produced by the engineered host cell according to the methods of the invention can be about 5% to about 25%, about 10% to about 25%, about 10% to about 22%, about 15% to about 27%, or about 18% to about 22%. In other embodiments, the yield is greater than 30%.

The term “productivity of the fatty acid or derivative thereof produced by a host cell” refers to the quantity of fatty acid or fatty acid derivative produced per unit volume of host cell culture per unit density of host cell culture. In any aspect of the compositions and methods described herein, the productivity of a fatty acid or a fatty acid derivative such as an olefin, a fatty aldehyde, a fatty alcohol, an alkane, a fatty ester, or a ketone produced by an engineered host cells is at least about at least about 3 mg/L/OD₆₀₀, at least about 6 mg/L/OD₆₀₀, at least about 9 mg/L/OD₆₀₀, at least about 12 mg/L/OD₆₀₀, or at least about 15 mg/L/OD₆₀₀. Alternatively, or in addition, the productivity is about 50 mg/L/OD₆₀₀ or less, about 40 mg/L/OD₆₀₀ or less, about 30 mg/L/OD₆₀₀ or less, or about 20 mg/L/OD₆₀₀ or less. Thus, the productivity can be bounded by any two of the above endpoints. For example, the productivity can be about 3 to about 30 mg/L/OD₆₀₀, about 6 to about 20 mg/L/OD₆₀₀, or about 15 to about 30 mg/L/OD₆₀₀.

In the compositions and methods of the invention, the production and isolation of a desired fatty acid or derivative thereof (e.g., acyl-CoA, fatty acids, terminal olefins, fatty aldehydes, fatty alcohols, alkanes, alkenes, wax esters, ketones and internal olefins) can be enhanced by altering the expression of one or more genes involved in the regulation of fatty acid, fatty ester, alkane, alkene, olefin fatty alcohol production, degradation and/or secretion in the engineered host cell.

### Characterization and Utility of Fatty Acids and Derivatives Thereof

Bioproducts (e.g., fatty alcohols) comprising biologically produced organic compounds, particularly fatty alcohols biologically produced using the fatty acid biosynthetic pathway, have not been produced from renewable sources and, as such, are new compositions of matter.

The hydrocarbons (and/or fatty aldehydes) described herein can be used as or converted into a fuel or as a specialty chemical. One of ordinary skill in the art will appreciate that, depending upon the intended purpose of the fuel or specialty chemical, different hydrocarbons (and/or fatty aldehydes) can be produced and used. For example, a branched hydrocarbon may be desirable for automobile fuels that are intended to be used in cold climates. In addition, when hydrocarbons are used as a feedstock for fuel and specialty chemical production, one of ordinarly skill in the art will appreciate that the characteristics of the hydrocarbon will affect the characteristics of the fuel or specialty chemicals produced. Hence the characteristics of the fuel or specialty chemical product can be selected for by producing particular hydrocarbons (and/or fatty aldehydes) for use as a feedstock.

Using the methods described herein, biofuels having desired fuel qualities can be produced from hydrocarbons (and/or fatty aldehydes). These thus represent a new source of biofuels, which can be used as jet fuels, diesel, or gasoline. Some biofuels made using hydrocarbons (and/or fatty aldehydes) thus prepared have not been produced from renewable sources and are new compositions of matter. These new fuels or specialty chemicals can be distinguished from fuels or specialty chemicals derived from petrochemical carbon on the basis of dual carbon-isotopic fingerprinting. Additionally, the specific source of biosourced carbon (e.g., glucose vs. glycerol) can be determined by dual carbon-isotopic fingerprinting (see, e.g., U.S. Pat. No. 7,169,588, which is herein incorporated by reference).

The ability to distinguish bioproducts from petroleum based organic compounds is beneficial in tracking these materials in commerce. For example, organic compounds or chemicals comprising both biologically based and petroleum based carbon isotope profiles may be distinguished from organic compounds and chemicals made only of petroleum based materials. Hence, the instant materials may be followed in commerce on the basis of their unique carbon isotope profile.

These new bioproducts can be distinguished from organic compounds derived from petrochemical carbon on the basis of dual carbon-isotopic fingerprinting (¹³C/¹²C) or ¹⁴C dating. Additionally, the specific source of biosourced carbon (e.g., glucose vs. glycerol) can be determined by dual carbon-isotopic fingerprinting (see, e.g., U.S. Pat. No. 7,169,588, which is herein incorporated by reference).

Bioproducts can be distinguished from petroleum based organic compounds by comparing the stable carbon isotope ratio (¹³C/¹²C) in each fuel. The ¹³C/¹²C ratio in a given bioproduct is a consequence of the ¹³C/¹²C ratio in atmospheric carbon dioxide at the time the carbon dioxide is fixed. It also reflects the precise metabolic pathway. Regional variations also occur. Petroleum, C₃ plants (the broadleaf), C₄ plants (the grasses), and marine carbonates all show significant differences in ¹³C/¹²C and the corresponding δ¹³C values. Furthermore, lipid matter of C₃ and C₄ plants analyze differently than materials derived from the carbohydrate components of the same plants as a consequence of the metabolic pathway.

Within the precision of measurement, ¹³C shows large variations due to isotopic fractionation effects, the most significant of which for bioproducts is the photosynthetic mechanism. The major cause of differences in the carbon isotope ratio in plants is closely associated with differences in the pathway of photosynthetic carbon metabolism in the plants, particularly the reaction occurring during the primary carboxylation (i.e., the initial fixation of atmospheric CO₂). Two large classes of vegetation are those that incorporate the “C₃” (or Calvin-Benson) photosynthetic cycle and those that incorporate the “C₄” (or Hatch-Slack) photosynthetic cycle.

Both C₄ and C₃ plants exhibit a range of ¹³C/¹²C isotopic ratios, but typical values are about −7 to about −13 per mil for C₄ plants and about −19 to about −27 per mil for C₃ plants (see, e.g., Stuiver et al., Radiocarbon 19:355, 1977). Coal and petroleum fall generally in this latter range. The ¹³C measurement scale was originally defined by a zero set by Pee Dee Belemnite (PDB) limestone, where values are given in parts per thousand deviations from this material. The “δ¹³C” values are expressed in parts per thousand (per mil), abbreviated, %₀₀, and are calculated as follows:

δ¹³C(%₀₀)=[(¹³C/¹²C)ₛₐₘₚₗₑ−(¹³C/¹²C)_{standard}]/(¹³C/¹²C)_{standard}×1000

Since the PDB reference material (RM) has been exhausted, a series of alternative RMs have been developed in cooperation with the IAEA, USGS, NIST, and other selected international isotope laboratories. Notations for the per mil deviations from PDB is δ¹³C. Measurements are made on CO₂ by high precision stable ratio mass spectrometry (IRMS) on molecular ions of masses 44, 45, and 46.

The compositions described herein include bioproducts produced by any of the methods described herein. Specifically, the bioproduct can have a δ¹³C of about −28 or greater, about −27 or greater, −20 or greater, −18 or greater, −15 or greater, −13 or greater, −10 or greater, or −8 or greater. For example, the bioproduct can have a δ¹³C of about −30 to about −15, about −27 to about −19, about −25 to about −21, about −15 to about −5, about −13 to about −7, or about −13 to about −10. In other instances, the bioproduct can have a δ¹³C of about −10, −11, −12, or −12.3.

Bioproducts can also be distinguished from petroleum based organic compounds by comparing the amount of ¹⁴C in each compound. Because ¹⁴C has a nuclear half life of 5730 years, petroleum based fuels containing “older” carbon can be distinguished from bioproducts which contain “newer” carbon (see, e.g., Currie, “Source Apportionment of Atmospheric Particles”, Characterization of Environmental Particles, J. Buffle and H. P. van Leeuwen, Eds., 1 of Vol. I of the IUPAC Environmental Analytical Chemistry Series (Lewis Publishers, Inc) (1992) 3-74).

The basic assumption in radiocarbon dating is that the constancy of ¹⁴C concentration in the atmosphere leads to the constancy of ¹⁴C in living organisms. However, because of atmospheric nuclear testing since 1950 and the burning of fossil fuel since 1850, ¹⁴C has acquired a second, geochemical time characteristic. Its concentration in atmospheric CO₂, and hence in the living biosphere, approximately doubled at the peak of nuclear testing, in the mid-1960s. It has since been gradually returning to the steady-state cosmogenic (atmospheric) baseline isotope rate (¹⁴C/¹²C) of about 1.2×10⁻¹², with an approximate relaxation “half-life” of 7-10 years. (This latter half-life must not be taken literally; rather, one must use the detailed atmospheric nuclear input/decay function to trace the variation of atmospheric and biospheric ¹⁴C since the onset of the nuclear age.)

It is this latter biospheric ¹⁴C time characteristic that holds out the promise of annual dating of recent biospheric carbon. ¹⁴C can be measured by accelerator mass spectrometry (AMS), with results given in units of “fraction of modern carbon” (f_{M}). f_{M} is defined by National Institute of Standards and Technology (NIST) Standard Reference Materials (SRMs) 4990B and 4990C. As used herein, “fraction of modern carbon” or “f_{M}” has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

The compositions described herein include bioproducts that can have an f_{M} ¹⁴C of at least about 1. For example, the bioproduct can have an f_{M} ¹⁴C of at least about 1.01, an f_{M} ¹⁴C of about 1 to about 1.5, an f_{M} ¹⁴C of about 1.04 to about 1.18, or an f_{M} ¹⁴C of about 1.111 to about 1.124.

Another measurement of ¹⁴C is known as the percent of modern carbon, pMC. For an archaeologist or geologist using ¹⁴C dates, AD 1950 equals “zero years old”. This also represents 100 pMC. “Bomb carbon” in the atmosphere reached almost twice the normal level in 1963 at the peak of thermo-nuclear weapons. Its distribution within the atmosphere has been approximated since its appearance, showing values that are greater than 100 pMC for plants and animals living since AD 1950. It has gradually decreased over time with today's value being near 107.5 pMC. This means that a fresh biomass material, such as corn, would give a ¹⁴C signature near 107.5 pMC. Petroleum based compounds will have a pMC value of zero. Combining fossil carbon with present day carbon will result in a dilution of the present day pMC content. By presuming 107.5 pMC represents the ¹⁴C content of present day biomass materials and 0 pMC represents the ¹⁴C content of petroleum based products, the measured pMC value for that material will reflect the proportions of the two component types. For example, a material derived 100% from present day soybeans would give a radiocarbon signature near 107.5 pMC. If that material was diluted 50% with petroleum based products, it would give a radiocarbon signature of approximately 54 pMC.

A biologically based carbon content is derived by assigning “100%” equal to 107.5 pMC and “0%” equal to 0 pMC. For example, a sample measuring 99 pMC will give an equivalent biologically based carbon content of 93%. This value is referred to as the mean biologically based carbon result and assumes all the components within the analyzed material originated either from present day biological material or petroleum based material.

A bioproduct described herein can have a pMC of at least about 50, 60, 70, 75, 80, 85, 90, 95, 96, 97, 98, 99, or 100. In other instances, a bioproduct described herein can have a pMC of between about 50 and about 100; about 60 and about 100; about 70 and about 100; about 80 and about 100; about 85 and about 100; about 87 and about 98; or about 90 and about 95. In yet other instances, a bioproduct described herein can have a pMC of about 90, 91, 92, 93, 94, or 94.2.

The fatty alcohols described herein can be used as or converted into a surfactant or detergent composition. One of ordinary skill in the art will appreciate that, depending upon the intended purpose of the surfactant or detergent, different fatty alcohols can be produced and used. For example, when the fatty alcohols described herein are used as a feedstock for surfactant or detergent production, one of ordinary skill in the art will appreciate that the characteristics of the fatty alcohol feedstock will affect the characteristics of the surfactant or detergent produced. Hence, the characteristics of the surfactant or detergent product can be selected for by producing particular fatty alcohols for use as a feedstock.

Fuel additives are used to enhance the performance of a fuel or engine. For example, fuel additives can be used to alter the freezing/gelling point, cloud point, lubricity, viscosity, oxidative stability, ignition quality, octane level, and/or flash point. In the United States, all fuel additives must be registered with Environmental Protection Agency. The names of fuel additives and the companies that sell the fuel additives are publicly available by contacting the EPA or by viewing the agency's website. One of ordinary skill in the art will appreciate that the fatty alcohol-based biofuels described herein can be mixed with one or more fuel additives to impart a desired quality.

The fatty alcohol-based surfactants and/or detergents described herein can be mixed with other surfactants and/or detergents well known in the art.

In some examples, the mixture can include at least about 10%, 15%, 20%, 30%, 40%, 50%, or 60% by weight of the fatty alcohol. In other examples, a surfactant or detergent composition can be made that includes at least about 5%, 10%, 20%, 30%, 40%, 50%, 60%, 70%, 80%, 85%, 90% or 95% of a fatty alcohol that includes a carbon chain that is 8, 10, 12, 13, 14, 15, 16, 17, 18, 19, 20, 21 or 22 carbons in length. Such surfactant or detergent compositions can additionally include at least one additive selected from a surfactant; a microemulsion; at least about 5%, 10%, 15%, 20%, 30%, 40%, 50%, 60%, 70%, 80%, 85%, 90%, or 95% of surfactant or detergent from nonmicrobial sources such as plant oils or petroleum.

The hydrocarbon (and/or fatty aldehyde)-based biofuel described herein can be mixed with other fuels, such as various alcohols, such as ethanol and butanol, and petroleum derived products, such as gasoline, diesel, or jet fuel.

In some examples, the mixture can include at least about 10%, 15%, 20%, 30%, 40%, 50%, or 60% by weight of the hydrocarbon (and/or fatty aldehydes). In other examples, a biofuel composition can be made that includes at least about 5%, 10%, 20%, 30%, 40%, 50%, 60%, 70%, 80%, 85%, 90% or 95% of a hydrocarbon such as an alkane or an alkene that includes a carbon chain that is 8, 10, 12, 13, 14, 15, 16, 17, 18, 19, 20, 21 or 22 carbons in length. Such biofuel composition can additionally include at least one additive selected from a cloud point lowering additive that can lower the cloud point to less than about 5° C., or 0° C.; a surfactant, a microemulsion; at least about 5%, 10%, 20%, 30%, 40%, 50%, 60%, 70%, 80%, 85%, 90% or 95% diesel fuel from triglycerides; petroleum-derived gasoline; or diesel fuel from petroleum.

Although the foregoing has been described in some detail by way of illustration and example for purposes of clarity and understanding, it will be apparent to those skilled in the art that certain changes and modifications may be practiced. Various aspects of the invention have been achieved by a series of experiments, some of which are described by way of the following non-limiting examples. Therefore, the description and examples should not be construed as limiting the scope of the invention, which is delineated by the appended description of exemplary embodiments.

### EXAMPLES

### Example 1

An AlrA enzyme from Acinetobacter sp. M-1 has been shown to catalyze the reduction of fatty aldehyde into fatty alcohols in vitro at neutral or low pH conditions. (Tani et al. Appl. Environ. Microbiol. 66(12):5231-5 (2000)). However, E. coli fatty alcohol biosynthetic polypeptides, which are capable of catalyzing the reduction of fatty aldehydes to fatty alcohols, were not identified, although it has been reported that E. coli constitutively expresses such a reductase activity. (Naccarato et al., Lipids 9(6):419-28 (1974)). It had also been reported that the E. coli reductase activity was NADPH-dependent. Id.

A BLAST search of the Acinetobacter baylyi ADP1 genomic and protein databases for homologs of Acinetobacter sp. M-1 AlrA revealed an Acinetobacter baylyi ADP1 homolog, AlrAadp1 (GenPept Accession Number CAG 70248.1), has about 79% identity to the Acinetobacter sp. M-1 AlrA.

This example describes an experiment verifying that co-expression of a heterologous carboxylic acid reductase from Acinetobacter baylyi ADP1, AlrAadp1 (a homolog of Acinetobacter sp. M-1 AlrA) and a CarB homolog resulted in fatty alcohol production in E. coli.

### CAR Plasmid Construction

Three E. coli expression plasmids were constructed to express the genes encoding the CAR homologs listed in Table 7.

TABLE 7
CAR-like Protein and the corresponding coding sequences.

| Genpept Acc. | Locus_tag | Annotation in GenBank | Gene name |
|---|---|---|---|
| NP_217106 | Rv 2590 | Probable fatty-acid-CoA | fadD9 |
| | | ligase (FadD9) | |
| ABK75684 | MSMEG | NAD dependent epimerase/ | carA |
| | 2956 | dehydratase family protein | |
| YP_889972.1 | MSMEG | NAD dependent epimerase/ | carB |
| | 5739 | dehydratase family protein | |

The fadD9 gene was amplified from genomic DNA of Mycobacterium tuberculosis H37Rv (obtained from The University of British Columbia, and Vancouver, BC Canada) using the primers fadD9F and FadDR (see Table 8). The PCR product was first cloned into PCR-blunt (Invitrogen) and then released as an NdeI-AvrII fragment. The NdeI-AvrII fragment was then cloned between the NdeI and AvrII sites of pACYCDuet-1 (Novogen) to generate pACYCDuet-1-fadD9.

The carA gene was amplified from the genomic DNA of Mycobacterium smegmatis MC2 155 (obtained from the ATCC (ATCC 23037D-5)) using primers CARMCaF and CARMCaR (see Table 8). The carB gene was amplified from the genomic DNA of Mycobacterium smegmatis MC2 155 (obtained from the ATCC (ATCC 23037D-5)) using primers CARMCbF and CARMCbR (see Table 8). Each PCR product was first cloned into PCR-blunt and then released as an NdeI-AvrII fragment. Each of the two fragments was then subcloned between the NdeI and AvrII sites of pACYCDuet-1 (Novogen) to generate pACYCDuet-1-carA and pACYCDuet-1-carB.

TABLE 8
Primers used to amplify genes encoding CAR
homologs

| | |
|---|---|
| fadD9F | cat ATGTCGATCAACGATCAGCGACTGAC |
| | (SEQ ID NO: 211) |
| fadD9R | cctagg TCACAGCAGCCCGAGCAGTC |
| | (SEQ ID NO: 212) |
| CARMCaF | cat ATGACGATCGAAACGCG |
| | (SEQ ID NO: 213) |
| CARMCaR | cctagg TTACAGCAATCCGAGCATCT |
| | (SEQ ID NO: 214) |
| CARMCbF | cat ATGACCAGCGATGTTCAC |
| | (SEQ ID NO: 215) |
| CARMCbR | cctagg TCAGATCAGACCGAACTCACG |
| | (SEQ ID NO: 216) |

Construction of plasmid pETDuet-1-'tesA-alrAadp1 was carried out with the protocol below. The plasmid pETDuet-1-'tesA-alrAadp1 was prepared by inserting the alrAadp1 gene (gene locus-tag=“ACIAD3612”), a homolog of Acinetobacter baylyi ADP1, into the NcoI and HindIII sites of pETDuet-1-'tesA.

The gene alrAadp1 was amplified from the genomic DNA of Acinetobacter baylyi ADP1 by a two-step PCR procedure. The first set of PCR reactions eliminated an internal NcoI site at by 632-636 with the following primer pairs:

ADP1 Alr mut1 reverse:
(SEQ ID NO: 217)
5′-GACCACGTGATCGGCCCCCATAGCTTTGAGCTCATC
ADP1 Alr1 mut1 forward:
(SEQ ID NO: 218)
5′-GATGAGCTCAAAGCTATGGGGGCCGATCACGTGGTC

The PCR products were then isolated, purified using the Qiagen gel extraction kit, and used as inputs for a second PCR reaction with the following primers to produce full-length AlrAadp1 with a C→T mutation at position 633:

NcoI ADP1 Alr1 forward:
(SEQ ID NO: 219)
5′-AATACCATGGCAACAACTAATGTGATTCATGCTTATGCTGCA
HindIII ADP1 Alr1 reverse:
(SEQ ID NO: 220)
5′-ATAAAAGCTTTTAAAAATCGGCTTTAAGTACAATCCGATAAC

### Evaluation of Fatty Alcohol Production

In order to evaluate the affect of carboxylic acid reductases and alcohol dehydrogenases on the production of fatty alcohols, various combinations of the prepared plasmids were transformed in the E. coli strain C41 (DE3, ΔfadE) (described in PCT/US08/058,788).

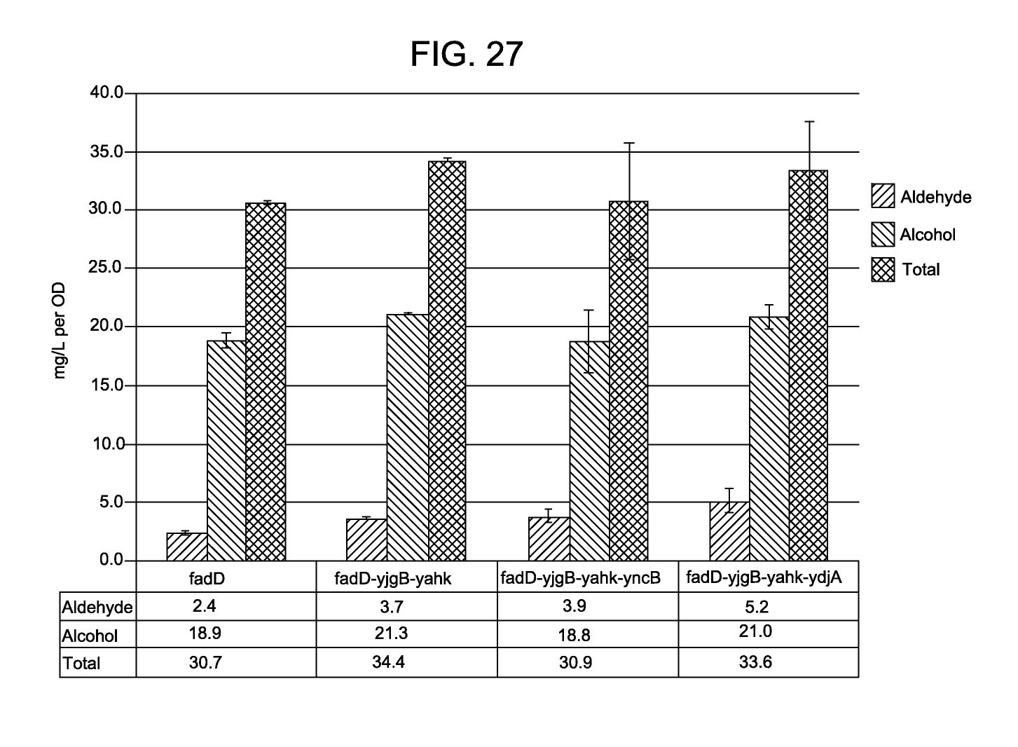

For example, the plasmid pACYCDuet-1-carA, encoding the CAR homolog carA, was co-transformed with pETDuet-1-'tesA-alrAadp1 (see, e.g., FIG. 27A). The plasmid pACYCDuet-1-carB, encoding the CAR homolog, carB, was co-transformed with pETDuet-1-'tesA. In addition, pACYCDuet-1-carB was also separately co-transformed with pETDuet-1-'tesA-alrAadp1. As a control, pACYCDuet-1-carB was co-transformed with the empty vector pETDuet-1 (see, e.g., FIG. 27A). The plasmid pACYCDuet-1-fadD9, encoding the CAR homolog fadD9, was also co-transformed with pETDuet-1-'tesA. In addition, pACYCDuet-1-fadD9 was also separately co-transformed with pETDuet-1-'tesA-alrAadp1. As a control, pACYCDuet-1-fadD9 was co-transformed with the empty vector pETDuet-1 (see, e.g., FIG. 27A).

The E. coli transformants were grown in 3 mL of LB medium supplemented with carbenicillin (100 mg/L) and chloramphenicol (34 mg/L) at 37° C. After overnight growth, 15 μL of culture was transferred into 2 mL of fresh LB medium supplemented with carbenicillin and chloramphenicol. After 3.5 hours of growth, 2 mL of culture were transferred into a 125 mL flask containing 20 mL of M9 medium with 2% glucose and with carbenicillin and chloramphenicol. When the OD₆₀₀ of the culture reached 0.9, 1 mM of IPTG was added to each flask. After 20 hours of growth at 37° C., 20 mL of ethyl acetate (with 1% of acetic acid, v/v) was added to each flask to extract the fatty alcohols produced during the fermentation. The crude ethyl acetate extract was directly analyzed with GC/MS as described herein.

The expression of carA or carB with the leaderless tesA and alrAadp1 resulted in fatty alcohol titers of greater than 700 mg/L and reduced fatty aldehyde production (see, e.g., FIG. 27A). Likewise, fadD9 co-expressed with the leaderless tesA and alrAadp1 produced over 300 mg/L of fatty alcohol. When expressed without the leaderless tesA, neither carB nor fadD9 produced more than 10 mg/L of fatty alcohols (possibly resulting from the accumulation of free fatty acids in the cell due to endogenous tesA). Taken together, this data indicates that fatty acids are the substrates for these CAR homologs and that overexpression of a thioesterase, such as 'tesA (to release fatty acids from acyl-ACP), achieves significant production of fatty alcohols.

Depending upon the CAR homolog expressed in E. coli strain C41 (DE3, ΔfadE) (described below in Example 2), different mixtures of fatty alcohols were produced. Different compositions of fatty alcohols were observed among the three CAR homologs evaluated (see Table 9). FadD9 produced more C₁₂ fatty alcohols relative to other fatty alcohols with carbon chain lengths greater than 12. Both CarA and CarB produced a wider range in chain length of fatty alcohols than was observed when expressing FadD9.

TABLE 9
Acyl-composition of fatty alcohols produced by recombinant E.coli strains.

| Expressed with | | | | | | | |
|---|---|---|---|---|---|---|---|
| TesA* | Acyl-composition of fatty alcohols (%) | | | | | | |
| and AlrAadp1 | C10:0 | C12 | C14:1 | C14:0 | C16:1 | C16:0 | C18:1 |
| CarA | trace | 38 | 13 | 27 | 16 | 4 | 3 |
| FadD9 | trace | 63 | 14 | 16 | 7 | trace | trace |
| CarB | trace | 32 | 11 | 41 | 12 | trace | trace |

*the leaderless TesA. C12, including C12:0 and C12:1 fatty alcohols

### Quantification and Identification of Fatty Alcohols

GC/MS was performed using an Agilent 5975B MSD system equipped with a 30 m×0.25 mm (0.10 μm film) DB-5 column. The column temperature was 3 min isothermal at 100° C. The column was programmed to rise from 100° C. to 320° C. at a rate of 20° C./min. When the final temperature was reached, the column remained isothermal for 5 minutes at 320° C. The injection volume was 1 μL. The carrier gas, helium, was released at 1.3 mL/min. The mass spectrometer was equipped with an electron impact ionization source. The ionization source temperature was set at 300° C.

Prior to quantification, various alcohols were identified using two methods. First, the GC retention time of each compound was compared to the retention time of a known standard, such as a cetyl alcohol, dodecanol, tetradecanol, octadecanol, or cis-9-octadecenol. Second, identification of each compound was confirmed by matching the compound's mass spectrum to a standard's mass spectrum in the mass spectra library (e.g., C12:0, C12:1, C13:0, C14:0, C14:1, C15:0. C16:0, C16:1, C17:0, C18:0 and C18:1 alcohols).

### Example 2

This example describes the identification of a fatty alcohol biosynthetic polypeptide, YjgB, in E. coli.

E. coli contains multiple enzymes that catalyze the reversible oxidoreduction of fatty aldehydes and fatty alcohols. A BLAST search and comparison of the E. coli K12 genomic and protein databases for homologs of Acinetobacter sp. M-1 AlrA revealed that the E. coli enzyme YjgB might be the closest homolog with an about 57% sequence identity. This example sought to verify the fatty alcohol biosynthetic activity of E. coli YjgB by overexpressing YjgB with a CarB in E. coli and measure the accumulation of fatty aldehyde and production of fatty alcohols.

The plasmid pETDuet-1-'tesA-yjgB carrying 'tesA and yjgB (a putative alcohol dehydrogenase; GenBank accession number, NP_{—}418690; GenPept accession number AAC77226) from the E. coli K12 strain was prepared.

The gene yjgB (GenBank accession number, NP_{—}418690) insert was amplified using PCR from the genomic DNA of E. coli K-12 using the following primers.

NcoI YjgB forward:
(SEQ ID NO: 221)
aatccTGGCATCGATGATAAAAAGCTATGCCGCAAAAG
HindIII YjgB reverse:
(SEQ ID NO: 222)
ataaaagctTTCAAAAATCGGCTTTCAACACCACGCGG

The PCR product was then subcloned into the NcoI and HindIII sites of pETDuet-1-'tesA to generate pETDuet-1-'tesA-yjgB.

In order to evaluate the affect of carboxylic acid reductases and alcohol dehydrogenases on the production of fatty alcohols, various combinations of the prepared plasmids were transformed in the E. coli strain C41 (DE3, ΔfadE) (described in PCT/US08/058,788).

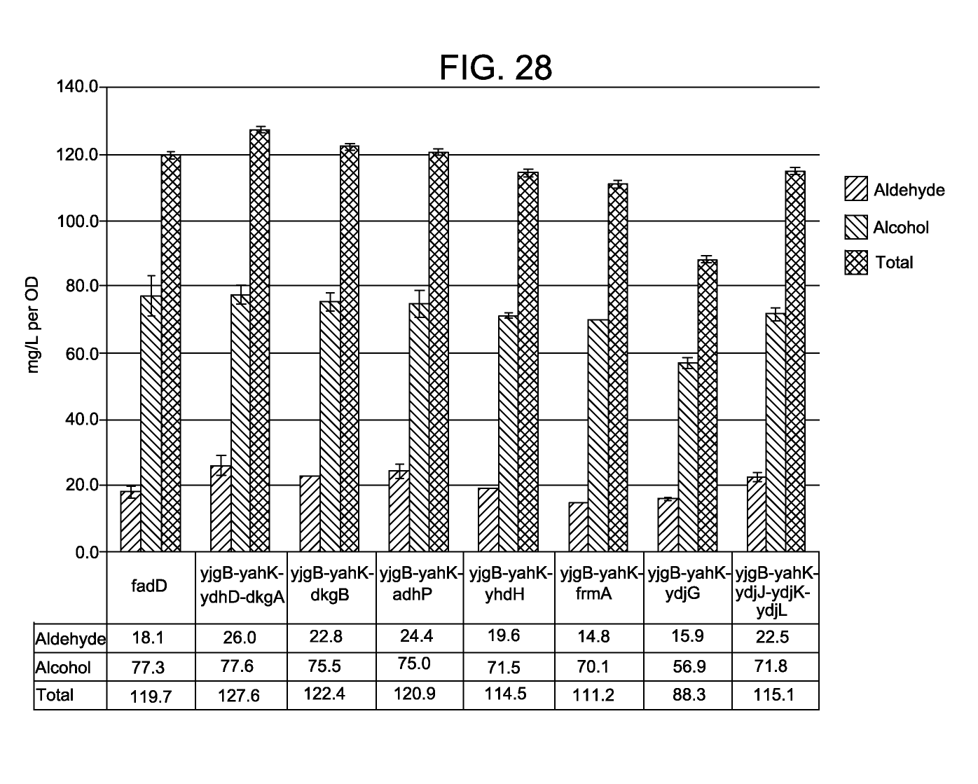

The plasmid pACYCDuet-1-carB, encoding the CAR homolog carB, was co-transformed with pETDuet-1-'tesA. In addition, pACYCDuet-1-carB was also separately co-transformed with pETDuet-1-'tesA-yjgB. As a control, pACYCDuet-1-carB was co-transformed with the empty vector pETDuet-1 (see, e.g., FIG. 28).

The plasmid pACYCDuet-1-fadD9, encoding the CAR homolog fadD9, was co-transformed with pETDuet-1-'tesA. In addition, pACYCDuet-1-fadD9 was also separately co-transformed with pETDuet-1-'tesA-yjgB. As a control, pACYCDuet-1-fadD9 was co-transformed with the empty vector pETDuet-1 (see, e.g., FIG. 28).

As an additional control, pETDuet-1-'tesA-yjgB was co-transformed with the empty vector pACYCDuet-1.

The E. coli transformants were grown in 3 mL of LB medium supplemented with carbenicillin (100 mg/L) and chloramphenicol (34 mg/L) at 37° C. After overnight growth, 15 μL of culture was transferred into 2 mL of fresh LB medium supplemented with carbenicillin and chloramphenicol. After 3.5 hours of growth, 2 mL of culture were transferred into a 125 mL flask containing 20 mL of M9 medium with 2% glucose and with carbenicillin and chloramphenicol. When the OD₆₀₀ of the culture reached 0.9, 1 mM of IPTG was added to each flask. After 20 hours of growth at 37° C., 20 mL of ethyl acetate (with 1% of acetic acid, v/v) was added to each flask to extract the fatty alcohols produced during the fermentation. The crude ethyl acetate extract was directly analyzed with GC/MS as described herein.

The measured retention times were 6.79 minutes for cis-5-dodecen-1-ol, 6.868 minutes for 1-dodecanol, 8.058 minutes for cis-7-tetradecen-1-ol, 8.19 minutes for 1-tetradecanol, 9.208 minutes for cis-9-hexadecen-1-ol, 9.30 minutes for 1-hexadecanol, and 10.209 minutes for cis-11-octadecen-1-ol.

As can be concluded from this example, the production of fatty alcohols from fatty aldehydes in the E. coli strains described above may have been catalyzed by more than one endogenous fatty alcohol biosynthetic polypeptides. On the other hand, it has been demonstrated that overexpression of YjgB with CarB and leaderless TesA significantly reduced the accumulation of fatty aldehydes, as compared to control strains that did not overexpress YjgB. But it was also noted that overexpression of YjgB appeared to reduce the overall fatty alcohol production.

### Example 3

This example describes the identification of other fatty alcohol biosynthetic polypeptides in E. coli.

A reverse genetic approach was used to identify potential fatty alcohol biosynthetic genes in E. coli MG1655 cells by expressing the acyl-ACP reductase YP_{—}400611 from Synechococcus elongatus (Synpcc7942_{—}1594) (SEQ ID NO:137). Four 3 mL LB cultures were grown overnight at 37° C., and 55 μL of stationary phase cultures were used to inoculate four independent 5.5 mL of LB. Those 5.5 mL cultures were then grown to an OD₆₀₀ of 0.8-1.0 and were then used to inoculate a corresponding number of 2 L baffled shakeflasks, each with 500 mL Hu-9 minimal media. 20 hrs after induction the cells were pelleted at 4,000×g for 20 min. The cell pellet was resuspended in 30 mL of 100 mM phosphate buffer at pH 7.2 with 1× Bacterial Protease Arrest (G Biosciences). The cells were lysed in a French press at 15,000 psi with two passes through the instrument. The cell debris was then removed by centrifuging at 10,000×g for 20 mins. The cell lysate was loaded onto two HiTrapQ columns (GE Healthcare) connected in series. The following buffers were used to elute proteins: (A) 50 mM Tris, pH 7.5 and (B) 50 mM Tris, pH 7.5 with 1 M NaCl. A gradient from 0% B to 100% B was run over 5 column volumes at a flow rate of 3 mL/min while 4 mL fractions were collected.

The fractions were assayed for alcohol biosynthetic enzymatic (e.g., aldehyde reductase/alcohol dehydrogenase) activity by taking 190 μL of a protein fraction and adding 5 μL of a 20 mM NADPH (Sigma) solution and 5 μL of a 20 mM dodecanal (Fluka) solution in DMSO. The reactions were incubated at 37° C. for 1 hr. They were then extracted with 100 μL of ethyl acetate and analyzed for dodecanol via GC/MS. Fractions eluting around 350 mM NaCl contained a fatty alcohol biosynthetic enzyme activity.

Fractions containing fatty alcohol biosynthetic enzyme activity were pooled and loaded onto a 1 mL ResourceQ column (GE Healthcare). The same conditions used for the HiTrapQ column were used, except 0.5 mL fractions were collected. Protein fractions demonstrating a capacity of converting fatty aldehydes to fatty alcohols were then pooled and concentrated using Amicon (Milipore) protein concentrators (10,000 kDa cutoffs) to a volume of 1 mL. The solution was then loaded onto a HiPrep 200 size exclusion column (GE Healthcare). A buffer solution containing 50 mM Tris, pH 7.5, and 150 mM NaCl was run through the column at a rate of 0.3 mL per min. 2 mL fractions were collected. Two protein fractions were identified as having fatty alcohol biosynthetic enzyme activity. These two fractions, plus fractions before and after these two fractions, were loaded onto a polyacrylamide gel and stained with SimplySafe Commassie stain (Invitrogen).

Comparing the bands in the active and inactive fractions, one protein band, which appeared in the active fraction, was not seen in the inactive fraction. This protein band was cut from gel and submitted to the Stanford Mass Spectroscopy Facility for LC/MS/MS protein sequencing. One of the proteins identified in this analysis was YahK. E. coli YahK was determined to be the closest paralogs of YjgB, with about 31% sequence identity to the latter.

### Example 4

This example describes the verification of YjgB and YahK as fatty alcohol biosynthetic polypeptides.

Construction of fadD Deletion Strain

The fadD gene of E. coli MG1655 was deleted using the lambda red system (Datsenko et al., Proc. Natl. Acad. Sci. USA. 97: 6640-6645 (2000)) as follows:

The chloramphenicol acetyltransferase gene from pKD3 was amplified with the primers fad1: (5′-TAACCGGCGTCTGACGACTGACTTAACGCTCAGGCTTTATT GTCCACTTTGTGTAGGCTGGAGCTGCTTCG-3′) (SEQ ID NO:223), and fad2: (5′-CATTTGGGGTTGCGATGACGACGAACACGCATTTTAGAGGTGAAGAATTGCATATG AATATCCTCCTTTAGTTCC-3′) (SEQ ID NO:224).

This PCR product was electroporated into E. coli MG1655 (pKD46). The cells were plated on L-chloramphenicol (30 μg/mL)(L-Cm) and grown overnight at 37° C. Individual colonies were picked on to another L-Cm plate and grown at 42° C. These colonies were then patched to L-Cm and L-carbenicillin (100 mg/mL) (L-Cb) plates and grown at 37° C. overnight. Colonies that were Cm^{R} and Cb^{S} were evaluated further by PCR to ensure the PCR product inserted at the correct site.

PCR verification was performed on colony lysates of these bacteria using the primers fadF (5′-CGTCCGTGGTAATCATTTGG-3′) (SEQ ID NO:225) and fadR (5′-TCGCAACCTTTTCGTTGG-3′) (SEQ ID NO:226). Expected size of the ΔfadD::Cm deletion was about 1200 bp. The chloramphenicol resistance gene was eliminated using a FLP helper plasmid as described in Datsenko et al., Proc. Natl. Acad. Sci. USA 97:6640-6645 (2000). PCR verification of the deletion was performed with primers fadF and fadR. The MG1655 ΔfadD strain was unable to grow on M9+oleate agar plates (oleate as carbon source). It was also unable to grow in M9+oleate liquid media. The growth defect was complemented by an E. coli fadD gene supplied in trans (in pCL1920-Ptrc).

Construction of MG1655(DE3, ΔfadD) Strain

To generate a T7-responsive strain, the λDE3 Lysogenization Kit (Novagen) was utilized, which is designed for site-specific integration of λDE3 prophage into an E. coli host chromosome, such that the lysogenized host can be used to express target genes cloned in T7 expression vectors. λDE3 is a recombinant phage carrying the cloned gene for T7 RNA polymerase under lacUV5 control. Briefly, the host strain was cultured in LB supplemented with 0.2% maltose, 10 mM MgSO₄, and antibiotics at 37° C. to an OD₆₀₀ of 0.5. Next, 10⁸ pfu λDE3, 10⁸ pfu Helper Phage, and 10⁸ pfu Selection Phage were incubated with 10 λL host cells. The host/phage mixture was incubated at 37° C. for 20 min to allow phage to adsorb to host. Finally, the mixture was pipetted onto an LB plate supplemented with antibiotics. The mixture was spread evenly using plating beads, and the plates were inverted plates and incubated at 37° C. overnight.

λDE3 lysogen candidates were evaluated by their ability to support the growth of the T7 Tester Phage. T7 Tester Phage is a T7 phage deletion mutant that is completely defective unless active T7 RNA polymerase is provided by the host cell. The T7 Tester Phage makes very large plaques on authentic λDE3 lysogens in the presence of IPTG, while much smaller plaques are observed in the absence of inducer. The relative size of the plaques in the absence of IPTG is an indication of the basal level expression of T7 RNA polymerase in the lysogen, and can vary widely between different host cell backgrounds.

The following procedure was used to determine the presence of DE3 lysogeny. First, candidate colonies were grown in LB supplemented with 0.2% maltose, 10 mM MgSO₄, and antibiotics at 37° C. to an OD₆₀₀ of 0.5. An aliquot of T7 Tester Phage was then diluted in 1× Phage Dilution Buffer to a titer of 2×10³ pfu/mL. In duplicate tubes, 100 μL host cells were mixed with 100 μL diluted phage. The host/phage mixture was incubated at room temperature for 10 min to allow phage to adsorb to host. Next, 3 mL of molten top agarose was added to each tube containing host and phage. The contents of one duplicate were plated onto an LB plate and the other duplicate onto an LB plate supplemented with 0.4 mM IPTG (isopropyl-b-thiogalactopyranoside) to evaluate induction of T7 RNA polymerase. Plates were allowed to sit undisturbed for 5 min until the top agarose hardened. The plates were then inverted at 30° C. overnight.

Construction of MG1655(DE3, ΔfadD, yjgB::kan) Strain

The yjgB knockout strain, MG1655(DE3, ΔfadD, yjgB::kan), was constructed by using the following lambda red system (Datsenko et al., Proc. Natl. Acad. Sci. USA 97:6640-6645 (2000)):

The kanamycin resistant gene from pKD13 was amplified with the primers yjgBRn: (5′-GCGCCTCAGATCAGCGCTGCGAATGATTTTCAAAAATCGGCTTTCAACACTG TAGGCTGGAGCTGCTTCG-3′) (SEQ ID NO:227), and yjgBFn: (5′-CTGCCATGCTCTA CACTTCCCAAACAACACCAGAGAAGGACCAAAAAATGATTCCGGGGATCCGTCGAC C-3′) (SEQ ID NO:228). The PCR product was then electroporated into E. coli MG1655 (DE3, ΔfadD)/pKD46. The cells were plated on kanamycin (50 μg/mL) (L-Kan) and grown overnight at 37° C. Individual colonies were picked on to another L-Kan plate and grown at 42° C. These colonies were then patched to L-Kan and carbenicillin (100 mg/mL) (L-Cb) plates and grown at 37° C. overnight. Colonies that were kan^{R} and Cb^{S} were evaluated further by PCR to ensure the PCR product was inserted at the correct site.

PCR verification was performed on colony lysates of these bacteria using the primers BF (5′-gtgctggcgataCGACAAAACA-3′) (SEQ ID NO:229) and BR (5′-CCCCGCCCTGCCATGCTCTACAC-3′) (SEQ ID NO:230). The expected size of the yjgB::kan knockout was about 1450 bp.

In Example 2, a fadE deletion strain was used for fatty aldehyde and fatty alcohol production from 'TesA, CAR homologs, and endogenous YjgB in E. coli. Here, to demonstrate that CAR homologs used fatty acids instead of acyl-CoA as a substrate, the gene encoding for acyl-CoA synthase in E. coli (fadD) was deleted so that the fatty acids produced were not activated with CoA. E. coli strain MG1655(DE3, ΔfadD) was transformed with pETDuet-1-'tesA and pACYCDuet-1-carB. The transformants were evaluated for fatty alcohol production using the methods described herein. These transformants produced about 360 mg/L of fatty alcohols (dodecanol, dodecenol, tetredecanol, tetredecenol, cetyl, hexadecenol, and octadecenol).

### Confirming YjgB as a Fatty Alcohol Biosynthetic Polypeptide

To confirm that YjgB was an alcohol dehydrogenase responsible for converting fatty aldehydes into their corresponding fatty alcohols, pETDuet-1-'tesA and pACYCDuet-1-fadD9 were co-transformed into either MG1655(DE3, ΔfadD) or MG1655(DE3, ΔfadD, yjgB::kan). At the same time, MG1655(DE3, ΔfadD, yjgB::kan) was transformed with both pETDuet-1-'tesA-yjgB and pACYCDuet-1-fadD9.

The E. coli transformants were grown in 3 mL of LB medium supplemented with carbenicillin (100 mg/L) and chloramphenicol (34 mg/L) at 37° C. After overnight growth, 15 μL of culture was transferred into 2 mL of fresh LB medium supplemented with carbenicillin and chloramphenicol. After 3.5 hrs of growth, 2 mL of culture was transferred into a 125 mL flask containing 20 mL of M9 medium with 2% glucose, carbenicillin, and chloramphenicol. When the OD₆₀₀ of the culture reached 0.9, 1 mM of IPTG was added to each flask. After 20 hrs of growth at 37° C., 20 mL of ethyl acetate (with 1% of acetic acid, v/v) was added to each flask to extract the fatty alcohols produced during the fermentation. The crude ethyl acetate extract was directly analyzed with GC/MS as described herein.

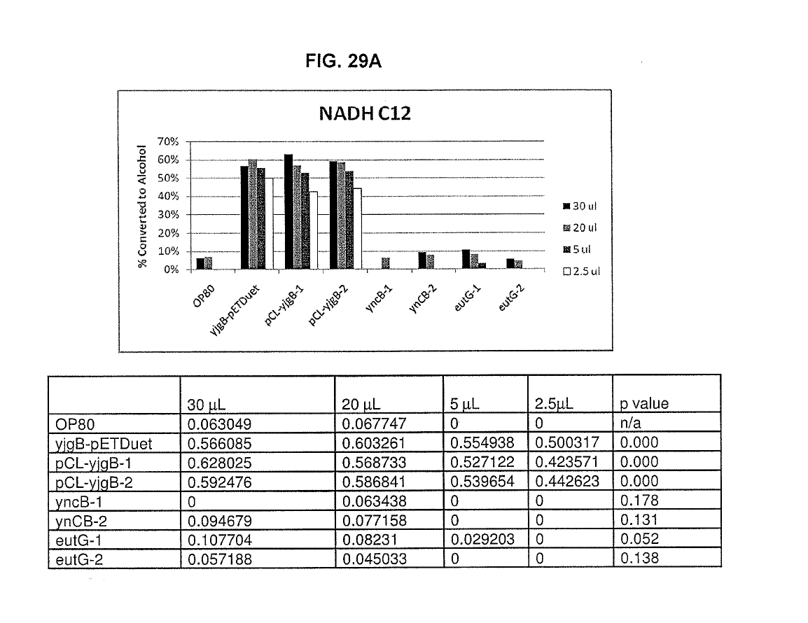

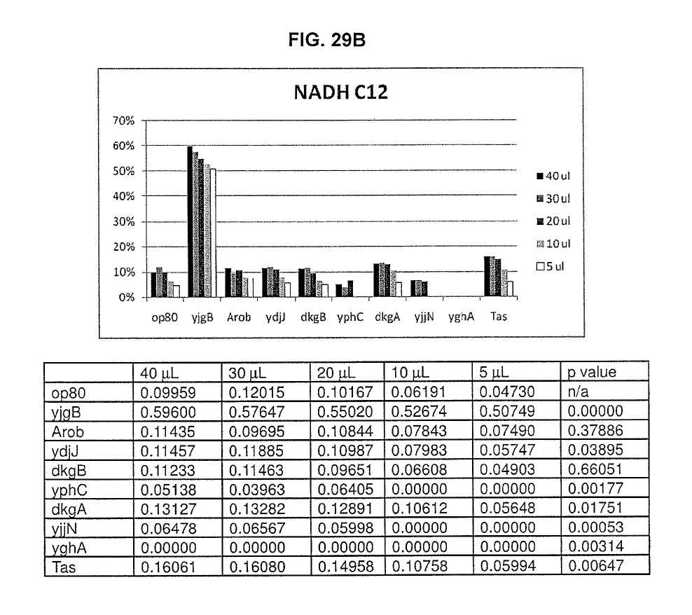

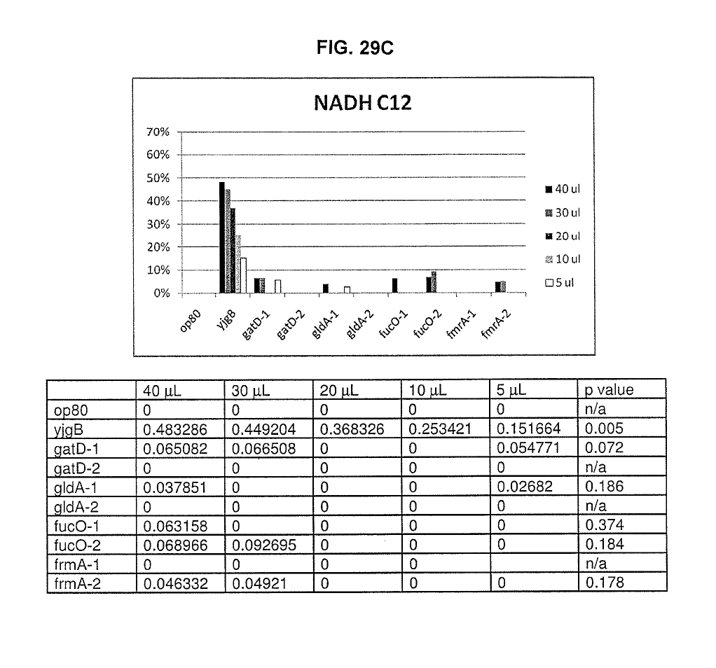

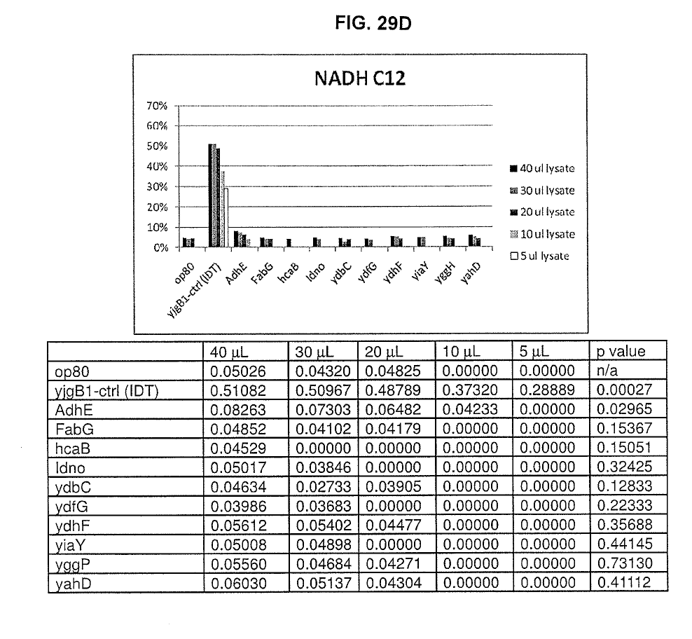

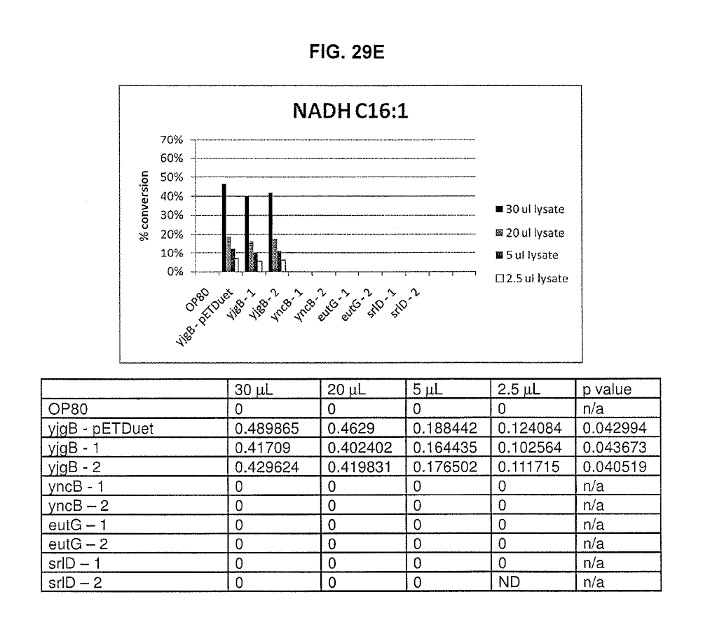

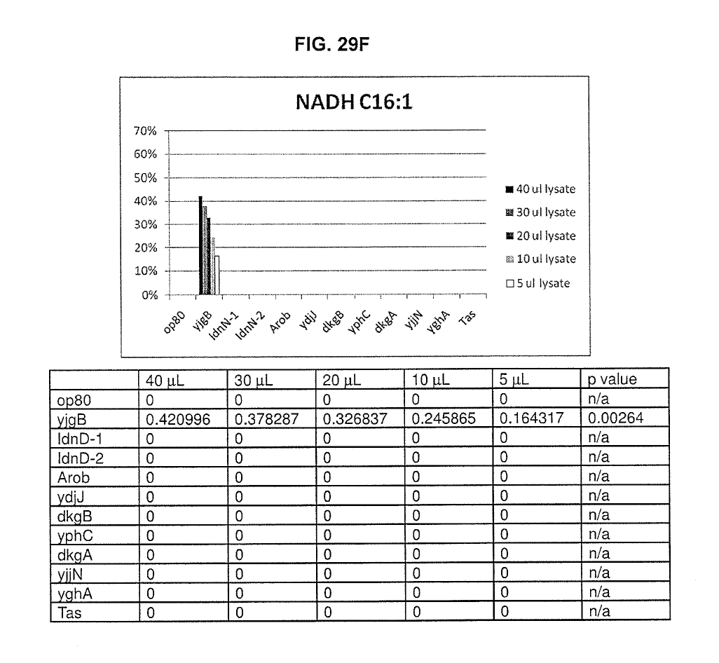

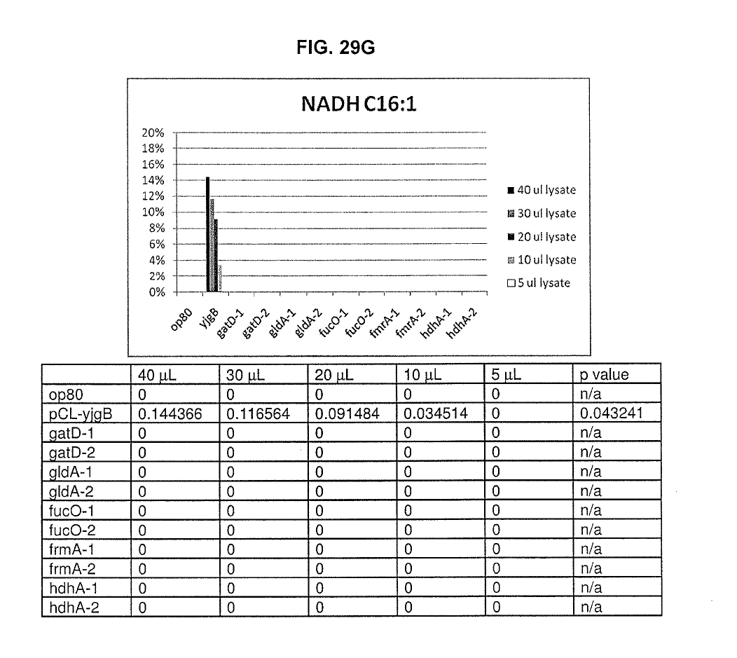

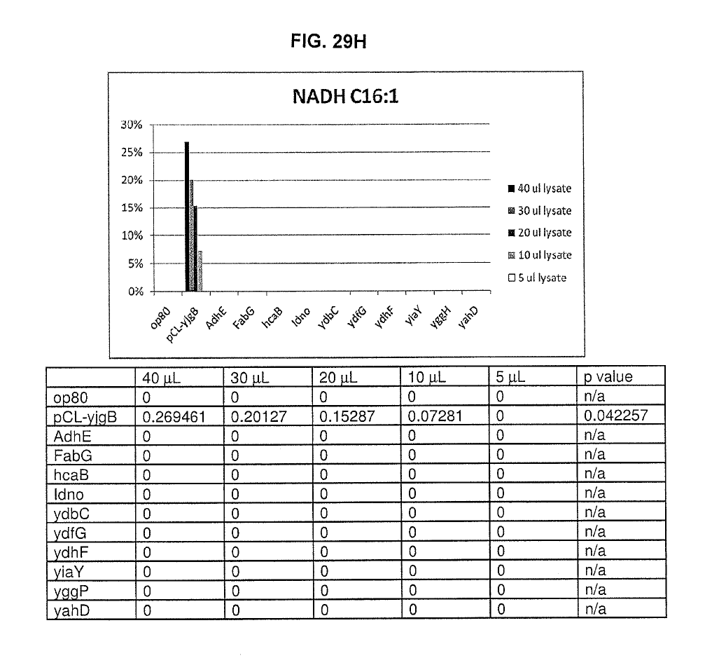

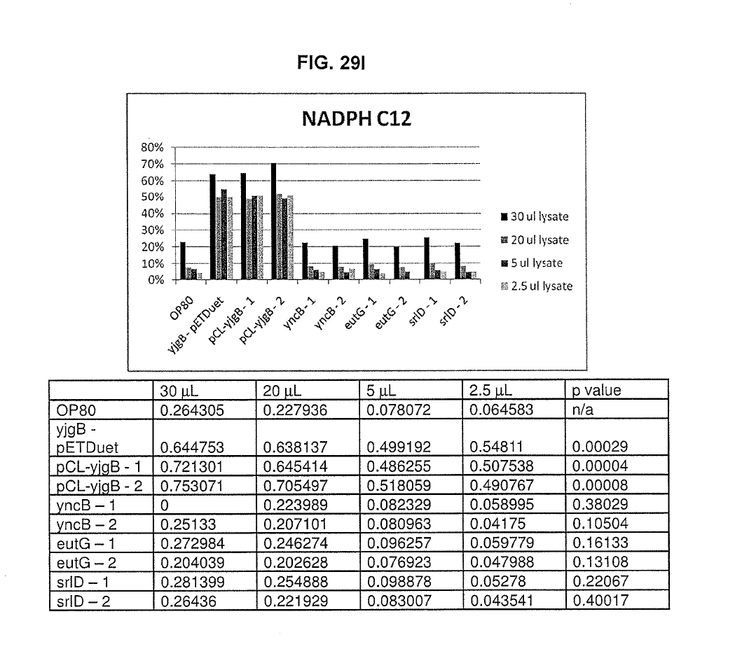

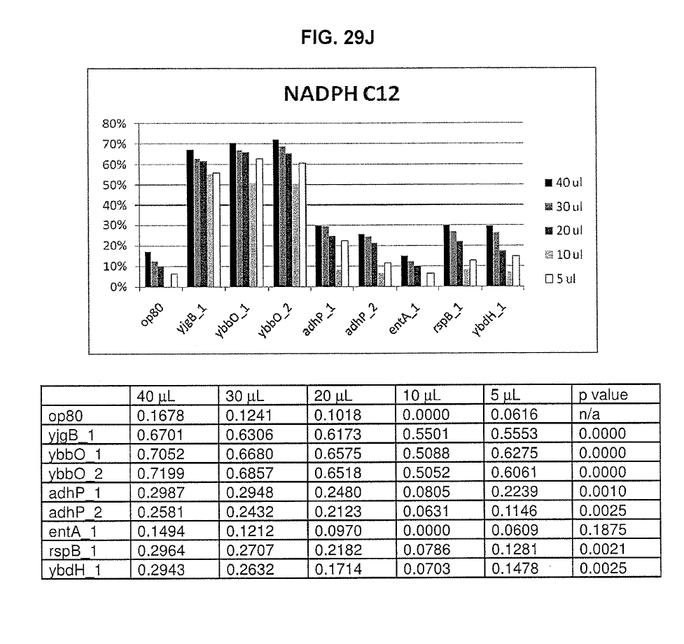

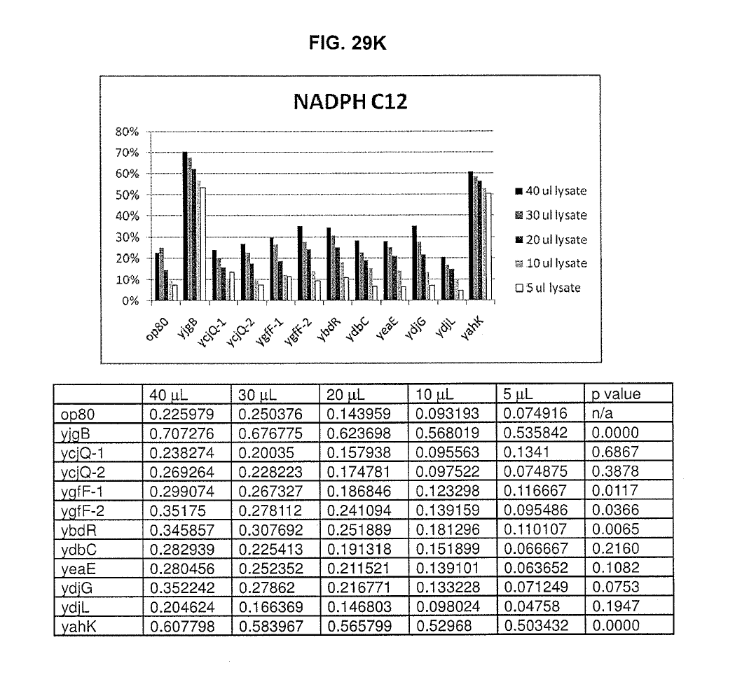

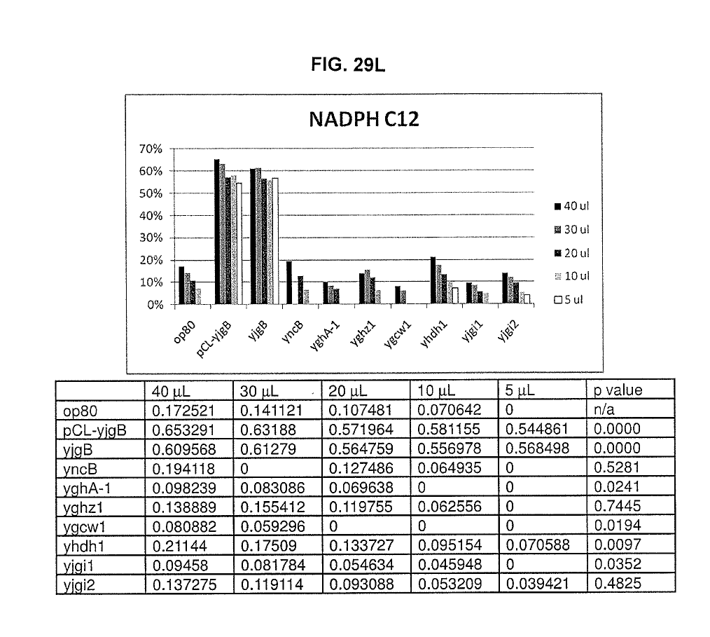

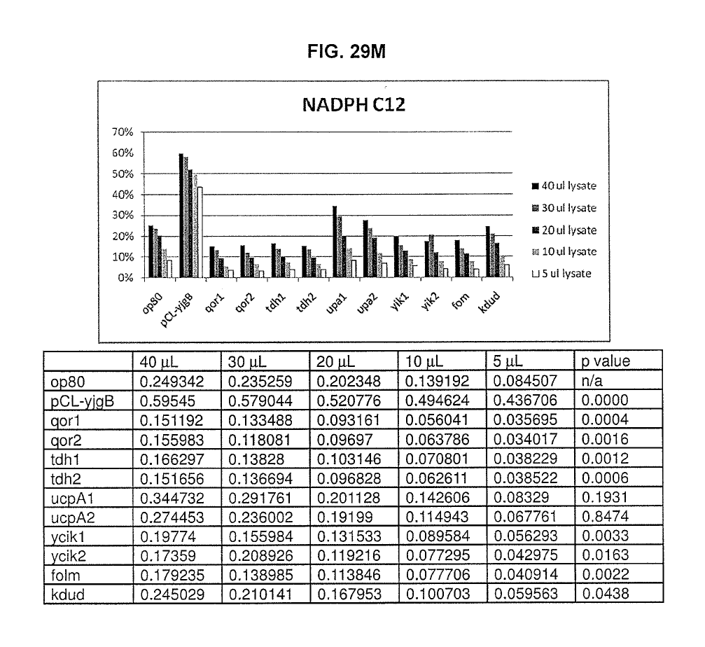

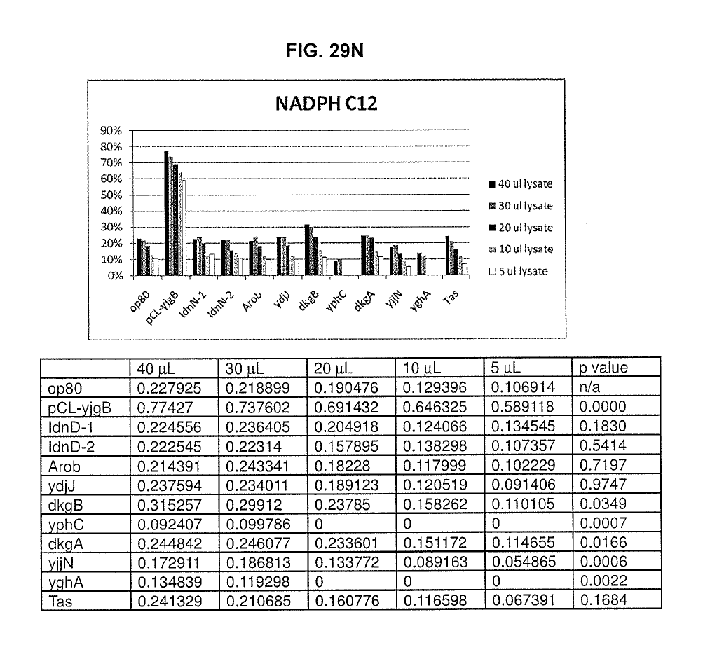

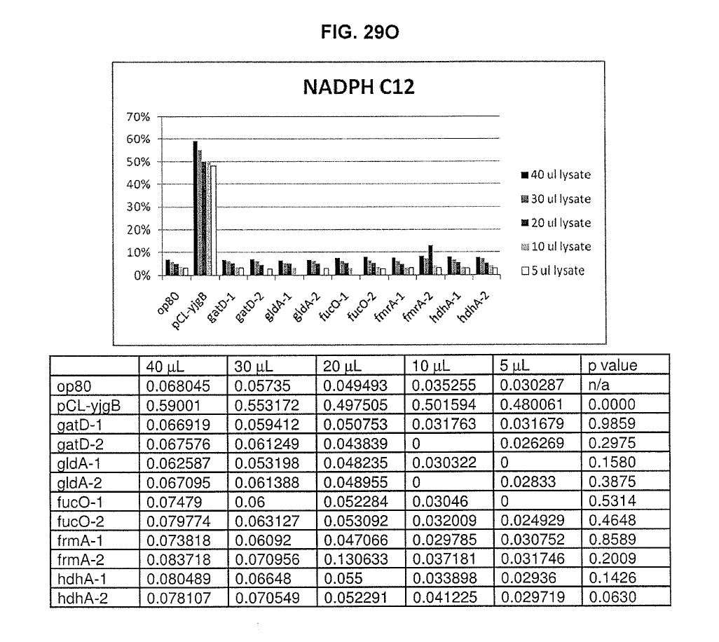

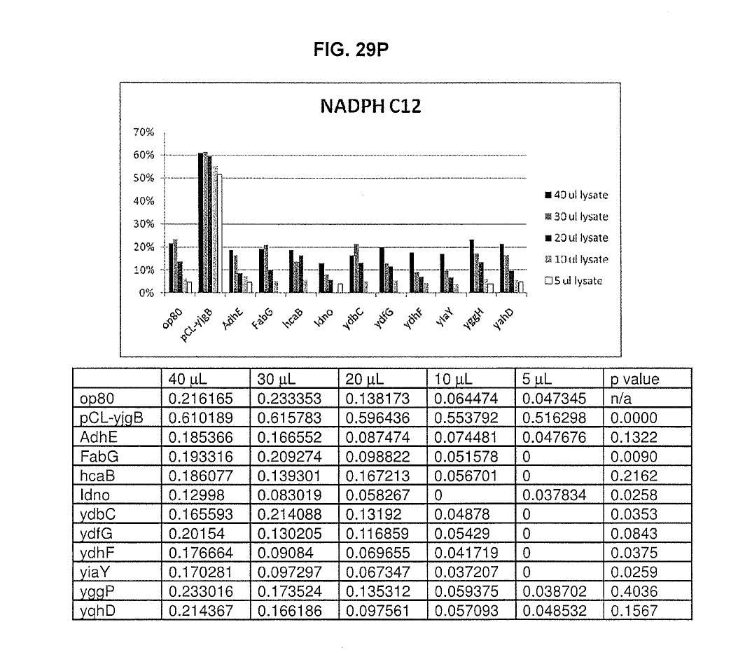

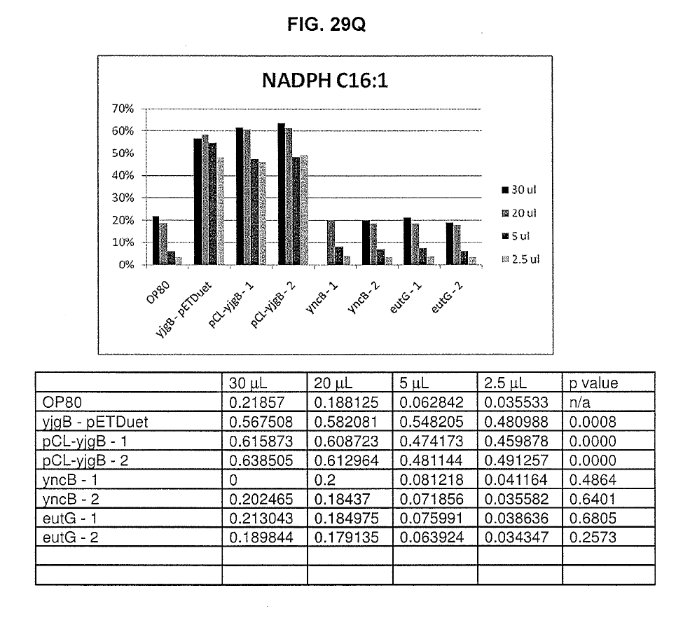

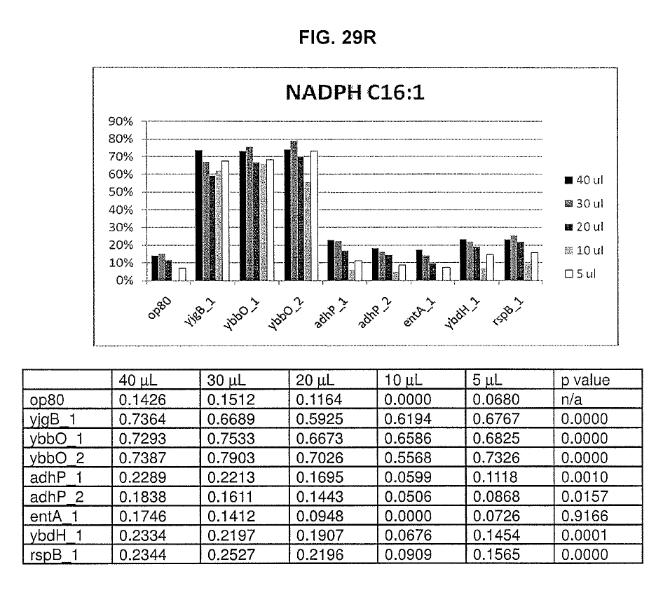

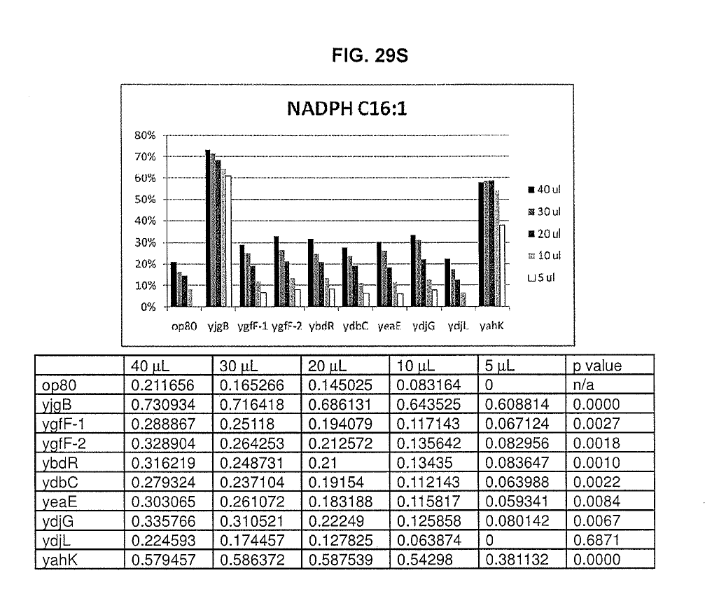

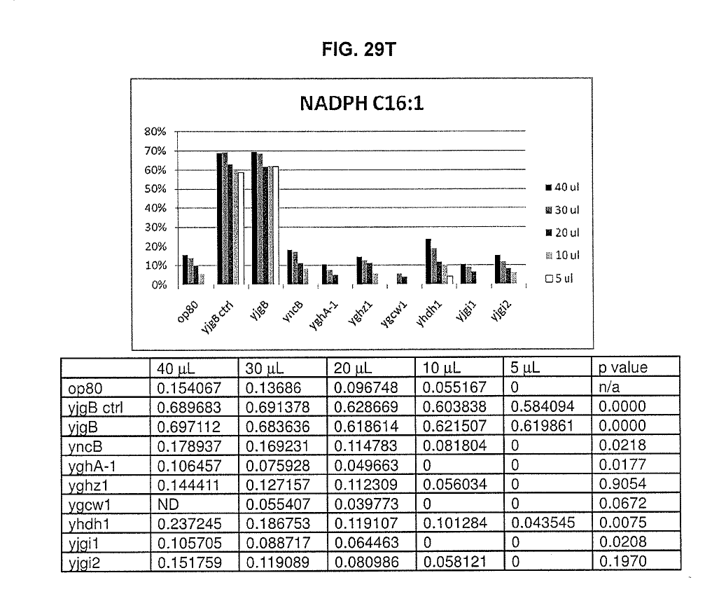

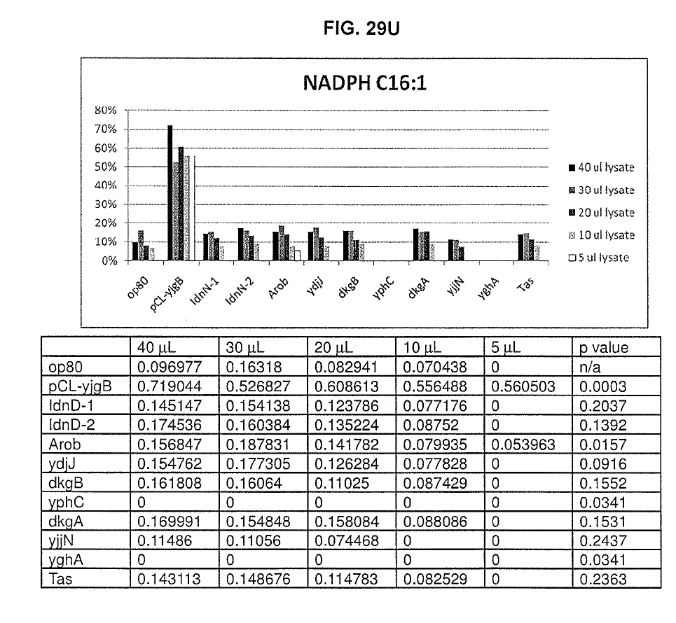

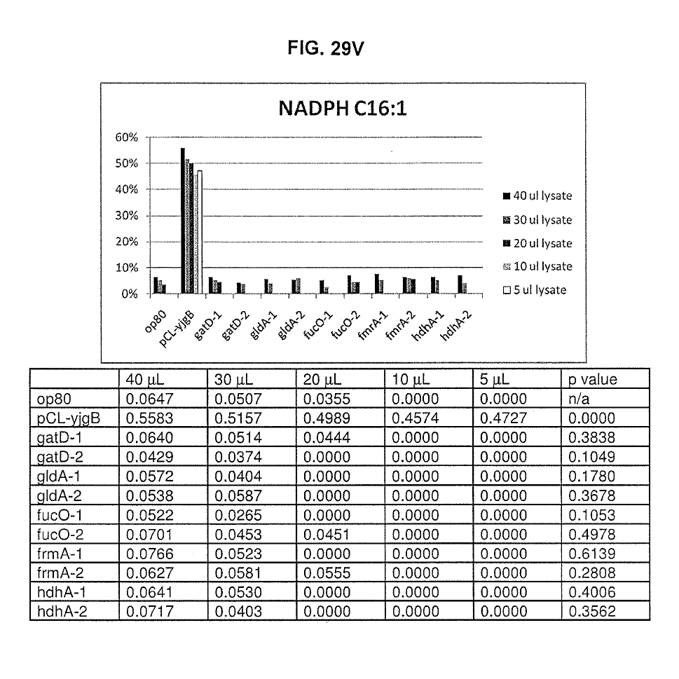

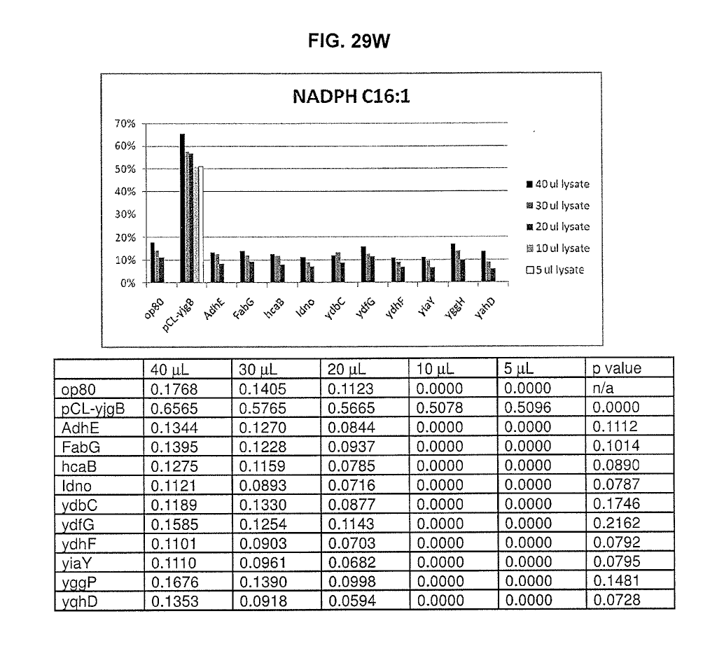

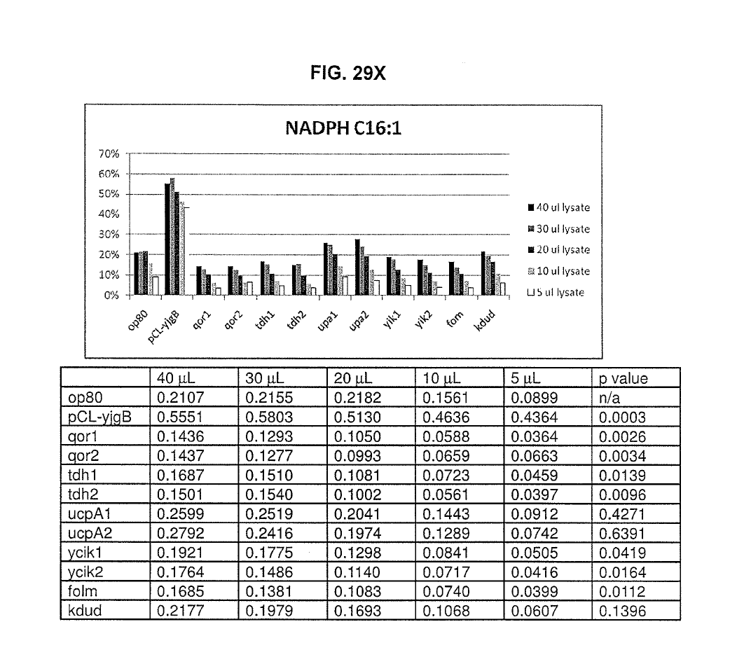

The yjgB knockout strain resulted in significant accumulation of dodecanal and a lower fatty alcohol titer (FIG. 29). The expression of yjgB from plasmid pETDuet-1-'tesA-yjgB in the yjgB knockout strain effectively removed the accumulation of dodecanal (FIG. 29). Dodecanal accumulated in the yjgB knockout strain, but it was not observed in either the wild-type strain (MG1655(DE3, ΔfadD)) or the yjgB knockout strain with the yjgB expression plasmid. The arrows in FIG. 29 indicate the GC trace of dodecanal (C12:0 aldehyde).

This data confirms that YjgB was involved in converting dodecanal into dodecanol, although there may be other alcohol dehydrogenase(s) present in E. coli to convert other aldehydes into alcohols.

### Confirming YahK as a Fatty Alcohol Biosynthetic Polypeptide

To verify that YahK was indeed an alcohol dehydrogenase, yahK was knocked out in E. coli MG1655(DE3, ΔfadD, ΔyjgB) (control strain). The yahK knock-out strain MG1655(DE3, ΔfadD, Δyjg,B ΔyahK) was constructed with the lambda red system (Datsenko et al., supra) using the following primers: yahK_F: (CATATCAGGCGTTGCCAAATACACATAGCTAATCAGGAGTAAACACAATG) (SEQ ID NO:231); and yahK_R: (AATCGCACACTAACAGACTGAAAAAATTAATA AATACCCTGTGGTTTAAC) (SEQ ID NO:232).

This ΔyahK strain and the control strain, both expressing the acyl-ACP reductase YP_{—}400611, were cultured under conditions described above. Cell free lysates were made from both strains, and each lysate was assayed for fatty alcohol biosynthetic activity as discussed above.

The ΔyahK strain did not convert dodecanal to dodecanol, while the wild type strain had this activity. For additional verification, each lysate was run on a HiTrapQ column as described above. The wild type lysate appeared to have fatty alcohol biosynthetic activity in fractions eluting around 350 mM NaCl, while the ΔyahK lysate appeared to have no fatty alcohol biosynthetic activity in this region.

### Example 5

This Example Describes the Identification of Further Fatty Alcohol Biosynthetic Polypeptides in E. coli

### Bioinformatics

It was reasoned that potential fatty alcohol biosynthetic polypeptides in E. coli were most likely members of the following four protein families: Zn-dependent alcohol dehydrogenases (Pfam 00107 and 08240), Fe-dependent alcohol dehydrogenases (Pfam 00465), aldo-keto reductases (Pfam 00248) and short-chain dehydrogenases (Pfam 00106) (Pfam=protein family according to “pfam.sanger.ac.uk”). Further protein families that were likely to include potential alcohol biosynthetic polypeptides in E. coli may include, for example, the dehydroquinone synthase family (Pfam 01761), the phosphogluconate dehydrogenase family (Pfam 03446), the hydroxyacid dehydrogenase family (Pfam 02826, Pfam 00389), the aldehyde dehydrogenase family (Pfam 00171), the glutamyl-tRNA reductase family (Pfam 01488, Pfam 08501), the GFO/IDH/MOCA family (Pfam 01408, Pfam 02894), the mannitol dehydrogenase family (Pfam 01232, Pfam 08125), the IMP dehydrogenase family (Pfam 00478), the oxidoreductase family (Pfam 10722), the epimerase family (Pfam 001370), the alcohol oxidase family (Pfam 00732, Pfam 05199), the PQQ dehydrogenase family (Pfam 01011), the xanthine dehydrogenase family (Pfam 00941), the FAD/NAD(P)-binding oxidoreductase family (Pfam 01266), the flavin/NADH-binding oxidoreductase family (Pfam 01613), the FAD-linked oxidoreductase family (Pfam 01565, Pfam 02913), the ferredoxin reductase family (Pfam 00175, Pfam 00970, Pfam 00111), the anaerobic dehydrogenase family (Pfam 00384, Pfam 01568), the molybdenum-binding oxidoreductase family (Pfam 01315, Pfam 02738), the DMSO reductase family (Pfam 02976), the nitroreductase family (Pfam 00881), the FeS-binding oxidoreductase family (Pfam 00037, Pfam 07992), another oxidoreductase family (Pfam 00037, Pfam 01558, Pfam 01855, Pfam 02775, Pfam 10371), the Fe—S oxidoreductase family (Pfam 04055), the NADH-ubiquinone oxidoreductase family (Pfam 02508), the NAD(P)H:quinine oxidoreductase family (Pfam 05368), the NADH:ubiquinone oxidoreductase family (Pfam 01512, Pfam 10531, Pfam 10589), the glutathione reductase family (Pfam 02852, Pfam 07992), or a number of other predicted oxidoreductase families including, for example those within Pfam 03006, Pfam 03960, Pfam 00070. The potential families from which a fatty alcohol biosynthetic polypeptide of E. coli can be isolated are listed in Table 10 below.

TABLE 10
Protein families that may contain additional Fatty Alcohol Biosynthetic Polypeptides

| | | | |
|---|---|---|---|
| Gene | | | |
| (example) | Paralogs | Function | Pfam |

NAD(P) dependent

| | | | | |
|---|---|---|---|---|
| yiaE | 5 | oxidize/reduce 2-keto-carboxylic acids to 2- | pfam02826 | pfam00389 |
| B3553 | | hydroxy carboxylic acids | | |
| YdcW, | 14 | aldehyde dehydrogenase family (oxidize | pfam00171 | |
| AstD | | aldehydes to carboxylic acids) | | |
| AroE | 3 | dehydroshikimate reductase, NAD(P)-binding | pfam01488 | pfam08501 |
| yihU | 6 | predicted oxidoreductase with NAD(P)-binding | pfam03446 | |
| | | Rossmann-fold domain | | |
| mviM | 6 | predicted oxidoreductase with NAD(P)-binding | pfam01408 | pfam02894 |
| | | Rossmann-fold domain | | |
| uxuB | 5 | D-mannonate oxidoreductase, NAD-binding, | pfam01232 | pfam08125 |
| | | Rossman-type fold | | |
| yciW | 1 | predicted oxidoreductase | — | |
| ybjN | 1 | predicted oxidoreductase | pfam10722 | |
| gale | 11 | Epimerase, Rossman-fold | pfam01370 | |
| guaB/C | 2 | IMP dehydrogenase/GMP reductase | pfam00478 | |

Alcohol Oxidase (Flavo), NAD independent

| | | | | |
|---|---|---|---|---|
| BetA | 1 | choline to betaine aldehyde, cholin DH, | pfam00732 | pfam05199 |
| | | Flavoprotein, O2-dep. | | |

PQQ Dehydrogenase, NAD independent

| | | | |
|---|---|---|---|
| YfgL | 3 | outer membrane protein assembly | pfam01011 |
| YghJ | | predicted inner membrane lipoprotein | |
| gcd | | glucose dehydrogenase | |

Other:

| | | | | |
|---|---|---|---|---|
| xdhB | 3 | xanthine dehydrogenase, FAD-binding subunit | pfam00941 | |
| fixC | 18 | predicted oxidoreductase with FAD/NAD(P)- | pfam01266 | |
| | | binding domain | | |
| ycdH | 2 | predicted oxidoreductase, flavin:NADH | pfam01613 | |
| | | component | | |
| ydiJ | 6 | predicted FAD-linked oxidoreductase (6/3) | pfam01565 | pfam02913 |
| cbrA | 4 | predicted oxidoreductase with FAD/NAD(P)- | pfam01494 | |
| | | binding domain | | |
| hcr | 9 | ferredoxin(flavodoxin)-NADPH reductase | pfam00175 | pfam00970 |
| | | (7/6/9) | | pfam00111 |
| ydeP | 14 | Anaerobic dehydrogenases, typically | pfam00384 | pfam01568 |
| | | selenocysteine-containing, molybdenum | | |
| | | (13/14) | | |
| yagR | 3 | predicted oxidoreductase with molybdenum- | pfam01315 | pfam02738 |
| | | binding domain | | |
| ynfH | 2 | oxidoreductase, membrane subunit, DMSO | pfam04976 | |
| | | reductase | | |
| ydjA | 4 | predicted oxidoreductase, nitroreductase (FAD | pfam00881 | |
| | | dep.) | | |
| aegA | 16 | fused predicted oxidoreductase: FeS binding | pfam00037 | pfam07992 |
| | | subunit/NAD/FAD-binding subunit | | |
| ydbK | ? | Acting on the aldehyde or oxo group of donors. | pfam00037 | pfam01558 |
| | | With an iron-sulfur protein as acceptor | | pfam01855 |
| | | | pfam02775 | pfam10371 |
| yhcC | 19 | predicted Fe—S oxidoreductase, radical SAM | pfam04055 | |
| | | protein | | |
| rsxE | 2 | NADH-ubiquinone oxidoreductase | pfam02508 | |
| ytfG | 1 | NAD(P)H:quinone oxidoreductase | pfam05368 | |
| nuoF/C | 1 | NADH:ubiquinone oxidoreductase | pfam01512 | pfam10531 |
| | | | | pfam10589 |
| gor | 4 | glutathione reductase, pyruvate dehydrogenase | pfam02852 | pfam07992 |
| | | complex: dihydrolipoamideDH (E3) | | |
| yqfA | 1 | predicted oxidoreductase, inner membrane | pfam03006 | |
| | | subunit, channel protein, hemolysin fam. | | |
| yfgD | | | pfam03960 | |
| ygfK | | | pfam00070 | |

The following 8 candidates were chosen for initial experimental analysis: yahK, yjgB, adhP, dkgA, dkgB, yhdH, ydjL, and yqhD (Table 12).

To determine if these genes could reduce fatty aldehydes to fatty alcohols, these 8 genes were cloned into a pET-Duet vector along with E. coli 'tesA. These genes were then transformed into E. coli (DE3) MG1655 ΔyjgBΔyahK cells. Next 3 mL overnight starter cultures were grown in LB with carbanecillin (100 mg/L) at 37° C. A control strain lacking a candidate alcohol dehydrogenase was also included in the experiment. 1 mL of each overnight culture was used to inoculate 50 mL of fresh LB with carbanecillin. The cultures were shaken at 37° C. until reaching an OD₆₀₀ of 0.8-1. The cultures were then transferred to 18° C., induced with 1 mM IPTG, and shaken overnight.

Cell free lysates were prepared by centrifuging the cultures at 4,000×g for 20 mins. The cultures were then resuspended in 1 mL of Bugbuster (Novagen) and gently shaken at room temperature for 5 min. The cell debris was removed by spinning at 15,000×g for 10 min. The resulting lysates were assayed for alcohol dehydrogenase activity by mixing 88 μL of lysate, 2 of 40 mM cis-11-hexadecenal in DMSO, and 10 μL of 20 mM NADPH. The samples were incubated at 37° C. for 30 min. and were then extracted with 100 μL of ethyl acetate. The extracts were analyzed using GC/MS.

All proteins showed significantly better conversion of cis-11-hexadecenal to cis-11-hexadecanol as compared with the 'TesA only control (see Table 11). These results were confirmed in assays using dodecanal instead of cis-11-hexadecenal as the substrate (see Table 11).

To investigate how these enzymes contribute to fatty alcohol dehydrogenase activity in E. coil under production conditions, first the yjgB yahK double knock-out strain in MG1655(DE3, ΔfadD) (described above) was tested by transforming it with a plasmid expressing acyl-ACP reductase YP_{—}400611 and analyzing fatty aldehyde and fatty alcohol titers. The test strain also contained a plasmid expressing a decarbonylase. This double knock-out mutant showed slightly higher fatty aldehyde titers in several experiments (see, e.g., FIG. 30), confirming that these two putative alcohol dehydrogenases contribute to fatty alcohol dehydrogenase activity in E. coli under production conditions. Next, two additional genes, yncB and ydjA, were deleted in the yjgB yahK double mutant. YdjA, which is not a member of the four protein families mentioned above, demonstrated slightly elevated fatty aldehyde levels (see FIG. 30), indicating that it may also contribute to fatty alcohol dehydrogenase activity in E. coli under production conditions.

### Overexpression of Select Fatty Alcohol Biosynthetic Polypeptide Candidates

Additionally, the active fatty alcohol dehydrogenases from Table 11 were also deleted in MG1655 (DE3, ΔfadD, Δyjg,B ΔyahK) and tested as described above. Several of these deletion strains showed slightly elevated fatty aldehyde levels, suggesting that these may also contribute to fatty alcohol dehydrogenase activity in E. coli under production conditions (see FIG. 31).

TABLE 11
Overexpression of putative fatty alcohol dehydrogenase genes

| | GC/MS Assay | | NADPH assay |
|---|---|---|---|
| | % conversion to corresponding alcohol | | Initial rate (slope) |
| Substrate | Dodecanal | cis 11-hexadecanal | cis 11-hexadecenal |
| Overexpression | | | |
| None | 9 | 12 | 0.2 |
| YjgB | 54 | 89 | 24.8 |
| YahK | 47 | 87 | 28.3 |
| AdhP | 52 | 45 | 4.1 |
| YdjL | 51 | 23 | 0.14 |
| YhdH | 59 | 74 | 13.7 |
| YqhD | 55 | 23 | 7.3 |
| YafB (dkgB) | 52 | 65 | 9.4 |
| YqhE (dkgA) | 45 | 50 | 9.6 |

### Example 6

This example describes an overexpression study of a more comprehensive set of putative fatty alcohol biosynthetic polypeptides in E. coli

A larger and more comprehensive set of putative fatty alcohol biosynthetic polypeptides were selected for an overexpression study to identify the members of various protein families that contribute to the reduction of fatty aldehydes to fatty alcohols in E. coli. Specifically, each of the fatty alcohol biosynthetic genes in Table 12 below were overexpressed and analyzed for fatty aldehyde conversion and/or fatty alcohol production.

TABLE 12
Putative Fatty Alcohol Biosynthetic Genes That Were
Overexpressed (including members of the 4 families
mentioned above, with the most likely candidates
for fatty alcohol biosynthetic genes)

| Alcohol Dehydrogenases | Pfam |
|---|---|
| Zn-dependent (17) | |
| yjgB | 00107, 08240 |
| yahK | 00107, 08240 |
| adhP | 00107, 08240 |
| ydjL | 00107, 08240 |
| ydjJ | 00107, 08240 |
| yjgV (idnD) | 00107, 08240 |
| tdh | 00107, 08240 |
| yjjN | 00107, 08240 |
| rspB | 00107, 08240 |
| gatD | 00107, 08240 |
| yphC | 00107, 08240 |
| yhdH | 00107, 08240 |
| ycjQ | 00107 |
| yncB | 00107 |
| QOR | 00107, 08240 |
| ADH3 (frmA) | 00107, 08240 |
| ybdR | 00107, 08240 |
| yggP | 00107, 08240 |
| Fe-dependent (5) | |
| yiaY | 00465 |
| fucO | 00465 |
| eutG | 00465 |
| yqhD | 00465 |
| adhE | 00465 |
| Aldo-Keto Reductase (9) | |
| yafB (dkgB) | 00248 |
| ydjG | 00248 |
| yeaE | 00248 |
| yqhE (dkgA) | 00248 |
| yajO | 00248 |
| yghZ | 00248 |
| tas | 00248 |
| ydhF | 00248 |
| ydbC | 00248 |
| | Pfam |
| Short-Chain Dehydrogenase | |
| ybbO | pfam00106 |
| yohF | pfam00106 |
| yciK | pfam00106 |
| ygfF | pfam00106 |
| yghA | pfam00106 |
| yjgI | pfam00106 |
| ydfG | pfam00106 |
| ygcW | pfam00106 |
| ucpA | pfam00106 |
| entA | pfam00106 |
| folM | pfam00106 |
| hdhA | pfam00106 |
| hcaB | pfam00106 |
| srlD | pfam00106 |
| kduD | pfam00106 |
| idnO | pfam00106 |
| fabG | pfam00106 |
| fabI | pfam00106 |
| DHQ Synthase | |
| ybdH | pfam01761 |
| gldA | pfam01761 |
| aroB | pfam01761 |

Each gene was cloned into the expression vector OP-80 (SEQ ID NO:233), which was digested with the restriction enzymes NcoI and EcoRI. The genes were amplified using PCR from E. coli MG1655 genomic DNA using the primers listed in Table 13.

TABLE 13
primers

| Name | Sequence |
|---|---|
| adhE_f | TAAGGAGGAATAAACCATGGCTGTTACTAATGTCGCTGAACTTAACGC |
| | (SEQ ID NO: 234) |
| adhE_r | CGGGCCCAAGCTTCGAATTTTAAGCGGATTTTTTCGCTTTTTTCTCAGCTTTAGC |
| | (SEQ ID NO: 235) |
| adhP_f | TAAGGAGGAATAAACCATGAAGGCTGCAGTTGTTACGAAGGATCATC |
| | (SEQ ID NO: 236) |
| adhP_r | CGGGCCCAAGCTTCGAATTTTAGTGACGGAAATCAATCACCATGCGGC |
| | (SEQ ID NO: 237) |
| aroB_f | TAAGGAGGAATAAACCATGGAGAGGATTGTCGTTACTCTCGGGG |
| | (SEQ ID NO: 238) |
| aroB_r | CGGGCCCAAGCTTCGAATTTTACGCTGATTGACAATCGGCAATGGC |
| | (SEQ ID NO: 239) |
| dkgA_f | TAAGGAGGAATAAACCATGGCTAATCCAACCGTTATTAAGCTACAGGATG |
| | (SEQ ID NO: 240) |
| dkgA_r | CGGGCCCAAGCTTCGAATTTTAGCCGCCGAACTGGTCAGGATCGG |
| | (SEQ ID NO: 241) |
| dkgB_f | TAAGGAGGAATAAACCATGGCTATCCCTGCATTTGGTTTAGGTAC |
| | (SEQ ID NO: 242) |
| dkgB_r | CGGGCCCAAGCTTCGAATTTTAATCCCATTCAGGAGCCAGACCTTC |
| | (SEQ ID NO: 243) |
| entA_f | TAAGGAGGAATAAACCATGGATTTCAGCGGTAAAAATGTCTGGGTAAC |
| | (SEQ ID NO: 244) |
| entA_r | CGGGCCCAAGCTTCGAATTTTATGCCCCCAGCGTTGAGCC |
| | (SEQ ID NO: 245) |
| eutG_f | TAAGGAGGAATAAACCATGCAAAATGAATTGCAGACCGCGCTC |
| | (SEQ ID NO: 246) |
| eutG_r | CGGGCCCAAGCTTCGAATTTTATTGCGCCGCTGCGTACAGG |
| | (SEQ ID NO: 247) |
| fabI_f | TAAGGAGGAATAAACCATGGGTTTTCTTTCCGGTAAGCGCATTC |
| | (SEQ ID NO: 248) |
| fabI_r | CGGGCCCAAGCTTCGAATTTTATTTCAGTTCGAGTTCGTTCATTGCAGCAATG |
| | (SEQ ID NO: 249) |
| folM_f | TAAGGAGGAATAAACCATGGGTAAAACCCAGCCCTTGCCAATATTAATTAC |
| | (SEQ ID NO: 250) |
| folM_r | CGGGCCCAAGCTTCGAATTTTAACGCAGATGACGACCGCCATC |
| | (SEQ ID NO: 251) |
| frmA_f | TAAGGAGGAATAAACCATGAAATCACGTGCTGCCGTTGCATTTG |
| | (SEQ ID NO: 252) |
| frmA_r | CGGGCCCAAGCTTCGAATTTCAGTAACGAATTACGGTTCGAATGGATTTGCC |
| | (SEQ ID NO: 253) |
| fucO_f | TAAGGAGGAATAAACCATGATGGCTAACAGAATGATTCTGAACGAAACGG |
| | (SEQ ID NO: 254) |
| fucO_r | CGGGCCCAAGCTTCGAATTTTACCAGGCGGTATGGTAAAGCTCTACAATATCC |
| | (SEQ ID NO: 255) |
| gatD_f | TAAGGAGGAATAAACCATGAAATCAGTGGTGAATGATACTGATGGTATCGTG |
| | (SEQ ID NO: 256) |
| gatD_r | CGGGCCCAAGCTTCGAATTTCAGGGAATGAGCAACACTTTGCCC |
| | (SEQ ID NO: 257) |
| gldA_f | TAAGGAGGAATAAACCATGGACCGCATTATTCAATCACCGGGTAAATAC |
| | (SEQ ID NO: 258) |
| gldA_r | CGGGCCCAAGCTTCGAATTTTATTCCCACTCTTGCAGGAAACGCTGAC |
| | (SEQ ID NO: 259) |
| hdhA_f | TAAGGAGGAATAAACCGTGTTTAATTCTGACAACCTGAGACTCGACG |
| | (SEQ ID NO: 260) |
| hdhA_r | CGGGCCCAAGCTTCGAATTTTAATTGAGCTCCTGTACCCCACCACC |
| | (SEQ ID NO: 261) |
| idnD_f | TAAGGAGGAATAAACCATGCAAGTGAAAACACAGTCCTGCGTTG |
| | (SEQ ID NO: 262) |
| idnD_r | CGGGCCCAAGCTTCGAATTTTAGAAAACAAGCTGGACTTTTGCTGCCTG |
| | (SEQ ID NO: 263) |
| idnO_f | TAAGGAGGAATAAACCATGAACGATCTATTTTCACTGGCAGGAAAAAATATCTTGATTAC |
| | (SEQ ID NO: 264) |
| idnO_r | CGGGCCCAAGCTTCGAATTTTAAACAGCCACTAACATGCCGCCATC |
| | (SEQ ID NO: 265) |
| kduD_f | TAAGGAGGAATAAACCATGATTTTAAGTGCATTTTCTCTCGAAGGTAAAGTTGCG |
| | (SEQ ID NO: 266) |
| kduD_r | CGGGCCCAAGCTTCGAATTTTAACGCGCCAGCCAACCG |
| | (SEQ ID NO: 267) |
| qor_f | TAAGGAGGAATAAACCATGGCAACACGAATTGAATTTCACAAGCACG |
| | (SEQ ID NO: 268) |
| qor_r | CGGGCCCAAGCTTCGAATTTTATGGAATCAGCAGGCTGGAACCTTG |
| | (SEQ ID NO: 269) |
| rspB_f | TAAGGAGGAATAAACCATGAAAAGCATATTAATTGAAAAACCGAATCAACTGGC |
| | (SEQ ID NO: 270) |
| rspB_r | CGGGCCCAAGCTTCGAATTTATTCAGAAAAAGTGAGTAAGACTTTGCAGCAATGTTTTTG |
| | (SEQ ID NO: 271) |
| srlD_f | TAAGGAGGAATAAACCATGAATCAGGTTGCCGTTGTCATCGG |
| | (SEQ ID NO: 272) |
| srlD_r | CGGGCCCAAGCTTCGAATTTCAGAACATCACCTGACCGCCG |
| | (SEQ ID NO: 273) |
| tdh_f | TAAGGAGGAATAAACCATGAAAGCGTTATCCAAACTGAAAGCGGAAG |
| | (SEQ ID NO: 274) |
| tdh_r | CGGGCCCAAGCTTCGAATTTTAATCCCAGCTCAGAATAACTTTCCCGGAC |
| | (SEQ ID NO: 275) |
| ucpA_f | TAAGGAGGAATAAACCATGGGTAAACTCACGGGCAAGACAG |
| | (SEQ ID NO: 276) |
| ucpA_r | CGGGCCCAAGCTTCGAATTTCAGATACCGACGCTAACCGTCTCC |
| | (SEQ ID NO: 277) |
| yahK_f | TAAGGAGGAATAAACCATGAAGATCAAAGCTGTTGGTGCATATTCCG |
| | (SEQ ID NO: 278) |
| yahK_r | CGGGCCCAAGCTTCGAATTTCAGTCTGTTAGTGTGCGATTATCGATAACAAAACG |
| | (SEQ ID NO: 279) |
| yajO_f | TAAGGAGGAATAAACCATGCAATACAACCCCTTAGGAAAAACCGAC |
| | (SEQ ID NO: 280) |
| yajO_r | CGGGCCCAAGCTTCGAATTTTATTTAAATCCTACGACAGGATGCGGTTTATACGG |
| | (SEQ ID NO: 281) |
| ybbO_f | TAAGGAGGAATAAACCATGACTCATAAAGCAACGGAGATCCTGACAG |
| | (SEQ ID NO: 282) |
| ybbO_r | CGGGCCCAAGCTTCGAATTTCACCCCTGCAATATTTTGTCCATCACG |
| | (SEQ ID NO: 283) |
| ybdH_f | TAAGGAGGAATAAACCATGCCTCACAATCCTATCCGCGTG |
| | (SEQ ID NO: 284) |
| ybdH_r | CGGGCCCAAGCTTCGAATTTCAGGCTTTAAACGATTCCACTTTTTTGAACGC |
| | (SEQ ID NO: 285) |
| ybdR_f | TAAGGAGGAATAAACCATGAAAGCATTGACTTATCACGGCCCAC |
| | (SEQ ID NO: 286) |
| ybdR_r | CGGGCCCAAGCTTCGAATTTCATATTGTTCCCCCCGGCATCG |
| | (SEQ ID NO: 287) |
| yciK_f | TAAGGAGGAATAAACCATGCATTACCAGCCAAAACAAGATTTACTCAATGATC |
| | (SEQ ID NO: 288) |
| yciK_r | CGGGCCCAAGCTTCGAATTTCATTGGGAAATTCCTGGTTTACGGCC |
| | (SEQ ID NO: 289) |
| ycjQ_f | TAAGGAGGAATAAACCATGAAAAAGTTAGTAGCCACAGCACCGC |
| | (SEQ ID NO: 290) |
| ycjQ_r | CGGGCCCAAGCTTCGAATTTTAAAACGTAACGCCCATTTTGATGCTCTGTTC |
| | (SEQ ID NO: 291) |
| ydbC_f | TAAGGAGGAATAAACCATGAGCAGCAATACATTTACTCTCGGTACAAAATC |
| | (SEQ ID NO: 292) |
| ydbC_r | CGGGCCCAAGCTTCGAATTTTATTCTCGCGAAATACCATCCAACGTAGACAAC |
| | (SEQ ID NO: 293) |
| ydfG_f | TAAGGAGGAATAAACCATGATCGTTTTAGTAACTGGAGCAACGGCAG |
| | (SEQ ID NO: 294) |
| ydfG_r | CGGGCCCAAGCTTCGAATTTTACTGACGGTGGACATTCAGTCCG |
| | (SEQ ID NO: 295) |
| ydhF_f | TAAGGAGGAATAAACCATGGTTCAGCGTATTACTATTGCGCCG |
| | (SEQ ID NO: 296) |
| ydhF_r | CGGGCCCAAGCTTCGAATTTTACGGTACGTCGTACCCCAGTG |
| | (SEQ ID NO: 297) |
| ydjG_f | TAAGGAGGAATAAACCATGAAAAAGATACCTTTAGGCACAACGGATATTACGC |
| | (SEQ ID NO: 298) |
| ydjG_r | CGGGCCCAAGCTTCGAATTTTAACGCTCCAGGGCCTCTGC |
| | (SEQ ID NO: 299) |
| ydjJ_f | TAAGGAGGAATAAACCATGAAAAATTCAAAAGCAATATTGCAGGTGCCG |
| | (SEQ ID NO: 300) |
| ydjJ_r | CGGGCCCAAGCTTCGAATTAATCGCTAATTTTAATAACGCCTTTAATAATGTCGCGTTTG |
| | (SEQ ID NO: 301) |
| ydjL_f | TAAGGAGGAATAAACCATGAAAGCACTGGCTCGGTTTGGC |
| | (SEQ ID NO: 302) |
| ydjL_r | CGGGCCCAAGCTTCGAATTTTATTCATCAAAGTCGTAAGTCATGATCACTTTGATTGCG |
| | (SEQ ID NO: 303) |
| yeaE_f | TAAGGAGGAATAAACCATGCAACAAAAAATGATTCAATTTAGTGGCGATGTCTC |
| | (SEQ ID NO: 304) |
| yeaE_r | CGGGCCCAAGCTTCGAATTTCACACCATATCCAGCGCAGTTTTTCC |
| | (SEQ ID NO: 305) |
| ygcW_f | TAAGGAGGAATAAACCATGTCAATCGAATCTCTCAATGCGTTCTCAATG |
| | (SEQ ID NO: 306) |
| ygcW_r | CGGGCCCAAGCTTCGAATTTTAGCGCACTAAATAACCGCCATCAACC |
| | (SEQ ID NO: 307) |
| ygfF_f | TAAGGAGGAATAAACCATGGCTATAGCACTTGTGACTGGTGG |
| | (SEQ ID NO: 308) |
| ygfF_r | CGGGCCCAAGCTTCGAATTTTATTTCCCGCCCGCCAAATCG |
| | (SEQ ID NO: 309) |
| yggP_f | TAAGGAGGAATAAACCATGAAAACCAAAGTTGCTGCTATTTATGGCAAGC |
| | (SEQ ID NO: 310) |
| yggP_r | CGGGCCCAAGCTTCGAATTTCATTGCGCGGCCTCCC |
| | (SEQ ID NO: 311) |
| yghA_f | TAAGGAGGAATAAACCATGTCTCATTTAAAAGACCCGACCACGCAG |
| | (SEQ ID NO: 312) |
| yghA_r | CGGGCCCAAGCTTCGAATTTTAACCTAAATGCTCGCCGCCG |
| | (SEQ ID NO: 313) |
| yghZ_f | TAAGGAGGAATAAACCATGGTCTGGTTAGCGAATCCCGAAC |
| | (SEQ ID NO: 314) |
| yghZ_r | CGGGCCCAAGCTTCGAATTTCATTTATCGGAAGACGCCTGCCAC |
| | (SEQ ID NO: 315) |
| yhdH_f | TAAGGAGGAATAAACCATGCAGGCGTTACTTTTAGAACAGCAGG |
| | (SEQ ID NO: 316) |
| yhdH_r | CGGGCCCAAGCTTCGAATTTTAGTTAACCTTCACCAGCGTGCGAC |
| | (SEQ ID NO: 317) |
| yiaY_f | TAAGGAGGAATAAACCATGGCAGCTTCAACGTTCTTTATTCCTTCTG |
| | (SEQ ID NO:3 18) |
| yiaY_r | CGGGCCCAAGCTTCGAATTTTACATCGCTGCGCGATAAATCGCC |
| | (SEQ ID NO: 319) |
| yjgB_f | TAAGGAGGAATAAACCATGTCGATGATAAAAAGCTATGCCGCAAAAGAAG |
| | (SEQ ID NO: 320) |
| yjgB_r | CGGGCCCAAGCTTCGAATTTCAAAAATCGGCTTTCAACACCACGC |
| | (SEQ ID NO: 321) |
| yjgI_f | TAAGGAGGAATAAACCATGGGCGCTTTTACAGGTAAGACAGTTC |
| | (SEQ ID NO: 322) |
| yjgI_r | CGGGCCCAAGCTTCGAATTTTATGCGCCAAACGCGCCATC |
| | (SEQ ID NO: 323) |
| yjjN_f | TAAGGAGGAATAAACCATGTCTACGATGAATGTTTTAATTTGCCAGCAGC |
| | (SEQ ID NO: 324) |
| yjjN_r | CGGGCCCAAGCTTCGAATTTCAGAAAGTAATTACGCCTTTAATTAACTCACGATTGTTAA |
| | (SEQ ID NO: 325) |
| yncB_f | TAAGGAGGAATAAACCATGGGGCAACAAAAGCAGCGTAATC |
| | (SEQ ID NO: 326) |
| yncB_r | CGGGCCCAAGCTTCGAATTTTAATCATCACCCGCCACGCG |
| | (SEQ ID NO: 327) |
| yohF_f | TAAGGAGGAATAAACCATGGCACAGGTTGCGATTATTACCGC |
| | (SEQ ID NO: 328) |
| yohF_r | CGGGCCCAAGCTTCGAATTCTATTCTGGGTTGAACTGTGGATTCGCC |
| | (SEQ ID NO: 329) |
| tas_f | TAAGGAGGAATAAACCATGCAATATCACCGTATACCCCACAGTTCG |
| | (SEQ ID NO: 330) |
| tas_r | CGGGCCCAAGCTTCGAATTTTATGGTGCCGGATAAGTATAAACCTGATGCAC |
| | (SEQ ID NO: 331) |
| hcaB_f | TAAGGAGGAATAAACCATGAGCGATCTGCATAACGAGTCCATTTTTATTAC |
| | (SEQ ID NO: 332) |
| hcaB_r | CGGGCCCAAGCTTCGAATTTTAAAGATCCAGCCCAGCCGCTAC |
| | (SEQ ID NO: 333) |
| fabG_f | TAAGGAGGAATAAACCATGAATTTTGAAGGAAAAATCGCACTGGTAACCG |
| | (SEQ ID NO: 334) |
| fabG_r | CGGGCCCAAGCTTCGAATTTCAGACCATGTACATCCCGCCG |
| | (SEQ ID NO: 335) |
| yphC_f | TAAGGAGGAATAAACCATGAAAACGATGCTGGCAGCTTATTTACCAG |
| | (SEQ ID NO: 336) |
| yphC_r | CGGGCCCAAGCTTCGAATTTTAATCCGGGAAGTTAATCACAACTTTCCCGC |
| | (SEQ ID NO: 337) |
| yqhD_f | TAAGGAGGAATAAACCATGAACAACTTTAATCTGCACACCCCAACC |
| | (SEQ ID NO: 338) |
| yqhD_r | CGGGCCCAAGCTTCGAATTTTAGCGGGCGGCTTCGTATATACG |
| | (SEQ ID NO: 339) |

Each primer was designed to contain 15 bases of overlap with the expression vector, enabling restrictionless cloning using the InFusion cloning kit (Clontech). Excess nucleotides and primers were removed from the PCR products using the ZR-96 DCC kit (Zymo Research). After ligation of the PCR products into the linearized OP-80, the resulting DNA was transformed into NEB Turbo competent cells (New England Biolabs, Inc. Ipswich, Mass.), and plated onto LB agar medium supplemented with 100 μg/mL spectinomycin and 1% (w/v) glucose. Plasmid clones containing the appropriate inserts were identified using PCR, verified by sequencing and mini-prepped.

The sequence verified plasmids were transformed into the expression strain, E. coli (DE3) ΔyjgB yahK ΔydhD ΔdkgA, and plated onto LB agar medium supplemented with 100 μg/mL spectinomycin and 1% (w/v) glucose. Individual colonies were picked and grown overnight at 37° C. in LB liquid medium supplemented with 100 μg/mL spectinomycin and 1% (w/v) glucose. The culture was then diluted 1:1000 into fresh LB with 100 μg/mL spectinomycin and 1% (w/v) glucose and grown in a shaker for 5-6 hours at 37° C. The culture was then induced with 1 mM isopropyl β-D-1-thiogalactopyranoside (IPTG) and grown in a shaker for 18 hours at 18° C.

The cells were subsequently harvested by centrifugation for 10 minutes at 4,500 rpm. The supernatant was discarded and the cells were resuspended in 2.5 mL BugBuster lysis reagent (Novagen). The cell suspensions were placed on a vertical rotator for 45 minutes at 4° C. to lyse the cells. Cell debris were removed by centrifugation for 10 minutes at 4,500 rpm, and the clarified lysates were used for activity assays.

Each sample was evaluated in vitro to determine its ability to convert dodecanal or 11-cis-hexadecenal into dodecanol or 11-cis-hexadecenol, respectively, using the cell lysates as described above. The negative control consisted of a lysate prepared from cells transformed with an empty OP-80 expression vector.

Each reaction contained 5-40 μL of cell lysate, 20 μL 20 mM dodecanal or 11-cis-hexadecenal, 10 μL 20 mM NADH or NADPH, and sufficient dilution buffer (100 mM sodium phosphate, pH 7.0, 0.25% (v/v) Triton X-100) to bring the total volume to 400 μL. The mixture was incubated for 2 hours at 37° C. with constant shaking at 250 rpm.

To prepare samples for analysis, 40 μL 1 M HCl and 400 μL butyl acetate was added. Tetracosane (a C₂₋₄ alkane) was added as an internal standard (at 500 mg/L). The mixture was shaken for 15 minutes at 2,000 rpm, then centrifuged at 4,500 rpm for 10 minutes at 20° C.

A 50 μL sample of the organic phase was derivatized with BSTFA (N,O-bis(trimethylsilyl)trifluoroacetamide) and analyzed on a GC/FID equipped with a Trace UFC-1 column (Thermo Scientific). Samples were run using a split ratio of 1:300 and a program consisting of an initial temperature of 140° C. for 0.3 minute, a ramp up of 150° C./min to 300° C., then holding at a constant temperature of 300° C. for 0.05 minutes.

The percentage of aldehyde substrate that had been converted to alcohol was calculated for each sample, and a paired t-test was used to identify candidates that had converted the most aldehyde into alcohol as compared to the negative control, using a p value of less than or equal to about 0.05. The candidate that displayed statistically significant levels of fatty alcohol biosynthetic enzyme activity were identified and listed below in Table 14.

TABLE 14
Fatty alcohol biosynthetic polypeptides identified
using various substrates
Fatty Alcohol Biosynthetic Polypeptides Identified
Using Dodecanal and NADH
adhP
dkgA
ydjJ
ygdS (Tas)
yjgB
Fatty Alcohol Biosynthetic Polypeptides Identified
Using Dodecanal and NADPH
adhP
dkgA
dkgB
rspB
yahK
ybbO
ybdH
ybdR
ygfF
yhdH
yjgB
Fatty Alcohol Biosynthetic Polypeptides Identified
using 11-cis-hexadecenal & NADH
yjgB
Fatty Alcohol Biosynthetic Polypeptides Identified
Using 11-cis-hexadecenal and NADPH
adhP
aroB
rspB
yahK
ybbO
ybdH
ygfF
ybdR
ydbC
ydjG
yeaE
yhdH
yjgB
yncB

It is claimed:

1. A microorganism engineered to produce a fatty acid derivative, said microorganism comprising, polynucleotide sequences encoding:
(a) a thioesterase (EC 3.1.1.5);
(b) a fatty aldehyde biosynthetic polypeptide; and
(c) a fatty alcohol biosynthetic polypeptide,
wherein expression of said polypeptides is modified relative to the corresponding wild type polypeptide, and said microorganism produces an increased titer of the fatty acid derivative relative to a wild type microorganism.

2. The engineered microorganism according to claim 1, wherein said fatty aldehyde biosynthetic polypeptide has at least 90% sequence identity to the amino acid sequence presented as SEQ ID NO: 41, 43, 45, 47, 49, 51, 53, 55, 57, 59, 61, 63, 65, 69, 71, 73, 75, 77, 79, 81, 83, 85, 87, 89, 91, 93, 97, 99, 101, 103, 105, 107, 109, 111, 113, 115, 117, 119, 121, 123, 125, or 127.

3. The engineered microorganism according to claim 1, wherein said fatty aldehyde biosynthetic polypeptide comprises an amino acid sequence motif with a sequence presented as (1) SEQ ID NO:129, SEQ ID NO:130, SEQ ID NO:131, and SEQ ID NO:132; (2) SEQ ID NO:133; SEQ ID NO:134; SEQ ID NO:135; SEQ ID NO: 136; or (3) SEQ ID NO:129, SEQ ID NO:131, SEQ ID NO:132 or SEQ ID NO:133.

4. The engineered microorganism according to claim 1, wherein said fatty aldehyde biosynthetic polypeptide is encoded by a polynucleotide having at least 90% sequence identity to the nucleotide sequence presented as SEQ ID NO: 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, or 128.

5. A microorganism engineered to produce a fatty acid derivative, said microorganism comprising, polynucleotide sequences encoding:
(a) an acyl-ACP reductase polypeptide; and
(b) a fatty alcohol biosynthetic polypeptide,
wherein expression of said polypeptides is modified relative to the corresponding wild type polypeptide and said microorganism produces an increased titer of the fatty acid derivative relative to a wild type microorganism.

6. The engineered microorganism according to claim 5, wherein the acyl-ACP reductase polypeptide comprises an amino acid sequence having at least 90% sequence identity to a sequence presented as SEQ ID NO: 137, 139, 141, 143, 145, 147, 149, 151, or 153.

7. The engineered microorganism according to claim 5, wherein the acyl-ACP reductase polypeptide comprises an amino acid motif presented as SEQ ID NO:155, 156, 157, 158, 159, 160, 161, 162, 163, 164, or 165.

8. The engineered microorganism according to claim 5, wherein the acyl-ACP reductase polypeptide is encoded by a polynucleotide having at least 90% sequence identity to a sequence presented as SEQ ID NO: 138, 140, 142, 144, 146, 148, 150, 152, or 154.

9. A method of producing a fatty alcohol, the method comprising;
culturing an engineered microorganism according to claim 3, in the presence of a carbon source, under conditions wherein said fatty alcohol is produced at a titer of at least 300 mg/L.

10. A method of producing a fatty alcohol, the method comprising;
culturing an engineered microorganism according to claim 7, in the presence of a carbon source, under conditions wherein said fatty alcohol is produced at a titer of at least 300 mg/L.

11. The method according to claim 10, wherein the engineered microorganism is modified to express an attenuated level of an acyl-CoA synthase (EC 2.3.1.86).

12. The method according to claim 10, wherein the fatty alcohol biosynthetic polypeptide is a fatty aldehyde reductase or alcohol dehydrogenase (EC1.1.1.1) and the expression of polypeptide is increased relative to the corresponding wild type polypeptide.

13. The method according to claim 10, wherein the fatty alcohol biosynthetic polypeptide has at least 90% sequence identity to a polypeptide sequence selected from the group consisting of SEQ ID NO:1, 3, 5, 7, 9, 11, 13, 15, 17, 19, 21, 23, 25, 27, 29, 31, 33, 35, 37, and 39.

14. The method according to claim 10, wherein fatty alcohol biosynthetic polypeptide is encoded by a polynucleotide having at least 90% sequence identity to the nucleotide sequence presented as SEQ ID NO: 2, 4, 6, 8, 10, 12, 14, 16, 18, 20, 22, 24, 26, 28, 30, 32, 34, 36, 38, or 40.

15. The method of claim 10, wherein the fatty alcohol is spontaneously secreted from the microorganism, actively transported into the extracellular environment, or passively transported into the extracellular environment.

16. The method of claim 10, further comprising isolating the fatty alcohol from the culture.

17. The method of claim 10, wherein the fatty alcohol comprises a C₆-C₁₈ fatty alcohol.

18. The method of claim 10, wherein the fatty alcohol is a C₆, C₈, C₁₀, C₁₂, C₁₃, C₁₄, C₁₅, C₁₆, C₁₇, or C₁₈ fatty alcohol.

19. The method of claim 10, wherein the hydroxyl group is in the primary (C₁) position.

20. The method of claim 10, wherein the fatty alcohol is an unsaturated fatty alcohol.

21. The method of claim 20, wherein the unsaturated fatty alcohol is C10:1, C12:1, C14:1, C16:1, or C18:1.

22. The method of claim 20, wherein the fatty alcohol is unsaturated at the omega-7 position.

23. The method of claim 20, wherein the unsaturated fatty alcohol comprises a cis double bond.

24. The method of claim 10, wherein the fatty alcohol is a saturated fatty alcohol.

25. The method of claim 10, wherein the microorganism is selected from the group consisting of a yeast cell, a fungus cell, a filamentous fungi cell, and a bacterial cell.

26. An engineered microorganism according to claim 3, wherein the fatty alcohol biosynthetic polypeptide is a fatty aldehyde reductase or alcohol dehydrogenase (EC1.1.1.1) and the gene encoding said polypeptide is knocked-out.

27. An engineered microorganism according to claim 7, wherein the fatty alcohol biosynthetic polypeptide is a fatty aldehyde reductase or alcohol dehydrogenase (EC1.1.1.1) and the gene encoding said polypeptide is knocked-out.

28. The engineered microorganism according to claim 27, further comprising a polynucleotide sequence encoding a hydrocarbon biosynthetic polypeptide, having at least 90% sequence identity to the amino acid sequence of SEQ ID NO:166, 168, 170, 172, 174, 176, 178, 180, 182, 184, 186, 188, 190, 192, 194, 196, 198, or 200.

29. The engineered microorganism according to claim 27, wherein the hydrocarbon biosynthetic polypeptide has the amino acid sequence of SEQ ID NO:166, 168, 170, 172, 174, 176, 178, 180, 182, 184, 186, 188, 190, 192, 194, 196, 198, or 200 with one or more amino acid substitutions, additions, deletions, or insertions.

30. The engineered microorganism according to claim 27, wherein the hydrocarbon biosynthetic polypeptide has amino acid sequence having the amino acid motif sequences of (1) SEQ ID NO:202; (2) SEQ ID NO:203 or SEQ ID NO:204, or SEQ ID NO:205; (3) SEQ ID NO:206, and any one of SEQ ID NO:203, SEQ ID NO:204, SEQ ID NO:205; or (4) SEQ ID NO:207 or SEQ ID NO:208, or SEQ ID NO:209, or SEQ ID NO:210; wherein the hydrocarbon biosynthetic polypeptide has decarbonylase activity.

31. A method of producing a hydrocarbon, the method comprising;
culturing an engineered microorganism according to claim 30, in the presence of a carbon source, under conditions wherein said hydrocarbon is spontaneously secreted from the microorganism, actively transported into the extracellular environment, or passively transported into the extracellular environment.

32. The method according to claim 31, wherein the engineered microorganism is modified to express an attenuated level of an acyl-CoA synthase (EC 2.3.1.86).

33. The method of claim 31, wherein the hydrocarbon is secreted by the microorganism.

34. The method of claim 31, wherein the hydrocarbon is an alkane.

35. The method of claim 34, wherein the alkane comprises a C₁₃-C₂₁ alkane.

36. The method of claim 34, wherein the alkane is selected from the group consisting of tridecane, methyltridecane, nonadecane, methylnonadecane, heptadecane, methylheptadecane, pentadecane, and methylpentadecane.

37. The method of claim 31, further comprising culturing the microorganism in the presence of a saturated fatty acid derivative.

38. The method of claim 37, wherein the saturated fatty acid derivative is a C₁₄-C₂₂ saturated fatty acid derivative.

39. The method of claim 37, wherein the saturated fatty acid derivative is selected from the group consisting of 2-methylicosanal, icosanal, octadecanal, tetradecanal, 2-methyloctadecanal, stearaldehyde, palmitaldehyde, and their derivatives.

40. The method of claim 31, wherein the hydrocarbon is an alkene.

41. The method of claim 40, wherein the alkene comprises a C₁₃-C₂₂ alkene.

42. The method of claim 40, wherein the alkene is selected form the group consisting of pentadecene, heptadecene, methylpentadecene, and methylheptadecene.

43. The method of claim 31, further comprising culturing the microorganism in the presence of an unsaturated fatty acid derivative.

44. The method of claim 43, wherein the unsaturated fatty acid derivative is a C₁₄-C₂₂ unsaturated fatty acid derivative.

45. The method of claim 43, wherein the unsaturated fatty acid derivative is selected from the group consisting of octadecenal, hexadecenal, methylhexadecenal, and methyloctadecenal.

46. The method of claim 31, wherein the microorganism is selected from the group consisting of a yeast cell, a fungus cell, a filamentous fungi cell, and a bacterial cell

47. A hydrocarbon produced by the method of claim 31.

48. A biofuel comprising the hydrocarbon of claim 47.

49. The biofuel of claim 48, wherein the biofuel is a diesel, gasoline, or jet fuel.

50. The biofuel of claim 49, wherein the hydrocarbon has δ¹³C of −15.4 or greater.

51. The biofuel of claim 50, wherein the hydrocarbon has a f_{M}¹⁴C of at least 1.003.
